SEQUENCE LISTING (155 sequences)

SEQ ID NO: 1 - protein, 111 aa, Homo sapiens
MSLLPRRAPPVSMRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 2 - DNA, 336 bp, Homo sapiens
  source 1..336: mol_type = other DNA
atgtccctgctcccacgccgcgcccctccggtcagcatgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatag

SEQ ID NO: 3 - protein, 242 aa, Homo sapiens
MPRGFTWLRYLGIFLGVALGNEPLEMWPLTQNEECTVTGFLRDKLQYRSRLQYMKHYFPINYKISVPYEGVFRIANVTRLQRAQVSERELRYLWVLVSLSATESVQDVLLEGHPSWKYLQEVETLLLNVQQGLTDVEVSPKVESVLSLLNAPGPNLKLVRPKALLDNCFRVMELLYCSCCKQSSVLNWQDCEVPSPQSCSPEPSLQYAATQLYPPPPWSPSSPPHSTGSVRPVRAQGEGLLP

SEQ ID NO: 4 - DNA, 729 bp, Homo sapiens
  source 1..729: mol_type = other DNA
atgccccggggcttcacctggctgcgctatcttgggatcttccttggcgtggccttggggaatgagcctttggagatgtggcccttgacgcagaatgaggagtgcactgtcacgggttttctgcgggacaagctgcagtacaggagccgacttcagtacatgaaacactacttccccatcaactacaagatcagtgtgccttacgagggggtgttcagaatcgccaacgtcaccaggctgcagagggcccaggtgagcgagcgggagctgcggtatctgtgggtcttggtgagcctcagtgccactgagtcggtgcaggacgtgctgctcgagggccacccatcctggaagtacctgcaggaggtggagacgctgctgctgaatgtccagcagggcctcacggatgtggaggtcagccccaaggtggaatccgtgttgtccctcttgaatgccccagggccaaacctgaagctggtgcggcccaaagccctgctggacaactgcttccgggtcatggagctgctgtactgctcctgctgtaaacaaagctccgtcctaaactggcaggactgtgaggtgccaagtcctcagtcttgcagcccagagccctcattgcagtatgcggccacccagctgtaccctccgcccccgtggtcccccagctccccgcctcactccacgggctcggtgaggccggtcagggcacagggcgagggcctcttgccctga

SEQ ID NO: 5 - protein, 176 aa, Homo sapiens
MFHVSFRYIFGLPPLILVLLPVASSDCDIEGKDGKQYESVLMVSIDQLLDSMKEIGSNCNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 6 - DNA, 534 bp, Homo sapiens
  source 1..534: mol_type = other DNA
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 7 - protein, 94 aa, Homo sapiens
MAPLKMLALVTLLLGASLQHIHAARGTNVGRECCLEYFKGAIPLRKLKTWYQTSEDCSRDAIVFVTVQGRAICSDPNNKRVKNAVKYLQSLERS

SEQ ID NO: 8 - DNA, 285 bp, Homo sapiens
  source 1..285: mol_type = other DNA
atggccccactgaagatgctggccctggtcaccctcctcctgggggcttctctgcagcacatccacgcagctcgagggaccaatgtgggccgggagtgctgcctggagtacttcaagggagccattccccttagaaagctgaagacgtggtaccagacatctgaggactgctccagggatgccatcgtttttgtaactgtgcagggcagggccatctgttcggaccccaacaacaagagagtgaagaatgcagttaaatacctgcaaagccttgagaggtcttga

SEQ ID NO: 9 - DNA, 53 bp, synthetic construct
  source 1..53: mol_type = other DNA
cagccctgggcatccaccgacagacagccctgggcatccaccgacggcgccgg

SEQ ID NO: 10 - DNA, 53 bp, synthetic construct
  source 1..53: mol_type = other DNA
gcacggccacagacagccctcagcgcacggccacagacagccctgggcatggg

SEQ ID NO: 11 - DNA, 53 bp, synthetic construct
  source 1..53: mol_type = other DNA
agctgggcgcggattcgccgccggagctgggcgcggattcgccgaggcacagg

SEQ ID NO: 12 - DNA, 53 bp, synthetic construct
  source 1..53: mol_type = other DNA
gcggattcgccgaggcaccggggcgcggattcgccgaggcaccgaggcacagg

SEQ ID NO: 13 - DNA, 20 bp, synthetic construct
  source 1..20: mol_type = other DNA
gaggttcatgagccctcaaa

SEQ ID NO: 14 - DNA, 20 bp, synthetic construct
  source 1..20: mol_type = other DNA
tcttggtgattaactccatc

SEQ ID NO: 15 - DNA, 20 bp, synthetic construct
  source 1..20: mol_type = other DNA
ccataggcgtgggacctcgt

SEQ ID NO: 16 - DNA, 20 bp, synthetic construct
  source 1..20: mol_type = other DNA
atcccacagcttgtggcggg

SEQ ID NO: 17 - DNA, 20 bp, synthetic construct
  source 1..20: mol_type = other DNA
tctcgccatcggtcgctctc

SEQ ID NO: 18 - DNA, 21 bp, synthetic construct
  source 1..21: mol_type = other DNA
ggcctctcagaagcatcacta

SEQ ID NO: 19 - DNA, 18 bp, synthetic construct
  source 1..18: mol_type = other DNA
gccaagccctgagcataa

SEQ ID NO: 20 - DNA, 16 bp, synthetic construct
  source 1..16: mol_type = other DNA
cccaacgccccgaact

SEQ ID NO: 21 - DNA, 21 bp, synthetic construct
  source 1..21: mol_type = other DNA
gcagaagagctgctacgtgaa

SEQ ID NO: 22 - DNA, 16 bp, synthetic construct
  source 1..16: mol_type = other DNA
gccgcaccggaatcct

SEQ ID NO: 23 - DNA, 22 bp, synthetic construct
  source 1..22: mol_type = other DNA
ttgagcagaagagctgctacgt

SEQ ID NO: 24 - DNA, 20 bp, synthetic construct
  source 1..20: mol_type = other DNA
gtccaatcctggtgatgtcc

SEQ ID NO: 25 - DNA, 19 bp, synthetic construct
  source 1..19: mol_type = other DNA
gttttcggccctgagatgt

SEQ ID NO: 26 - DNA, 20 bp, synthetic construct
  source 1..20: mol_type = other DNA
agtgtccagggatgaggaag

SEQ ID NO: 27 - DNA, 20 bp, synthetic construct
  source 1..20: mol_type = other DNA
ggggaaccgtctgaaacatt

SEQ ID NO: 28 - DNA, 20 bp, synthetic construct
  source 1..20: mol_type = other DNA
gaacagatttgtccttcccg

SEQ ID NO: 29 - DNA, 20 bp, synthetic construct
  source 1..20: mol_type = other DNA
ccatgattcgatgctcacat

SEQ ID NO: 30 - DNA, 19 bp, synthetic construct
  source 1..19: mol_type = other DNA
atggcacccgaatggtcag

SEQ ID NO: 31 - DNA, 23 bp, synthetic construct
  source 1..23: mol_type = other DNA
ctccagtgtatagcctctctctg

SEQ ID NO: 32 - DNA, 18 bp, synthetic construct
  source 1..18: mol_type = other DNA
agcttggcactgctcctg

SEQ ID NO: 33 - DNA, 20 bp, synthetic construct
  source 1..20: mol_type = other DNA
gtttaccacagaacatggcg

SEQ ID NO: 34 - DNA, 20 bp, synthetic construct
  source 1..20: mol_type = other DNA
attgggatcatcttgctggt

SEQ ID NO: 35 - DNA, 19 bp, synthetic construct
  source 1..19: mol_type = other DNA
cctggtgttcacagttgcc

SEQ ID NO: 36 - DNA, 23 bp, synthetic construct
  source 1..23: mol_type = other DNA
cctgggaagctgttatcttcaag

SEQ ID NO: 37 - DNA, 20 bp, synthetic construct
  source 1..20: mol_type = other DNA
cctcctcgacccacttctga

SEQ ID NO: 38 - DNA, 20 bp, synthetic construct
  source 1..20: mol_type = other DNA
gtgccatcagagcagtctgt

SEQ ID NO: 39 - DNA, 20 bp, synthetic construct
  source 1..20: mol_type = other DNA
acccaaaccgaagtcatagc

SEQ ID NO: 40 - DNA, 22 bp, synthetic construct
  source 1..22: mol_type = other DNA
gaagatggttatcgtcaccacc

SEQ ID NO: 41 - DNA, 21 bp, synthetic construct
  source 1..21: mol_type = other DNA
cgttccaggcattgtaccact

SEQ ID NO: 42 - DNA, 20 bp, synthetic construct
  source 1..20: mol_type = other DNA
ccatgggtgaaggctactgt

SEQ ID NO: 43 - DNA, 21 bp, synthetic construct
  source 1..21: mol_type = other DNA
agaggcttttcatgctcaaca

SEQ ID NO: 44 - DNA, 20 bp, synthetic construct
  source 1..20: mol_type = other DNA
gggagaagtttgtgcatggt

SEQ ID NO: 45 - DNA, 20 bp, synthetic construct
  source 1..20: mol_type = other DNA
ctgggggtctccgtgataat

SEQ ID NO: 46 - DNA, 21 bp, synthetic construct
  source 1..21: mol_type = other DNA
gaccccgagacggagtatttc

SEQ ID NO: 47 - DNA, 20 bp, synthetic construct
  source 1..20: mol_type = other DNA
tgcactggtcaggcaagttc

SEQ ID NO: 48 - DNA, 18 bp, synthetic construct
  source 1..18: mol_type = other DNA
gggtgccaaacacctcag

SEQ ID NO: 49 - DNA, 18 bp, synthetic construct
  source 1..18: mol_type = other DNA
aggttgtaccggccatgc

SEQ ID NO: 50 - DNA, 20 bp, synthetic construct
  source 1..20: mol_type = other DNA
taagaagaagcagtgccgcc

SEQ ID NO: 51 - DNA, 20 bp, synthetic construct
  source 1..20: mol_type = other DNA
tttccccttggtgtcgtagc

SEQ ID NO: 52 - DNA, 20 bp, synthetic construct
  source 1..20: mol_type = other DNA
acttccagtacggtctcccg

SEQ ID NO: 53 - DNA, 19 bp, synthetic construct
  source 1..19: mol_type = other DNA
gcagcgcatctcaggttgt

SEQ ID NO: 54 - DNA, 20 bp, synthetic construct
  source 1..20: mol_type = other DNA
tgcaggagacaagttctgga

SEQ ID NO: 55 - DNA, 20 bp, synthetic construct
  source 1..20: mol_type = other DNA
gacggcatccaggttatcag

SEQ ID NO: 56 - DNA, 19 bp, synthetic construct
  source 1..19: mol_type = other DNA
cctggtcccatgccaaacc

SEQ ID NO: 57 - DNA, 23 bp, synthetic construct
  source 1..23: mol_type = other DNA
cccttgaaagcataagtcttccc

SEQ ID NO: 58 - DNA, 20 bp, synthetic construct
  source 1..20: mol_type = other DNA
cagcctcttctcattcctgc

SEQ ID NO: 59 - DNA, 20 bp, synthetic construct
  source 1..20: mol_type = other DNA
agggtctgggccatagaact

SEQ ID NO: 60 - DNA, 22 bp, synthetic construct
  source 1..22: mol_type = other DNA
atgcagtacattggagaaggtg

SEQ ID NO: 61 - DNA, 20 bp, synthetic construct
  source 1..20: mol_type = other DNA
cgtctctcggctgcctattt

SEQ ID NO: 62 - DNA, 18 bp, synthetic construct
  source 1..18: mol_type = other DNA
gtgcgacctccaacctga

SEQ ID NO: 63 - DNA, 21 bp, synthetic construct
  source 1..21: mol_type = other DNA
ggctcatctcacctcacatct

SEQ ID NO: 64 - DNA, 18 bp, synthetic construct
  source 1..18: mol_type = other DNA
ctcttcgccagccttctg

SEQ ID NO: 65 - DNA, 19 bp, synthetic construct
  source 1..19: mol_type = other DNA
ttgctccagaccatctgct

SEQ ID NO: 66 - DNA, 20 bp, synthetic construct
  source 1..20: mol_type = other DNA
ggctggaaggcccatatctg

SEQ ID NO: 67 - DNA, 22 bp, synthetic construct
  source 1..22: mol_type = other DNA
cagcagggacattgaatcttct

SEQ ID NO: 68 - DNA, 27 bp, synthetic construct
  source 1..27: mol_type = other DNA
tcaaccaaactatatatcaggatgtgg

SEQ ID NO: 69 - DNA, 27 bp, synthetic construct
  source 1..27: mol_type = other DNA
cgagtaggcatacatgtcaaattttac

SEQ ID NO: 70 - DNA, 25 bp, synthetic construct
  source 1..25: mol_type = other DNA
gctaccaaactggatataatcagga

SEQ ID NO: 71 - DNA, 24 bp, synthetic construct
  source 1..24: mol_type = other DNA
ccaggtagctatggtactccagaa

SEQ ID NO: 72 - DNA, 21 bp, synthetic construct
  source 1..21: mol_type = other DNA
ctcccagccaggtgtcatttt

SEQ ID NO: 73 - DNA, 21 bp, synthetic construct
  source 1..21: mol_type = other DNA
cttggacccaggtctctttgg

SEQ ID NO: 74 - DNA, 22 bp, synthetic construct
  source 1..22: mol_type = other DNA
actgttgcctctcgtacataca

SEQ ID NO: 75 - DNA, 21 bp, synthetic construct
  source 1..21: mol_type = other DNA
gaggaggttcacagccctttt

SEQ ID NO: 76 - DNA, 22 bp, synthetic construct
  source 1..22: mol_type = other DNA
catccagagcttgagtgtgacg

SEQ ID NO: 77 - DNA, 22 bp, synthetic construct
  source 1..22: mol_type = other DNA
ggcttcagggtcaaggcaaact

SEQ ID NO: 78 - DNA, 22 bp, synthetic construct
  source 1..22: mol_type = other DNA
tgagaccatccagagcttgacg

SEQ ID NO: 79 - DNA, 22 bp, synthetic construct
  source 1..22: mol_type = other DNA
ccttgggggttgaggcaaactt

SEQ ID NO: 80 - DNA, 21 bp, synthetic construct
  source 1..21: mol_type = other DNA
tgccctacggtggaagtcata

SEQ ID NO: 81 - DNA, 22 bp, synthetic construct
  source 1..22: mol_type = other DNA
tgcattccgcttagctttcttt

SEQ ID NO: 82 - DNA, 22 bp, synthetic construct
  source 1..22: mol_type = other DNA
gactggcatagtcggcaatgga

SEQ ID NO: 83 - DNA, 22 bp, synthetic construct
  source 1..22: mol_type = other DNA
caaagaggaggtcagccactga

SEQ ID NO: 84 - DNA, 22 bp, synthetic construct
  source 1..22: mol_type = other DNA
aaaccagaccctggaactgcac

SEQ ID NO: 85 - DNA, 22 bp, synthetic construct
  source 1..22: mol_type = other DNA
gcctggcatttcagagtctgct

SEQ ID NO: 86 - DNA, 20 bp, synthetic construct
  source 1..20: mol_type = other DNA
gagtctccatgcagctacgg

SEQ ID NO: 87 - DNA, 19 bp, synthetic construct
  source 1..19: mol_type = other DNA
cgcttctcttcaatccggt

SEQ ID NO: 88 - DNA, 20 bp, synthetic construct
  source 1..20: mol_type = other DNA
tccgtcgtaacctgttgagt

SEQ ID NO: 89 - DNA, 20 bp, synthetic construct
  source 1..20: mol_type = other DNA
tggccagtgactctgtgtct

SEQ ID NO: 90 - DNA, 19 bp, synthetic construct
  source 1..19: mol_type = other DNA
ggctgtttgactccccttc

SEQ ID NO: 91 - DNA, 20 bp, synthetic construct
  source 1..20: mol_type = other DNA
gggcacactcttcctcctct

SEQ ID NO: 92 - DNA, 20 bp, synthetic construct
  source 1..20: mol_type = other DNA
ggccagctgagcttaaagaa

SEQ ID NO: 93 - DNA, 20 bp, synthetic construct
  source 1..20: mol_type = other DNA
gtggtgattcaaaactgcca

SEQ ID NO: 94 - DNA, 25 bp, synthetic construct
  source 1..25: mol_type = other DNA
gccagcttcggagagttctgggatt

SEQ ID NO: 95 - DNA, 25 bp, synthetic construct
  source 1..25: mol_type = other DNA
cgggcacgaagtgcaatggtcttta

SEQ ID NO: 96 - DNA, 17 bp, synthetic construct
  source 1..17: mol_type = other DNA
gtgagagtggcggggtc

SEQ ID NO: 97 - DNA, 20 bp, synthetic construct
  source 1..20: mol_type = other DNA
cccaacgcaccgaatagtta

SEQ ID NO: 98 - DNA, 19 bp, synthetic construct
  source 1..19: mol_type = other DNA
gccattagcgcatcacagt

SEQ ID NO: 99 - DNA, 19 bp, synthetic construct
  source 1..19: mol_type = other DNA
accgaggcactcagaggag

SEQ ID NO: 100 - DNA, 23 bp, synthetic construct
  source 1..23: mol_type = other DNA
ttggatcacagcgatacaaactt

SEQ ID NO: 101 - DNA, 21 bp, synthetic construct
  source 1..21: mol_type = other DNA
agcgcacgccaataaagacat

SEQ ID NO: 102 - DNA, 22 bp, synthetic construct
  source 1..22: mol_type = other DNA
gctcccaaatataggtggcact

SEQ ID NO: 103 - DNA, 20 bp, synthetic construct
  source 1..20: mol_type = other DNA
ccaacttgatcctcaggggc

SEQ ID NO: 104 - DNA, 21 bp, synthetic construct
  source 1..21: mol_type = other DNA
agggaccaaaacctgcattga

SEQ ID NO: 105 - DNA, 20 bp, synthetic construct
  source 1..20: mol_type = other DNA
actcaccaactaagcagcgg

SEQ ID NO: 106 - DNA, 20 bp, synthetic construct
  source 1..20: mol_type = other DNA
aagccaaagtacccgcactg

SEQ ID NO: 107 - DNA, 20 bp, synthetic construct
  source 1..20: mol_type = other DNA
gacctcggtacctggacacg

SEQ ID NO: 108 - DNA, 20 bp, synthetic construct
  source 1..20: mol_type = other DNA
aggagaatggacctgcaagc

SEQ ID NO: 109 - DNA, 20 bp, synthetic construct
  source 1..20: mol_type = other DNA
gaagtgcaagtgatgcgtcc

SEQ ID NO: 110 - DNA, 21 bp, synthetic construct
  source 1..21: mol_type = other DNA
ctcgtgcagcctataaaccct

SEQ ID NO: 111 - DNA, 21 bp, synthetic construct
  source 1..21: mol_type = other DNA
tcctcgtgttcactttcaccg

SEQ ID NO: 112 - DNA, 20 bp, synthetic construct
  source 1..20: mol_type = other DNA
gtggatccctcaaccaagaa

SEQ ID NO: 113 - DNA, 20 bp, synthetic construct
  source 1..20: mol_type = other DNA
taggttcccagagtgcccta

SEQ ID NO: 114 - DNA, 19 bp, synthetic construct
  source 1..19: mol_type = other DNA
cggcggaggaaaactgtct

SEQ ID NO: 115 - DNA, 21 bp, synthetic construct
  source 1..21: mol_type = other DNA
tcggctgggtaagaaatctga

SEQ ID NO: 116 - DNA, 21 bp, synthetic construct
  source 1..21: mol_type = other DNA
tgaggaggagcatgaatggag

SEQ ID NO: 117 - DNA, 20 bp, synthetic construct
  source 1..20: mol_type = other DNA
ttctgctggcaaagtgggac

SEQ ID NO: 118 - DNA, 21 bp, synthetic construct
  source 1..21: mol_type = other DNA
gtgcaatggtgtgcctgacta

SEQ ID NO: 119 - DNA, 24 bp, synthetic construct
  source 1..24: mol_type = other DNA
aagtctgggactactcagtctttc

SEQ ID NO: 120 - DNA, 20 bp, synthetic construct
  source 1..20: mol_type = other DNA
gctctgggaagccagaagtt

SEQ ID NO: 121 - DNA, 20 bp, synthetic construct
  source 1..20: mol_type = other DNA
gacctcctgtgcctttaccc

SEQ ID NO: 122 - DNA, 20 bp, synthetic construct
  source 1..20: mol_type = other DNA
ttttgacatcctgtgctggc

SEQ ID NO: 123 - DNA, 20 bp, synthetic construct
  source 1..20: mol_type = other DNA
ccagtccctgcatccaagtc

SEQ ID NO: 124 - DNA, 22 bp, synthetic construct
  source 1..22: mol_type = other DNA
ggtgatctcagatagctcaggc

SEQ ID NO: 125 - DNA, 20 bp, synthetic construct
  source 1..20: mol_type = other DNA
aatcacggtacttgggaggc

SEQ ID NO: 126 - DNA, 22 bp, synthetic construct
  source 1..22: mol_type = other DNA
tgcttagctgagatggtggtct

SEQ ID NO: 127 - DNA, 23 bp, synthetic construct
  source 1..23: mol_type = other DNA
cagtcttgttacacgatccagcc

SEQ ID NO: 128 - DNA, 22 bp, synthetic construct
  source 1..22: mol_type = other DNA
actgctatgtctgtcaggaaca

SEQ ID NO: 129 - DNA, 24 bp, synthetic construct
  source 1..24: mol_type = other DNA
ccaagatccagacagtccactaaa

SEQ ID NO: 130 - DNA, 21 bp, synthetic construct
  source 1..21: mol_type = other DNA
gaccagcctgacagatttcta

SEQ ID NO: 131 - DNA, 21 bp, synthetic construct
  source 1..21: mol_type = other DNA
ctatcactccaagcgcagatt

SEQ ID NO: 132 - DNA, 21 bp, synthetic construct
  source 1..21: mol_type = other DNA
cgctatgaagaagtttatctt

SEQ ID NO: 133 - DNA, 21 bp, synthetic construct
  source 1..21: mol_type = other DNA
ccgtggattctgaacgtactt

SEQ ID NO: 134 - DNA, 21 bp, synthetic construct
  source 1..21: mol_type = other DNA
ccgaagaacttcactctctta

SEQ ID NO: 135 - DNA, 21 bp, synthetic construct
  source 1..21: mol_type = other DNA
acgcctttgggaagtattatt

SEQ ID NO: 136 - DNA, 21 bp, synthetic construct
  source 1..21: mol_type = other DNA
ccacagtccatccactcattt

SEQ ID NO: 137 - DNA, 21 bp, synthetic construct
  source 1..21: mol_type = other DNA
cgtctcaactgttccttggtt

SEQ ID NO: 138 - DNA, 21 bp, synthetic construct
  source 1..21: mol_type = other DNA
cccatcaaagactcgctgtaa

SEQ ID NO: 139 - DNA, 21 bp, synthetic construct
  source 1..21: mol_type = other DNA
cggttagatgagcttgagaaa

SEQ ID NO: 140 - DNA, 21 bp, synthetic construct
  source 1..21: mol_type = other DNA
ctgtccaatgtcaaccggaat

SEQ ID NO: 141 - DNA, 21 bp, synthetic construct
  source 1..21: mol_type = other DNA
acgtgaacaccaagtgcatta

SEQ ID NO: 142 - DNA, 21 bp, synthetic construct
  source 1..21: mol_type = other DNA
gctggaaatgaagccaaagta

SEQ ID NO: 143 - DNA, 21 bp, synthetic construct
  source 1..21: mol_type = other DNA
ctgcgaggagaagatggttat

SEQ ID NO: 144 - DNA, 21 bp, synthetic construct
  source 1..21: mol_type = other DNA
agaagacattgaggtgtattt

SEQ ID NO: 145 - DNA, 21 bp, synthetic construct
  source 1..21: mol_type = other DNA
ggagtaccctgaagctataac

SEQ ID NO: 146 - DNA, 20 bp, synthetic construct
  source 1..20: mol_type = other DNA
tcactgtcttgtacccttgt

SEQ ID NO: 147 - DNA, 20 bp, synthetic construct
  source 1..20: mol_type = other DNA
gaggttcctaagagtgctgg

SEQ ID NO: 148 - DNA, 21 bp, synthetic construct
  source 1..21: mol_type = other DNA
cagagatcgtgtattgagatt

SEQ ID NO: 149 - DNA, 21 bp, synthetic construct
  source 1..21: mol_type = other DNA
aagaacgattatccattcaaa

SEQ ID NO: 150 - DNA, 22 bp, synthetic construct
  source 1..22: mol_type = other DNA
gtatcccaaagtccagggcact

SEQ ID NO: 151 - DNA, 22 bp, synthetic construct
  source 1..22: mol_type = other DNA
tgccaaggggaaggacatcatt

SEQ ID NO: 152 - DNA, 16 bp, synthetic construct
  source 1..16: mol_type = other DNA
ggcgtgtgcatcaatg

SEQ ID NO: 153 - DNA, 24 bp, synthetic construct
  source 1..24: mol_type = other DNA
gaaaggaaagtcagggacattggg

SEQ ID NO: 154 - DNA, 17 bp, synthetic construct
  source 1..17: mol_type = other DNA
gctgccttgtctctctc

SEQ ID NO: 155 - DNA, 20 bp, synthetic construct
  source 1..20: mol_type = other DNA
atggcctagggattccagca

(19) United States
(12) Patent Application Publication
(10) Pub. No.: US 2024/0382563 A1
(43) Pub. Date: Nov. 21, 2024

(54) PROMOTING IMMUNE SURVEILLANCE AGAINST CANCER CELLS

(71) Applicant: Mayo Foundation for Medical Education and Research, Rochester, MN (US)

(72) Inventors: Jan M.A. van Deursen, Rochester, MN (US); Hu Li, Rochester, MN (US); Ines Sturmlechner, Rochester, MN (US); Nathaniel Eames David, San Francsico, CA (US)

(21) Appl. No.: 18/580,920

(22) PCT Filed: Jul. 21, 2022
(86) PCT No.: PCT/US2022/073986

§ 371 Date: Jan. 19, 2024

Related U.S. Application Data
(60) Provisional application No. 63/224,177, filed on Jul. 21, 2021.

(51) Int. Cl. A61K 38/19; A61K 38/20; A61K 39/395

(52) U.S. Cl. CPC A61K 38/195; A61K 38/20; A61K 39/39558

(57) ABSTRACT

This document provides methods and materials involved in promoting immune surveillance against cancer cells. For example, methods and materials administering one or more chemokine (C-X-C motif) ligand 14 (CXCL 14) polypeptides (and/or nucleic acids designed to encode a CXCL 14 polypeptide) to cancer cells within a mammal (e.g., a human) having cancer to promote immune surveillance against the cancer cells are provided.

### CROSS-REFERENCE TO RELATED APPLICATIONS

This application claims the benefit of U.S. Patent Application Ser. No. 63/224,177, filed on Jul. 21, 2021. The disclosure of the prior application is considered part of (and is incorporated by reference in) the disclosure of this application.

### SEQUENCE LISTING

This application contains a Sequence Listing that has been submitted electronically as an XML file named “07039-2067WO1.XML.” The XML file, created on Jul. 7, 2022, is 137,000 bytes in size. The material in the XML file is hereby incorporated by reference in its entirety.

### TECHNICAL FIELD

This document relates to methods and materials involved in promoting immune surveillance against cancer cells. For example, one or more chemokine (C-X-C motif) ligand 14 (CXCL14) polypeptides (and/or nucleic acids designed to encode a CXCL14 polypeptide) can be administered to cancer cells within a mammal (e.g., a human) having cancer to promote immune surveillance against the cancer cells.

### BACKGROUND INFORMATION

Cellular senescence is a tumor-protective mechanism in which cycling-competent cells undergo permanent cell-cycle arrest in response to persistent or irreparable cellular stresses or damage (Kang et al., Nature, 479:547-551 (2011); Eggert et al., Cancer Cell, 30:533-547 (2016); Kuilman et al., Cell, 133:1019-1031 (2008); and Tasdemir et al., Cancer Discov., 6:612-629 (2016)).

### SUMMARY

This document provides methods and materials for promoting immune surveillance against cancer cells. For example, one or more (e.g., one, two, three, four, or more) agents having the ability to increase a level of a CXCL14 polypeptide can be administered to a mammal (e.g., a human) having cancer to promote immune surveillance against the cancer cells. In some cases, one or more CXCL14 polypeptides (and/or one or more nucleic acids designed to encode a CXCL14 polypeptide) can be delivered to a mammal (e.g., a human) having cancer to promote immune surveillance against cancer cells. In some cases, one or more agents that can modulate a signaling pathway in which a P21 polypeptide can hypophosphorylate a retinoblastoma (RB) polypeptide to induce expression of P21-activated secretory phenotype (PASP) polypeptides (a PASP pathway) to increase expression of a CXCL14 polypeptide can be administered to a mammal (e.g., a human) having cancer to promote immune surveillance against cancer cells. In some cases, the methods and materials provided herein can be used to treat a mammal (e.g., a human) having cancer.

Immune cells identify and destroy damaged cells to prevent them from causing cancer or other pathologies, but how remains poorly understood. As demonstrated herein, stressed cells such as cancer cells activate a PASP pathway in which a P21 polypeptide can hypophosphorylate a RB polypeptide to induce expression of PASP polypeptides including a CXCL14 polypeptide (see, e.g., FIG. 25). Also as demonstrated herein, a CXCL14 polypeptide can recruit macrophages to cells having an elevated level of P21 polypeptides and can place such cells under immune surveillance in which the macrophages will disengage if cells undergo cellular repair mechanisms, but will polarize towards an M1 phenotype and mount and recruit a cytotoxic T cell response to destroy the cells if they fail to undergo cellular repair mechanisms or otherwise adapt to the stress they are experiencing.

Having the ability to promote immune surveillance as described herein (e.g., by increasing a level of a CXCL14 polypeptide in one or more cancer cells within a mammal (e.g., a human) having cancer) can be an effective mechanism by which to treat the mammal. In general, one aspect of this document features methods for inducing immune surveillance against a cancer cell within a mammal having cancer. The methods can include, or consist essentially of, administering to a mammal having cancer a composition including a CXCL14 polypeptide and a targeting moiety, where the targeting moiety targets the composition to a cancer cell within the mammal. The mammal can be a human. The cancer can be liver cancer, colorectal cancer, breast cancer, head and neck cancer, or cervical cancer. The targeting moiety can include an antibody or a single-chain variable fragment (scFv). The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for inducing immune surveillance against a cancer cell within a mammal having cancer. The methods can include, or consist essentially of, administering to a mammal having cancer a composition including a targeting moiety and nucleic acid encoding a CXCL14 polypeptide, where the targeting moiety targets the composition to a cancer cell within the mammal, and where the cancer cell expresses the CXCL14 polypeptide, thereby inducing immune surveillance against the cancer cell. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for inducing immune surveillance against a cancer cell within a mammal having cancer. The methods can include, or consist essentially of, administering to a mammal having cancer a composition including: (a) nucleic acid encoding a fusion polypeptide comprising a deactivated Cas (dCas) polypeptide and a transcriptional activator polypeptide; (b) nucleic acid encoding a helper activator polypeptide; (c) nucleic acid encoding a nucleic acid molecule comprising (i) a nucleic acid sequence that is complementary to a target sequence that encodes at least a portion of a CXCL14 polypeptide, and (ii) a nucleic acid sequence that can bind the helper activator polypeptide; and (d) a targeting moiety, where the targeting moiety targets the composition to a cancer cell within the mammal, and where the cancer cell increases expression of an endogenous CXCL14 polypeptide. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for treating cancer in a mammal. The methods can include, or consist essentially of, administering to a mammal having cancer a composition including a CXCL14 polypeptide and a targeting moiety, where the targeting moiety targets the composition to a cancer cell within the mammal. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for treating cancer in a mammal. The methods can include, or consist essentially of, administering to a mammal having cancer a composition including a targeting moiety and nucleic acid encoding a CXCL14 polypeptide, where the targeting moiety targets the composition to a cancer cell within the mammal, and where the cancer cell expresses the CXCL14 polypeptide. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for treating cancer in a mammal. The methods can include, or consist essentially of, administering to a mammal having cancer a composition including: (a) nucleic acid encoding a fusion polypeptide comprising a dCas polypeptide and a transcriptional activator polypeptide; (b) nucleic acid encoding a helper activator polypeptide; (c) nucleic acid encoding a nucleic acid molecule comprising (i) a nucleic acid sequence that is complementary to a target sequence within a Cxcl14 gene, and (ii) a nucleic acid sequence that can bind the helper activator polypeptide; and (d) a targeting moiety, where the targeting moiety targets the composition to a cancer cell within the mammal, and where the cancer cell increases expression of an endogenous CXCL14 polypeptide. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for inducing immune surveillance against a cancer cell within a mammal having cancer. The methods can include, or consist essentially of, administering to a mammal having cancer a composition including a p21 polypeptide and a targeting moiety, where the targeting moiety targets the composition to a cancer cell within the mammal. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for inducing immune surveillance against a cancer cell within a mammal having cancer. The methods can include, or consist essentially of, administering to a mammal a composition including a targeting moiety and nucleic acid encoding a p21 polypeptide, where the targeting moiety targets the composition to a cancer cell within the mammal. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for inducing immune surveillance against a cancer cell within a mammal having cancer. The methods can include, or consist essentially of, administering to a mammal having cancer a composition including a targeting moiety and an inhibitor of phosphorylation of a RB polypeptide, where the targeting moiety targets the composition to a cancer cell within the mammal. The inhibitor of phosphorylation of a RB polypeptide can be an inhibitor of a CDK2 polypeptide. The inhibitor of the CDK2 polypeptide can be dinaciclib, GW8510, or seliciclib. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for inducing immune surveillance against a cancer cell within a mammal having cancer. The methods can include, or consist essentially of, administering to a mammal having cancer a composition including a hypophosphorylated RB polypeptide and a targeting moiety, where the targeting moiety targets the composition to a cancer cell within the mammal. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for treating cancer in a mammal. The methods can include, or consist essentially of, administering to a mammal having cancer a composition comprising a p21 polypeptide and a targeting moiety, where the targeting moiety targets the composition to a cancer cell within the mammal. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for treating cancer in a mammal. The methods can include, or consist essentially of, administering to a mammal having cancer a composition including a targeting moiety and nucleic acid encoding a p21 polypeptide, where the targeting moiety targets the composition to a cancer cell within the mammal, and wherein the cancer cell expresses the p21 polypeptide. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for treating cancer in a mammal. The methods can include, or consist essentially of, administering to a mammal having cancer a composition including a targeting moiety and an inhibitor of phosphorylation of a RB polypeptide, where the targeting moiety targets the composition to a cancer cell within the mammal. The inhibitor of phosphorylation of a RB polypeptide can be an inhibitor of a CDK2 polypeptide. The inhibitor of the CDK2 polypeptide can be dinaciclib, GW8510, or seliciclib. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for treating cancer in a mammal. The methods can include, or consist essentially of, administering to a mammal having cancer a composition including a targeting moiety and a hypophosphorylated RB polypeptide, where the targeting moiety targets the composition to a cancer cell within the mammal. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for inducing immune surveillance against a cancer cell within a mammal having cancer. The methods can include, or consist essentially of, administering to a mammal having cancer a composition including a CXCL14 polypeptide, an IL-34 polypeptide, and a targeting moiety, where the targeting moiety targets the composition to a cancer cell within the mammal. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

In another aspect, this document features methods for treating cancer in a mammal. The methods can include, or consist essentially of, administering to a mammal having cancer a composition including a CXCL14 polypeptide, an IL-34 polypeptide, and a targeting moiety, where the targeting moiety targets the composition to a cancer cell within the mammal. The mammal can be a human. The cancer can be liver cancer, colorectal cancer, breast cancer, head and neck cancer, or cervical cancer. The targeting moiety can include an antibody or a scFv. The cancer cell can include a mutant p53 gene. The method can include identifying the mammal as having cancer cells including a mutant p53 gene. The cancer cell can include a decreased level of expression of a PASP polypeptide. The PASP polypeptide can be a CXCL14 polypeptide, an IL-34 polypeptide, an IL-7 polypeptide, or a CCL17 polypeptide. The method of any one of claims 1-27, wherein said method comprises identifying said mammal as having cancer cells comprising a decreased level of expression of a PASP polypeptide. The method can include identifying the mammal as having cancer cells including a decreased level of a CXCL14 polypeptide. The composition can be in the form of a viral vector, a conjugate, a liposome, a polymeric micelle, a microsphere, or a nanoparticle. The components of the composition can be covalently attached. The components of the composition can be non-covalently attached.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1J. P21-activated RB interacts with STAT and SMAD transcription factors (TFs) at select gene promoters to establish a bioactive secretome. FIG. 1A) Venn diagrams of RNA-seq data depicting downregulated SASP factors with depletion of p21 or Rb in the indicated irradiation induced senescent mouse embryonic fibroblasts (IR-MEFs). FIG. 1B) Heatmaps of commonly downregulated SASP factors indicated in FIG. 1A. FIG. 1C) Overrepresentation analyses for TFs implicated in PASP factor expression. Bolded TFs are significantly activated in SNCs and inhibited upon shp21 and shRb. FDR, false discovery rate. FIG. 1D) Identification of SASP genes that bind RB, and TF motif analysis of RB peaks underlying secreted factors in OI-senescent IMR-90 cells. FIG. 1E) Representative RB occupancy plots at PASP genes. FIG. 1F) Timeline and Venn diagrams based on RNAseq depicting significantly upregulated secreted factors (SFs). FIG. 1G) Timeline and Venn diagrams comparing significantly downregulated SFs upon p21 or Rb depletion. FIG. 1H) Functional annotation analyses of 84 PASP factors indicated in FIG. 1G) displaying overrepresented functional clusters. GF, growth factor. FIG. 1I) Schematic of CM production and transwell migration assay of peritoneal immune cells in the presence of CM. FIG. 1J) Representative images and quantitation of adherent macrophages in the bottom transwell chamber. Data represent means±SEM. ns, not significant. **P<0.01. One-way ANOVA with Sidak's correction (FIG. 1J).

FIGS. 2A-2M. P21-induced immunosurveillance requires PASP factor CXCL14. FIG. 2A) Venn diagrams depicting significantly upregulated PASP factors. FIG. 2B) Transwell migration assay with CM in the presence of CXCL14-neutralizing or IgG antibodies. FIG. 2C) as in FIG. 2B but with CM from shRNA-transduced MEFs. FIG. 2D) Schematic of L-p21 and Ai14 transgenes and P21-OE induction in hepatocytes via Cre-encoding adenovirus. FIG. 2E) RT-qPCR on flow-sorted Tom⁺ hepatocytes. FIG. 2F) Representative picture and quantification of Tom⁺ hepatocytes joined by ≥3 F4/80⁺ cells. FIG. 2G) As in FIG. 2F but assessing livers from mice treated with CXCL14-neutralizing or IgG control antibodies. FIG. 2H) Representative image and quantification of Tom hepatocytes associated with ≥1 B220⁺ cells. FIG. 21) Representative picture and quantification of Tom⁺ hepatocytes associated with ≥1 CD38⁺ cells. FIG. 2J) Proportion of Tom⁺ and healthy (not dying) hepatocytes. FIG. 2K) Representative picture and quantification of dying Tom⁺ hepatocytes. FIG. 2L) Representative picture and quantification of Tom⁺ hepatocytes associated with ≥1 iNOS⁺ cells. FIG. 2M) As in FIG. 2L but assessing dying P21-OE Tom⁺ hepatocytes. Scale bars, 10 μm (FIGS. 2F, 2H, 2I, 2K and 2L). Data represent means±SEM. ns, not significant. *P<0.05; **P<0.01; ***P<0.001. One-way ANOVA with Sidak's correction (FIGS. 2B, 2C, 2F to 2L) and unpaired two-tailed 1-tests (FIG. 2E).

FIGS. 3A-3J. P21 induced by oncogenic RAS places cells under immunesurveillance. FIG. 3A) (Left) Schematic representation of L-KRAS^{G12V} and Ai14 transgenes, and p21- and Rb-conditional knockout alleles. Blue triangles denote LoxP sites. (Right) Schematic of the experimental design. FIG. 3B) Proportion of Tom⁺ p21⁺ hepatocytes among Tom⁺ hepatocytes at indicated days after adeno-Cre injection. FIG. 3C) Quantification of Tom hepatocytes joined by ≥3 F4/80⁺ macrophages. P21high, cells with elevated P21 staining; P21low, cells with baseline or background level P21 staining. FIG. 3D) RT-qPCR on flow-sorted Tom⁺ hepatocytes. FIG. 3E) Proportion of hepatocytes that is Tom⁺ and appears healthy (not dying). FIG. 3F) Quantification of dying Tom⁺ hepatocytes. FIG. 3G) As in FIG. 3C but for hepatocytes with ≥1 iNOS⁺ cells. FIG. 3H) As in FIG. 3C but for hepatocytes with ≥1 CD38 cells. FIG. 3I) Representative image and quantitation of Tom⁺ hepatocyte clusters. FIG. 3J) Proportion of Tom⁺ EdU⁺ hepatocytes in- or outside Tom⁺ clusters. Scale bar, 20 μm. FIG. 3I). Data represent means±SEM. ns, not significant. *P<0.05; **P<0.01; ***P<0.001. Two-way ANOVA with Sidak's correction (D12 and D28 in FIGS. 3B, and 3E to 3H), one-way ANOVA with Sidak's correction (D4 in FIGS. 3B to 3D, and 3I) or unpaired two-tailed 1-test (FIG. 3J).

FIGS. 4A-4J. P21 places cells under immunosurveillance to establish a timer mechanism that controls cell fate. FIG. 4A) schematic overview of CM preparations from dox-inducible P21-OE MEFs. FIG. 4B) Western blot for P21. PonS served as loading control. FIG. 4C) Transwell macrophage migration with CM from indicated MEFs. FIG. 4D) RT-qPCR of the indicated MEFs. FIG. 4E) (Top) Schematic representation of the iL-p21 and Ai139 transgenes. Blue triangles denote LoxP sites. (Bottom) Schematic of the experimental design with fluorescent markers for transgenic P21 expression and repression indicated. FIG. 4F) Rates of P21 overexpression (P21⁺) among hepatocytes that were positive for Tom and eGFP (P21-OE “ON”) or only Tom (P21-OE “OFF”). FIG. 4G) Representative image of a P21-OE hepatocyte surrounded by three macrophages, and quantification of fluorescent hepatocytes joined by ≥3 F4/80⁺ macrophages. FIG. 4H) Assessment of fluorescent hepatocytes associated with ≥1 iNOS⁺ cells. FIG. 4I) As FIG. 4H but assessing cells with ≥1 CD38⁺ cells. FIG. 4J) Representative image of a 6d^{ON}+2d^{OFF} dying hepatocyte and quantification of death rates among fluorescent hepatocytes. Scale bars, 10 μm (FIGS. 4G and 4J). Data represent means±SEM. ns, not significant. *P<0.05; **P<0.01; ***P<0.001. Two-way ANOVA with Sidak's correction (FIGS. 4C, 4D, and 4F to 4J).

FIGS. 5A-5M. Enrichment and validation of SNCs generated via distinct stressors. FIG. 5A) Bright field images of flow-sorted MEFs before or after irradiation and stained for SA-β-Gal. FIG. 5B) Quantification of SA-β-Gal cells in flow-sorted fractions of the indicated MEF cultures. FIG. 5C) Images of 53BP1-immuno-labelled MEFs from the indicated cultures. FIG. 5D) Quantification of cells with >1 53BP1 foci. FIG. 5E) Images of p21-immuno-labelled MEFs. FIG. 5F) Quantification of cells with nuclear P21 in indicated flow-sorted MEF cultures. FIG. 5G) Growth curves of IR, REP and OI-senescent MEFs and corresponding C1 control cultures. FIG. 5H) Expression of senescence markers in the indicated flow-sorted MEFs as determined by RT-qPCR. FIG. 5I) Quantification of SA-β-Gal⁺ IMR-90 cells in the indicated cultures. FIG. 5J) Quantification of EdU⁺ IMR-90 cells, which were allowed to incorporate EdU for 48 hours. FIGS. 5K and 5L) Gene expression of senescence markers as assessed by RT-qPCR. FIG. 5M) Flow-sorted L13KRAS^{G12V} MEFs 10 days after transduction with pTSIN-Cre or empty vector (EV) virus analyzed for the indicated senescence markers. Abbreviations: C1, proliferating control; C2, non-SNCs examined 2 days after IR or OI; IR, irradiation-induced SNCs; REP, serially passaged SNCs; OI, KRAS^{G12V}-induced SNCs. Scale bars, 100 μm (FIG. 5A) and 10 μm (FIGS. 5C and 5E). Data represent means±SEM. For MEF experiments independent MEF lines were used (FIGS. 5A to 5H and 5M), for IMR-90 experiments technical replicates are depicted (FIGS. 51 to 5L). ns, not significant. *P<0.05; **P<0.01; ***P<0.001 (paired two-tailed 1-tests (REP) or one-way ANOVA with Sidak's correction (IR, OI) (FIGS. 5B, 5D, 5F, and 5H to 5L), two-way ANOVA with Bonferroni correction (FIG. 5G) and paired two-tailed 1-tests (FIG. 5M).

FIGS. 6A-6J. Senescence-associated super enhancer identification in senescent MEFs, IMR-90 cells, and liver cells. FIG. 6A) Strategy to identify senescence-associated super enhancers and nearby genes that are activated in the senescent state. FIG. 6B) Venn diagrams depicting numbers of shared and distinct senescence-associated super enhancers between IR, REP, and OI MEF datasets and IMR-90 IR-SNCs dataset. Forty commonly shared MEF senescence-associated super enhancers are located nearby 50 senescence-associated super enhancer-controlled genes, whereas 562 IMR-90 senescence-associated super enhancers are adjacent to 872 senescence-associated super enhancer-controlled genes, of which the 11 depicted genes are shared between MEFs and IMR-90 cells. Three of these are also significantly upregulated in IMR-90 OI-SNCs (*). FIG. 6C) Representative H3K27Ac occupancy plots at the Cdkn1a locus in the indicated conditions in MEFs (top) and IMR-90 cells (bottom). Black bars denote senescence-associated super enhancer location. Y-axes depict cpm (counts per million mapped reads). Note that unlike C1 IR and C1 REP MEFs, which grew unperturbed, C1 control OI-senescent MEFs were infected with pLenti-ER-KRAS^{G12V} virus, selected for hygromycin resistance, and cultured in the absence of 4′-OHT. FIG. 6D) Schematic of L-KRAS^{G12V} and Ai14 transgenes, expressing KRAS^{G12V} and tdTomato (Tom), respectively. Blue triangles denote LoxP sites. FIG. 6E) Schematic of in vivo SNC generation experiments using Ai14;L-KRAS^{G12V} mice and Ai14 control mice. Mice were injected with (′re-encoding adenovirus via the tail vein to remove the floxed transcriptional stop cassette (L) from L-KRAS^{G12V} and Ai14 in liver cells. FIG. 6F) (Left) Representative cryo-section images of indicated mice 8 days after adeno-Cre recombination. (Right) Quantification of Tom⁺ liver cells 8 days after adeno-Cre recombination. FIG. 6G) Quantification of Tom⁺ cells that are EdU⁺ in indicated livers 8 days after adeno-Cre recombination. FIG. 6H) Representative flow cytometry profile and gating strategy of single liver cell suspensions of Ai14; L-KRAS^{G12V} mice. FIG. 6I) Expression of senescence markers in flow-sorted liver cells 8 days after adeno-Cre recombination as determined by RT-qPCR. FIG. 6J) H3K27Ac ChIP-qPCR of flow-sorted liver cells. PCR was performed in indicated regions of the Cdkn1a MEF-senescence-associated super enhancer marked in the red box. Scale bar, 20 μm (FIG. 6F). Data represent means±SEM. ns, not significant. *P<0.05; **P<0.01. Unpaired two-tailed 1-tests (FIGS. 6F, 6G, 6I, and 6J). Abbreviations: SE, super enhancer; SASE, senescence-associated super enhancer.

FIGS. 7A-7J. Sustained cell-cycle arrest of SNCs requires P21 and RB. FIG. 7A) Western blot for P21 on IR-senescent MEF lysates 3 days after transduction with the indicated shRNAs (two independent shRNAs were used, denoted as −1 and −2). PonS served as loading control. FIG. 7B) Expression of p21 in SNCs transduced with the indicated shRNAs. FIG. 7C) Percentage of EdU⁺ senescent MEFs transduced with the indicated shRNAs. EdU was present during the final 48 hours. FIG. 7D) As FIG. 7C but for IMR-90 SNCs. FIG. 7E) Heatmap depicting log 2 fold expression changes in shp21 versus shSer (box color) and the significance per SASP factor (box size) in SNCs 3 days after knockdown as assessed by RT-qPCR. FIG. 7F) as in FIG. 7A but with Rb knockdown. FIG. 7G) as in FIG. 7B but with Rb knockdown. FIG. 7H) as in FIG. 7C but with Rb knockdown.

FIG. 7I) as in FIG. 7D but with Rb knockdown. FIG. 7J) as in FIG. 7E but with Rb knockdown. FC, fold change. Due to the experimental setup some shSer control values are used for both shp21 and shRb comparisons, when they were run in the same experiment. Data represent means±SEM. ns, not significant. *P<0.05; **P<0.01; ***P<0.001. One-way ANOVA with Sidak's correction (FIGS. 7B to 7D, 7G to 7I, and IR SNCs in FIGS. 7E and 7J) paired two-tailed 1-tests (REP and OI SNCs in FIGS. 7E and 7J).

FIGS. 8A-8D. The SASP is complex and varies with senescence-inducing stressor. FIG. 8A) Unbiased assessment of SASP factors in IR-, REP, and OI-senescent MEFs by identifying genes within mouse GO annotation “Extracellular Space” that are transcriptionally upregulated in SNCs compared to their proliferating counterparts based on RNA-seq. 112 SASP factors were significantly upregulated (indicated as ↑*) for all three senescence-inducing stressors. FIG. 8B) Hierarchical clustering of DESeq2-normalized gene expression of senescent MEFs and proliferating counterparts (using 1-Pearson correlation as distance and average linkage). FIG. 8C) Heatmaps of SASP factors identified in FIG. 8A showing log 2 fold expression changes (box color) in SNCs compared to proliferating controls using RNA-seq data and the significance per SASP factor (box size). Bolded factors were used in RT-qPCR experiments shown in FIG. 7. FIG. 8D) as FIG. 8A but with RNA-1 seq data from IMR-90 IR-SNCs and human GO annotation “Extracellular Space”.

FIGS. 9A-9F. SNCs enter S phase when p21 or Rb are depleted. FIG. 9A) Hierarchical clustering of DESeq2-normalized gene expression acquired from IR-senescent MEFs transduced with indicated shRNAs using 1-Pearson correlation as distance and average linkage. FIG. 9B) Classification of significantly enriched gene sets with positive normalized enrichment score (NES) determined by gene set enrichment analysis (GSEA). Numbers inside the bars indicate the number of individual gene sets from a total of 178 or 164 significantly enriched (false discovery rate, FDR<0.05) gene sets after p21 or Rb knockdown, respectively. FIG. 9C) (Left) Enrichment plots of cell-cycle and mitosis-related gene sets identified in the GSEA, and (right) corresponding heatmap depicting row-scaled z-scores of gene expression for leading-edge genes. FIG. 9D) As in FIG. 9C for E2F mediated regulation of DNA replication. FIG. 9E) As in FIG. 9C but using RNA-seq from IMR-90 IR SNCs transduced with the shP21, shRB or shScr. FIG. 9F) As in FIG. 9E for E2F-mediated regulation of DNA replication.

FIGS. 10A-10C. RB binds to STAT and SMAD TFs to promote PASP factor expression. FIG. 10A) Western blots of immunocomplexes precipitated from IR-senescent MEFs with the indicated antibodies and probed for RB. RB is able to form a complex with SMAD2, SMAD3, STAT1 and STAT6. FIG. 10B) Western blot of IR-senescent MEFs after TF knockdown. FIG. 10C) Relative expression of secreted factors in IR-senescent MEFs after TF knockdown as assessed by RT-qPCR demonstrating the requirement for STAT and SMAD TFs to continued secreted factor expression. Data represent means±SEM. ns, not significant. **P<0.01; ***P<0.001. Paired two-tailed 1-tests (FIG. 10C).

FIGS. 11A-11G. Cell-cycle arrest and the PASP are concurrently established prior to senescence. FIG. 11A) Expression of p21 and Rb in the indicated MEFs as assessed by RT-qPCR. MEFs were transduced with the indicated shRNAs at D2 and D3. FIG. 11B) Western blots of the indicated MEF lysates probed for P21 or RB. Ponceau S (PonS) staining served as loading control. FIG. 11C) Quantification of EdU⁺ MEFs at the indicated times after IR. EdU was present for 24 hours. FIG. 11D) Quantification of SA-β-Gal⁺ cells in the indicated MEF cultures. FIG. 11E) Heatmap of 84 common P21- and RB-controlled PASP factors depicting log 2 fold expression changes based on RNA-seq indicated in FIG. 1G. FIG. 11F) RT-qPCR of selected PASP factors in MEF cultures after the indicated timepoints post-IR. PASP factors induction mirrors P21 induction, with gradual increase at least until D6 post-IR. FIG. 11G) Functional annotation analyses of 84 PASP factors indicated in FIG. 11E displaying more granularly the 34 immune system-related overrepresented functional clusters indicated in FIG. 1H. Points within each cluster represent individual annotations. The total number of annotations per cluster is indicated. Data represent means±SEM. ns, not significant. *P<0.05; **P<0.01; ***P<0.001. One-way ANOVA with Sidak's correction (FIGS. 11A, 11C, 11D, and 11F).

FIGS. 12A-12H. The PASP promotes fibroblast and macrophage migration. FIG. 12A) Transwell migration assay using peritoneal immune cells in the presence of CM collected from the indicated MEF cultures. Quantitation of suspension cells (lymphocytes) in the bottom transwell chamber. Lymphocytes recruitment remains unchanged in the presence of non-senescent or senescent CM. CM-NS, conditioned medium of non-senescent IR-MEFs; CM-S, conditioned medium of IR-senescent MEFs. FIG. 12B) Schematic of intraperitoneal CM injection experiments in wild type mice to test if the PASP can elicit immune cells into the peritoneum. FIG. 12C) Flow cytometry quantification of all cells in the peritoneal lavage isolated from wildtype mice 4 days after injection of indicated CM. FIG. 12D) As in FIG. 12C but displaying only CD11B⁺ cells (macrophages). FIG. 12E) As in FIG. 12C but displaying only B220⁺ cells (B lymphocytes). FIG. 12F) As in FIG. 12C but displaying only TCRβ⁺ cells (T lymphocytes). P21 and RB are needed for efficient macrophage recruitment into the peritoneum. FIG. 12G) (Left) Representative images of MEFs migrating into the scratch space illustrating that the PASP promotes fibroblast migration. Red line depicts edge of scratch. (Right) Quantitation of MEF migration into the denuded area in the presence of the CM indicated in FIG. 12A 2 hours post-1 scratching. FIG. 12H) Scratch assay using MEFs treated with CM from cultures indicated in FIG. 12A. Scratch widths at 12 hours, 24 hours, and 36 hours are depicted as percentage of initial scratch width at 0 hours. Scale bar, 50 μm in FIG. 12G. Data represent means±SEM. ns, not significant. *P<0.05; **P<0.01; ***P<0.001. One-way ANOVA with Sidak's correction (FIGS. 12A and 12C to 12G) and two-way ANOVA with Sidak's correction (FIG. 12H).

FIGS. 13A-13H. RELA is a minor contributor to the PASP and not involved in macrophage migration. FIG. 13A) RT-qPCR of indicated genes in IR-senescent MEF cultures transduced with independent shRNAs against Rela (NFkB P65) or scrambled shRNA control (shScr). IR-senescent MEFs were transduced with shRNAs at D11 and D12 and were harvested for experimentation at D13, reminiscent to shp21 and shRb experiments in IR-SNCs. Rela depletion had no impact on p21 and p16 transcript levels. FIGS. 13B and 13C) Quantification of SA-β-Gal and EdU⁺ cells in the IR-senescent MEF cultures indicated in FIG. 13A. Rela depletion did not impact key SNC properties. FIG. 13D) RT-qPCR of RELA transcriptional targets that encode secreted factors. FIG. 13E) RNA-seq based assessment of RELA-dependent SASP factors in IR-senescent MEFs. (Top) Schematic of the experimental design. (Bottom) Venn diagram depicting numbers of shared and distinct SASP factors downregulated in IR-SNC MEFs depleted for the indicated genes. RNA-seq data for shRela depict commonly downregulated genes in shRela-1 versus shSer and shRela-2 versus shScr, and that the shp21 and shRb RNA-seq data were the same as in FIG. 1. Expression of most PASP factors does not require RELA. FIG. 13F) Heatmap of 29 RELA-dependent IR-senescent SASP factors indicated in FIG. 13E depicting log 2 fold expression changes. The 9 SASP factors commonly downregulated in shp21, shRb and shRela versus respective shScr are indicated. FIG. 13G) Functional annotation analyses of 29 RELA-dependent SASP factors indicated in FIG. 13E and FIG. 13F displaying overrepresented functional clusters. Points within each cluster represent individual annotations. The total number of annotations per cluster is indicated. FDR, false discovery rate. The highest number of annotations are related to the immune system. FIG. 13H) Transwell migration assay using murine peritoneal immune cells in the presence of CM collected from cycling MEFs, or IR-senescent MEFs (CM-S) transduced with indicated shRNAs. Quantitation of adherent macrophages (left) and suspension cells (lymphocytes) (right) in the bottom chamber of the transwell. Both Rela shRNAs show that the RELA-dependent arm of the SASP has no effect on macrophage or lymphocyte migration. Data represent means±SEM. ns, not significant. *P<0.05; **P<0.01. One-way ANOVA with Sidak's correction (FIGS. 13A to 13D and 13H).

FIGS. 14A-14N. P21-OE induces cell-cycle arrest and a PASP that stimulates fibroblast and macrophage migration. FIG. 14A) Western blot of cycling MEFs transduced with viral particles containing pTSIN lentiviral vector with p21-Myc-Flag or without EV and probed with an anti-Myc-tag antibody. PonS staining served as loading control. FIG. 14B) RT-qPCR of p21 or p16 in the indicated MEFs 4 (D4) or 10 (D10) days after viral transduction, demonstrating that P21-OE does not cause P16 to be elevated at D4 but does so at D10. FIG. 14C) Quantification of SA-β-Gal⁺ cells in cultures indicated in FIG. 14B, demonstrating the presence of SNCs at D10. FIG. 14D) Cell proliferation of the indicated MEFs (cells were seeded 3 or 7 days after viral infection and counted every 24 hours). FIG. 14E) Western blots of immunocomplexes precipitated from the chromatin fraction of D4 P21-OE MEFs with the indicated antibodies and probed for RB, showing that, upon P21-OE, RB interacts with SMAD and STAT TFs at chromatin. FIG. 14F) Functional annotation analysis of the 295 PASP factors identified in D4 P21-OE MEFs indicated in FIG. 2A. Points within each functional cluster represent individual annotations. The total number of annotations per cluster is indicated. FDR, false discovery rate. The highest number of annotations are related to the immune system and migration/adhesion. FIGS. 14G and 14H) Scratch assay with CMs from the indicated cultures demonstrating that P21-OE is sufficient to provoke fibroblast migration. Quantification of wildtype MEFs migrating into the scratch space 2 hours post-scratching (FIG. 14G) and measurements of scratch widths at 12 hours, 24 hours, and 36 hours after scratching (FIG. 14H). FIGS. 14I and 14J) Transwell migration of murine peritoneal immune cells in the presence of CM harvested from the MEF cultures indicated in FIG. 14G. Representative images and quantitation of adherent macrophages (FIG. 14I) and suspension cells (lymphocytes) (FIG. 14J) in the bottom chamber of the transwell. P21-OE CM attracts macrophages, but not lymphocytes. FIGS. 14K to 14N) Intraperitoneal CM injection experiments in wild type mice with CM harvested from the indicated MEF cultures. Flow cytometry quantification of all cells in the peritoneal lavage 4 days after CM injection (FIG. 14K), CD11B⁺ cells (macrophages) (FIG. 14L), B220⁺ cells (B cells) (FIG. 14M) and TCRβ⁺ cells (T cells) (FIG. 14N). P21-OE facilitates immune cell recruitment into the peritoneum. Due to the experimental setup the “non-injected” group in FIGS. 14K to 14N is the same as in FIGS. 12C to 12F, as all condition were assessed in the same experiment. Scale bar, 100 μm (FIG. 14I). Data represent means±SEM (FIGS. 14B, 14C, and 14G to 14N). ns, not significant. *P<0.05; **P<0.01; ***P<0.001. One-way ANOVA with Sidak's correction (FIGS. 14B, 14C, 14G, and 14I to 14N), two-way ANOVA with Bonferroni correction (FIG. 14D), and two-way ANOVA with Sidak's correction (FIG. 14H).

FIGS. 15A-15C. CXCL14 inactivation does not impact lymphocyte migration. FIG. 15A) Quantification of migrated lymphocytes in a transwell migration assay using peritoneal immune cells in the presence of CM from the indicated MEFs and with addition of the indicated antibodies. FIG. 15B) Knockdown efficiency of Cxcl14 in D4P21-OE MEFs with two independent shRNAs targeting Cxcl14 in as analyzed by RT-qPCR. FIG. 15C) Quantification of migrated lymphocytes in a transwell migration assay using peritoneal immune cells in the presence of CM from the indicated MEFs. Data represent means±SEM. ns, not significant. *P<0.05. One-way ANOVA with Sidak's correction (FIGS. 15A to 15C).

FIGS. 16A-16N. P21-OE in HDFs and HUVECs induces a PASP that contains CXCL14 and promotes macrophage migration. FIG. 16A) Western blot of HDFs transduced with pTSIN lentiviral vector containing p21-Myc-Flag or EV 4 days after viral infection and probed with a P21 antibody. PonS staining served as loading control. FIG. 16B) Quantification of EdU™ HDFs that were allowed to incorporate EdU for 24 hours. P21-OE efficiently induces cell cycle arrest of HDFs. FIG. 16C) Quantification of SA B-Gal⁺ cells in cultures indicated in FIG. 16B. FIGS. 16D and 16E) Quantification of migrated macrophages (FIG. 16D) or lymphocytes (FIG. 16E) in a transwell migration assay using murine peritoneal immune cells in the presence of CM from HDF cultures indicated in FIG. 16B. P21 induction provokes macrophage recruitment, but not lymphocyte migration. FIG. 16F) RT-qPCR of P16 in HDFs indicated in FIG. 16B. FIG. 16G) RT-qPCR of selected PASP factors in HDFs indicated in FIG. 16B. P21-OE causes a PASP in HDFs that includes CXCL14. FIG. 16H) As in FIG. 16A but using HUVECs. FIG. 16I) As in FIG. 16B but using HUVECs. FIG. 16J) As in FIG. 16C but using HUVECs. FIGS. 16K and 16L) As in FIGS. 16D and 16E but using CM harvested from HUVEC cultures. FIG. 16M) As in FIG. 16F but using HUVECs. FIG. 16N) As in FIG. 16G but using HUVECs. Data represent means #SEM. For HDF experiments independent HDF lines were used (FIGS. 16A to 16C, 16F, and 16G), for HUVEC experiments technical replicates are depicted (FIGS. 16H to 16J, 16M, and 15N). ns, not significant. *P<0.05; **P<0.01; ***P<0.001. Paired two-tailed 1-tests (FIGS. 16B, 16C, 16F, and 16G), one sample two-tailed 1-tests (FIGS. 16D, 16E, 16K, and 16L) or unpaired two-tailed 1-tests (FIGS. 16I, 16J, 16M, and 16N).

FIGS. 17A-17F. D4 P21-OE hepatocytes are non-senescent when adjoined by macrophages. FIG. 17A) Assessment of EdU incorporation rates in Tom⁺ hepatocytes of Ai14;L-p21 or Ai14 mice 4 days after adeno-Cre injection. EdU was injected at D2 and D3. P21-OE arrests hepatocytes that are cycling. FIG. 17B) (Left) Representative immunofluorescence images of Lamin B1-labelled Ai14 and Ai14;L-p21 hepatocytes. (Right) quantification of Tom⁺ Lamin B1″ Ai14 and L-p21;Ai14 hepatocytes at the indicated days after adeno-Cre injection. FIG. 17C) As in FIG. 17B but assessing the proportion of Tom⁺ cells with higher HMGB1 levels in the nucleus than in the cytoplasm (N>C). Markers of cellular senescence are overserved D8 post-adeno-Cre. FIG. 17D) (Top) FACS gating strategy to collect Tom⁺ hepatocytes after collagenase perfusion. (Bottom) Representative images of the collected hepatocytes. FIG. 17E) Representative image and quantification of Tom⁺ hepatocytes joined by 1 or more NKp46⁺ cells (NK cells) in livers indicated in FIG. 17B. NK cells are not recruited by P21-OE. FIG. 17F) Representative image and quantification of dying Tom⁺ hepatocytes at D8 post-adeno-Cre injection surrounded by ≥3 F4/80⁺ cells (macrophages, MP), ≥1 CD38⁺ cells (T cells, T), ≥1 B220⁺ cells (B cells, B) or ≥1 NKp46⁺ cells (NK cells, NK). Scale bars, 10 μm (FIGS. 17B, 17C, 17E, and 17F) and 20 μm (FIG. 17D). Data represent means±SEM. ns, not significant. **P<0.01; ***P<0.001. Unpaired two-tailed t-test (FIG. 17A) or one-way ANOVA with Sidak's correction (FIGS. 17B, 17C, and 17E).

FIGS. 18A-18G. CD8⁺ T cells eliminate P21-OE hepatocytes. FIG. 18A) Representative images and quantifications of Tom⁺ hepatocytes joined by 1 or more CD4⁺ or CD8α^{′} cells (T cells) 8 days after adeno-Cre administration in Ai14;L-p21 mice. FIG. 18B) As in FIG. 18A but assessing dying Tom⁺ hepatocytes. Both, CD4⁺ and CD8α⁺ T cells are recruited to healthy as well as dying P21-OE hepatocytes. FIG. 18C) Schematic and timeline of CD8a depletion experiment in Ai14 and Ai14;L-p21 mice. CD8α-neutralizing antibody or PBS (control) was injected intraperitoneally 5 times (D0, D1, D2, D6 and D12), whereas adeno-Cre was injected via the tail vein at D7. Livers and spleens were harvested 8 days post-adeno-Cre injection (experimental day 15). FIG. 18D) Representative flow cytometry profiles and gating strategy to quantify T cell subsets in spleens from mice treated with CD8α-neutralizing antibody or PBS (control). FIG. 18E) Flow cytometry quantification of total CD4⁺ or CD8α⁺ T cell numbers in spleens from indicated mice showing depletion of CD8α⁺ T cells. FIG. 18F) Quantification of healthy hepatocytes that are Tom⁺ in livers indicated in FIG. 18E. P21-OE hepatocyte numbers remain preserved when CD8α⁺ T cell are diminished. FIG. 18G) Quantification of Tom⁺ hepatocytes that were dying in livers indicated in FIG. 18E. P21-OE hepatocytes of mice subjected to CD8α⁺ T cell depletion are not subject to immunoclearance. Scale bars, 10 μm (FIGS. 18A and 18B). Data represent means±SEM. ns, not significant. *P<0.05; **P<0.01; ***P<0.001. Unpaired two-tailed 1-tests (FIG. 18A) or one-way ANOVA with Sidak's correction (FIGS. 18E to 18G).

FIGS. 19A-19I. D4 P16-OE MEFs do not produce a secretome that promotes macrophage migration. FIG. 19A) WT MEFs transduced with lentiviral particles containing pTSIN-p16-Myc-Flag or pTSIN (EV) analyzed for p16 or p21 transcript levels at D4 or D10 after transfection using RT-qPCR. FIG. 19B) Quantification of SA-β-Gal⁺ cells in cultures indicated in FIG. 19A. FIG. 19C) Cell proliferation of the indicated MEFs (cells were seeded 3 or 7 days after viral infection and counted every 24 hours). EV data in FIGS. 19A to 19E) are the same data as displayed in FIG. 14, because P21- and P16-overexpression were performed in parallel. FIG. 19D) Timeline of RNA-seq experiments. FIG. 19E) Venn diagrams comparing significantly upregulated SFs upon P16- or P21-overexpression versus EV control. D4 P16-OE MEFs produce a substantial number of SFs consisting largely of PASP factors. However, these P16-OE-associated SFs represent only 183 of 295 PASP factors. P21-OE and EV control data are the same RNA-seq data as displayed in FIG. 2 and FIG. 14. FIG. 19F) Heatmap of 112 PASP factors indicated in FIG. 19E that are exclusively induced in D4 P21-OE MEFs, including Cxcl14. FIG. 19G) Functional annotation analyses on SFs of D4 P16-OE MEFs. Points within each functional cluster represent individual annotations. The total number of annotations per cluster is indicated. FDR, false discovery rate. P16-OE SFs play roles in similar biological processes as the PASP, but the PASP has considerably more immune system-related annotations. FIG. 19H) As in FIG. 19G but for SFs that are unique for D4 P21-OE. FIG. 19I) Transwell migration assay using peritoneal immune cells in the presence of CM collected from indicated MEF cultures. Quantitation of adherent macrophages (left) and suspension cells (lymphocytes) (right) in the bottom chamber of the transwell. The D4 P16-OE MEF secretome does not stimulate macrophage migration, unlike D10 P16-OE MEFs that have elevated p21 and are senescent. Data represent means±SEM. ns, not significant. *P<0.05; **P<0.01; ***P<0.001. One-way ANOVA with Sidak's correction (FIGS. 19A, 19B, and 19I) or two-way ANOVA with Bonferroni correction (FIG. 19C).

FIGS. 20A-20H. P16-OE hepatocytes are not placed under immunosurveillance. FIG. 20A) (Top) Schematic of the L-p16 and Ai14 transgenes. Blue triangles denote LoxP sites. (Bottom) Approach to induce P16-OE in mouse hepatocytes via tail-vein injection of (′re-encoding adenovirus. FIG. 20B) EdU incorporation rates in the indicated D4 Tom⁺ hepatocytes indicating that P16-OE hepatocytes are subject to proliferative arrest. FIG. 20C) RT-qPCR for PASP factors on RNA isolated from the indicated flow-sorted D4 Tom⁺ hepatocytes. All PASP factors but Cxcl14 and Ssc5d were commonly induced in both D4 P21-OE MEFs and D4 P16-OE MEFs. FIG. 20D) Quantification of D8 Tom⁺ hepatocytes with elevated P21 levels. FIG. 20E) (Left) Quantification of Lamin B1 expression in the indicated Tom⁺ hepatocytes. (Right) Quantification of D8 Tom⁺ hepatocytes with higher nuclear than cytoplasmic (N>C) HMGB1 levels (right) in the indicated livers. Both markers indicate that D8 P16-OE hepatocytes are non-senescent. FIG. 20F) Quantification of Tom⁺ hepatocytes joined by 3 or more F4/80⁺ macrophages at indicated days after adeno-Cre administration. Consistent with the lack of Cxcl14 induction, P16-OE hepatocytes fail to attract macrophages. FIG. 20G) Quantification of healthy hepatocytes that are Tom⁺ in the indicated livers. FIG. 20H) Quantification of Tom⁺ hepatocytes that are dying in the indicated livers. Data represent means±SEM. ns, not significant. *P<0.05; **P<0.01; ***P<0.001. Unpaired two-tailed 1-tests (FIGS. 20B to 20E) or one-way ANOVA with Sidak's correction (FIGS. 20F to 20H).

FIGS. 21A-21H. D4 P27-OE MEFs are arrested and yield a secretome that lacks CXCL14 and fails to stimulate macrophage migration. FIG. 21A) WT MEFs transduced with lentiviral particles containing pTSIN-p27-Myc-Flag or pTSIN (EV) analyzed for P27 expression by western blotting. PonS staining served as loading control. FIG. 21B) RT-qPCR analysis of RNA from the indicated MEF cultures for p16, p21 and p27 transcript levels, indicating that p21 and p16 expression remains at baseline in D4 P27-OE MEFs. FIG. 21C) Quantification of EdU⁺ MEFs 24 hours after EdU administration, indicating that P27-OE result in cell-cycle arrest. Legend is as in FIG. 21B. FIG. 21D) Quantification of SA-β-Gal⁺ cells in cultures indicated in FIG. 21B. Prolonged P27-OE can induce cellular senescence. FIG. 21E) RT-qPCR of select PASP factors in MEFs indicated in FIG. 21B. Core PASP factors are not elevated in D4 P27-OE MEFs (D10 P27-OE MEFs are senescent and have elevated p21 and Cxcl14 transcript levels). FIG. 21F) Timeline and Venn diagrams depicting numbers of shared and distinct SFs upregulated in the indicated MEFs. P21-OE, P16-OE and EV control RNA-seq data are the same as in in FIG. 2, FIG. 14, or FIG. 19. The P27-OE SF signature partly resembles that of P16-OE and P21-OE, but with fewer engaged factors than either. FIG. 21G) Functional annotation analyses on 81 SFs of D4 P27-OE MEFs. Points within each cluster represent individual annotations. The total number of annotations per cluster is indicated. FDR, false discovery rate. FIG. 21H) Transwell migration assay using murine peritoneal immune cells in the presence of CM collected from indicated MEF cultures. Quantitation of adherent macrophages (left) and suspension cells (lymphocytes) (right) in the bottom chamber of the transwell. CM of D4 P27-OE MEFs does not stimulate macrophage migration. Data represent means±SEM. ns, not significant. *P<0.05; **P<0.01; ***P<0.001. One-way ANOVA with Sidak's correction (FIGS. 21B to 21E and 21H).

FIGS. 22A-22F. D4 and D12 KRAS^{G12V} Tom⁺ hepatocytes with or without P21 analyzed for cell cycling and senescence. FIG. 22A) Quantitation of Myc-tag-positive Tom⁺ hepatocytes in indicated livers demonstrating that Tom is a reliable marker for KRAS^{G12V} expression. FIG. 22B) PCR-based assessment of Cre-mediated inactivation of the p21floxed or Rbfloxed alleles in livers of the indicated mice (samples receiving adeno-Cre were the same as samples used in other panels of this figure and FIG. 3 and contained ˜5% Tom⁺ hepatocytes). PCR primers spanning floxed exons (p21 exon 2, or Rb exon 19) were used. FIG. 22C) EdU incorporation rates in Tom⁺ hepatocytes of mice designated in FIG. 22D indicating that KRAS^{G12V} expression inhibits cell-cycle entry at D12 and D28 regardless of P21 status, while cycling is increased at D4 when P21 is lacking. FIG. 22D) (Left) Representative images of Tom⁺ hepatocytes stained for phospho-Serine10 Histone H3 (pHH3⁺) to illustrate typically staining patterns in G2 and mitosis. (Right) Quantification of Tom⁺ hepatocytes in G2 or M phase in the indicated livers using pHH3 staining. The data obtained indicate although P21 inactivation increased S-phase entry at D4 (not at D12 and D28), these hepatocytes did not actually engage in cell proliferation as M phase rates were not increased. FIG. 22E) (Left) Quantification of Lamin B1 expression in the Tom⁺ hepatocytes indicated in FIG. 22C. (Right) Quantification of Tom⁺ hepatocytes with higher nuclear than cytoplasmic (N>C) HMGB1 levels in mice indicated in FIG. 22C. FIG. 22F) Representative DIC images and quantification of SA-β-Gal⁺ hepatocytes in livers indicated in FIG. 22C). Scale bars, 20 μm (FIGS. 22A and 22F) or 10 μm (FIG. 22D). Data represent means±SEM. ns, not significant. *P<0.05; **P<0.01; ***P<0.001. Unpaired two-tailed 1-test (FIG. 22A), two-way ANOVA with Sidak's correction (FIGS. 22C to 22F).

FIGS. 23A-23J. P21-OE cells promptly establish a PASP that is reversible with normalization of P21 levels. FIG. 23A) Western blot of a dilution series of pTRIPZ-p21-Myc-Flag samples induced with doxycycline for 2 days (2d^{ON}) and compared to D2 IR-induced MEFs. Blot was probed with a P21 antibody and PonS served as loading control. FIG. 23B) Quantification of EdU⁺ MEFs in the indicated conditions. EdU was allowed to be incorporated for 24 hours. After P21-normalization, MEFs return proliferation. FIG. 23C) as in FIG. 23B but after samples harvest after D4. FIG. 23D) Transwell migration assay using peritoneal immune cells in the presence of indicated CM. Migrated suspension cells (lymphocytes) were quantified. FIG. 23E) Scratch assay using CM from indicated MEF cultures indicated in FIG. 23D. Continued P21-OE is required for continued, accelerated scratch closure. FIG. 23F) Western Blot showing P21 levels in the indicated conditions after dox induction. FIG. 23G) Quantification of EdU⁺ MEFs in the indicated conditions. EdU was allowed to be incorporated for 12 hours. P21 establishes cell cycle arrest within 24 hours post-OE. FIG. 23H) Transwell migration assay using peritoneal immune cells in the presence of CM collected from cultures indicated in FIG. 23G. Quantitation of adherent macrophages (left) and suspension cells (lymphocytes) (right) in the bottom chamber of the transwell. Macrophage engagement is induced 24 hours post-P21-OE. FIGS. 23I and 23J) Gene expression analyses via RT-qPCR of selected E2F transcriptional targets (FIG. 23I) and PASP factors (FIG. 23J) in conditions indicated in (FIG. 23G). RB-mediated repression of E2F targets and activation of PASP genes occurs within 24 hours post-P21-OE. Data represent means±SEM. ns, not significant. *P<0.05; **P<0.01, ***P<0.001. Two-way ANOVA with Sidak's correction (FIGS. 23B to 23E) or one-way ANOVA with Sidak's correction (FIGS. 23G to 23J).

FIGS. 24A-24E. P21-OE in hepatocytes is tightly controllable with the iL-1 p21 transgene. FIG. 24A) Representative images of a 2d^{ON} Tom⁺ eGFP⁺ Ai139;iL-p21 hepatocyte immuno-labelled for P21 (the cell shown is representative for data presented in FIG. 4F. FIG. 24B) (Top) Quantification of fluorescent hepatocytes that are Myc-tag⁺ in the indicated mice. In the absence of doxycycline (“ON”) Tom⁺ eGFP⁺ hepatocytes were selected for quantification, and in the presence of doxycycline (“OFF”) Tom⁺ hepatocytes. (Bottom) Representative image of a 2d^{ON} Tom⁺ eGFP⁺ Ai139;iL7 p21 hepatocyte immuno-labelled with a Myc-tag antibody. Dox-administration efficiently quenched P21 transgene expression. FIG. 24C) As in FIG. 24B but quantifying the proportion of fluorescent Lamin B1⁺ hepatocytes. FIG. 24D) As in FIG. 24B but quantifying the proportion of fluorescent hepatocytes with higher HMGB1 levels in the nucleus than in the cytoplasm (N>C). FIG. 24E) Quantification of SA-β-Gal⁺ hepatocytes in livers indicated in FIG. 24B. Scale bars, 10 μm (FIGS. 24A and 24B). Data represent means #SEM. ns, not significant. ***P<0.001. Two-way ANOVA with Sidak's correction (FIGS. 24B to 24E).

FIG. 25. Model for how P21 can coordinate cell-cycle arrest and immunosurveillance of stressed cells through RB hypophosphorylation. Stress-activated P53 induces expression of p21, which, as a potent inhibitor of cyclin-CDK complexes, yields hypophosphorylated RB. In this configuration, RB can repress the transcriptional activity of E2F TFs that are bound to the promoters of genes required for cell-cycle progression through. In parallel, hypophosphorylated RB can bind to and activate STAT and SMAD transcription factors at select promoters to create a bioactive secretome, the PASP, which, places stressed cells under immediate immunosurveillance through chemoattraction of macrophages. CXCL14 functions as a key macrophage-recruiting protein in the PASP. By attracting macrophages, P21 sets a biological timer that allows for a period of stress management (damage repair or stress adaptation) that in hepatocytes spans about four days. Stressed cells that recuperate and normalize P21 within this period cease to produce a PASP, disengage macrophages, and resume their normal activities. The timer expires when the immune system transitions from a surveillance to a clearance mode. This transition is characterized by macrophage polarization towards an M1 phenotype and recruitment of T lymphocytes. It was found that clearance of stressed cells that fail to recuperate and normalize P21 after the timer expires is executed by cytotoxic CD8⁺ T cells. It is shows that P21 induced by mitogenic stress caused by oncogenic KRAS provides a first-line of immunosurveillance for transformed cells at risk for tumorigenesis.

FIGS. 26A and 26B. FIG. 26A) An amino acid sequence of an exemplary CXCL14 polypeptide (SEQ ID NO:1). FIG. 26B) An exemplary nucleic acid encoding a CXCL14 polypeptide (SEQ ID NO:2).

FIGS. 27A and 27B. FIG. 27A) An amino acid sequence of an exemplary IL-34 polypeptide (SEQ ID NO:3). FIG. 27B) An exemplary nucleic acid encoding an IL-34polypeptide (SEQ ID NO:4).

FIGS. 28A and 28B. FIG. 28A) An amino acid sequence of an exemplary IL-7 polypeptide (SEQ ID NO:5). FIG. 28B) An exemplary nucleic acid encoding an IL-7 polypeptide (SEQ ID NO:6).

FIGS. 29A and 29B. FIG. 29A) An amino acid sequence of an exemplary CCL17 polypeptide (SEQ ID NO:7). FIG. 29B) An exemplary nucleic acid encoding a CCL17 polypeptide (SEQ ID NO:8).

### DETAILED DESCRIPTION

This document provides methods and materials for promoting immune surveillance against cancer cells. For example, one or more (e.g., one, two, three, four, or more) agents having the ability to increase a level of a CXCL14 polypeptide can be administered to a mammal (e.g., a human) having cancer to promote immune surveillance against cancer cells. In some cases, one or more CXCL14 polypeptides (and/or one or more nucleic acids designed to encode a CXCL14 polypeptide) can be delivered to a mammal (e.g., a human) having cancer to promote immune surveillance against cancer cells. In some cases, one or more agents that can modulate a PASP pathway to increase expression of a CXCL14 polypeptide can be administered to a mammal (e.g., a human) having cancer to promote immune surveillance against cancer cells. In some cases, the methods and materials provided herein can be used to treat a mammal (e.g., a human) having cancer.

In some cases, one or more (e.g., one, two, three, four, or more) agents that can increase a level of a CXCL14 polypeptide expressed by cancer cells and/or that can increase the presence of a CXCL14 polypeptide within the location of cancer cells (e.g., within 1 to 10 mm of a tumor) can be administered to a mammal (e.g., a human) in need thereof (e.g., a human having cancer) to induce immune surveillance against cancer cells present within a mammal, thereby resulting in the number of cancer cells within the mammal being reduced.

In some cases, one or more (e.g., one, two, three, four, or more) agents that can increase a level of a CXCL14 polypeptide expressed by cancer cells and/or within the vicinity of cancer cells (e.g., within 1 to 10 mm of cancer cells) can be administered to a mammal (e.g., a human) in need thereof (e.g., a human having cancer) to recruit one or more macrophages to cancer cells present within a mammal. In some cases, the materials and methods described herein can be used to increase the number of macrophages present at a tumor site within a mammal having cancer by, for example, 10, 20, 30, 40, 50, 60, 70, 80, 90, 95, or more percent.

In some cases, one or more (e.g., one, two, three, four, or more) agents that can increase a level of a CXCL14 polypeptide expressed by cancer cells and/or within the vicinity of cancer cells (e.g., within 1 to 10 mm of cancer cells) can be administered to a mammal (e.g., a human) in need thereof (e.g., a human having cancer) to polarize (e.g., activate) one or more macrophages to cancer cells present within a mammal. In some cases, the materials and methods described herein can be used to increase the number of polarized macrophages present at a tumor site within a mammal having cancer by, for example, 10, 20, 30, 40, 50, 60, 70, 80, 90, 95, or more percent.

In some cases, one or more (e.g., one, two, three, four, or more) agents that can increase a level of a CXCL14 polypeptide expressed by cancer cells and/or within the vicinity of cancer cells (e.g., within 1 to 10 mm of cancer cells) can be administered to a mammal (e.g., a human) in need thereof (e.g., a human having cancer) to recruit one or more cytotoxic T cells (e.g., CD4⁺ T cells and CD8⁺ T cells) to cancer cells present within a mammal. In some cases, the materials and methods described herein can be used to increase the number of cytotoxic T cells present at a tumor site within a mammal having cancer by, for example, 10, 20, 30, 40, 50, 60, 70, 80, 90, 95, or more percent.

In some cases, one or more (e.g., one, two, three, four, or more) agents that can increase a level of a CXCL14 polypeptide expressed by cancer cells and/or within the vicinity of cancer cells (e.g., within 1 to 10 mm of cancer cells) can be administered to a mammal (e.g., a human) in need thereof (e.g., a human having cancer) to reduce or eliminate the number of cancer cells present within a mammal. For example, the materials and methods described herein can be used to reduce the number of cancer cells present within a mammal having cancer by, for example, 10, 20, 30, 40, 50, 60, 70, 80, 90, 95, or more percent. For example, the materials and methods described herein can be used to reduce the size (e.g., volume) of one or more tumors present within a mammal having cancer by, for example, 10, 20, 30, 40, 50, 60, 70, 80, 90, 95, or more percent.

In some cases, one or more (e.g., one, two, three, four, or more) agents that can increase a level of a CXCL14 polypeptide expressed by cancer cells and/or within the vicinity of cancer cells (e.g., within 1 to 10 mm of cancer cells) can be administered to a mammal (e.g., a human) in need thereof (e.g., a human having cancer) to induce apoptosis of one or more cancer cells within the mammal. In some cases, the materials and methods described herein can be used to increase the level of apoptosis of one or more cancer cells within a mammal having cancer by, for example, 10, 20, 30, 40, 50, 60, 70, 80, 90, 95, or more percent.

In some cases, one or more (e.g., one, two, three, four, or more) agents that can increase a level of a CXCL14 polypeptide expressed by cancer cells and/or within the vicinity of cancer cells (e.g., within 1 to 10 mm of cancer cells) can be administered to a mammal (e.g., a human) in need thereof (e.g., a human having cancer) to improve survival of the mammal. For example, disease-free survival (e.g., relapse-free survival) can be improved using the materials and methods described herein. For example, progression-free survival can be improved using the materials and methods described herein. In some cases, the materials and methods described herein can be used to improve the survival of a mammal having cancer by, for example, 10, 20, 30, 40, 50, 60, 70, 80, 90, 95, or more percent.

Any appropriate mammal having a cancer can be treated as described herein. Examples of mammals having a cancer that can be treated as described herein include, without limitation, humans, non-human primates (e.g., monkeys), dogs, cats, horses, cows, pigs, sheep, mice, and rats. In some cases, a human having a cancer can be treated as described herein.

When treating a mammal (e.g., a human) having a cancer as described herein, the cancer can be any type of cancer. In some cases, a cancer can be a blood cancer (e.g., lymphomas and leukemias). In some cases, a cancer can include one or more solid tumors. In some cases, a cancer can be a primary cancer. In some cases, a cancer can be a metastatic cancer. In some cases, a cancer can include one or more cancer cells having a mutant p53 gene and/or expressing a mutant p53 polypeptide (e.g., as compared to a p53 gene and/or a p53 polypeptide typically seen in the same tissue type of a comparable mammal that does not have cancer). In some cases, a cancer can include one or more cancer cells having a decreased level of one or more PASP polypeptides (e.g., as compared to a level of a PASP polypeptide typically seen in the same tissue type of a comparable mammal that does not have cancer). Examples of cancers that can be treated as described herein include, without limitation, liver cancers, colorectal cancers, breast cancers, head and neck cancers, and cervical cancers.

In some cases, the methods described herein can include identifying a mammal (e.g., a human) as having a cancer. Any appropriate method can be used to identify a mammal as having a cancer. For example, imaging techniques and/or biopsy techniques can be used to identify mammals (e.g., humans) having cancer.

In some cases, the methods described herein can include identifying a mammal (e.g., a human) as having cancer cells and as being likely to response to increased immune surveillance against cancer cells by, for example, identifying that the cancer cells include a mutant p53 gene and/or express a mutant p53 polypeptide. Any appropriate method can be used to identify the presence of a mutant p53 gene and/or a mutant p53 polypeptide. For example, sequencing techniques (e.g., RNA seq), PCR based techniques, and/or immunoblotting can be used to identify the presence of a mutant p53 gene and/or a mutant p53 polypeptide.

In some cases, the methods described herein can include identifying a mammal (e.g., a human) as having cancer cells and as being likely to response to increased immune surveillance against cancer cells by, for example, identifying that the cancer cells have a decreased level of expression of one or more PASP polypeptides (e.g., a CXCL14 polypeptide and a IL-34 polypeptide). For example, a methods described herein can include identifying a mammal (e.g., a human) that has cancer cells as being likely to response to increased immune surveillance against cancer cells by, for example, identifying that the cancer cells have a decreased level of expression of a CXCL14 polypeptide. Any appropriate method can be used to identify the presence of a decreased level of expression of a particular PASP polypeptide. For EXAMPLE, western blotting, RT-qPCR, RNA-seq, and/or enzyme-linked immunosorbent assay (ELISA) can be used to identify the presence of a decreased level of expression of a particular PASP polypeptide. The term “decreased level” as used herein with respect to a level of expression of a PASP polypeptide refers to any level that is less than a reference level of expression of that polypeptide in a mammal (e.g., a human). The term “reference level” as used herein with respect to expression of a PASP polypeptide refers to the level of expression of the PASP polypeptide typically observed in a sample (e.g., a control sample) from one or more healthy mammals (e.g., mammals that do not have a cancer). Control samples can include, without limitation, samples from normal (e.g., healthy) mammals, primary cell lines derived from normal (e.g., healthy mammals), and non-tumorigenic cells lines. It will be appreciated that levels from comparable samples are used when determining whether or not a particular level is an increased level.

A mammal (e.g., a human) having a cancer can be administered or instructed to self-administer any one or more (e.g., one, two, three, four, or more) agents that can increase a level of a CXCL14 polypeptide expressed by cancer cells and/or within the vicinity of cancer cells (e.g., within 1 to 10 mm of cancer cells). An agent that can increase a level of a CXCL14 polypeptide expressed by cancer cells and/or within the vicinity of cancer cells (e.g., within 1 to 10 mm of cancer cells) can be any type of molecule. Examples of compounds that can increase a level of a CXCL14 polypeptide expressed by cancer cells and/or within the vicinity of cancer cells (e.g., within 1 to 10 mm of cancer cells) include, without limitation, nucleic acids, polypeptides (e.g., CXCL14 polypeptides such as CXCL14 polypeptide conjugated to antibodies having the ability to bind to cancer cells), and small molecules, and pharmaceutically acceptable salts of a small molecule.

In some cases when treating a mammal (e.g., a human) having cancer, the mammal can be administered or instructed to self-administer any one or more CXCL14 polypeptides. Any appropriate CXCL14 polypeptide (and/or nucleic acid designed to encode a CXCL14 polypeptide) can be administered to a mammal (e.g., a human) having cancer as described herein. Examples of CXCL14 polypeptides and nucleic acids encoding CXCL14 polypeptides include, without limitation, human CXCL14 polypeptides, nucleic acids encoding a human CXCL14 polypeptide, and those set forth in the National Center for Biotechnology Information (NCBI) databases at, for example, accession no. Q548T5, accession no. Q91V02, accession no. Q9JHH7, and accession no. B3KQU8.

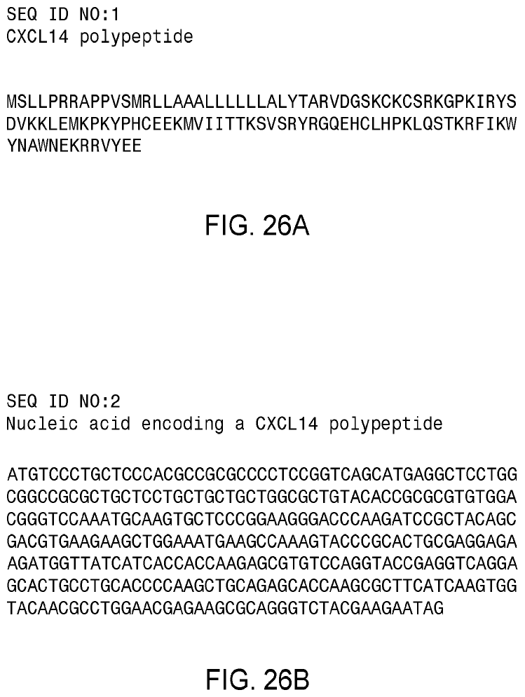

In some cases, a CXCL14 polypeptide can have an amino acid sequence set forth in SEQ ID NO:1 (see, e.g., FIG. 26A). In some cases, a nucleic acid encoding a CXCL14 polypeptide can have an nucleotide sequence set forth in SEQ ID NO:2 (see, e.g., FIG. 26B).

In some cases, a variant of a CXCL14 polypeptide can be used in place of or in addition to a CXCL14 polypeptide. A variant of a CXCL14 polypeptide can have the amino acid sequence of a naturally-occurring CXCL14 polypeptide with one or more (e.g., e.g., one, two, three, four, five, six, seven, eight, nine, ten, or more) amino acid deletions, additions, substitutions, or combinations thereof, provided that the variant retains the function of a naturally-occurring CXCL14 polypeptide (e.g., to recruit macrophages).

Amino acid substitutions can be made, in some cases, by selecting substitutions that do not differ significantly in their effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of substitutions that can be used herein for SEQ ID NO: 1 include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

In some cases, a variant of a CXCL14 polypeptide can be designed to include the amino acid sequence set forth in SEQ ID NO: 1 with one or more (e.g., one, two, three, four, five, six, or more) non-conservative substitutions. Non-conservative substitutions typically entail exchanging a member of one of the classes described above for a member of another class. Whether an amino acid change results in a functional polypeptide can be determined by assaying the specific activity of the polypeptide using, for example, the methods described herein.

In some cases, a variant of a CXCL14 polypeptide having an amino acid sequence with at least 85% (e.g., at least 86%, 87%, 88%, 89%, 90%, 91%, 92%, 93%, 94%, 95%, 96%, 97%, 98%, or 99.0%) sequence identity to the amino acid sequence set forth in SEQ ID NO: 1, provided that it includes at least one amino acid addition, deletion, or substitution with respect to SEQ ID NO: 1, can be used as described herein. Percent sequence identity is calculated by determining the number of matched positions in aligned amino acid sequences, dividing the number of matched positions by the length of an aligned amino acid sequence, and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number (e.g., SEQ ID NO:1) is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq-i c:\seq1.txt-j c:\seq2.txt-p blastn-o c:\output.txt-q-1-r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches by the length of the sequence set forth in the identified sequence (e.g., SEQ ID NO:1), followed by multiplying the resulting value by 100. For example, an amino acid sequence that has 106 matches when aligned with the sequence set forth in SEQ ID NO: 1 is 95 percent identical to the sequence set forth in SEQ ID NO:1 (i.e., 106÷111×100=95.5). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

In some cases, one or more (e.g., one, two, three, four, or more) agents that can increase a level of a PASP polypeptide other than a CXCL14 polypeptide expressed by cancer cells and/or that can increase the presence of a PASP polypeptide other than a CXCL14 polypeptide within the location of cancer cells (e.g., within 1 to 10 mm of a tumor) can be administered to a mammal (e.g., a human) in need thereof (e.g., a human having cancer) can be used in place of or in addition to one or more (e.g., one, two, three, four, or more) agents that can increase a level of a CXCL14 polypeptide expressed by cancer cells and/or that can increase the presence of a CXCL14 polypeptide within the location of cancer cells (e.g., within 1 to 10 mm of a tumor) can be administered to a mammal (e.g., a human) in need thereof (e.g., a human having cancer). Examples of PASP polypeptides other than a CXCL14 polypeptide include, without limitation, IL-34 polypeptides, IL-7 polypeptides, and CCL17 polypeptides. In some cases, a PASP polypeptide other than a CXCL14 polypeptide can be as described in Example 1.

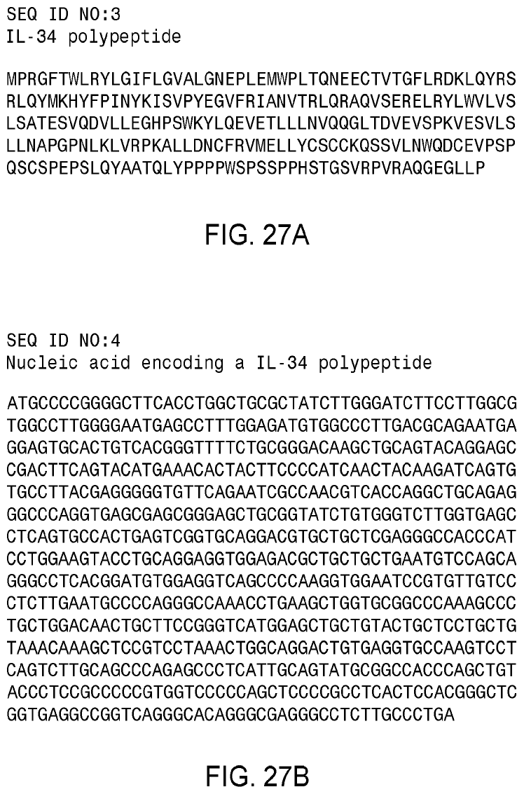

When a PASP polypeptide other than a CXCL14 polypeptide is an IL-34 polypeptide, the IL-34 polypeptide can be any appropriate IL-34 polypeptide. Examples of IL-34 polypeptides and nucleic acids encoding IL-34 polypeptides include, without limitation, human IL-34 polypeptides, nucleic acids encoding a human IL-34 polypeptide, and those set forth in the NCBI databases at, for example, accession no. P13232-1 and accession no. NP_000871.1. In some cases, an IL-34 polypeptide can have an amino acid sequence set forth in SEQ ID NO:3 (see, e.g., FIG. 27A). In some cases, a nucleic acid encoding an IL-34 polypeptide can have an nucleotide sequence set forth in SEQ ID NO:4 (see, e.g., FIG. 27B).

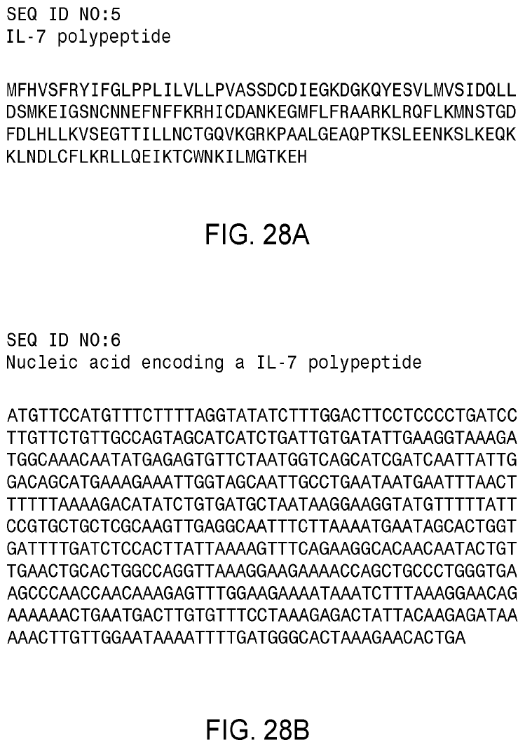

When a PASP polypeptide other than a CXCL14 polypeptide is an IL-7 polypeptide, the IL-7 polypeptide can be any appropriate IL-7 polypeptide. Examples of IL-7 polypeptides and nucleic acids encoding IL-7 polypeptides include, without limitation, human IL-7 polypeptides, nucleic acids encoding a human IL-7 polypeptide, and those set forth in the NCBI databases at, for example, accession no. Q6ZMJ4, accession no. NP_689669, and accession no. NP_001166243. In some cases, an IL-7 polypeptide can have an amino acid sequence set forth in SEQ ID NO:5 (see, e.g., FIG. 28A). In some cases, a nucleic acid encoding an IL-7 polypeptide can have an nucleotide sequence set forth in SEQ ID NO: 6 (see, e.g., FIG. 28B).

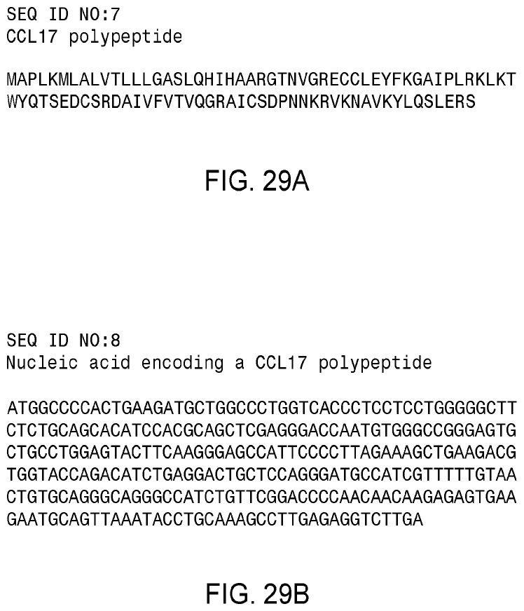

When a PASP polypeptide other than a CXCL14 polypeptide is a CCL17 polypeptide, the CCL17 polypeptide can be any appropriate CCL17 polypeptide. Examples of CCL17 polypeptides and nucleic acids encoding CCL17 polypeptides include, without limitation, human CCL17 polypeptides, nucleic acids encoding a human CCL17 polypeptide, and those set forth in the NCBI databases at, for example, accession no. Q92583 and accession no. NP_002978. In some cases, a CCL17 polypeptide can have an amino acid sequence set forth in SEQ ID NO:7 (see, e.g., FIG. 29A). In some cases, a nucleic acid encoding an IL-7 polypeptide can have an nucleotide sequence set forth in SEQ ID NO:8 (see, e.g., FIG. 29B).

Any appropriate method can be used to deliver one or more CXCL14 polypeptides (and/or nucleic acids designed to encode a CXCL14 polypeptide) to a mammal. In some cases, when one or more CXCL14 polypeptides (and/or nucleic acids designed to encode a CXCL14 polypeptide) are administered to a mammal (e.g., a human), the one or more CXCL14 polypeptides (and/or nucleic acids designed to encode a CXCL14 polypeptide) can be administered to one or more cancer cells within a mammal (e.g., a human) having cancer. In some cases, when one or more CXCL14 polypeptides (and/or nucleic acids designed to encode a CXCL14 polypeptide) are administered to a mammal (e.g., a human), the one or more CXCL14 polypeptides (and/or nucleic acids designed to encode a CXCL14 polypeptide) can be administered to a tumor site (e.g., a tumor microenvironment) within a mammal (e.g., a human) having cancer.

Any appropriate method can be used to obtain a CXCL14 polypeptide. For example, a CXCL14 polypeptide can be obtained by synthesizing the polypeptide of interest using appropriate polypeptide synthesizing techniques.

When one or more nucleic acids designed to encode a CXCL14 polypeptide are administered to a mammal (e.g., a human), the nucleic acid can be in the form of a vector (e.g., a viral vector or a non-viral vector).

When nucleic acid encoding a CXCL14 polypeptide is administered to a mammal, the nucleic acid can be used for transient expression of a CXCL14 polypeptide or for stable expression of a CXCL14 polypeptide. In cases where a nucleic acid encoding a CXCL14 polypeptide is used for stable expression of a CXCL14 polypeptide, the nucleic acid encoding a CXCL14 polypeptide can be engineered to integrate into the genome of a cell. Nucleic acid can be engineered to integrate into the genome of a cell using any appropriate method. For example, gene editing techniques (e.g., CRISPR or TALEN gene editing) can be used to integrate nucleic acid designed to encode a CXCL14 polypeptide into the genome of a cell.

When a vector used to deliver nucleic acid encoding a CXCL14 polypeptide to a mammal (e.g., a human) is a viral vector, any appropriate viral vector can be used. A viral vector can be derived from a positive-strand virus or a negative-strand virus. A viral vector can be derived from a virus with a DNA genome or a RNA genome. In some cases, a viral vector can be a chimeric viral vector. In some cases, a viral vector can infect dividing cells. In some cases, a viral vector can infect non-dividing cells. Examples virus-based vectors that can be used to deliver nucleic acid encoding a CXCL14 polypeptide to a mammal (e.g., a human) include, without limitation, virus-based vectors based on adenoviruses, AAVs, Sendai viruses, retroviruses, or lentiviruses.

When a vector used to deliver nucleic acid encoding a CXCL14 polypeptide to a mammal (e.g., a human) is a non-viral vector, any appropriate non-viral vector can be used. In some cases, a non-viral vector can be an expression plasmid (e.g., a cDNA expression vector).

In addition to nucleic acid encoding a CXCL14 polypeptide, a vector (e.g., a viral vector or a non-viral vector) can contain one or more regulatory elements operably linked to the nucleic acid encoding a CXCL14 polypeptide. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector depends on several factors, including, without limitation, inducibility, targeting, and the level of expression desired. For example, a promoter can be included in a vector to facilitate transcription of a nucleic acid encoding a CXCL14 polypeptide. A promoter can be a naturally occurring promoter or a recombinant promoter. A promoter can be ubiquitous or inducible (e.g., in the presence of tetracycline), and can affect the expression of a nucleic acid encoding a polypeptide in a general or tissue-specific manner. Examples of promoters that can be used to drive expression of a CXCL14 polypeptide in cells include, without limitation, PGK promoters, CMV promoters, and CAGS promoters. As used herein, “operably linked” refers to positioning of a regulatory element in a vector relative to a nucleic acid encoding a polypeptide in such a way as to permit or facilitate expression of the encoded polypeptide. For example, a vector can contain a promoter and nucleic acid encoding a CXCL14 polypeptide. In this case, the promoter is operably linked to a nucleic acid encoding a CXCL14 polypeptide such that it drives expression of the CXCL14 polypeptide in cells.

In some cases, expression of a CXCL14 polypeptide delivered using nucleic acid can be directed to cancer cells using one or more regulatory elements (e.g., promotors such as cancer-specific promotors; microRNA target sequences that are blocked or degraded in non-cancer cells to prevent expression in those non-cancer cells; or protein degradation sequences active in normal cells but not in cancer cells (e.g., ubiquitin-mediated degradation)) to regulate the expression of a CXCL14 polypeptide within cancer cells. Examples of cancer-specific promotors include, without limitation, APF promotors for hepatocellular cancer cells and CEA promotors for epithelial cancer cells.

Nucleic acid encoding a CXCL14 polypeptide can be produced by techniques including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., cDNA, genomic DNA, or RNA) encoding a CXCL14 polypeptide.

In some cases when treating a mammal (e.g., a human) having cancer, the mammal can be administered or instructed to self-administer any one or more gene therapy components designed for targeted gene activation of nucleic acid encoding a CXCL14 polypeptide (e.g., the endogenous Cxcl14 gene) to increase the level of CXCL14 polypeptides expressed by cancer cells and/or within the vicinity of cancer cells (e.g., within 1 to 10 mm of cancer cells). Gene therapy components designed for targeted gene activation of nucleic acid encoding a CXCL14 polypeptide (e.g., the endogenous Cxcl14 gene) to increase the level of CXCL14 polypeptides expressed by cancer cells and/or within the vicinity of cancer cells (e.g., within 1 to 10 mm of cancer cells) can be part of any appropriate targeted gene activation system. Examples of targeted gene activation systems that can be designed to increase expression of nucleic acid encoding a CXCL14 polypeptide include, without limitation, clustered regularly interspaced short palindromic repeat (CRISPR)-Cas9-based targeted gene activation (CRISPRa) and demethylating enzymes. For example, one or more nucleic acid molecules designed to encode the components of a targeted gene activation system designed to activate transcription of nucleic acid encoding a CXCL14 polypeptide (e.g., the endogenous Cxcl14 gene) can be administered to a mammal (e.g., a human) having cancer to increase the level of CXCL14 polypeptides expressed by cancer cells and/or within the vicinity of cancer cells (e.g., within 1 to 10 mm of cancer cells). For example, one or more the components of a targeted gene activation system designed to activate transcription of nucleic acid encoding a CXCL14 polypeptide (e.g., the endogenous Cxcl14 gene) can be administered to a mammal (e.g., a human) having cancer to increase the level of CXCL14 polypeptides expressed by cancer cells and/or within the vicinity of cancer cells (e.g., within 1 to 10 mm of cancer cells). In some cases, a targeted gene activation system can include (a) a fusion polypeptide including a deactivated Cas (dCas) polypeptide and a transcriptional activator polypeptide, (b) one or more helper activator polypeptides, and (c) a nucleic acid molecule including (i) a nucleic acid sequence that is complementary to a target sequence within a Cxcl14 gene, and (ii) a nucleic acid sequence that can bind the one or more helper activator polypeptides. For example, nucleic acid designed to increase a level of CXCL14 polypeptides within a mammal can include (a) nucleic acid that can encode a fusion polypeptide including a dCas polypeptide and a transcriptional activator polypeptide, (b) nucleic acid that can encode one or more helper activator polypeptides, and (c) nucleic acid that can encode a nucleic acid molecule including (i) a nucleic acid sequence that is complementary to a target sequence within a Cxcl14 gene, and (ii) a nucleic acid sequence that can bind the one or more helper activator polypeptides.

A fusion polypeptide including a dCas polypeptide and a transcriptional activator polypeptide in a targeted gene activation system designed to activate transcription of a Cxcl14 gene (e.g., resulting in an increased level of CXCL14 polypeptides) can include any appropriate dCas polypeptide. Examples of dCas polypeptides that can be included in a fusion polypeptide including a dCas polypeptide and a transcriptional activator polypeptide that can be used as a targeted gene activation system designed to activate transcription of a Cxcl14 gene can include, without limitation, deactivated Cas9 (dCas9) polypeptides (e.g., deactivated Streptococcus pyogenes Cas9 (dSpCas9), deactivated Staphylococcus aureus Cas9 (dSaCas9), and deactivated Campylobacter jejuni Cas9 (dCjCas9)), and deactivated Cas phi (dCasΦ) polypeptides.

A fusion polypeptide including a dCas polypeptide and a transcriptional activator polypeptide in a targeted gene activation system designed to activate transcription of a Cxcl14 gene (e.g., resulting in an increased level of CXCL14 polypeptides) can include any appropriate transcriptional activator polypeptide. In some cases, a transcriptional activator polypeptide can recruit an RNA polymerase. In some cases, a transcriptional activator polypeptide can recruit one or more transcription factors and/or transcription co-factors (e.g., RNA polymerase co-factors). Examples of transcriptional activator polypeptides that can be included in a fusion polypeptide including a dCas polypeptide and a transcriptional activator polypeptide that can be used in a targeted gene activation system designed to activate transcription of a Cxcl14 gene can include, without limitation, dCAS9, VP64, dCAS-VPR, and dCAS9-SAM.

A fusion polypeptide including a dCas polypeptide and a transcriptional activator polypeptide in a targeted gene activation system designed to activate transcription of a Cxcl14 gene (e.g., resulting in an increased level of CXCL14 polypeptides) can include the dCas polypeptide and the transcriptional activator polypeptide in any orientation. In some cases, a transcriptional activator polypeptide can be fused to the N-terminus of a dCas polypeptide. In some cases, a transcriptional activator polypeptide can be fused to the C-terminus of a dCas polypeptide.

A targeted gene activation system designed to activate transcription of a Cxcl14 gene (e.g., resulting in an increased level of CXCL14 polypeptides) can include any appropriate helper activator polypeptide. Examples of helper activator polypeptides that can be used in a targeted gene activation system designed to activate transcription of a Cxcl14 gene can include, without limitation, dCAS9-CBP, SunTag-VP64, and SunTag-VPR. In some cases, a helper activator polypeptide can include two or more (e.g., two, three, or more) helper activator polypeptides. For example, a helper activator polypeptide can be a fusion polypeptide including two or more helper activator polypeptides. For example, a helper activator polypeptide can be a complex including two or more helper activator polypeptide.

A targeted gene activation system designed to activate transcription of a Cxcl14 gene (e.g., resulting in an increased level of CXCL14 polypeptides) can include any appropriate nucleic acid molecule including (i) a nucleic acid sequence that is complementary to a target sequence within a Cxcl14 gene, and (ii) a nucleic acid sequence that can bind the helper activator polypeptide. In some cases, a nucleic acid molecule including (i) a nucleic acid sequence that is complementary to a target sequence within a Cxcl14 gene, and (ii) a nucleic acid sequence that can bind the helper activator polypeptide that can be used in a targeted gene activation system designed to activate transcription of a Cxcl14 gene can include a nucleic acid sequence that is complementary to a target sequence within a Cxcl14 gene. A nucleic acid sequence that is complementary to a target sequence within a Cxcl14 gene can include any appropriate nucleic acid sequence. A nucleic acid sequence that is complementary to a target sequence within a Cxcl14 gene can be complementary to (e.g., can be designed to target) any target sequence within a Cxcl14 gene (e.g., can target any location within a Cxcl14 gene). In some cases, a nucleic acid sequence that is complementary to a target sequence within a Cxcl14 gene can be a single stranded nucleic acid sequence. In some cases, a target sequence within a Cxcl14 gene can be in a promoter sequence of the Cxcl14 gene. Examples of nucleic acid sequences that are complementary to a target sequence within a Cxcl14 gene include, without limitation, nucleic acid sequences that can be encoded by a nucleic acid sequence including the sequence CAGCCCTGGGCATCCACCGACAGACAGCCCTGGGCATCCACCGACGGCGCCGG (SEQ ID NO:9) and a nucleic acid sequence including the sequence GCACGGCCACAGACAGCCCTCAGCGCACGGCCACAGACAGCCCTGGGCATGGG (SEQ ID NO:10).

In some cases, a nucleic acid molecule including (i) a nucleic acid sequence that is complementary to a target sequence within a Cxcl14 gene, and (ii) a nucleic acid sequence that can bind the helper activator polypeptide that can be used in a targeted gene activation system designed to activate transcription of a Cxcl14 gene can include any appropriate nucleic acid sequence that can bind the helper activator polypeptide.

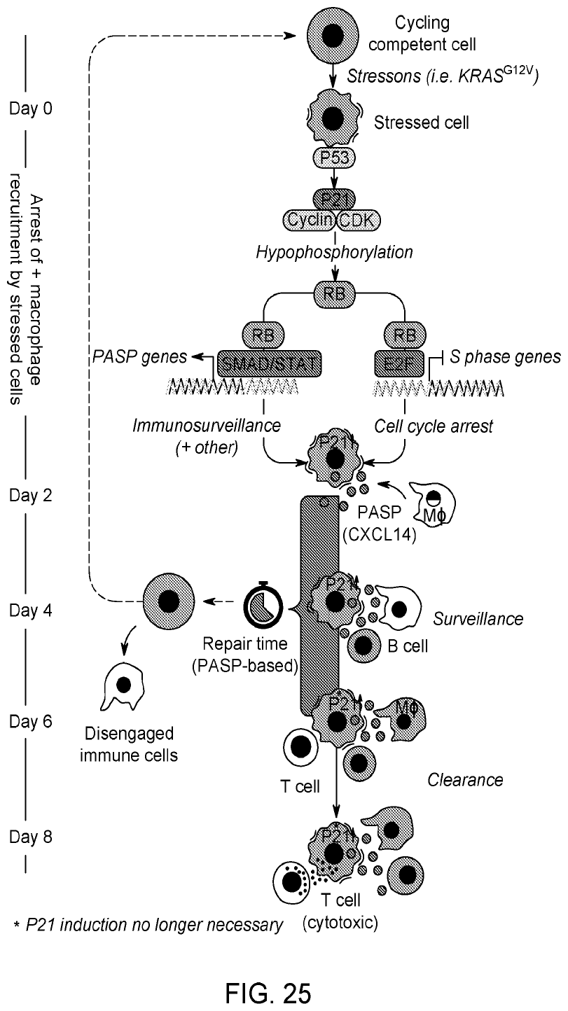

In some cases when treating a mammal (e.g., a human) having cancer, the mammal can be administered or instructed to self-administer any one or more agents that can modulate a PASP pathway to increase the level of CXCL14 polypeptides expressed by cancer cells and/or within the vicinity of cancer cells (e.g., within 1 to 10 mm of cancer cells). Any appropriate agent that can modulate a PASP pathway to increase the level of CXCL14 polypeptides expressed by cancer cells and/or within the vicinity of cancer cells (e.g., within 1 to 10 mm of cancer cells) can be administered to a mammal (e.g., a human) having cancer as described herein. In some cases, an agent that can modulate a PASP pathway to increase the level of CXCL14 polypeptides expressed by cancer cells and/or within the vicinity of cancer cells (e.g., within 1 to 10 mm of cancer cells) can increase a level of a p21 polypeptide. In some cases, an agent that can modulate a PASP pathway to increase the level of CXCL14 polypeptides expressed by cancer cells and/or within the vicinity of cancer cells (e.g., within 1 to 10 mm of cancer cells) can inhibit phosphorylation of a RB polypeptide. In some cases, an agent that can modulate a PASP pathway to increase the level of CXCL14 polypeptides expressed by cancer cells and/or within the vicinity of cancer cells (e.g., within 1 to 10 mm of cancer cells) can be a hypophosphorylated RB polypeptide. In some cases, an agent that can modulate a PASP pathway to increase the level of CXCL14 polypeptides expressed by cancer cells and/or within the vicinity of cancer cells (e.g., within 1 to 10 mm of cancer cells) can target a polypeptide shown in FIG. 25 that is upstream of a CXCL14 polypeptide.

When an agent that can modulate a PASP pathway to increase the level of CXCL14 polypeptides expressed by cancer cells and/or within the vicinity of cancer cells (e.g., within 1 to 10 mm of cancer cells) can increase a level of a p21 polypeptide, any appropriate agent that can increase a level of a p21 polypeptide can be administered to a mammal (e.g., a human) having cancer. For example, one or more p21 polypeptides (and/or nucleic acid designed to encode a p21 polypeptide) can be administered to a mammal (e.g., a human) having cancer as described herein. Examples of p21 polypeptides and nucleic acids encoding p21 polypeptides include, without limitation, those set forth in the NCBI databases at, for example, accession no. P38936 and accession no. 39689.

Any appropriate method can be used to deliver one or more p21 polypeptides (and/or nucleic acids designed to encode a p21 polypeptide) to a mammal. In some cases, when one or more p21 polypeptides (and/or nucleic acids designed to encode a p21 polypeptide) are administered to a mammal (e.g., a human), the one or more p21 polypeptides (and/or nucleic acids designed to encode a p21 polypeptide) can be administered to one or more cancer cells within a mammal (e.g., a human) having cancer. In some cases, when one or more p21 polypeptides (and/or nucleic acids designed to encode a p21 polypeptide) are administered to a mammal (e.g., a human), the one or more p21 polypeptides (and/or nucleic acids designed to encode a p21 polypeptide) can be administered to a tumor site (e.g., a tumor microenvironment) within a mammal (e.g., a human) having cancer.

Any appropriate method can be used to obtain a p21 polypeptide. For example, a p21 polypeptide can be obtained by synthesizing the polypeptide of interest using appropriate polypeptide synthesizing techniques.

When one or more nucleic acids designed to encode a p21 polypeptide are administered to a mammal (e.g., a human), the nucleic acid can be in the form of a vector (e.g., a viral vector or a non-viral vector).

When nucleic acid encoding a p21 polypeptide is administered to a mammal, the nucleic acid can be used for transient expression of a p21 polypeptide or for stable expression of a p21 polypeptide. In cases where a nucleic acid encoding a p21 polypeptide is used for stable expression of a p21 polypeptide, the nucleic acid encoding a p21 polypeptide can be engineered to integrate into the genome of a cell. Nucleic acid can be engineered to integrate into the genome of a cell using any appropriate method. For example, gene editing techniques (e.g., CRISPR or TALEN gene editing) can be used to integrate nucleic acid designed to encode a p21 polypeptide into the genome of a cell.

When a vector used to deliver nucleic acid encoding a p21 polypeptide to a mammal (e.g., a human) is a viral vector, any appropriate viral vector can be used. A viral vector can be derived from a positive-strand virus or a negative-strand virus. A viral vector can be derived from a virus with a DNA genome or a RNA genome. In some cases, a viral vector can be a chimeric viral vector. In some cases, a viral vector can infect dividing cells. In some cases, a viral vector can infect non-dividing cells. Examples virus-based vectors that can be used to deliver nucleic acid encoding a p21 polypeptide to a mammal (e.g., a human) include, without limitation, virus-based vectors based on adenoviruses, AAVs, Sendai viruses, retroviruses, or lentiviruses.

When a vector used to deliver nucleic acid encoding a p21 polypeptide to a mammal (e.g., a human) is a non-viral vector, any appropriate non-viral vector can be used. In some cases, a non-viral vector can be an expression plasmid (e.g., a cDNA expression vector).

In addition to nucleic acid encoding a p21 polypeptide, a vector (e.g., a viral vector or a non-viral vector) can contain one or more regulatory elements operably linked to the nucleic acid encoding a p21 polypeptide. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector depends on several factors, including, without limitation, inducibility, targeting, and the level of expression desired. For example, a promoter can be included in a vector to facilitate transcription of a nucleic acid encoding a p21 polypeptide. A promoter can be a naturally occurring promoter or a recombinant promoter. A promoter can be ubiquitous or inducible (e.g., in the presence of tetracycline), and can affect the expression of a nucleic acid encoding a polypeptide in a general or tissue-specific manner. Examples of promoters that can be used to drive expression of a p21 polypeptide in cells include, without limitation, CMV promoters, PGK promoters, and CAGS promoters. For example, a vector can contain a promoter and nucleic acid encoding a p21 polypeptide. In this case, the promoter is operably linked to a nucleic acid encoding a p21 polypeptide such that it drives expression of the p21 polypeptide in cells.

In some cases, expression of a p21 polypeptide delivered using nucleic acid can be directed to cancer cells using one or more regulatory elements (e.g., promotors such as cancer-specific promotors; microRNA target sequences that are blocked or degraded in non-cancer cells to prevent expression in those non-cancer cells; or protein degradation sequences active in normal cells but not in cancer cells (e.g., ubiquitin-mediated degradation)) to regulate the expression of a p21 polypeptide within cancer cells. Examples of cancer-specific promotors include, without limitation, APF promotors for hepatocellular cancer cells and CEA promotors for epithelial cancer cells.

Nucleic acid encoding a p21 polypeptide can be produced by techniques including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a p21 polypeptide.

When one or more gene therapy components designed for targeted gene activation of nucleic acid encoding a p21 polypeptide (e.g., the endogenous Cdkn1a gene) to increase the level of p21 polypeptides expressed by cancer cells and/or within the vicinity of cancer cells (e.g., within 1 to 10 mm of cancer cells). Gene therapy components designed for targeted gene activation of nucleic acid encoding a p21 polypeptide (e.g., the endogenous Cdkn1a gene) to increase the level of p21 polypeptides expressed by cancer cells and/or within the vicinity of cancer cells (e.g., within 1 to 10 mm of cancer cells) can be part of any appropriate targeted gene activation system. Examples of targeted gene activation systems that can be designed to increase expression of nucleic acid encoding a p21 polypeptide include, without limitation, CRISPRa and demethylating enzymes. For example, one or more nucleic acid molecules designed to encode the components of a targeted gene activation system designed to activate transcription of nucleic acid encoding a p21 polypeptide (e.g., the endogenous Cdkn1a gene) can be administered to a mammal (e.g., a human) having cancer to increase the level of p21 polypeptides expressed by cancer cells and/or within the vicinity of cancer cells (e.g., within 1 to 10 mm of cancer cells). For example, one or more the components of a targeted gene activation system designed to activate transcription of nucleic acid encoding a p21 polypeptide (e.g., the endogenous Cdkn1a gene) can be administered to a mammal (e.g., a human) having cancer to increase the level of p21 polypeptides expressed by cancer cells and/or within the vicinity of cancer cells (e.g., within 1 to 10 mm of cancer cells). In some cases, a targeted gene activation system can include (a) a fusion polypeptide including a dCas polypeptide and a transcriptional activator polypeptide, (b) one or more helper activator polypeptides, and (c) a nucleic acid molecule including (i) a nucleic acid sequence that is complementary to a target sequence within a Cdkn1a gene, and (ii) a nucleic acid sequence that can bind the one or more helper activator polypeptides. For example, nucleic acid designed to increase a level of p21 polypeptides within a mammal can include (a) nucleic acid that can encode a fusion polypeptide including a dCas polypeptide and a transcriptional activator polypeptide, (b) nucleic acid that can encode one or more helper activator polypeptides, and (c) nucleic acid that can encode a nucleic acid molecule including (i) a nucleic acid sequence that is complementary to a target sequence within a Cdkn1a gene, and (ii) a nucleic acid sequence that can bind the one or more helper activator polypeptides.

A fusion polypeptide including a dCas polypeptide and a transcriptional activator polypeptide in a targeted gene activation system designed to activate transcription of a Cdkn1a gene (e.g., resulting in an increased level of p21 polypeptides) can include any appropriate dCas polypeptide. Examples of dCas polypeptides that can be included in a fusion polypeptide including a dCas polypeptide and a transcriptional activator polypeptide that can be used as a targeted gene activation system designed to activate transcription of a Cxcl14 gene can include, without limitation, dCas9 polypeptides (e.g., dSpCas9, dSaCas9, and dCjCas9), and dCasΦ polypeptides.

A fusion polypeptide including a dCas polypeptide and a transcriptional activator polypeptide in a targeted gene activation system designed to activate transcription of a Cdkn1a gene (e.g., resulting in an increased level of p21 polypeptides) can include any appropriate transcriptional activator polypeptide. In some cases, a transcriptional activator polypeptide can recruit an RNA polymerase. In some cases, a transcriptional activator polypeptide can recruit one or more transcription factors and/or transcription co-factors (e.g., RNA polymerase co-factors). Examples of transcriptional activator polypeptides that can be included in a fusion polypeptide including a dCas polypeptide and a transcriptional activator polypeptide that can be used in a targeted gene activation system designed to activate transcription of a Cdkn1a gene can include, without limitation, dCAS9, VP64, dCAS-VPR, and dCAS9-SAM.

A fusion polypeptide including a dCas polypeptide and a transcriptional activator polypeptide in a targeted gene activation system designed to activate transcription of a Cdkn1a gene (e.g., resulting in an increased level of p21 polypeptides) can include the dCas polypeptide and the transcriptional activator polypeptide in any orientation. In some cases, a transcriptional activator polypeptide can be fused to the N-terminus of a dCas polypeptide. In some cases, a transcriptional activator polypeptide can be fused to the C-terminus of a dCas polypeptide.

A targeted gene activation system designed to activate transcription of a Cdkn1a gene (e.g., resulting in an increased level of p21 polypeptides) can include any appropriate helper activator polypeptide. Examples of helper activator polypeptides that can be used in a targeted gene activation system designed to activate transcription of a Cdkn1a gene can include, without limitation, dCAS9-CBP, SunTag-VP64, and SunTag-VPR. In some cases, a helper activator polypeptide can include two or more (e.g., two, three, or more) helper activator polypeptides. For example, a helper activator polypeptide can be a fusion polypeptide including two or more helper activator polypeptides. For example, a helper activator polypeptide can be a complex including two or more helper activator polypeptide.

A targeted gene activation system designed to activate transcription of a Cdkn1a gene (e.g., resulting in an increased level of p21 polypeptides) can include any appropriate nucleic acid molecule including (i) a nucleic acid sequence that is complementary to a target sequence within a Cdkn1a gene, and (ii) a nucleic acid sequence that can bind the helper activator polypeptide. In some cases, a nucleic acid molecule including (i) a nucleic acid sequence that is complementary to a target sequence within a Cdkn1a gene, and (ii) a nucleic acid sequence that can bind the helper activator polypeptide that can be used in a targeted gene activation system designed to activate transcription of a Cdkn1a gene can include a nucleic acid sequence that is complementary to a target sequence within a Cdkn1a gene. A nucleic acid sequence that is complementary to a target sequence within a Cdkn1a gene can include any appropriate nucleic acid sequence. A nucleic acid sequence that is complementary to a target sequence within a Cdkn1a gene can be complementary to (e.g., can be designed to target) any target sequence within a Cdkn1a gene (e.g., can target any location within a Cdkn1a gene). In some cases, a nucleic acid sequence that is complementary to a target sequence within a Cdkn1a gene can be a single stranded nucleic acid sequence. In some cases, a target sequence within a Cdkn1a gene can be in a promoter sequence of the Cdkn1a gene. Examples of nucleic acid sequences that are complementary to a target sequence within a Cdkn1a gene include, without limitation, nucleic acid sequences that can be encoded by a nucleic acid sequence including the sequence AGCTGGGCGCGGATTCGCCGCCGGAGCTGGGCGCGGATTCGCCGAGGCACAGG (SEQ ID NO:11) and a nucleic acid sequence including the sequence GCGGATTCGCCGAGGCACCGGGGCGCGGATTCGCCGAGGCACCGAGGCACAGG (SEQ ID NO:12).

In some cases, a nucleic acid molecule including (i) a nucleic acid sequence that is complementary to a target sequence within a Cdkn1a gene, and (ii) a nucleic acid sequence that can bind the helper activator polypeptide that can be used in a targeted gene activation system designed to activate transcription of a Cdkn1a gene can include any appropriate nucleic acid sequence that can bind the helper activator polypeptide.

When an agent that can modulate a PASP pathway to increase the level of CXCL14 polypeptides expressed by cancer cells and/or within the vicinity of cancer cells (e.g., within 1 to 10 mm of cancer cells) can inhibit (e.g., reduce or prevent) phosphorylation of a RB polypeptide, any appropriate inhibitor of phosphorylation of a RB polypeptide can be administered to a mammal (e.g., a human) having cancer. Examples of inhibitors of phosphorylation of a RB polypeptide include, without limitation, inhibitors of a CDK2 polypeptide, inhibitors of a CDK4 polypeptide, and inhibitors of a CDK6 polypeptide.

When an inhibitor of phosphorylation of a RB polypeptide is an inhibitor of a CDK2 polypeptide, any appropriate inhibitor of a CDK2 polypeptide can be administered to a mammal (e.g., a human) having cancer. An inhibitor of a CDK2 polypeptide can be an inhibitor of CDK2 polypeptide activity (e.g., anti-CDK2 antibodies such as neutralizing anti-CDK2 antibodies and small molecules that target a CDK2 polypeptide) or an inhibitor of CDK2 polypeptide expression (e.g., nucleic acid molecules designed to induce RNA interference of CDK2 polypeptide expression such as siRNA molecules and shRNA molecules). Examples of inhibitors of a CDK2 polypeptide include, without limitation, dinaciclib, GW8510, and seliciclib. In some cases, an inhibitor of a CDK2 polypeptide can be as described elsewhere (see, e.g., Sabnis et al., ACS Med. Chem. Lett., 11 (12): 2346-2347 (2020); and Al-Sanea et al., Molecules 26 (2): 412 (2021)).

When an agent that can modulate a PASP pathway to increase the level of CXCL14 polypeptides expressed by cancer cells and/or within the vicinity of cancer cells (e.g., within 1 to 10 mm of cancer cells) is a hypophosphorylated RB polypeptide, any appropriate hypophosphorylated RB polypeptide can be administered to a mammal (e.g., a human) having cancer. For example, one or more hypophosphorylated RB polypeptides (and/or nucleic acid designed to encode a hypophosphorylated RB polypeptide) can be administered to a mammal (e.g., a human) having cancer as described herein. In some cases, a hypophosphorylated RB polypeptide can have one or more phosphorylation sites within a RB polypeptide modified such that the RB polypeptide has reduced or eliminated phosphorylation (e.g., as compared to a RB polypeptide that lacks the one or more modifications). Examples of phosphorylation sites that can be modified such that a RB polypeptide has reduced or eliminated phosphorylation (e.g., as compared to a RB polypeptide that lacks the one or more modifications) include, without limitation, S230, S249, S232, T356, T373, S608, S612, S780, S788, S795, S807, S811, T821, and T826. Examples of hypophosphorylated RB polypeptides and nucleic acids encoding hypophosphorylated RB polypeptides include, without limitation, those set forth in the NCBI databases at, for example, accession no. P1305, accession no. P06400, accession no. P33568.

Any appropriate method can be used to deliver one or more hypophosphorylated RB polypeptides (and/or nucleic acids designed to encode a hypophosphorylated RB polypeptide) to a mammal. In some cases, when one or more hypophosphorylated RB polypeptides (and/or nucleic acids designed to encode a hypophosphorylated RB polypeptide) are administered to a mammal (e.g., a human), the one or more hypophosphorylated RB polypeptides (and/or nucleic acids designed to encode a hypophosphorylated RB polypeptide) can be administered to one or more cancer cells within a mammal (e.g., a human) having cancer. In some cases, when one or more hypophosphorylated RB polypeptides (and/or nucleic acids designed to encode a hypophosphorylated RB polypeptide) are administered to a mammal (e.g., a human), the one or more hypophosphorylated RB polypeptides (and/or nucleic acids designed to encode a hypophosphorylated RB polypeptide) can be administered to a tumor site (e.g., a tumor microenvironment) within a mammal (e.g., a human) having cancer.

Any appropriate method can be used to obtain a hypophosphorylated RB polypeptide. For example, a hypophosphorylated RB polypeptide can be obtained by synthesizing the polypeptide of interest using appropriate polypeptide synthesizing techniques.

When one or more nucleic acids designed to encode a hypophosphorylated RB polypeptide are administered to a mammal (e.g., a human), the nucleic acid can be in the form of a vector (e.g., a viral vector or a non-viral vector).

When nucleic acid encoding a hypophosphorylated RB polypeptide is administered to a mammal, the nucleic acid can be used for transient expression of a hypophosphorylated RB polypeptide or for stable expression of a hypophosphorylated RB polypeptide. In cases where a nucleic acid encoding a hypophosphorylated RB polypeptide is used for stable expression of a hypophosphorylated RB polypeptide, the nucleic acid encoding a hypophosphorylated RB polypeptide can be engineered to integrate into the genome of a cell. Nucleic acid can be engineered to integrate into the genome of a cell using any appropriate method. For example, gene editing techniques (e.g., CRISPR or TALEN gene editing) can be used to integrate nucleic acid designed to encode a hypophosphorylated RB polypeptide into the genome of a cell.

When a vector used to deliver nucleic acid encoding a hypophosphorylated RB polypeptide to a mammal (e.g., a human) is a viral vector, any appropriate viral vector can be used. A viral vector can be derived from a positive-strand virus or a negative-strand virus. A viral vector can be derived from a virus with a DNA genome or a RNA genome. In some cases, a viral vector can be a chimeric viral vector. In some cases, a viral vector can infect dividing cells. In some cases, a viral vector can infect non-dividing cells. Examples virus-based vectors that can be used to deliver nucleic acid encoding a p21 polypeptide to a mammal (e.g., a human) include, without limitation, virus-based vectors based on adenoviruses, AAVs, Sendai viruses, retroviruses, or lentiviruses.

When a vector used to deliver nucleic acid encoding a hypophosphorylated RB polypeptide to a mammal (e.g., a human) is a non-viral vector, any appropriate non-viral vector can be used. In some cases, a non-viral vector can be an expression plasmid (e.g., a cDNA expression vector).

In addition to nucleic acid encoding a hypophosphorylated RB polypeptide, a vector (e.g., a viral vector or a non-viral vector) can contain one or more regulatory elements operably linked to the nucleic acid encoding a hypophosphorylated RB polypeptide. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector depends on several factors, including, without limitation, inducibility, targeting, and the level of expression desired. For example, a promoter can be included in a vector to facilitate transcription of a nucleic acid encoding a hypophosphorylated RB polypeptide. A promoter can be a naturally occurring promoter or a recombinant promoter. A promoter can be ubiquitous or inducible (e.g., in the presence of tetracycline), and can affect the expression of a nucleic acid encoding a polypeptide in a general or tissue-specific manner. Examples of promoters that can be used to drive expression of a hypophosphorylated RB polypeptide in cells include, without limitation, PGK promoters, CMV promoters, and CAGS promoters. For example, a vector can contain a promoter and nucleic acid encoding a hypophosphorylated RB polypeptide. In this case, the promoter is operably linked to a nucleic acid encoding a hypophosphorylated RB polypeptide such that it drives expression of the hypophosphorylated RB polypeptide in cells.

In some cases, expression of a hypophosphorylated RB polypeptide delivered using nucleic acid can be directed to cancer cells using one or more regulatory elements (e.g., promotors such as cancer-specific promotors; microRNA target sequences that are blocked or degraded in non-cancer cells to prevent expression in those non-cancer cells; or protein degradation sequences active in normal cells but not in cancer cells (e.g., ubiquitin-mediated degradation)) to regulate the expression of a hypophosphorylated RB polypeptide within cancer cells. Examples of cancer-specific promotors include, without limitation, APF promotors for hepatocellular cancer cells and CEA promotors for epithelial cancer cells.

Nucleic acid encoding a hypophosphorylated RB polypeptide can be produced by techniques including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a hypophosphorylated RB polypeptide.

In some cases, a carrier molecule can be used to deliver one or more (e.g., one, two, three, four, or more) agents that can increase a level of a CXCL14 polypeptide expressed by cancer cells and/or that can increase the presence of a CXCL14 polypeptide within the location of cancer cells (e.g., within 1 to 10 mm of a tumor) to a mammal (e.g., a human) having cancer. Examples of carrier molecules that can be used to deliver one or more agents that can increase a level of a CXCL14 polypeptide expressed by cancer cells and/or that can increase the presence of a CXCL14 polypeptide within the location of cancer cells (e.g., within 1 to 10 mm of a tumor) to a mammal (e.g., a human) having cancer include, without limitation, liposomes, polymeric micelles, microspheres, nanoparticles, and polypeptides (e.g., antibodies). In some cases, one or more agents that can increase a level of a CXCL14 polypeptide expressed by cancer cells and/or that can increase the presence of a CXCL14 polypeptide within the location of cancer cells (e.g., within 1 to 10 mm of a tumor) to a mammal (e.g., a human) having cancer can be encapsulated within a carrier molecule. For example, when an agent that can increase a level of a CXCL14 polypeptide expressed by cancer cells and/or that can increase the presence of a CXCL14 polypeptide within the location of cancer cells (e.g., within 1 to 10 mm of a tumor) is a nucleic acid (e.g., a nucleic acid encoding a CXCL14 polypeptide), the nucleic acid can be encapsulated within a carrier molecule (e.g., a nanoparticle).

In some cases, one or more (e.g., one, two, three, four, or more) agents that can increase a level of a CXCL14 polypeptide expressed by cancer cells and/or that can increase the presence of a CXCL14 polypeptide within the location of cancer cells (e.g., within 1 to 10 mm of a tumor) can be targeted (e.g., can be designed to target) to one or more cancer cells within a mammal (e.g., a human) having cancer and being treated as described herein. For example, an agent that can increase a level of a CXCL14 polypeptide expressed by cancer cells and/or that can increase the presence of a CXCL14 polypeptide within the location of cancer cells (e.g., within 1 to 10 mm of a tumor) can include a targeting moiety that can direct the agent to one or more cancer cells within a mammal (e.g., a human) having cancer. When a carrier molecule is used to deliver one or more (e.g., one, two, three, four, or more) agents that can increase a level of a CXCL14 polypeptide expressed by cancer cells and/or that can increase the presence of a CXCL14 polypeptide within the location of cancer cells (e.g., within 1 to 10 mm of a tumor) to a mammal (e.g., a human) having cancer, the carrier molecule can be targeted (e.g., can be designed to target) to one or more cancer cells within a mammal (e.g., a human) having cancer and being treated as described herein.

In some cases, an agent that can increase a level of a CXCL14 polypeptide expressed by cancer cells and/or that can increase the presence of a CXCL14 polypeptide within the location of cancer cells (e.g., within 1 to 10 mm of a tumor) (and/or a carrier molecule used to deliver an agent that can increase a level of a CXCL14 polypeptide expressed by cancer cells and/or that can increase the presence of a CXCL14 polypeptide within the location of cancer cells (e.g., within 1 to 10 mm of a tumor)) can be conjugated to a targeting moiety that can direct the agent to one or more cancer cells within a mammal (e.g., a human) having cancer. For example, when an agent that can increase a level of a CXCL14 polypeptide expressed by cancer cells and/or that can increase the presence of a CXCL14 polypeptide within the location of cancer cells (e.g., within 1 to 10 mm of a tumor) is a polypeptide (e.g., a CXCL14 polypeptide), the polypeptide can be conjugated to a targeting moiety (e.g., an antigen binding polypeptide such as an antibody or a single-chain variable fragment (scFv)). In some cases, a CXCL14 polypeptide directly or indirectly conjugated (e.g., covalently conjugated) to a targeting moiety (e.g., a targeting moiety that binds to cancer cells) can be designed and used to increase the presence of a CXCL14 polypeptide within the location of cancer cells (e.g., within 1 to 10 mm of a tumor).

In some cases, an agent that can increase a level of a CXCL14 polypeptide (and/or a carrier molecule used to deliver an agent that can increase a level of a CXCL14 polypeptide) can be complexed to a targeting moiety that can direct the agent to one or more cancer cells within a mammal (e.g., a human) having cancer. For example, when an agent that can increase a level of a CXCL14 polypeptide is a nucleic acid (e.g., a nucleic acid encoding a CXCL14 polypeptide), the nucleic acid can be complexed with a targeting moiety (e.g., an antibody).

Any appropriate targeting moiety can be used to direct one or more agents that can increase a level of a CXCL14 polypeptide expressed by cancer cells and/or that can increase the presence of a CXCL14 polypeptide within the location of cancer cells (e.g., within 1 to 10 mm of a tumor) can include targeting moiety that can direct the agent to one or more cancer cells within a mammal (e.g., a human) having cancer. Examples of targeting moieties that can be used to direct one or more agents that can increase a level of a CXCL14 polypeptide expressed by cancer cells and/or that can increase the presence of a CXCL14 polypeptide within the location of cancer cells (e.g., within 1 to 10 mm of a tumor) include, without limitation, targeting polypeptides (e.g., antibodies) and ligands.

In some cases, a targeting moiety can be used as described herein to target an antigen (e.g., a cell-surface antigen) expressed by one or more cancer cells in a mammal (e.g., a human) having cancer. In some cases, an antigen can be a tumor antigen (e.g., a tumor-associate antigen (TAA) or a tumor-specific antigen (TSA)). Examples of antigens that can be expressed by a cancer cell and can be targeted by a targeting moiety that can be used to direct one or more agents that increase a level of a CXCL14 polypeptide expressed by cancer cells and/or that can increase the presence of a CXCL14 polypeptide within the location of cancer cells (e.g., within 1 to 10 mm of a tumor) include, without limitation, cluster of differentiation 19 (CD19; associated with B cell lymphomas, acute lymphoblastic leukemia (ALL), and chronic lymphocytic leukemia (CLL)), alphafetoprotein (AFP; associated with germ cell tumors and/or hepatocellular carcinoma), carcinoembryonic antigen (CEA; associated with bowel cancer, lung cancer, and/or breast cancer), CA-125 (associated with ovarian cancer), mucin 1 (MUC-1; associated with breast cancer), epithelial tumor antigen (ETA; associated with breast cancer), and melanoma-associated antigen (MAGE; associated with malignant melanoma).

In some cases, one or more agents that can increase a level of a CXCL14 polypeptide (and/or one or more carrier molecules including one or more agents that can increase a level of a CXCL14 polypeptide) can be formulated into a composition (e.g., a pharmaceutically acceptable composition) for administration to a mammal (e.g., a human) having cancer. For example, one or more agents that can increase a level of a CXCL14 polypeptide (and/or one or more carrier molecules including one or more agents that can increase a level of a CXCL14 polypeptide) can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, when a composition containing one or more agents that can increase a level of a CXCL14 polypeptide (and/or one or more carrier molecules including one or more agents that can increase a level of a CXCL14 polypeptide) is administered to a mammal (e.g., a human) having cancer, the composition can be designed for oral or parenteral (including, without limitation, a subcutaneous, intramuscular, intravenous, intradermal, intra-cerebral, intrathecal, or intraperitoneal (i.p.) injection) administration to the mammal. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition containing one or more agents that can increase a level of a CXCL14 polypeptide (and/or one or more carrier molecules including one or more agents that can increase a level of a CXCL14 polypeptide) can be administered to a mammal (e.g., a human) having cancer in any appropriate amount (e.g., any appropriate dose). An effective amount of a composition containing one or more agents that can increase a level of a CXCL14 polypeptide (and/or one or more carrier molecules including one or more agents that can increase a level of a CXCL14 polypeptide) can be any amount that can treat a mammal having cancer as described herein without producing significant toxicity to the mammal. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the cancer in the mammal being treated may require an increase or decrease in the actual effective amount administered.

A composition containing one or more agents that can increase a level of a CXCL14 polypeptide (and/or one or more carrier molecules including one or more agents that can increase a level of a CXCL14 polypeptide) can be administered to a mammal (e.g., a human) having cancer in any appropriate frequency. The frequency of administration can be any frequency that can treat a mammal having cancer without producing significant toxicity to the mammal. For example, the frequency of administration can be from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more agents that can increase a level of a CXCL14 polypeptide (and/or one or more carrier molecules including one or more agents that can increase a level of a CXCL14 polypeptide) can be administered to a mammal (e.g., a human) having cancer for any appropriate duration. An effective duration for administering or using a composition containing one or more inhibitors of XCL signaling can be any duration that can treat a mammal having cancer without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) having cancer can include administering to the mammal one or more agents that can increase a level of a CXCL14 polypeptide expressed by cancer cells and/or that can increase the presence of a CXCL14 polypeptide within the location of cancer cells (e.g., within 1 to 10 mm of a tumor) as the sole active ingredient to treat the mammal. For example, a composition containing one or more agents that can increase a level of a CXCL14 polypeptide expressed by cancer cells and/or that can increase the presence of a CXCL14 polypeptide within the location of cancer cells (e.g., within 1 to 10 mm of a tumor) can include the one or more agents that increase a level of a CXCL14 polypeptide expressed by cancer cells and/or that can increase the presence of a CXCL14 polypeptide within the location of cancer cells (e.g., within 1 to 10 mm of a tumor) as the sole active ingredient in the composition that is effective to treat a mammal having cancer.

In some cases, methods for treating a mammal (e.g., a human) having cancer as described herein (e.g., by administering one or more agents that can increase a level of a CXCL14 polypeptide) also can include administering to the mammal one or more (e.g., one, two, three, four, five or more) agents that can stimulate monocytes to differentiate into macrophages. Examples of agents that can stimulate monocytes to differentiate into macrophages and can be administered together with one or more agents that can increase a level of a CXCL14 polypeptide include, without limitation, IL-34 polypeptides, TNFα polypeptides, IL-17 polypeptides, and any combinations thereof.

In some cases, methods for treating a mammal (e.g., a human) having cancer as described herein (e.g., by administering one or more agents that can increase a level of a CXCL14 polypeptide) also can include administering to the mammal one or more (e.g., one, two, three, four, five or more) additional agents/therapies used to treat a cancer. Examples of additional agents that can be used to treat a cancer include, without limitation, chemotherapies, targeted therapies, immunotherapies, radiopharmaceuticals, and any combinations thereof. In cases where one or more agents that can increase a level of a CXCL14 polypeptide expressed by cancer cells and/or that can increase the presence of a CXCL14 polypeptide within the location of cancer cells (e.g., within 1 to 10 mm of a tumor) are used in combination with additional agents used to treat cancer, the one or more additional agents can be administered at the same time (e.g., in a single composition containing both one or more agents that can increase a level of a CXCL14 polypeptide and the one or more additional agents) or independently. For example, one or more agents that can increase a level of a CXCL14 polypeptide can be administered first, and the one or more additional agents administered second, or vice versa. Examples of therapies that can be used to treat cancer include, without limitation, surgery, and radiation therapy. In cases where one or more agents that can increase a level of a CXCL14 polypeptide expressed by cancer cells and/or that can increase the presence of a CXCL14 polypeptide within the location of cancer cells (e.g., within 1 to 10 mm of a tumor) are used in combination with one or more additional therapies used to treat cancer, the one or more additional therapies can be performed at the same time or independently of the administration of one or more agents that can increase a level of a CXCL14 polypeptide. For example, one or more agents that can increase a level of a CXCL14 polypeptide can be administered before, during, or after the one or more additional therapies are performed.

The invention will be further described in the following examples, which do not limit the scope of the invention described in the claims.

### Examples

### Example 1: P21 Induction Triggers Immunosurveillance

Immune cells identify and destroy damaged cells to prevent them from causing cancer or other pathologies, but how remains poorly understood.

This Example investigates the senescence program at a molecular mechanistic level and identifies senescence-associated super-enhancer-controlled genes that are conserved across species, cell types and senescence-inducing stressors.

### Results

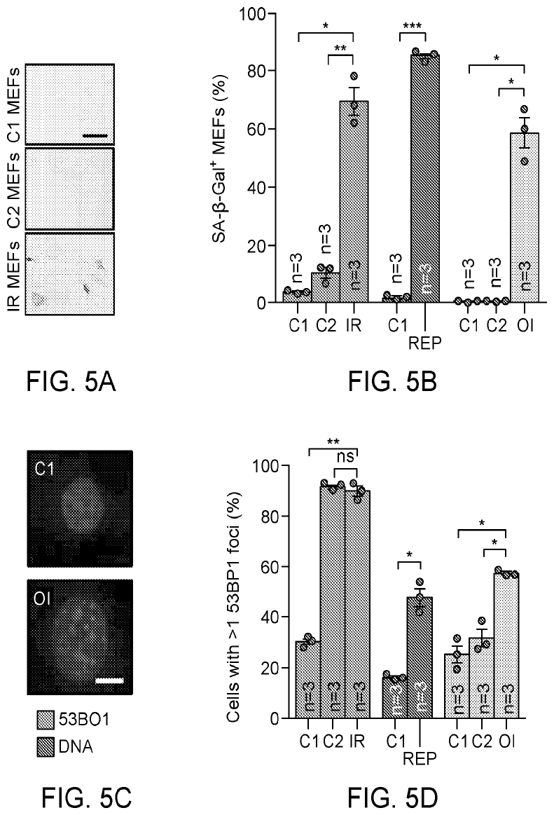

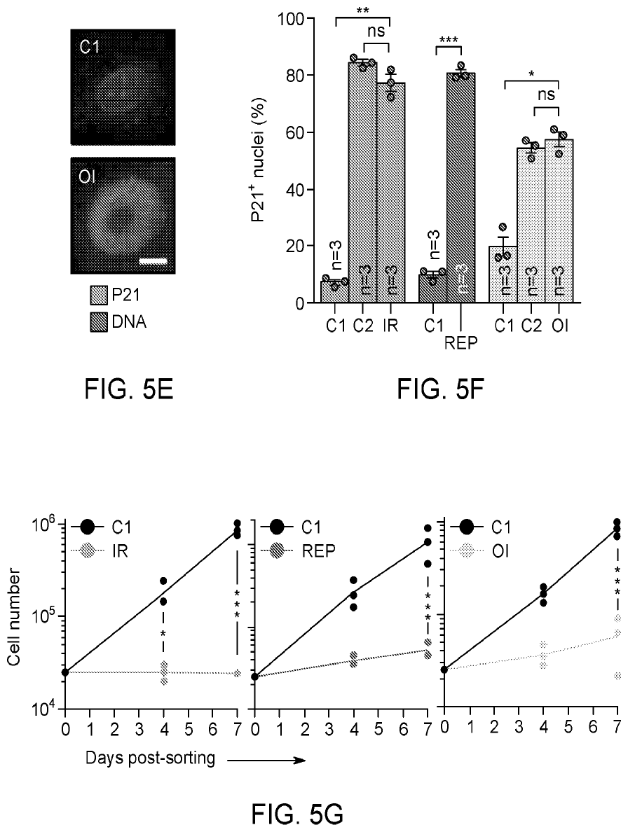

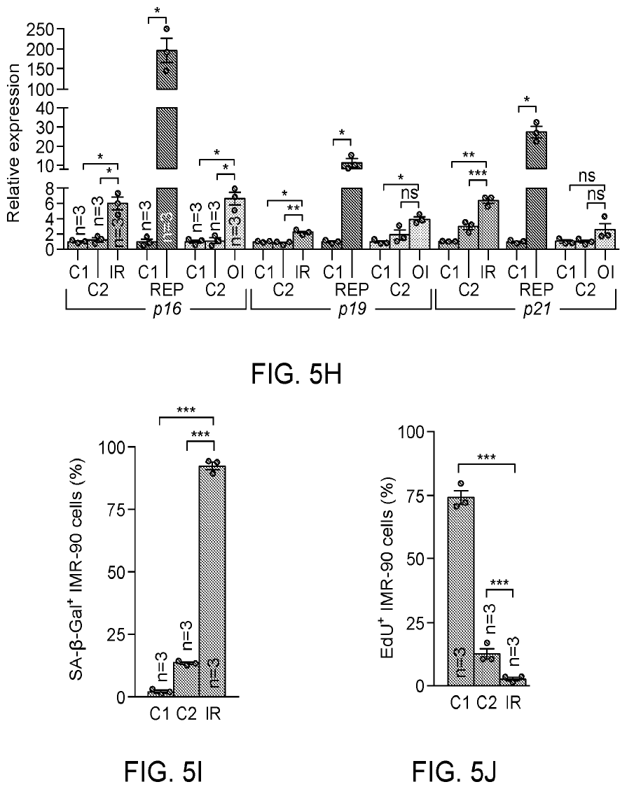

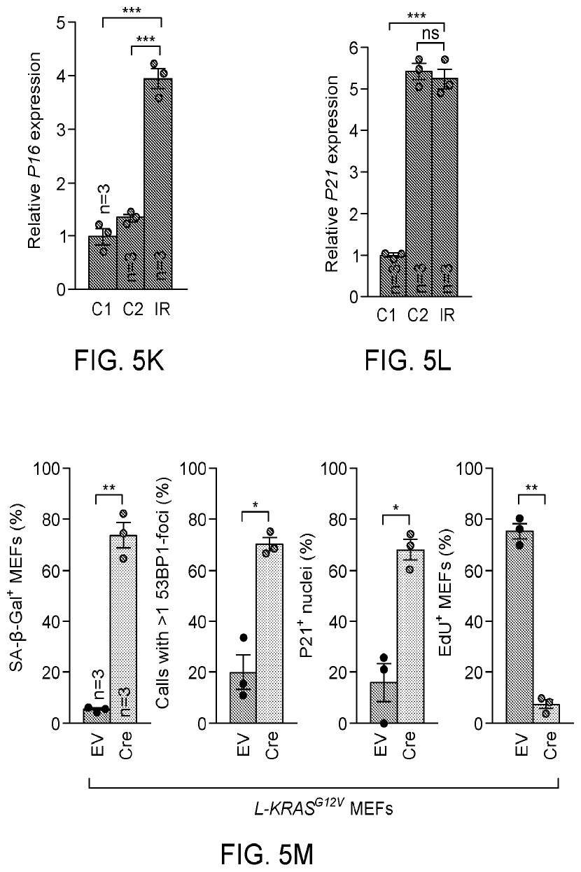

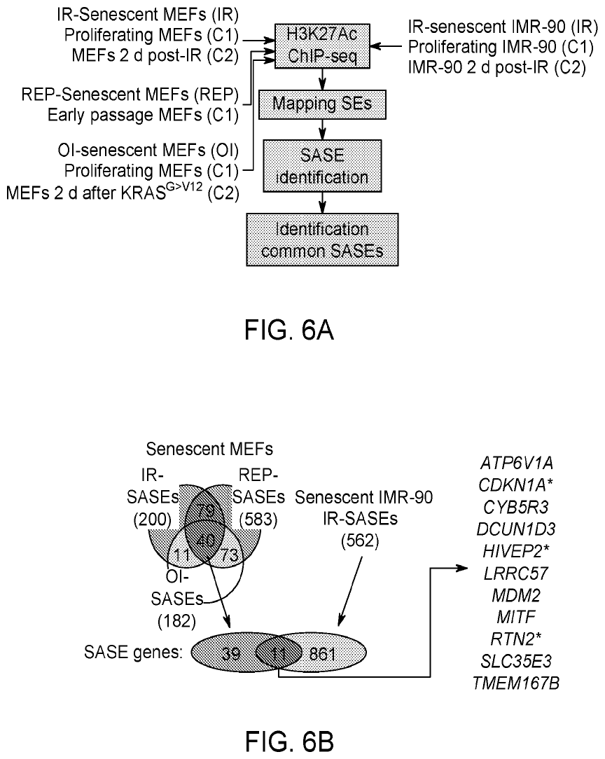

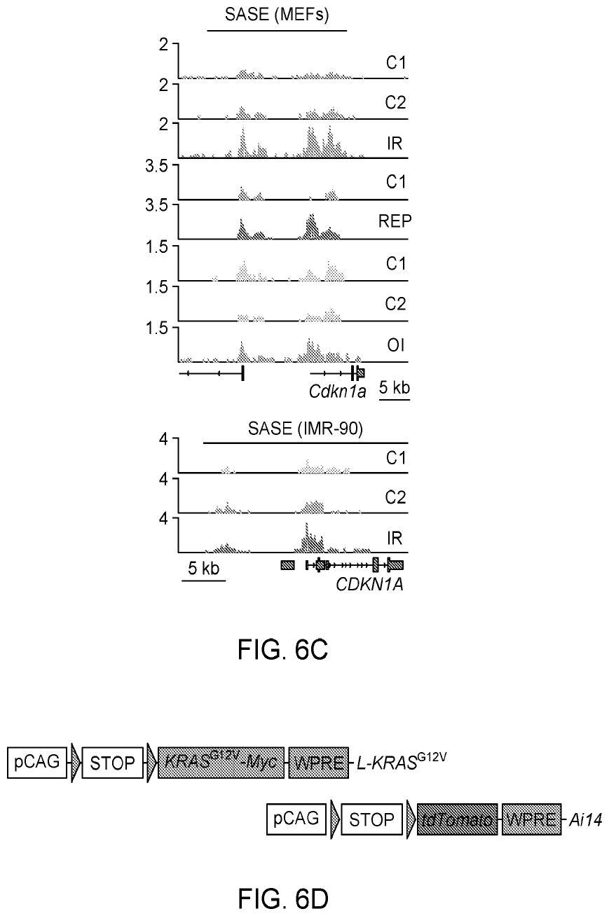

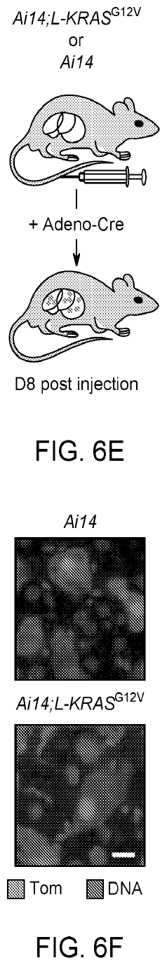

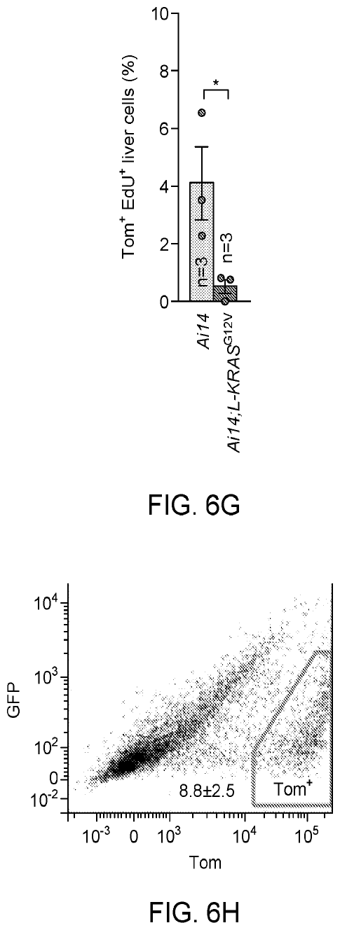

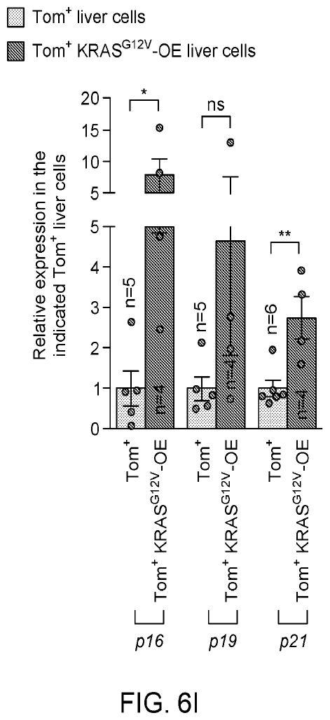

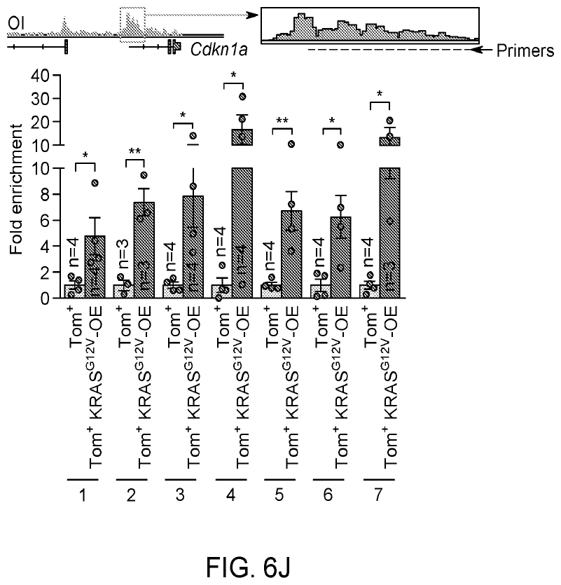

Primary mouse embryonic fibroblasts (MEFs) were exposed to 3 distinct senescence-inducing stressors: γ-irradiation (IR), extensive replication (REP), and oncogene-induced (OI) signaling by overexpression of KRAS^{G12V} (FIG. 5). The common super-enhancer changes as these cells transitioned to a senescent state were mapped, and transcriptionally activated genes associated with these super-enhancers were identified (FIGS. 6A and 6B). 50 such genes were uncovered (FIG. 6B), three of which were also associated with a senescence-associated super-enhancer and transcriptionally upregulated in senescent human fetal lung (IMR-90) cells generated by irradiation (FIGS. 6A to 6C) or KRAS^{G12V} overexpression, including Cdkn1a (encoding P21). H3K27Ac ChIP-qPCR on OI-senescent cells (SNCs) collected from mouse liver indicated that the senescence-associated super-enhancer identified near the Cdkn1a locus was conserved in vivo (FIGS. 6D to 6J).

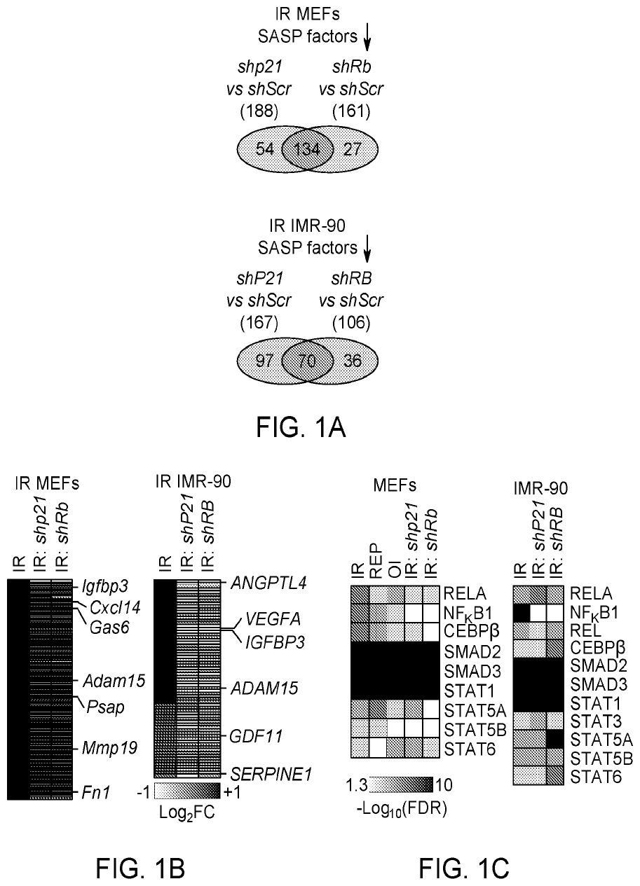

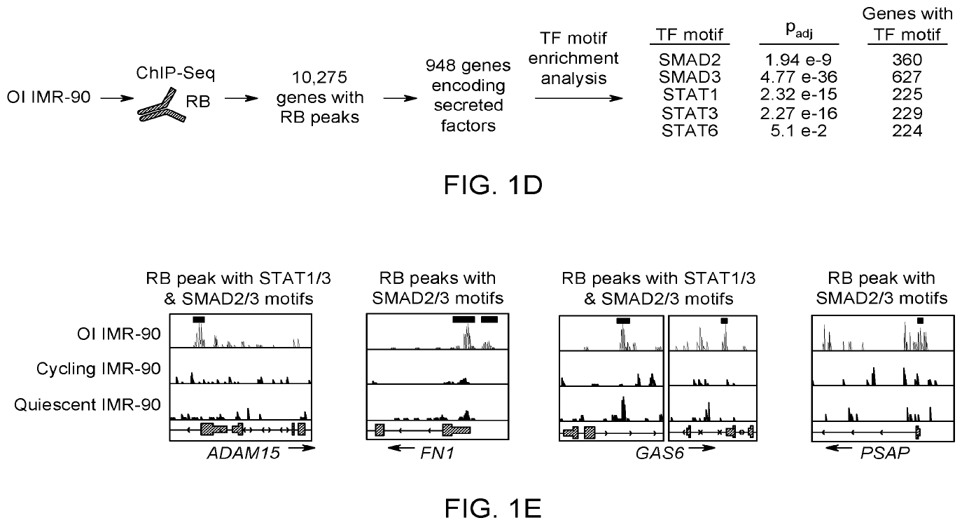

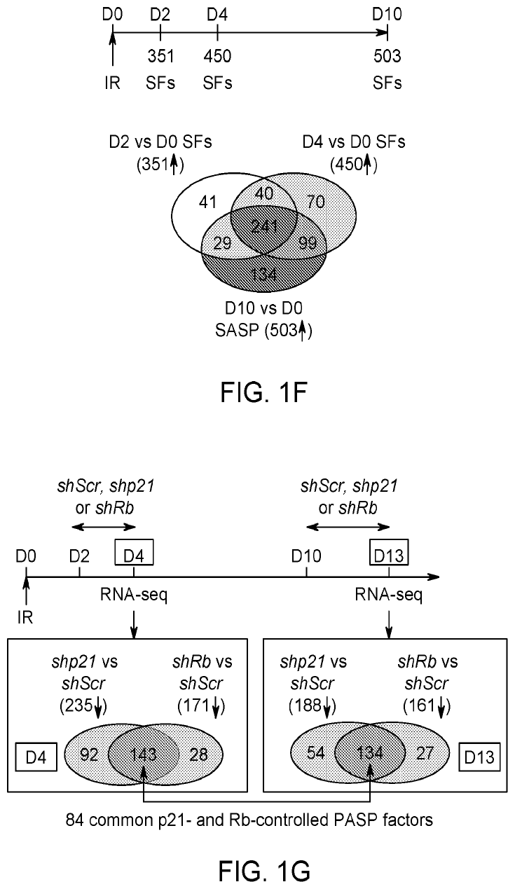

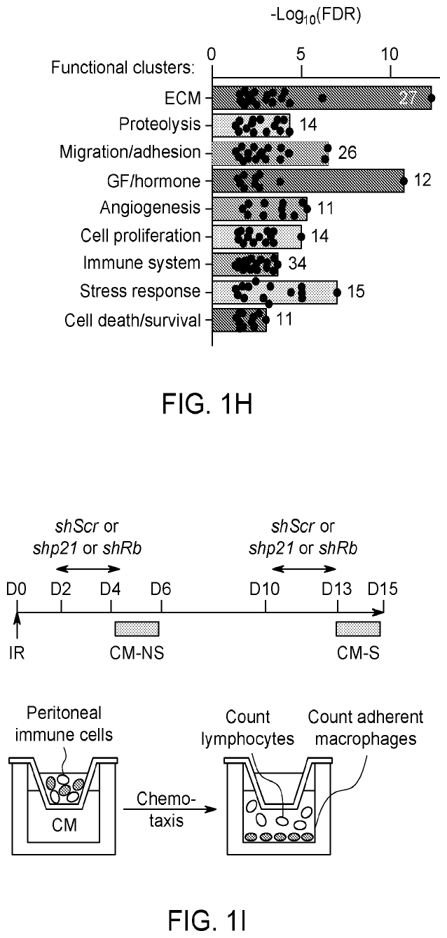

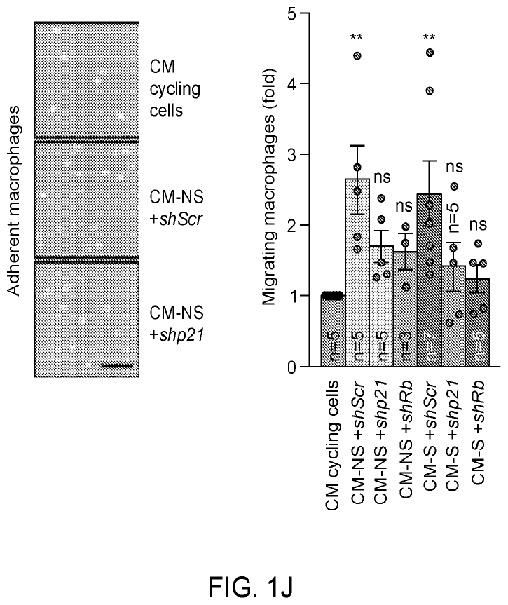

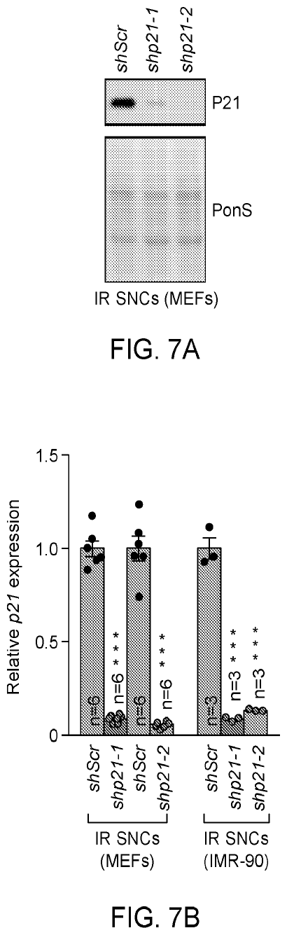

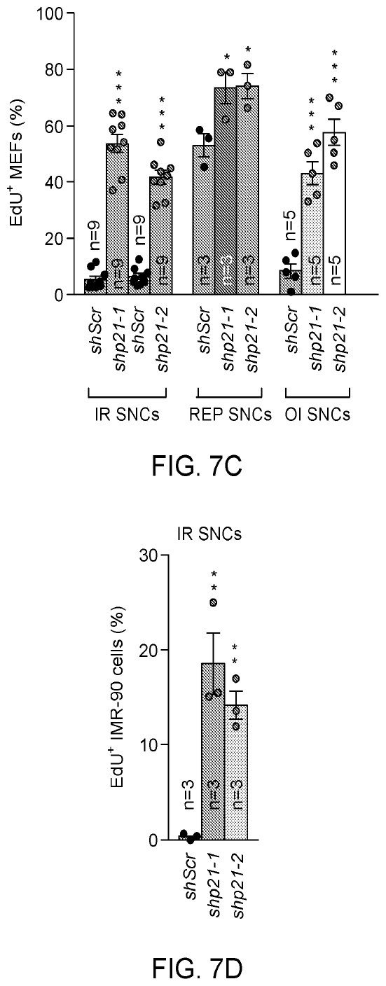

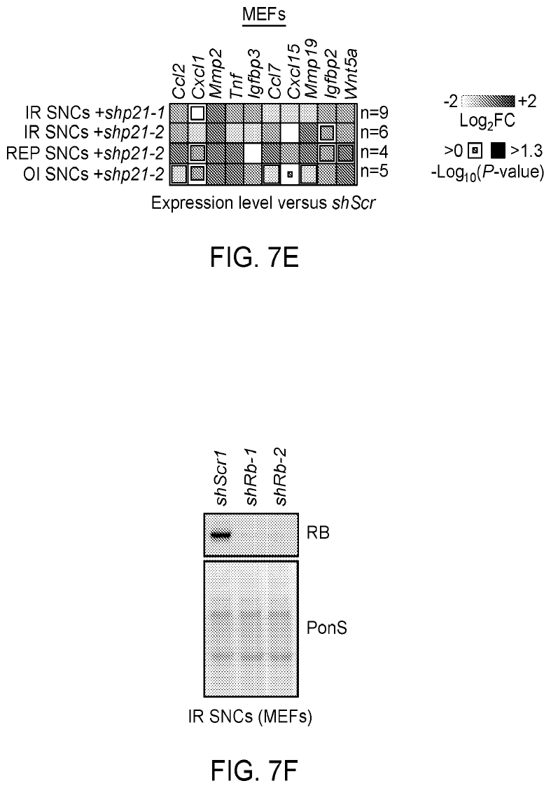

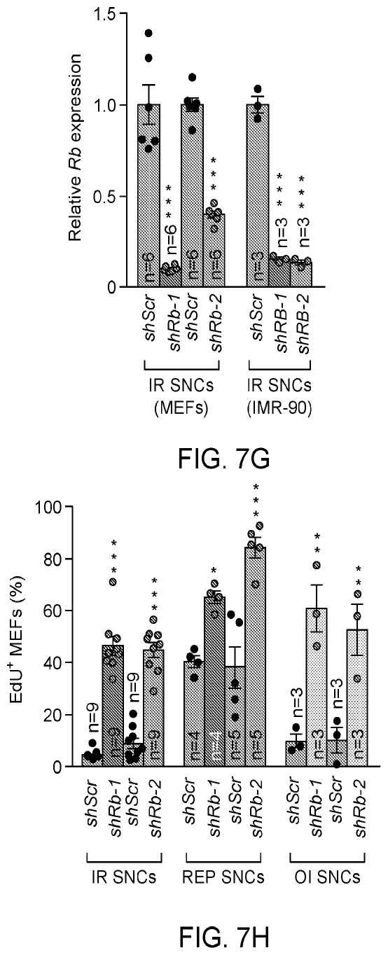

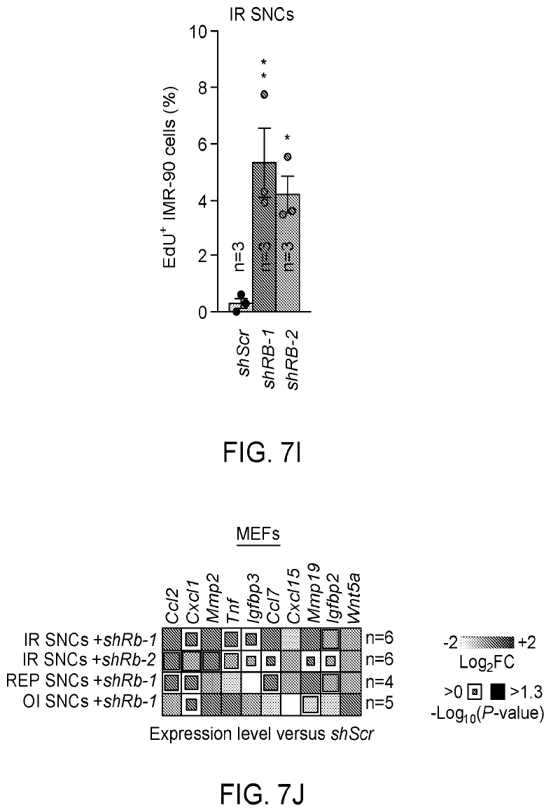

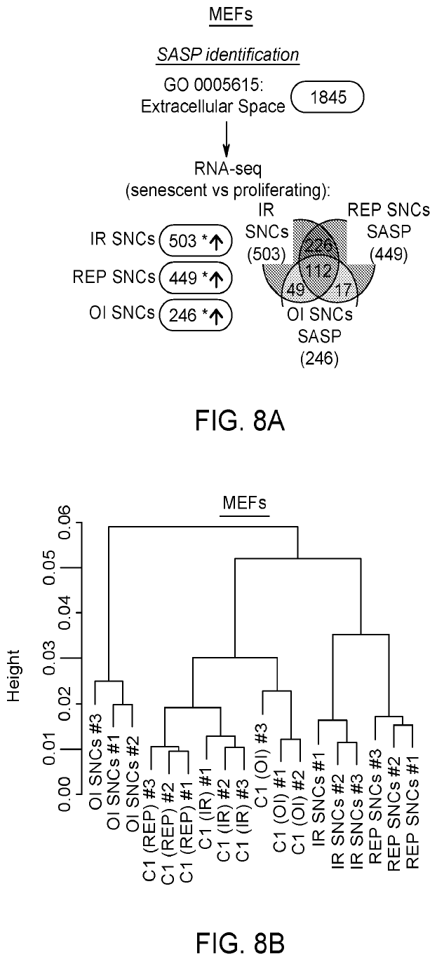

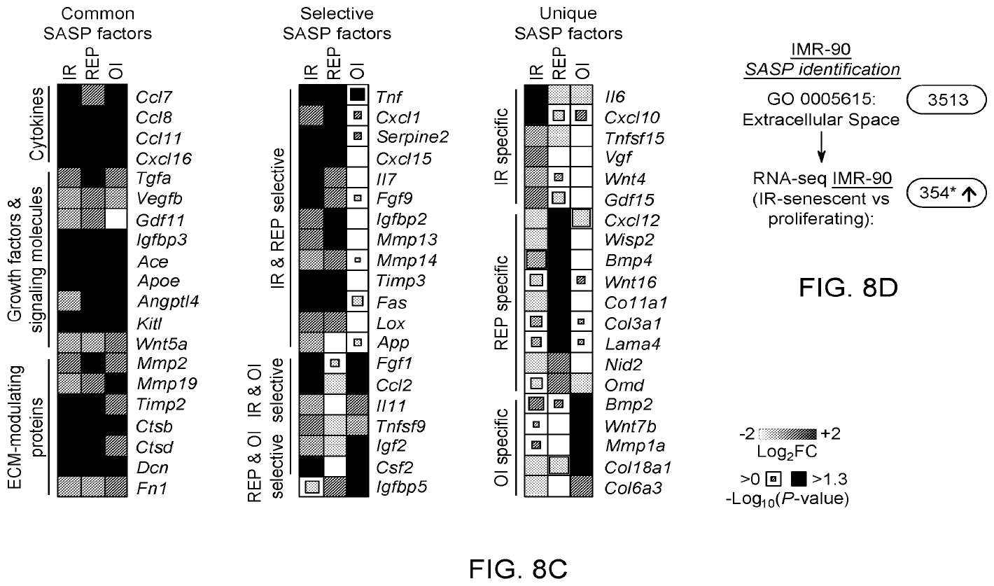

Fully SNCs in which P21 incorporated 5-ethynyl-2′-deoxyuridine was depleted (EdU; FIGS. 7A to 7D), indicating that sustaining P21 in the senescent state is important to prevent cell cycle reentry through continued transcriptional repression of E2F target genes via hypophosphorylation of RB. p21-depletion in SNCs also decreased expression of multiple SASP factors as determined by reverse transcription-quantitative polymerase chain reaction (RT-qPCR) for a panel of well-established SASP factors (FIG. 7E). Comprehensive transcriptomic analysis of IR-senescent MEFs using RNA-sequencing (RNA-seq) revealed that about a third of the SASP (188 of 503 factors) is P21-dependent (FIG. 1A, FIGS. 8A to 8C). Similarly, nearly half the SASP (167 of 354 factors) identified in IR-senescent IMR-90 cells were dependent on P21 (FIG. 1A and FIG. S4D), which prompted us to probe the mechanism(s) underlying these P21-dependent secretory phenotypes, hereafter referred to as P21-activated secretory phenotypes (PASPs).

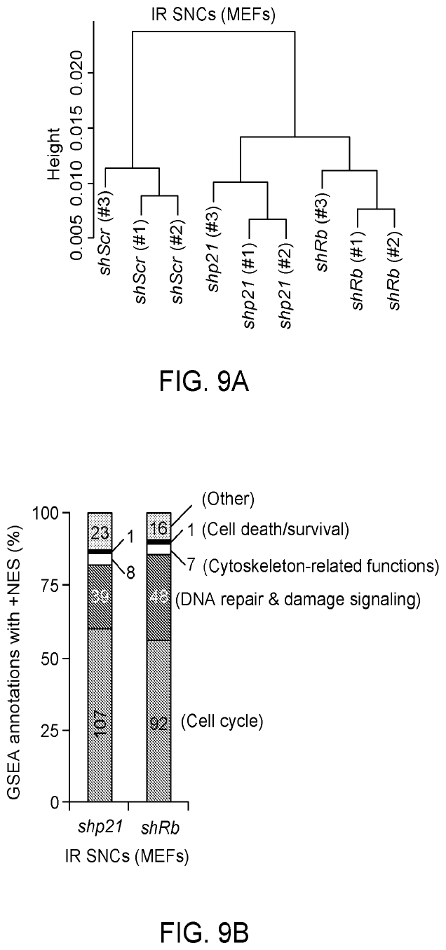

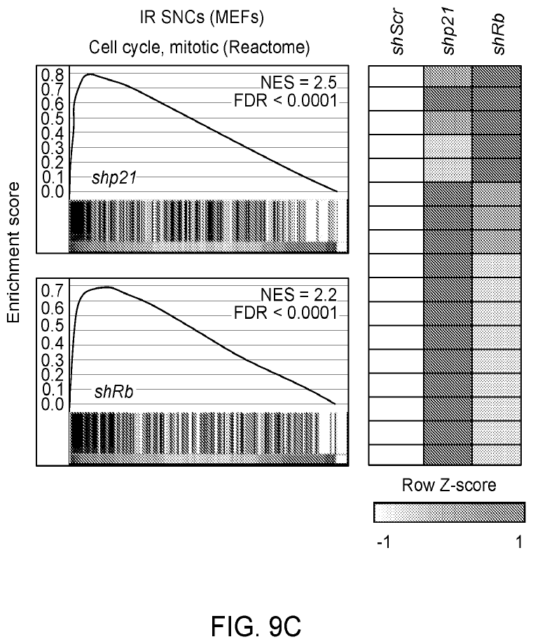

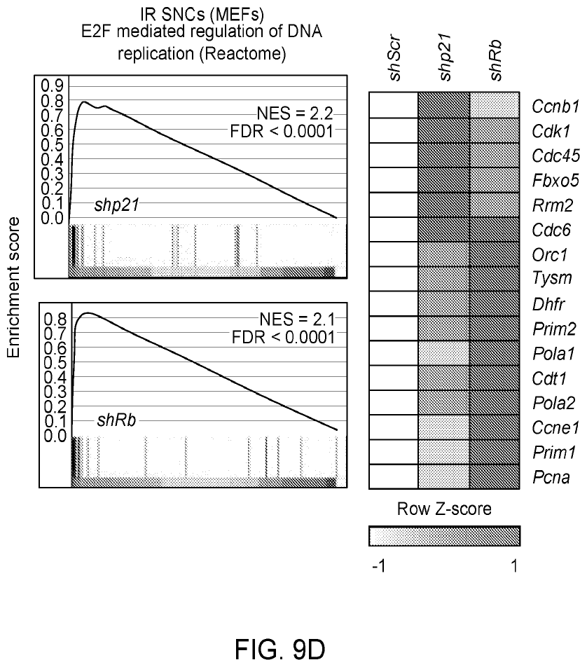

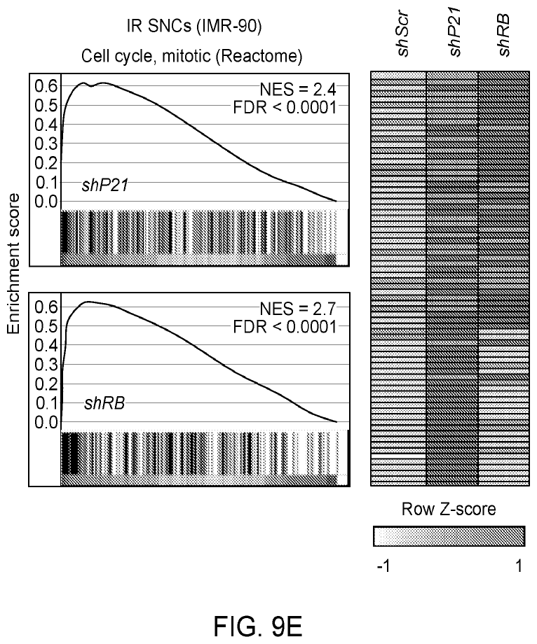

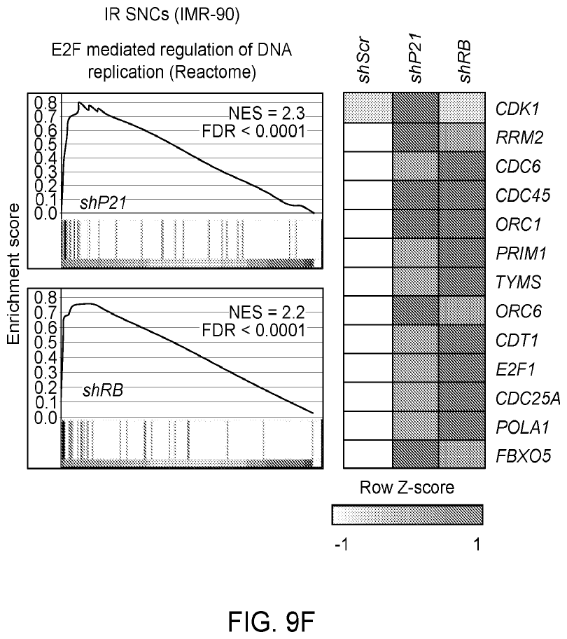

RB was first focused on, and it was found that RB depletion in SNCs not only activated E2F target genes (FIGS. 7F to 7I and FIG. 9) but also decreased expression of most of the SASP factors downregulated with p21 depletion (FIGS. 1A and 1B and FIG. 7J), suggesting that P21 confers its effect on the SASP through hypophosphorylation of RB. To explore how P21-mediated RB hypophosphorylation might activate SASP genes, transcription factors (TFs) that have been linked to the SASP, inflammation, or cytokine production were identified, and their transcriptional targets were used in overrepresentation analyses on RNA-seq data from IR-, REP, OI-senescent MEFs, IR-senescent IMR-90 cells, and their non-senescent counterparts. It was found that RELA, CEBPb, SMAD2, SMAD3, STAT1, STAT5A/B and STAT6 were consistently more active in SNCs than in non-SNCs regardless of senescence-inducing stressor or species (FIG. 1C). RELA, SMAD2, SMAD3, STAT1, and STAT6 lost this status when p21 or Rb were depleted (FIG. 1C), implying that hypophosphorylated RB enhances the activity of these TFs in SNCs to establish the PASP.

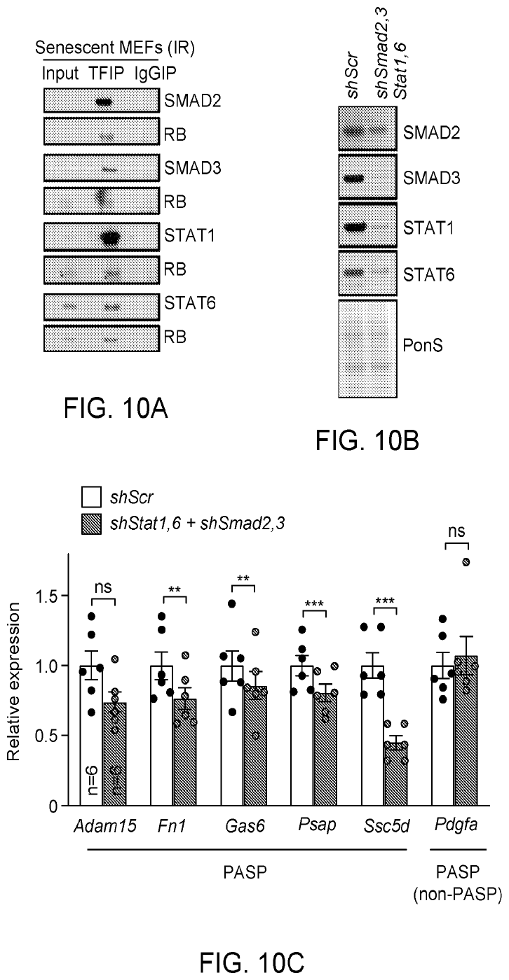

Analysis of publicly available RB ChIP-seq data from OI-senescent IMR-90 cells (Chicas et al., Cancer cell., 17:376-387 (2010)) revealed that RB peaks mapped to the promoter regions of 948 secreted factors (SFs) and that these peaks were enriched for binding sites of all TFs that we identified as instrumental in establishing the PASP, with exception of RELA (FIG. 1D). RB peaks mapped to promoter regions of 49 of 167 PASP genes identified in IR IMR-90 cells and associated with TFs critical for establishing the PASP (FIG. 1E). Most of these promoter regions had no such peaks when IMR-90 cells were cycling or quiescent. Furthermore, SMAD2, SMAD3, STAT1 and STAT6 co-immunoprecipitated RB from IR-senescent MEFs and co-depletion of SMAD2, SMAD3, STAT1 and STAT6 in IR-senescent MEFs reduced transcription of SASP genes where RB and these TFs colocalize in promoter regions (FIG. 10). Collectively, these data indicate that a P21-responsive RB pool interacts with specific STAT and SMAD TFs at PASP gene promoters to enhance their expression.

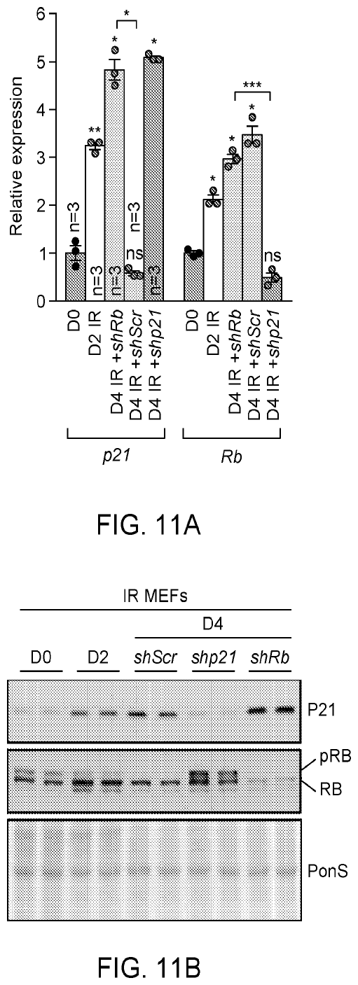

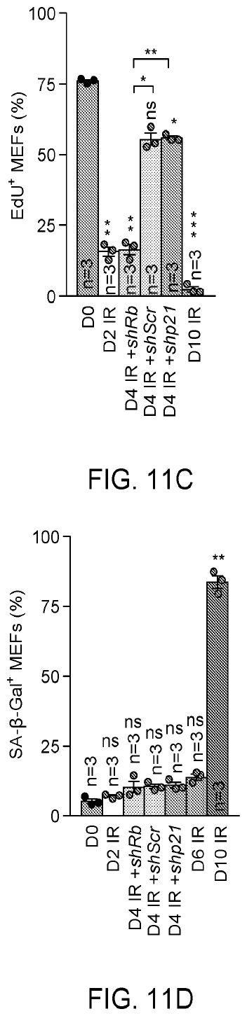

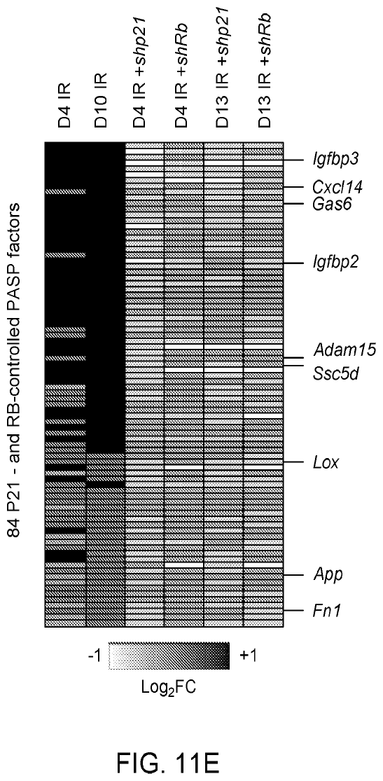

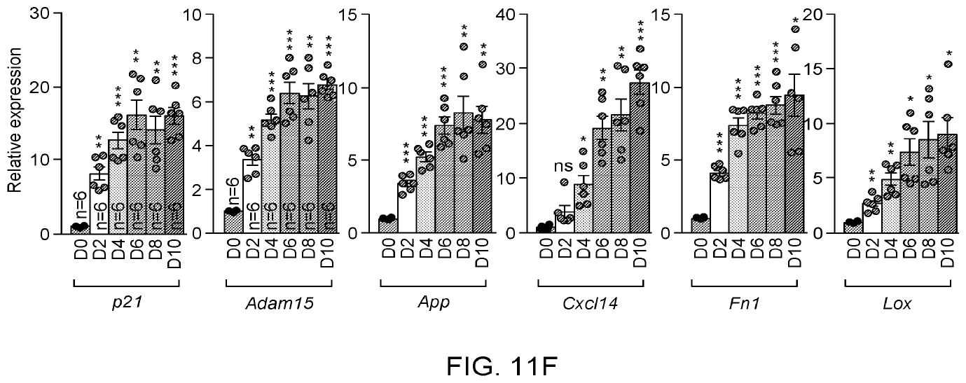

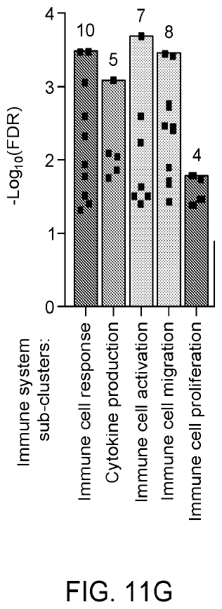

To determine whether the PASP is senescence-dependent, RNA-seq was performed on non-senescent MEFs with high P21 collected 2 or 4 days (D2 or D4) post-irradiation (FIG. 1F, FIGS. 11A to 11D). D2 and D4 IR MEFs upregulated 351 and 450 SFs, respectively, 241 of which were shared with D10 IR MEFs (FIG. 1F and Table 1). D4 IR MEFs depleted for p21 or Rb lost 235 and 171 of their secreted factors, respectively, indicating that the PASP is a senescence-independent phenomenon (FIG. 1G, FIGS. 11A to 11D). Eighty-four PASP factors were commonly lost in D4 and D10 IR MEFs when P21 or RB were depleted, indicating that the PASP of non-SNCs becomes an integral part of the SASP as cells advance to a senescent state (FIG. 1G and FIGS. 11E and 11F).

TABLE 1
Secretory phenotype of IR-induced, non-senescent MEFs including after p21 or Rb
knockdown.

| | | | | | | IR-D4 secreted |
|---|---|---|---|---|---|---|
| | | | | IR-D4 secreted | IR-D4 secreted | factors that are |
| | | | IR secreted | factors that are | factors that are | downregulated |
| MEFs | IR D2 | IR D4 | factors in | downregulated | downregulated | in shp21 vs. |
| (secreted | secreted | secreted | D2, D4 and | in shp21 vs. | in shRb vs. | shScr AND |
| factors) | factors | factors | D10 | shScr | shScr | shRb vs. shScr |
| | 4930486L24Rik | 1500015O10Rik | 4930486L24Rik | Ache | Ache | Ache |
| | Acpp | 4930486L2 | Acpp | Adam15 | Adam15 | Adam15 |
| | | 4Rik | | | | |
| | Adam15 | Ache | Adam15 | Adamts15 | Adam9 | Adamts2 |
| | Adam9 | Acpp | Adam9 | Adamts2 | Adamts2 | Adamts4 |
| | Adamts4 | Adam15 | Aebp1 | Adamts4 | Adamts4 | Adamtsl4 |
| | Adamts9 | Adam9 | Aga | Adamts5 | Adamtsl4 | Aebp1 |
| | Aebp1 | Adamts15 | Angpt2 | Adamtsl4 | Aebp1 | Angpt4 |
| | Aga | Adamts2 | Angpt4 | Aebp1 | Angpt4 | Angpt12 |
| | Angpt2 | Adamts4 | Apod | Aga | Angpt12 | Apoe |
| | Angpt4 | Adamts5 | Apoe | Agt | Apoe | App |
| | Angptl4 | Adamts9 | App | Angpt2 | App | Arsa |
| | Anxa1 | Adamtsl4 | Arg1 | Angpt4 | Arsa | Arsg |
| | Apod | Adcyap1 | Asah1 | Angptl2 | Arsg | Asah1 |
| | Apoe | Adm | Atrn | Aoc3 | Asah1 | Bdnf |
| | App | Aebp1 | B4galt1 | Apoe | Bcan | Bgn |
| | Areg | Aga | Bcan | App | Bdnf | Bmp1 |
| | Arg1 | Ager | Bdnf | Arsa | Bgn | C1s1 |
| | Asah1 | Agt | Bmp6 | Arsg | Bmp1 | Calr |
| | Atrn | Angpt1 | Bmp8a | Asah1 | Bmp8b | Capg |
| | Axl | Angpt2 | C1qa | Bdnf | C1s1 | Ccdc80 |
| | B4galt1 | Angpt4 | C1qtnf2 | Bgn | Calr | Cd63 |
| | Bcan | Angptl2 | C1qtnf3 | Bmp1 | Capg | Cdh13 |
| | Bdnf | Aoc3 | C1s1 | Bmp4 | Ccdc80 | Chid1 |
| | Bmp1 | Apoa2 | C2 | C1qa | Ccl25 | Chrd |
| | Bmp2 | Apob | C3 | C1qtnf1 | Cd63 | Cmtm3 |
| | Bmp6 | Apod | Capg | C1qtnf2 | Cdh13 | Col11a1 |
| | Bmp8a | Apoe | Cck | C1qtnf3 | Chid1 | Col12a1 |
| | Bmp8b | App | Ccl11 | C1qtnf6 | Chrd | Col1a1 |
| | Btc | Arg1 | Ccl2 | C1ra | Cmtm3 | Col1a2 |
| | Clqa | Arsa | Ccl25 | C1rl | Col11a1 | Col3a1 |
| | C1qc | Arsg | Ccl5 | C1s1 | Col12a1 | Col4a2 |
| | C1qtnf2 | Asah1 | Ccl6 | Calr | Col1a1 | Col5a2 |
| | C1qtnf3 | Atrn | Ccl7 | Capg | Col1a2 | Cpe |
| | C1s1 | B2m | Ccl9 | Ccdc80 | Col25a1 | Cpq |
| | C2 | B4galt1 | Cdh13 | Cck | Col3a1 | Cpxm1 |
| | C3 | Bcan | Cdnf | Ccl11 | Col4a2 | Cpxm2 |
| | C4b | Bdnf | Cela1 | Ccl7 | Col5a2 | Crtap |
| | Cap1 | Bgn | Cfh | Cd63 | Cpe | Cst3 |
| | Capg | Bmp1 | Chid1 | Cdh13 | Cpq | Cthrc1 |
| | Ccbe1 | Bmp2 | Clu | Cfh | Cpxm1 | Ctsb |
| | Cck | Bmp3 | Cmtm3 | Chid1 | Cpxm2 | Ctsd |
| | Ccl11 | Bmp4 | Col8a1 | Chrd | Crtap | Ctsh |
| | Ccl2 | Bmp5 | Colec10 | Cmtm3 | Cst3 | Ctsl |
| | Ccl25 | Bmp6 | Cp | Col11a1 | Ctgf | Ctsz |
| | Ccl5 | Bmp8a | Cpe | Col12a1 | Cthrc1 | Cxcl14 |
| | Ccl6 | Bmp8b | Cpm | Col1a1 | Ctsb | Dcn |
| | Ccl7 | Btd | Cpq | Col1a2 | Ctsd | Dkk3 |
| | Ccl9 | C1qa | Cpxm1 | Col3a1 | Ctsf | E130311K13Rik |
| | Cd40 | C1qtnf1 | Cpz | Col4al | Ctsh | Ecm1 |
| | Cd59b | C1qtnf2 | Creg1 | Col4a2 | Ctsl | Efemp2 |
| | Cd63 | C1qtnf3 | Csf1 | Col5a2 | Ctsz | Emilin1 |
| | Cdh13 | C1qtnf4 | Cst3 | Col8al | Cxcl14 | Fabp3 |
| | Cdnf | C1qtnf6 | Cstb | Cpa6 | Cxcl3 | Fbln2 |
| | Cela1 | C1qtnf7 | Ctsb | Cpb1 | Cyr61 | Fmod |
| | Cfh | C1ra | Ctsd | Cpe | Dcn | Fn1 |
| | Chid1 | C1rl | Ctsf | Cpq | Dkk3 | Fstl1 |
| | Clcf1 | C1s1 | Ctsh | Cpxml | E130311K13Rik | Fstl3 |
| | Clu | C2 | Ctsk | Cpxm2 | Ecm1 | Gabbr1 |
| | Cmtm3 | C3 | Ctsl | Crtap | Efemp2 | Gas6 |
| | Col18a1 | Calr | Ctso | Cst3 | Emilin1 | Gba |
| | Col4a1 | Capg | Ctss | Cthrc1 | Fabp3 | Glb1 |
| | Col4a2 | Ccdc80 | Ctsz | Ctsb | Fbln2 | Gpc4 |
| | Col8a1 | Cck | Cxcl1 | Ctsd | Fjx1 | Grem1 |
| | Colec10 | Ccl11 | Cxcl14 | Ctsh | Fmod | Gsn |
| | Comp | Ccl2 | Cxcl16 | Ctsk | Fn1 | Hist1h2bc |
| | Cp | Ccl25 | Dcn | Ctsl | Fstl1 | Hist1h2be |
| | Cpa4 | Ccl3 | Dkk2 | Ctsz | Fstl3 | Hspg2 |
| | Cpe | Ccl5 | Dpt | Cxcl14 | Gabbr1 | Htra1 |
| | Cpm | Ccl6 | Ecm1 | Cxcl16 | Gas6 | Igf2 |
| | Cpn1 | Ccl7 | Edn2 | Cxcl5 | Gba | Igfbp2 |
| | Cpq | Ccl8 | Efemp1 | Dag1 | Glb1 | Igfbp3 |
| | Cpxml | Ccl9 | Eng | Dcn | Gpc4 | Igfbp6 |
| | Cpz | Cd59b | Ereg | Dkk2 | Grem1 | Igfbp7 |
| | Creg1 | Cd63 | F3 | Dkk3 | Gsn | Itgbl1 |
| | Csf1 | Cdh13 | F5 | Dpt | Hist1h2bc | Jam3 |
| | Csf3 | Cdnf | Fabp3 | E130311K13Rik | Hist1h2be | Kcp |
| | Csn3 | Cela1 | Fas | Ecm1 | Hspg2 | Lama2 |
| | Cst3 | Cfh | Fgf10 | Efemp1 | Htra1 | Lama4 |
| | Cstb | Chid1 | Fgf2 | Efemp2 | Ifnar2 | Lama5 |
| | Ctgf | Chrd | Fgf7 | Emilin1 | Igf2 | Lamb1 |
| | Ctsb | Clu | Fjx1 | Emilin2 | Igf2r | Lamp2 |
| | Ctsd | Cmtm3 | Fn1 | Epdr1 | Igfbp2 | Lefty1 |
| | Ctsf | Cmtm4 | Frzb | F3 | Igfbp3 | Lgi4 |
| | Ctsh | Cnp | Fst | Fabp3 | Igfbp6 | Lingo1 |
| | Ctsk | Col11a1 | Fuca2 | Fbln2 | Igfbp7 | Lox |
| | Ctsl | Col12a1 | Gas6 | Fgf10 | Inhba | Loxl2 |
| | Ctso | Col1a1 | Gba | Fgf7 | Itgbl1 | Ltbp2 |
| | Ctss | Col1a2 | Gdf15 | Fmod | Jam3 | Lyz1 |
| | Ctsz | Col25a1 | Gdf6 | Fn1 | Kcp | Lyz2 |
| | Cxcl1 | Col3a1 | Gfer | Frzb | Lama2 | Man2b2 |
| | Cxcl14 | Col4a1 | Ggh | Fstl1 | Lama4 | Mfap4 |
| | Cxcl16 | Col4a2 | Gla | Fstl3 | Lama5 | Mfge8 |
| | Cxcl3 | Col5a2 | Glb1 | Gabbr1 | Lamb1 | Mmp14 |
| | Cyr61 | Col8a1 | Glb1l | Gas6 | Lamp2 | Mmp19 |
| | Dcn | Colec10 | Gldn | Gba | Lefty1 | Mmp2 |
| | Dkk2 | Cp | Gpc4 | Gdf11 | Lgi4 | Msln |
| | Dpt | Cpa4 | Gpx3 | Glb1 | Lingol | Nenf |
| | Ecm1 | Cpa6 | Grem1 | Gpc4 | Lox | Nov |
| | Edn1 | Cpb1 | Grn | Grem1 | Loxl2 | Npc2 |
| | Edn2 | Cpe | Gsn | Gsn | Ltbp2 | Nucb1 |
| | Efemp1 | Cpm | Hexb | Hgf | Lyz1 | Nucb2 |
| | Eng | Cpq | Hgf | Hist1h2bc | Lyz2 | Ogn |
| | Ereg | Cpxm1 | Hist1h2bc | Hist1h2be | Man2b2 | Pamr1 |
| | F3 | Cpxm2 | Hist1h2be | Hp | Mfap4 | Pcolce |
| | F5 | Cpz | Hyal1 | Hpgd | Mfge8 | Pcsk5 |
| | Fabp3 | Creg1 | Icam1 | Hspg2 | Mmp14 | Pdia4 |
| | Fap | Crtap | Igf1 | Htra1 | Mmp19 | Plat |
| | Fas | Csf1 | Igf2 | Igf1 | Mmp2 | Postn |
| | Fbln2 | Cst3 | Igf2r | Igf2 | Msln | Prelp |
| | Fbrs | Cst6 | Igfbp2 | Igfbp2 | Nenf | Prg4 |
| | Fgf10 | Cstb | Igfbp3 | Igfbp3 | Nid1 | Prss23 |
| | Fgf2 | Ctgf | Il15 | Igfbp6 | Nid2 | Rnase4 |
| | Fgf7 | Cthrc1 | Il1rap | Igfbp7 | Nov | S100a16 |
| | Fjx1 | Ctsb | Il4ra | Il4ra | Npc2 | Sema3b |
| | Flrt2 | Ctsd | Il6 | Inha | Nucb1 | Sema3c |
| | Fmod | Ctsf | Il7 | Inhbb | Nucb2 | Sema3f |
| | Fn1 | Ctsh | Inhba | Islr | Ogn | Sema4g |
| | Frzb | Ctsk | Inhbb | Itgbl1 | Pamr1 | Slit3 |
| | Fst | Ctsl | Islr | Jam3 | Pcolce | Smpd1 |
| | Fuca2 | Ctso | Itm2b | Kcp | Pcsk5 | Sod3 |
| | Gas6 | Ctss | Kitl | Lama2 | Pdia3 | Sparc |
| | Gba | Ctsz | Lama2 | Lama4 | Pdia4 | Srpx2 |
| | Gdf10 | Cxcl1 | Lamb1 | Lama5 | Pla2g15 | Ssc5d |
| | Gdf15 | Cxcl14 | Lamb2 | Lamb1 | Plat | Sulf1 |
| | Gdf6 | Cxcl15 | Lamp2 | Lamb2 | Postn | Tcn2 |
| | Gdnf | Cxcl16 | Lepr | Lamp2 | Ppbp | Tfpi |
| | Gfer | Cxcl3 | Lgals3bp | Lefty1 | Ppt1 | Tgfb2 |
| | Gfra1 | Cxcl5 | Lmcd1 | Lgi4 | Prelp | Tgfb3 |
| | Ggh | Cyr61 | Lrrn2 | Lingo1 | Prg4 | Thbs1 |
| | Ghr | Dag1 | Ltbp2 | Lox | Prss23 | Thbs2 |
| | Gla | Dcn | Lum | Loxl2 | Ptprz1 | Timp2 |
| | Glb1 | Dkk2 | Lyz1 | Loxl3 | Qsox1 | Timp3 |
| | Glbl1 | Dkk3 | Lyz2 | Lrfn1 | Rnase4 | Tinagl1 |
| | Gldn | Dpt | Man2b1 | Ltbp2 | S100a16 | Vcam1 |
| | Gpc4 | E130311K13Rik | Man2b2 | Lum | Sema3b | Wisp2 |
| | Gpx3 | Ecm1 | Manba | Lyz1 | Sema3c | Cd81 |
| | Grem1 | Edn2 | Masp1 | Lyz2 | Sema3f | Col20a1 |
| | Grem2 | Efemp1 | Mertk | Man2a1 | Sema4g | Galnt2 |
| | Grn | Efemp2 | Mgp | Man2b2 | Slit3 | Itm2a |
| | Gsn | Egf | Mmp13 | Masp1 | Smpd1 | Itm2c |
| | Hbegf | Emilin1 | Mmp14 | Matn2 | Sod3 | Naglu |
| | Hexb | Emilin2 | Mmp2 | Mfap4 | Sparc | Pcyox1 |
| | Hgf | Eng | Mmp3 | Mfap5 | Srpx2 | Snx18 |
| | Hgfac | Entpd1 | Mmrn2 | Mfge8 | Ssc5d | |
| | Hist1h2bc | Epdr1 | Nbl1 | Mmp14 | Sulf1 | |
| | Hist1h2be | Ereg | Nell2 | Mmp19 | Tcn2 | |
| | Hist1h2bg | F3 | Nid1 | Mmp2 | Tfpi | |
| | Hpx | F5 | Npc2 | Msln | Tgfb2 | |
| | Hspg2 | F8 | Nrn1 | Nbl1 | Tgfb3 | |
| | Hyal1 | Fabp3 | Nucb1 | Nenf | Thbs1 | |
| | Icam1 | Fam20b | Ogn | Nov | Thbs2 | |
| | Igf1 | Fas | Olfm1 | Npc2 | Timp2 | |
| | Igf2 | Fbln2 | Pam | Nucb1 | Timp3 | |
| | Igf2r | Fetub | Pamr1 | Nucb2 | Tinagl1 | |
| | Igfbp2 | Fgf10 | Pappa | Ogn | Tmsb4x | |
| | Igfbp3 | Fgf2 | Pcsk5 | Olfm1 | Tsku | |
| | Igfbp7 | Fgf7 | Pcsk6 | Pamr1 | Vcam1 | |
| | Il11 | Fgf9 | Pdgfd | Pappa | Wisp2 | |
| | Il15 | Fjx1 | Pf4 | Pcolce | Ccs | |
| | Il1rap | Fmod | Pla1a | Pcsk5 | Cd81 | |
| | Il34 | Fn1 | Pla2g6 | Pcsk6 | Cd9 | |
| | Il4ra | Frzb | Pla2g7 | Pdgfd | Col20a1 | |
| | Il6 | Fst | Plau | Pdia4 | Galnt2 | |
| | Il7 | Fstl1 | Pltp | Pla2g6 | Itm2a | |
| | Inhba | Fstl3 | Pm20d1 | Pla2g7 | Itm2c | |
| | Inhbb | Fuca2 | Pon3 | Plat | Naglu | |
| | Islr | Gabbr1 | Ppbp | Postn | Pcyox1 | |
| | Itm2b | Gas6 | Ppt1 | Prelp | Pkd1 | |
| | Kitl | Gba | Prelp | Prg4 | Rab11a | |
| | Klk8 | Gdf11 | Prg4 | Prss23 | Snx18 | |
| | Lama2 | Gdf15 | Pros1 | Psap | Srgn | |
| | Lamb1 | Gdf6 | Prss23 | Ptn | | |
| | Lamb2 | Gdf7 | Psap | Ptprg | | |
| | Lamc2 | Gfer | Ptprg | Ptx3 | | |
| | Lamp2 | Ggh | Rnase4 | Rarres2 | | |
| | Lepr | Gla | S100a13 | Rbp4 | | |
| | Lgals3bp | Glb1 | S100a16 | Rnase4 | | |
| | Lif | Glbl1 | S100a7a | S100a16 | | |
| | Lingo2 | Gldn | Scube3 | Sema3b | | |
| | Lipg | Gpc4 | Selp | Sema3c | | |
| | Liph | Gpx3 | Sema3b | Sema3d | | |
| | Lmcd1 | Grem1 | Sema3c | Sema3f | | |
| | Loxl3 | Grn | Sema3d | Sema4g | | |
| | Lrp2 | Gsdmd | Serpinb6a | Serpinb1a | | |
| | Lrrn1 | Gsn | Serpinb6b | Serpinb6b | | |
| | Lrrn2 | Hexb | Serpinb8 | Serpinb9b | | |
| | Ltbp2 | Hfe | Serpinb9 | Serping1 | | |
| | Lum | Hgf | Serpinb9b | Sfrp1 | | |
| | Lyz1 | Hist1h2bc | Serpine2 | Slit3 | | |
| | Lyz2 | Hist1h2be | Serping1 | Smpd1 | | |
| | Man2b1 | Hp | Serpini1 | Smpdl3a | | |
| | Man2b2 | Hpgd | Slit1 | Sod3 | | |
| | Manba | Hpx | Slit3 | Sparc | | |
| | Masp1 | Hspg2 | Slmap | Spon2 | | |
| | Mcam | Htra1 | Slpi | Srpx2 | | |
| | Mcpt8 | Hyal1 | Smpd1 | Ssc5d | | |
| | Mertk | Icam1 | Smpdl3a | Stc1 | | |
| | Metrnl | Ifnar2 | Sod3 | Sulf1 | | |
| | Mfge8 | Igf1 | Sorl1 | Svep1 | | |
| | Mgp | Igf2 | Spon2 | Tcn2 | | |
| | Mmp12 | Igf2r | Srpx2 | Tfpi | | |
| | Mmp13 | Igfbp2 | St14 | Tgfb2 | | |
| | Mmp14 | Igfbp3 | Stc1 | Tgfb3 | | |
| | Mmp2 | Igfbp6 | Sulf2 | Thbd | | |
| | Mmp3 | Igfbp7 | Tcn2 | Thbs1 | | |
| | Mmp9 | Il15 | Tfpi | Thbs2 | | |
| | Mmrn2 | Il17d | Tgfbr3 | Thbs4 | | |
| | Msln | Il1rap | Thbd | Timp2 | | |
| | Nbl1 | Il4ra | Thbs1 | Timp3 | | |
| | Nell1 | Il6 | Thbs2 | Tinagl1 | | |
| | Nell2 | Il7 | Timp2 | Tnfrsf1a | | |
| | Ngf | Inha | Timp3 | Tnfsf15 | | |
| | Nid1 | Inhba | Tinagl1 | Twsg1 | | |
| | Npc2 | Inhbb | Tnfsf15 | Vcam1 | | |
| | Npnt | Islr | Tnxb | Vcan | | |
| | Nppa | Itgbl1 | Trf | Vwc2 | | |
| | Nppb | Itm2b | Tsku | Wisp2 | | |
| | Nrg1 | Jam3 | Vnn1 | Wnt16 | | |
| | Nrn1 | Kcp | Vwc2 | Abi3bp | | |
| | Ntm | Kitl | Vwf | Aqp1 | | |
| | Nucb1 | Kng2 | Wfikkn2 | Cd81 | | |
| | Ogn | Lama2 | Wnt9a | Clca1 | | |
| | Olfm1 | Lama4 | Xdh | Cntfr | | |
| | Pam | Lama5 | Abca1 | Col20a1 | | |
| | Pamr1 | Lamb1 | Ccs | Galnt2 | | |
| | Pappa | Lamb2 | Cd274 | Gpc2 | | |
| | Pcsk5 | Lamp2 | Cd81 | Gpc6 | | |
| | Pcsk6 | Lbp | Clca1 | Itm2a | | |
| | Pdgfa | Lefty1 | Col20a1 | Itm2c | | |
| | Pdgfd | Lepr | Dhh | Naglu | | |
| | Pf4 | Lgals3bp | Itm2a | Pcyox1 | | |
| | Pgf | Lgi4 | Itm2c | Prrg2 | | |
| | Pla1a | Lingo1 | Ly96 | Rab4a | | |
| | Pla2g6 | Lipe | Naglu | Rab5b | | |
| | Pla2g7 | Liph | Pcyox1 | Snx18 | | |
| | Plau | Lmcd1 | Rab11a | | | |
| | Pltp | Lox | Rab4a | | | |
| | Pm20d1 | Loxl2 | Rab5b | | | |
| | Pon3 | Loxl3 | Serpinb10 | | | |
| | Ppbp | Lrfn1 | Snx18 | | | |
| | Ppt1 | Lrfn3 | Sord | | | |
| | Prelp | Lrrn1 | | | | |
| | Prg4 | Lrrn2 | | | | |
| | Prom1 | Lrrn3 | | | | |
| | Pros1 | Ltbp2 | | | | |
| | Prss23 | Lum | | | | |
| | Psap | Lyz1 | | | | |
| | Ptprg | Lyz2 | | | | |
| | Ptprz1 | Man2a1 | | | | |
| | Qsox1 | Man2b1 | | | | |
| | Ramp1 | Man2b2 | | | | |
| | Rnase4 | Manba | | | | |
| | S100a13 | Masp1 | | | | |
| | S100a16 | Matn2 | | | | |
| | S100a7a | Mcpt8 | | | | |
| | Scube3 | Mertk | | | | |
| | Sele | Mfap4 | | | | |
| | Selp | Mfap5 | | | | |
| | Sema3b | Mfge8 | | | | |
| | Sema3c | Mgp | | | | |
| | Sema3d | Mmp13 | | | | |
| | Sema3e | Mmp14 | | | | |
| | Sema4f | Mmp19 | | | | |
| | Serpinb2 | Mmp2 | | | | |
| | Serpinb6a | Mmp3 | | | | |
| | Serpinb6b | Mmrn2 | | | | |
| | Serpinb8 | Msln | | | | |
| | Serpinb9 | Nbl1 | | | | |
| | Serpinb9b | Nell2 | | | | |
| | Serpine1 | Nenf | | | | |
| | Serpine2 | Nid1 | | | | |
| | Serping1 | Nid2 | | | | |
| | Serpini1 | Nov | | | | |
| | Slit1 | Npc2 | | | | |
| | Slit3 | Nppa | | | | |
| | Slmap | Nrn1 | | | | |
| | Slpi | Nucb1 | | | | |
| | Smpd1 | Nucb2 | | | | |
| | Smpdl3a | Obscn | | | | |
| | Sod3 | Ogn | | | | |
| | Soga1 | Olfm1 | | | | |
| | Soga3 | Olfml2b | | | | |
| | Sorl1 | Pam | | | | |
| | Sparcl1 | Pamr1 | | | | |
| | Spink2 | Pappa | | | | |
| | Spon2 | Pcolce | | | | |
| | Srpx2 | Pcsk5 | | | | |
| | St14 | Pcsk6 | | | | |
| | Stc1 | Pdgfd | | | | |
| | Sulf2 | Pdia3 | | | | |
| | Tcn2 | Pdia4 | | | | |
| | Tfpi | Pecam1 | | | | |
| | Tgfa | Pf4 | | | | |
| | Tgfb2 | Pgf | | | | |
| | Tgfbr3 | Pi16 | | | | |
| | Thbd | Pla1a | | | | |
| | Thbs1 | Pla2g15 | | | | |
| | Thbs2 | Pla2g6 | | | | |
| | Timp1 | Pla2g7 | | | | |
| | Timp2 | Plat | | | | |
| | Timp3 | Plau | | | | |
| | Tinagl1 | Pltp | | | | |
| | Tmsb4x | Pm20d1 | | | | |
| | Tnc | Podn | | | | |
| | Tnfsf11 | Pon3 | | | | |
| | Tnfsf15 | Postn | | | | |
| | Tnxb | Ppbp | | | | |
| | Trf | Ppp1r1a | | | | |
| | Tsku | Ppt1 | | | | |
| | Tslp | Prelp | | | | |
| | Vegfc | Prg4 | | | | |
| | Vnn1 | Prl2c2 | | | | |
| | Vwc2 | Pros1 | | | | |
| | Vwf | Prss23 | | | | |
| | Wfikkn2 | Psap | | | | |
| | Wnt2 | Ptgis | | | | |
| | Wnt2b | Ptn | | | | |
| | Wnt4 | Ptprg | | | | |
| | Wnt5a | Ptprz1 | | | | |
| | Wnt9a | Ptx3 | | | | |
| | Xdh | Qsox1 | | | | |
| | Abca1 | Ramp1 | | | | |
| | Ahnak | Rarres2 | | | | |
| | Ccs | Rbp4 | | | | |
| | Cd274 | Rnase4 | | | | |
| | Cd81 | S100a13 | | | | |
| | Clca1 | S100a16 | | | | |
| | Cntfr | S100a7a | | | | |
| | Coch | S100b | | | | |
| | Col20a1 | Scube3 | | | | |
| | Dhh | Selp | | | | |
| | Itga4 | Sema3b | | | | |
| | Itm2a | Sema3c | | | | |
| | Itm2c | Sema3d | | | | |
| | Krt13 | Sema3f | | | | |
| | Loxl4 | Sema4a | | | | |
| | Ly96 | Sema4f | | | | |
| | Msr1 | Sema4g | | | | |
| | Naglu | Serpina3i | | | | |
| | Pcyox1 | Serpina3n | | | | |
| | Pdcd6ip | Serpinb1a | | | | |
| | Rab11a | Serpinb6a | | | | |
| | Rab4a | Serpinb6b | | | | |
| | Rab5b | Serpinb8 | | | | |
| | Rap2b | Serpinb9 | | | | |
| | Sdcbp | Serpinb9b | | | | |
| | Sema7a | Serpine2 | | | | |
| | Serpina3h | Serping1 | | | | |
| | Serpinb10 | Serpini1 | | | | |
| | Snx18 | Sfrp1 | | | | |
| | Sord | Sfrp4 | | | | |
| | | Slit1 | | | | |
| | | Slit3 | | | | |
| | | Slmap | | | | |
| | | Slpi | | | | |
| | | Smpd1 | | | | |
| | | Smpdl3a | | | | |
| | | Sod3 | | | | |
| | | Soga3 | | | | |
| | | Sorl1 | | | | |
| | | Spaca1 | | | | |
| | | Sparc | | | | |
| | | Sparcl1 | | | | |
| | | Spock3 | | | | |
| | | Spon2 | | | | |
| | | Spp1 | | | | |
| | | Srpx2 | | | | |
| | | Ssc5d | | | | |
| | | St14 | | | | |
| | | Stc1 | | | | |
| | | Sulf1 | | | | |
| | | Sulf2 | | | | |
| | | Svep1 | | | | |
| | | Tcn2 | | | | |
| | | Tctn1 | | | | |
| | | Tfpi | | | | |
| | | Tgfb2 | | | | |
| | | Tgfb3 | | | | |
| | | Tgfbr3 | | | | |
| | | Thbd | | | | |
| | | Thbs1 | | | | |
| | | Thbs2 | | | | |
| | | Thbs4 | | | | |
| | | Thpo | | | | |
| | | Timp1 | | | | |
| | | Timp2 | | | | |
| | | Timp3 | | | | |
| | | Tinagl1 | | | | |
| | | Tmsb4x | | | | |
| | | Tnfrsf11b | | | | |
| | | Tnfrsf1a | | | | |
| | | Tnfsf12 | | | | |
| | | Tnfsf15 | | | | |
| | | Tnfsf18 | | | | |
| | | Tnfsf9 | | | | |
| | | Tnxb | | | | |
| | | Trf | | | | |
| | | Tsku | | | | |
| | | Tslp | | | | |
| | | Twsg1 | | | | |
| | | Vcam1 | | | | |
| | | Vcan | | | | |
| | | Vegfb | | | | |
| | | Vnn1 | | | | |
| | | Vpreb1 | | | | |
| | | Vwc2 | | | | |
| | | Vwf | | | | |
| | | Wfdc1 | | | | |
| | | Wfikkn2 | | | | |
| | | Wisp2 | | | | |
| | | Wnt10a | | | | |
| | | Wnt11 | | | | |
| | | Wnt16 | | | | |
| | | Wnt9a | | | | |
| | | Xdh | | | | |
| | | Abca1 | | | | |
| | | Abi3bp | | | | |
| | | Ahnak | | | | |
| | | Aqp1 | | | | |
| | | Ccs | | | | |
| | | Cd274 | | | | |
| | | Cd81 | | | | |
| | | Cd9 | | | | |
| | | Clca1 | | | | |
| | | Cntfr | | | | |
| | | Col20a1 | | | | |
| | | Dhh | | | | |
| | | Enox2 | | | | |
| | | Galnt2 | | | | |
| | | Gpc2 | | | | |
| | | Gpc6 | | | | |
| | | Itga4 | | | | |
| | | Itm2a | | | | |
| | | Itm2c | | | | |
| | | Loxl4 | | | | |
| | | Ly96 | | | | |
| | | Naglu | | | | |
| | | Pcyox1 | | | | |
| | | Pkd1 | | | | |
| | | Prrg2 | | | | |
| | | Rab11a | | | | |
| | | Rab4a | | | | |
| | | Rab5b | | | | |
| | | Sdc1 | | | | |
| | | Serpinb10 | | | | |
| | | Slc2a4 | | | | |
| | | Snx18 | | | | |
| | | Sord | | | | |
| | | Srgn | | | | |
| | | Xpnpep2 | | | | |

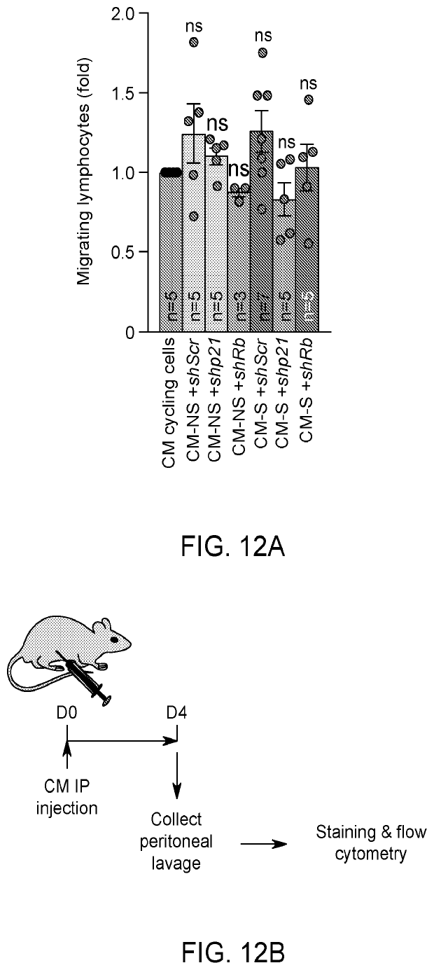

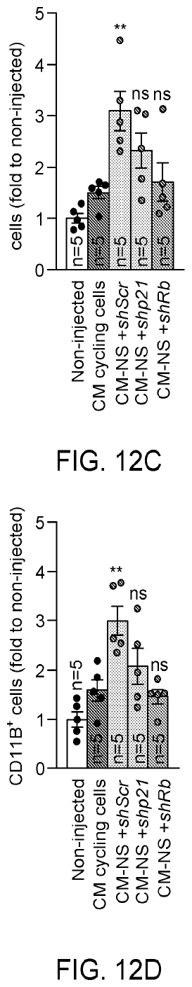

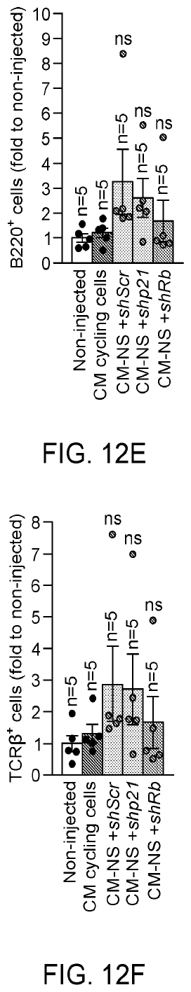

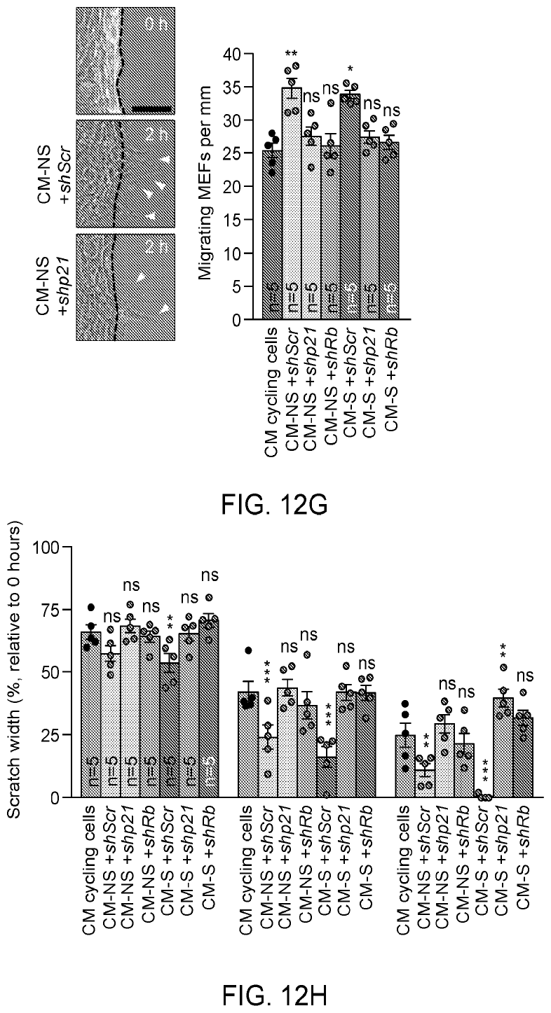

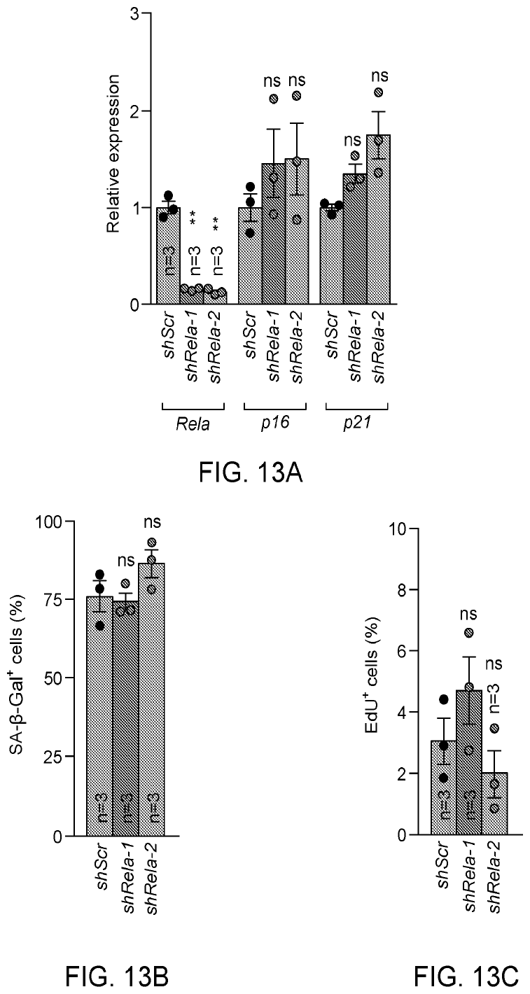

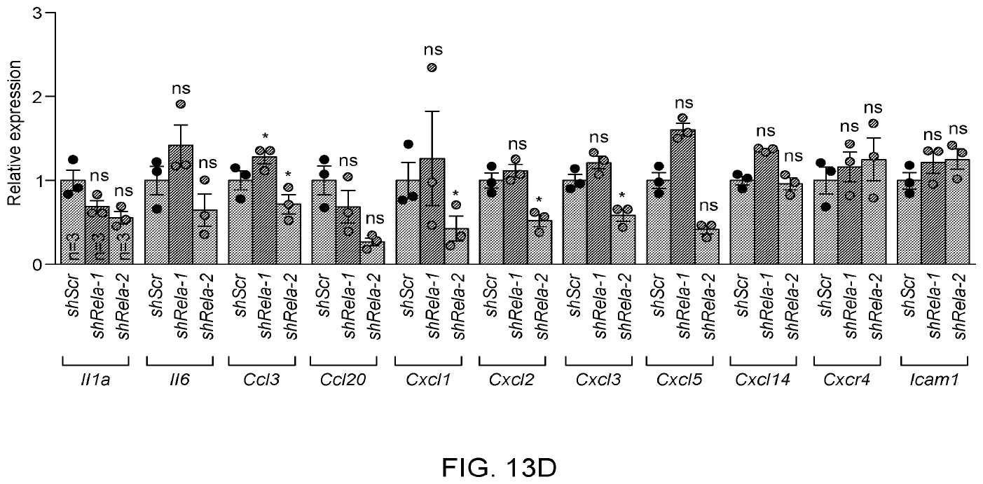

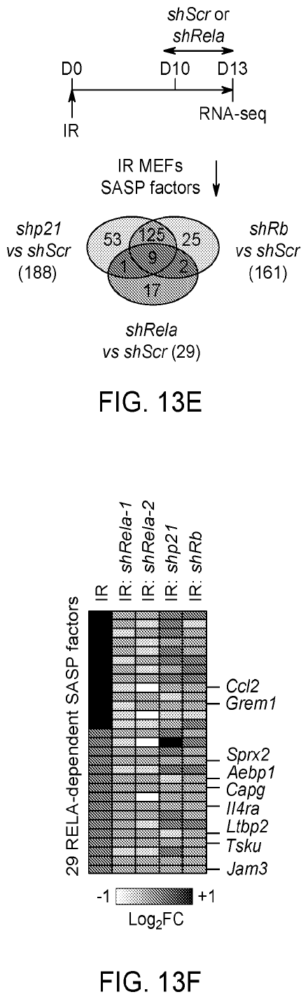

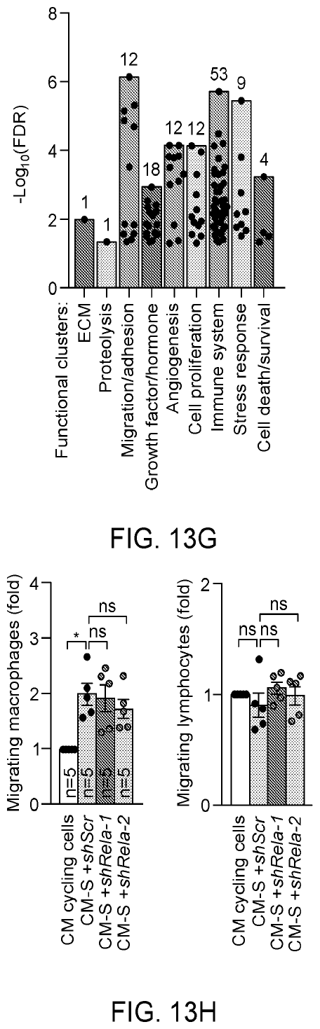

Functional annotation analysis on the 84 shared PASP factors indicated that several traits of SNCs might be P21-RB dependent, including features involving cell migration/adhesion and the immune system (FIG. 1H and FIG. 11G), raising speculation about a possible role of the PASP in immunosurveillance. To test this idea, the extent to which the PASP impacts the migratory behavior of mouse peritoneal immune cells was determined in a transwell system (FIG. 1I). Conditioned medium from D4 non-senescent (CM-NS) or D10 senescent (CM-S) IR MEFs promoted transwell migration of macrophages, a property that was lost with CM-NS and CM-S from p21- or Rb-depleted IR MEFs (FIG. 1J). None of the CMs impacted lymphocyte migration in this assay (FIG. 12A). In a second migration assay, macrophage numbers selectively increased in the peritoneal lavage 4 days after intraperitoneal injection of CM-NS, but not after injection of CM-NS from p21- or Rb-depleted IR MEFs (FIGS. 12B to 12F). The PASP also stimulated cell movement in a standard scratch assay on cultured MEFs (FIGS. 12G and 12H), indicating that its promigratory properties extend beyond macrophages. NFkB P65 (RELA) appeared to have no role in establishing the PASP or its macrophage-attracting properties (FIG. 13 and Table 2).

TABLE 2
Downregulated SASP factors upon knockdown of RELA
in IR-senescent MEFs.

| | | | IR-SASP |
|---|---|---|---|
| | | IR-SASP | factors that are |
| IR-SASP | | factors that | downregulated |
| factors that | | are down- | in shRela-1 |
| are | IR-SASP | regulated in | vs. shScr AND |
| down- | factors that | shRela-1 | shRela-2 vs. |
| regulated | are down- | vs. shScr | shScr AND shp21 |
| in | regulated in | AND | vs. shScr |
| shRela-1 | shRela-2 | shRela-2 vs. | AND shRb |
| vs. shScr | vs. shScr | shScr | vs. shScr |
| Lman2 | Thbd | Aebp1 | Aebp1 |
| Aebp1 | Ghr | Sdcbp | Ltbp2 |
| Cmtm3 | Aebp1 | Adm | Jam3 |
| Ssc5d | Slit3 | Ltbp2 | Tsku |
| Capg | Thbs2 | Jam3 | Il4ra |
| Tsku | Pla2g7 | Atrn | Srpx2 |
| C1qtnf3 | Sdcbp | Tsku | Capg |
| Rnase4 | Serping1 | Il4ra | Grem1 |
| Thbs1 | Adm | Kitl | Ccl2 |
| Smpd1 | Ltbp2 | Srpx2 | |
| Sdcbp | Ctsc | Mgp | |
| Mgp | Jam3 | Sema4d | |
| Atrn | Icam1 | Capg | |
| Srpx2 | C1qa | Lif | |
| Il4ra | Fn1 | Dkk2 | |
| Gfer | Rab4a | Serpinb2 | |
| Ltbp2 | Sod3 | Inhba | |
| Vegfc | Cd9 | Spp1 | |
| Grem2 | Rablla | Cxcl16 | |
| Itgbl1 | Atrn | Grem1 | |
| Cxcl16 | Lama2 | Mmp3 | |
| Spp1 | Lyz2 | Mmp 12 | |
| Cpa6 | Tinagl1 | Arg1 | |
| Timp2 | Mfap5 | Ccl6 | |
| Dkk3 | Clqc | C3 | |
| Fgf2 | Lamb1 | Serpina3i | |
| Trf | Ctss | Plau | |
| Jam3 | Pappa | Grem2 | |
| Adm | Tsku | Ccl2 | |
| Kitl | Il4ra | | |
| Arg1 | Angpt2 | | |
| Lif | Hgf | | |
| Igf2r | Sfrp2 | | |
| Ccl6 | Kitl | | |
| Nrn1 | Ppbp | | |
| Wnt9a | Hfe | | |
| Tnfrsf11b | Srpx2 | | |
| C3 | Cmtm4 | | |
| Pon3 | Serpinb6b | | |
| Inhba | Cd14 | | |
| Kcp | Nppb | | |
| Wnt11 | Mgp | | |
| Gsdmd | Ctsb | | |
| Cpm | Cnp | | |
| Mmp3 | Twsg1 | | |
| Cmtm6 | C1qb | | |
| Scube3 | Sulf2 | | |
| Flrt2 | Bmp6 | | |
| Pros1 | Col20a1 | | |
| Plau | Sema4d | | |
| Dkk2 | Ptx3 | | |
| S100b | Lgals3 | | |
| Olfm1 | Wnt4 | | |
| Wnt5a | Capg | | |
| Ccl2 | Serpina3n | | |
| Gusb | Prg4 | | |
| Angpt14 | Lif | | |
| Mmp12 | Entpd1 | | |
| Tgfa | Tnxb | | |
| Vnn1 | Il11 | | |
| Pdgfa | Cpxml | | |
| Serpinb2 | Xdh | | |
| Il18 | Il1rap | | |
| Serpina3i | Abi3bp | | |
| Sema4d | Dkk2 | | |
| Fam19a3 | Fgf10 | | |
| Grem1 | Il34 | | |
| Cxcl9 | Cxcl14 | | |
| Wnt6 | Serpinb2 | | |
| Cxcl10 | Prelp | | |
| Sfrp1 | Cck | | |
| Adam9 | Itgam | | |
| | Fam20b | | |
| | Inhba | | |
| | Prl2c2 | | |
| | Spp1 | | |
| | Csf1 | | |
| | Cxcl16 | | |
| | Msr1 | | |
| | Ccl9 | | |
| | Slpi | | |
| | Glb1 | | |
| | Cfb | | |
| | Cp | | |
| | Grem1 | | |
| | Anpep | | |
| | Tnfsf15 | | |
| | Mmp3 | | |
| | Stc1 | | |
| | Fgf7 | | |
| | F3 | | |
| | Scnnla | | |
| | Mmp12 | | |
| | Tac1 | | |
| | Btc | | |
| | Arg1 | | |
| | Hp | | |
| | Tgfbr3 | | |
| | Ccl6 | | |
| | Ccl3 | | |
| | Ccl8 | | |
| | C3 | | |
| | Il6 | | |
| | Serpina3i | | |
| | Cxcl5 | | |
| | Plau | | |
| | Grem2 | | |
| | Ccl11 | | |
| | Ccl12 | | |
| | Prss2 | | |
| | Ccl7 | | |
| | Cxcl2 | | |
| | Ccl2 | | |
| | Cxcl1 | | |
| | Edn2 | | |
| | Saa3 | | |
| | Tnfsf18 | | |
| | Proz | | |
| | Lcn2 | | |

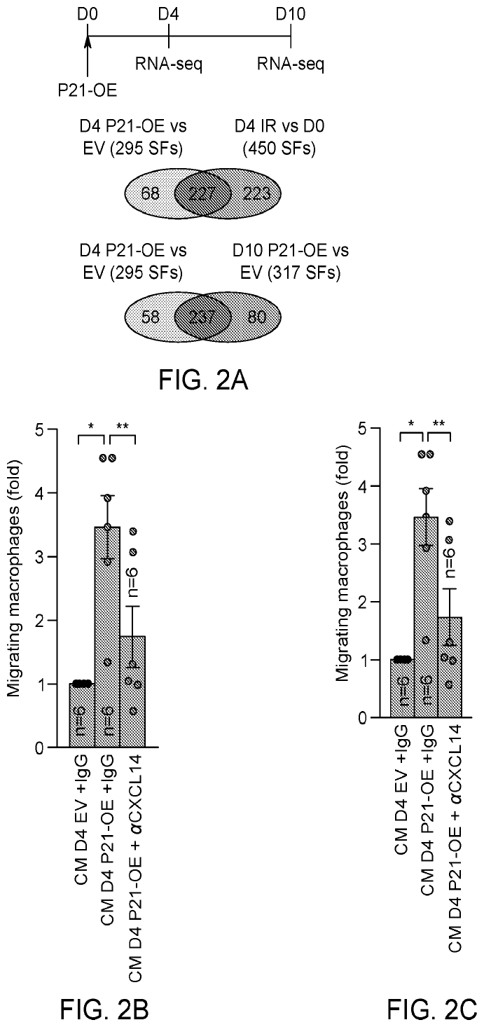

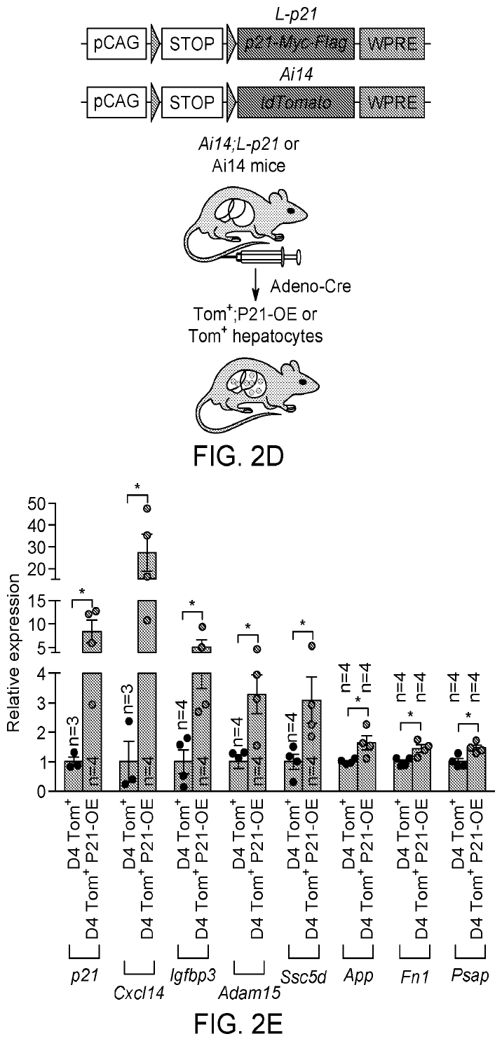

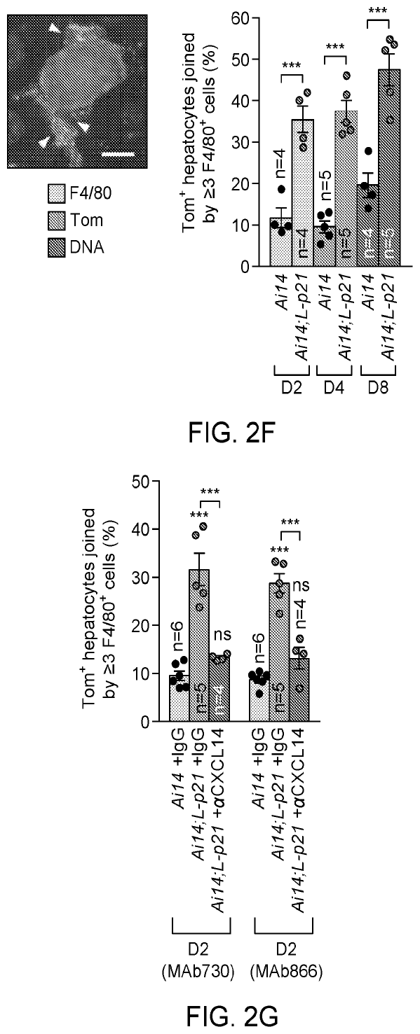

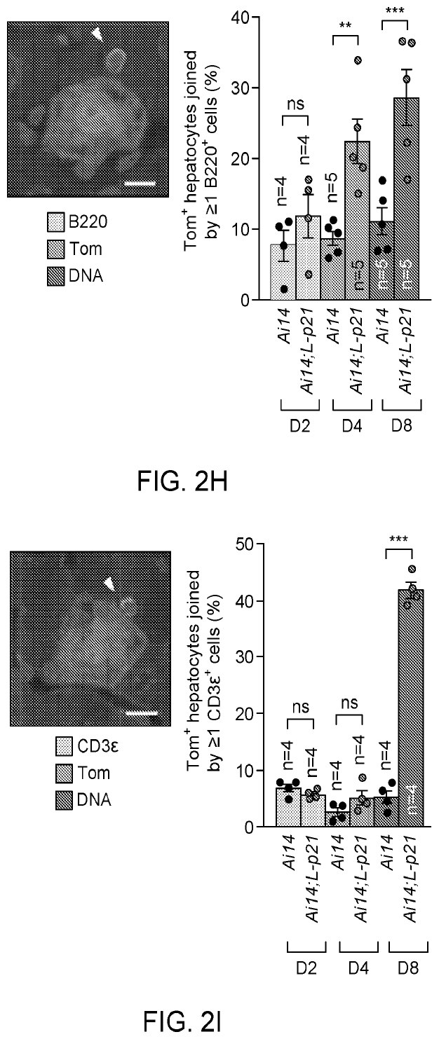

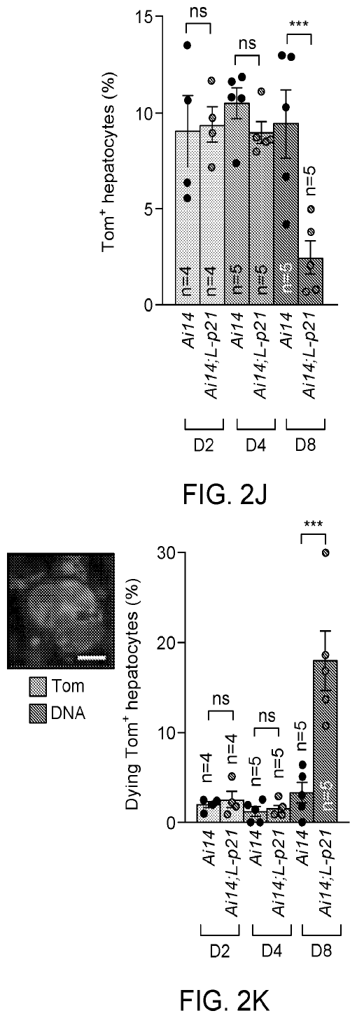

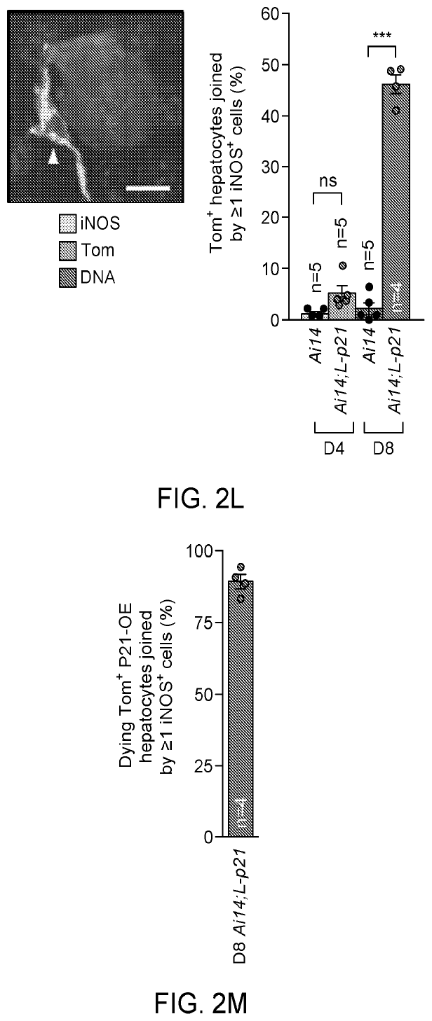

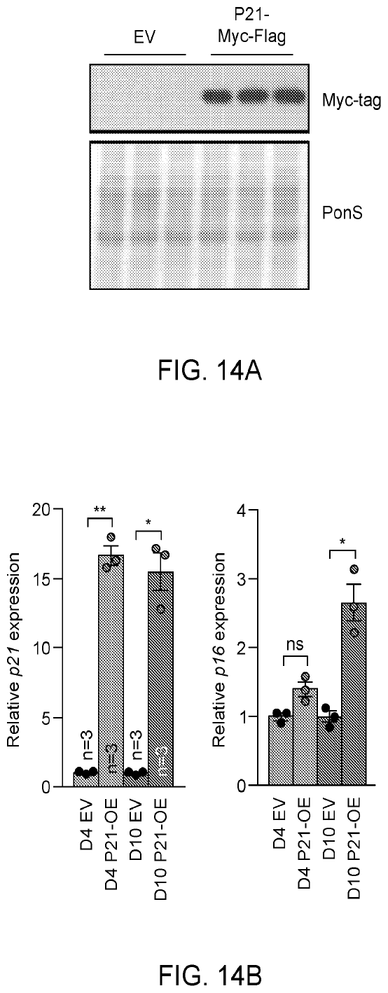

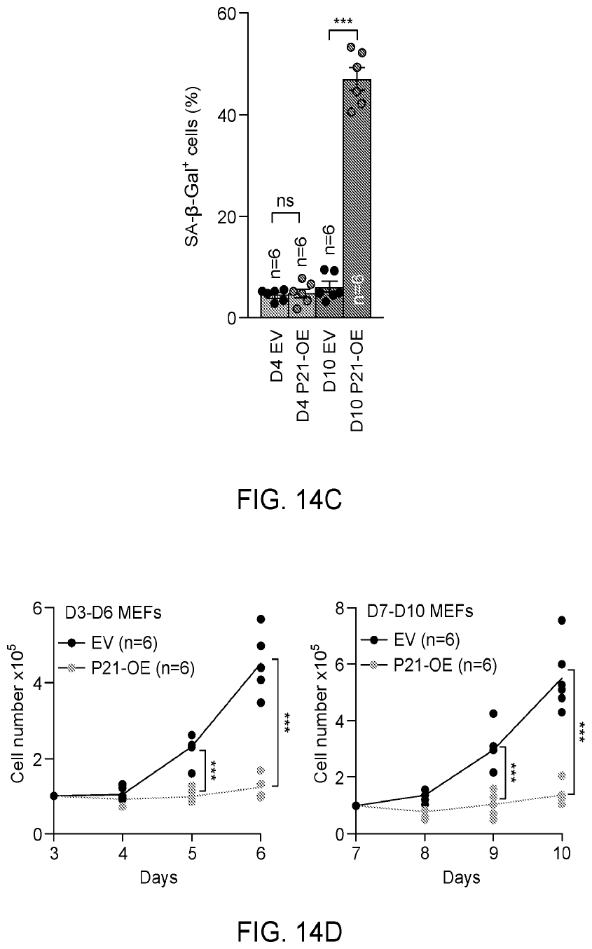

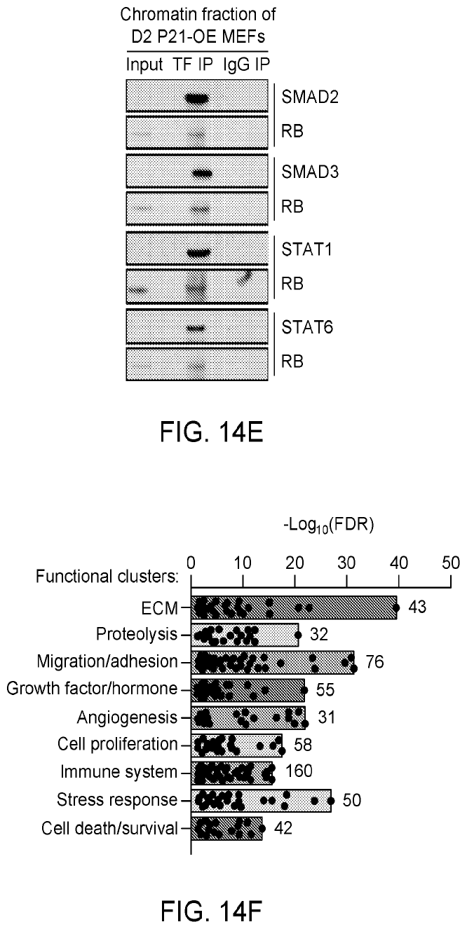

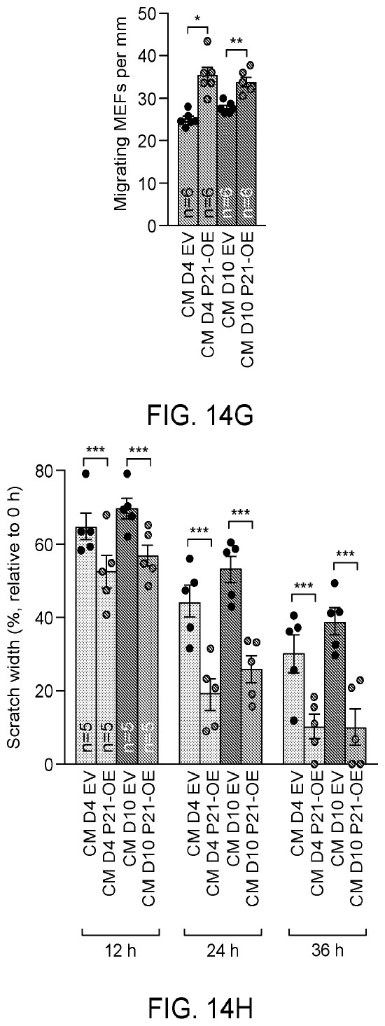

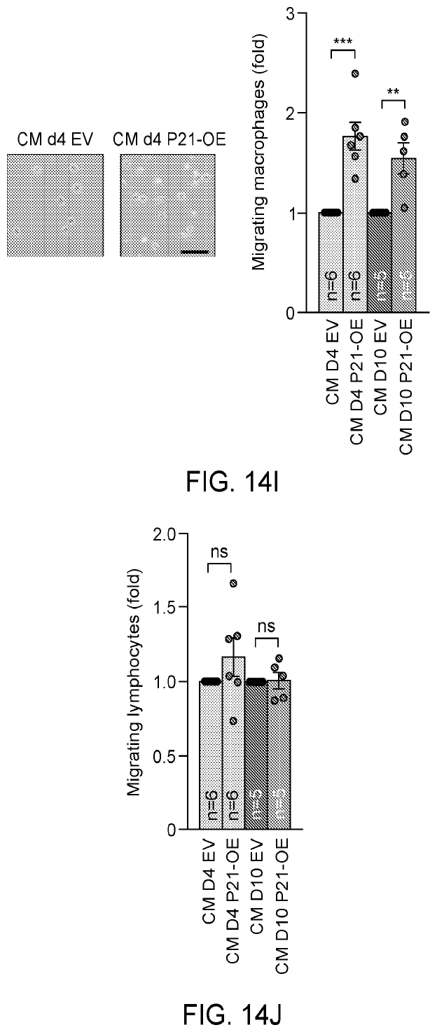

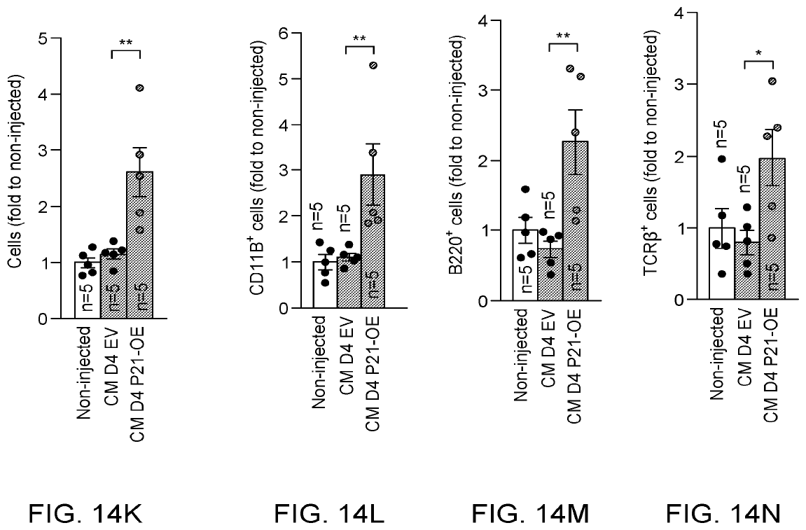

To determine whether the PASP requires an actual senescence-inducing stressor or merely elevated P21 levels, we transduced MEFs with a lentivirus harboring p21-Myc-Flag (FIG. 14A). P21-overexpressing (P21-OE) MEFs were subject to growth arrest, initially without elevated p16 and SA-β-Gal activity (D4), and later with these senescence markers (D10) (FIGS. 14B to 14D). D4 P21-OE MEFs upregulated 295 SFs, 227 of which were also upregulated in D4 IR MEFs, indicating that P21 induction is sufficient to yield a PASP (FIG. 2A and Table 3). SMAD2, SMAD3, STAT1 and STAT6 co-immunoprecipitated RB from the chromatin fraction of D4 P21-OE MEFs (FIG. 14E), further supporting that P21-induced hypophosphorylated RB interacts with STAT and SMAD TFs at select gene promoters to establish the PASP. PASP factors of D4 P21-OE MEFs were largely preserved in D10 P21-OE MEFs (FIG. 2A and Table 3), strengthening the conclusion that the PASP becomes an integral part of the SASP as cells senescence.

TABLE 3
Induction of secreted factor (SF) expression after P21-OE, P27-OE or P16-OE in MEFs.

| | | P21-OE D4 & | P16-OE | P16-OE | P16-OE D4 | P27- | | P27-OE D4 |
|---|---|---|---|---|---|---|---|---|
| | P21-OE D10 | D10 SFs | D4 | D10 | & D10 SFs | OE D4 | P27-OE D10 | & D10 SFs |
| P21-OE D4 SFs | SFs | overlap | SFs | SFs | overlap | SFs | SFs | overlap |
| 1500015O10Rik | 1500015O10Rik | 1500015O10Rik | Adam15 | Acpp | Adam15 | A2m | 1190002N15Rik | C1qtnf3 |
| Acpp | Acpp | Acpp | Adam9 | Adam10 | Adam9 | Acan | Adam15 | Ccdc80 |
| Adam15 | Adam10 | Adam15 | Adamts4 | Adam15 | Adamts4 | Adam12 | Adamts5 | Col8a1 |
| Adam9 | Adam15 | Adam9 | Adamts5 | Adam9 | Adamts5 | Anxa1 | Adamts9 | Cxcl14 |
| Adamts4 | Adam9 | Adamts4 | Adamtsl4 | Adamts4 | Aga | Bmp1 | Adamtsl4 | Ecm1 |
| Adamtsl4 | Adamts4 | Adamtsl4 | Aga | Adamts5 | Angpt4 | Bmp8b | Aga | Fn1 |
| Adm | Adamtsl4 | Aebp1 | Angpt4 | Aga | Angptl4 | Bmper | App | Gpc4 |
| Aebp1 | Adcyap1 | Aga | Angptl4 | Angpt4 | Anxa1 | C1qtnf3 | Arsa | Hist1h2bc |
| Aga | Aebp1 | Angpt4 | Anxa1 | Angptl4 | Aoc3 | C3 | B2m | Hspg2 |
| Agrn | Aga | Angptl4 | Anxa2 | Anxa1 | App | Calr | Bdnf | Igf2r |
| Angpt4 | Angpt4 | Anxa1 | Aoc3 | Aoc3 | Arsa | Ccdc80 | Bmp2 | Inhba |
| Angptl4 | Angptl2 | Anxa2 | Apob | App | Asahl | Ccl20 | Bmp4 | Lama5 |
| Anxa1 | Angptl4 | Anxa5 | Apoe | Arsa | Atrn | Cd40 | C1qtnf3 | Ltbp2 |
| Anxa2 | Anxa1 | Aoc3 | App | Asah1 | Bdnf | Col11a1 | Clqtnf4 | Nppb |
| Anxa5 | Anxa2 | Apob | Arsa | Atrn | Bgn | Col15a1 | Ccdc80 | Pappa |
| Aoc3 | Anxa5 | Apoe | Asah1 | Bdnf | Bmp1 | Col2a1 | Cd109 | Prg4 |
| Apob | Aoc3 | App | Atrn | Bgn | C1qtnf2 | Col4a1 | Cdh13 | Sema3c |
| Apoe | Apob | Arsa | Axl | Bmp1 | C1qtnf3 | Col4a2 | Cfh | Serpinb9b |
| App | Apoe | Asah1 | B2m | Btc | Ccbe1 | Col5a1 | Chid1 | Sod3 |
| Arg1 | Apoh | Atrn | Bdnf | C1qtnf2 | Ccdc80 | Col6a3 | Chrd | Thbs1 |
| Arsa | App | Axl | Bgn | C1qtnf3 | Ccl7 | Col8a1 | Clu | Timp3 |
| Asah1 | Arsa | B2m | Bmp1 | C1ra | Cd40 | Ctgf | Cmtm3 | Tinagl1 |
| Atrn | Arsg | B4galt1 | C1qa | C1s1 | Cd63 | Cxcl1 | Col23a1 | Tnfsf15 |
| Axl | Asah1 | Bcan | C1qtnf2 | Ccbe1 | Chid1 | Cxcl14 | Col8a1 | Snx18 |
| B2m | Atrn | Bdnf | C1qtnf3 | Ccdc80 | Clu | Ecm1 | Comp | |
| B4galt1 | Axl | Bgn | Ccbe1 | Cck | Cmtm3 | Edn1 | Cpa6 | |
| Bcan | B2m | Bmp1 | Ccdc80 | Ccl17 | Col4a1 | Ereg | Cpe | |
| Bdnf | B4galt1 | Bmp8b | Ccl2 | Ccl7 | Col4a2 | F5 | Cpm | |
| Bgn | Bcan | C1qa | Ccl7 | Cd40 | Col5a1 | Fam20a | Cpq | |
| Bmp1 | Bche | C1qc | Cd40 | Cd63 | Col5a2 | Fbln2 | Cpxm1 | |
| Bmp8b | Bdnf | C1qtnf2 | Cd63 | Cfh | Col8a1 | Fbn1 | Csf1 | |
| C1qa | Bgn | C1qtnf3 | Chid1 | Chid1 | Cpa6 | Fgf7 | Cst3 | |
| C1qc | Bmp1 | C1rl | Clu | Clu | Cpe | Fjx1 | Cst6 | |
| C1qtnf1 | Bmp8b | C1s1 | Cmtm3 | Cmtm3 | Cpm | Flrt2 | Ctsd | |
| C1qtnf2 | Btc | Cap1 | Cmtm7 | Col4a1 | Cpq | Fn1 | Ctsf | |
| C1qtnf3 | C1qa | Capg | Col1a1 | Col4a2 | Cpxm2 | Fst | Ctsh | |
| C1qtnf6 | C1qb | Cat | Col1a2 | Col5a1 | Creg1 | Gpc4 | Ctso | |
| C1rl | C1qc | Ccbe1 | Col4a1 | Col5a2 | Crlf1 | Hist1h2bc | Cxcl14 | |
| C1s1 | C1qtnf2 | Ccdc80 | Col4a2 | Col8a1 | Cst3 | Hspg2 | Ecm1 | |
| C3 | C1qtnf3 | Cck | Col5a1 | Cpa4 | Ctgf | Igf2r | Efemp1 | |
| Calr | C1ra | Ccl17 | Col5a2 | Cpa6 | Ctsb | Igfbp7 | Epdr1 | |
| Cap1 | C1rl | Ccl7 | Col8a1 | Cpe | Ctsd | Inhba | Fabp3 | |
| Capg | C1s1 | Cd40 | Cpa6 | Cpm | Ctsk | Lama1 | Fas | |
| Cat | C2 | Cd63 | Cpe | Cpq | Cts1 | Lama2 | Fgf10 | |
| Ccbe1 | Cap1 | Cfh | Cpm | Cpxm2 | Ctsz | Lama5 | Fgf2 | |
| Ccdc80 | Capg | Chid1 | Cpq | Creg1 | Ecm1 | Lamb1 | Fmod | |
| Cck | Cat | Clu | Cpxm2 | Crlf1 | Edn1 | Lox | Fn1 | |
| Ccl17 | Ccbe1 | Col4a1 | Creg1 | Cst3 | Efemp1 | Loxl2 | Fuca2 | |
| Ccl20 | Ccdc80 | Col4a2 | Crlf1 | Ctgf | Eng | Loxl3 | Gas6 | |
| Ccl7 | Cck | Col5a1 | Crtap | Ctsb | F3 | Ltbp2 | Gdf6 | |
| Cd40 | Ccl17 | Col5a2 | Csf1 | Ctsd | F5 | Mcpt8 | Ggh | |
| Cd63 | Ccl2 | Col8a1 | Cst3 | Ctsf | Fbln2 | Nid1 | Glb1 | |
| Cfh | Ccl7 | Cpa4 | Cstb | Ctsh | Fgf2 | Nppb | Gldn | |
| Chid1 | Cd40 | Cpa6 | Ctgf | Ctsk | Fjx1 | Pappa | Gpc4 | |
| Clu | Cd63 | Cpe | Ctsb | Ctsl | Flrt2 | Pla2g7 | Grem1 | |
| Cmtm7 | Cdh13 | Cpm | Ctsd | Ctsz | Fn1 | Ppbp | Gsn | |
| Col11a1 | Cdnf | Cpn1 | Ctsk | Cxcl14 | Fstl3 | Prg4 | Hist1h2bc | |
| Col1a1 | Cfh | Cpq | Ctsl | Dpysl3 | Fuca2 | Pxdn | Hist1h2be | |
| Col1a2 | Chid1 | Cpxml | Ctso | Ecm1 | Gas6 | Sema3c | Hspg2 | |
| Col4a1 | Clu | Creg1 | Ctsz | Edn1 | Gba | Serpinb9b | Igf2r | |
| Col4a2 | Cmtm3 | Crlf1 | Cyr61 | Efemp1 | Gdf15 | Serpine1 | Igfbp4 | |
| Col5a1 | Col4a1 | Crtap | Ecm1 | Eng | Glb1 | Slit2 | Il34 | |
| Col5a2 | Col4a2 | Csf1 | Edn1 | F3 | Gldn | Sod3 | Inhba | |
| Col8a1 | Col5a1 | Cst3 | Efemp1 | F5 | Gpc4 | Sulf1 | Islr | |
| Cpa4 | Col5a2 | Cst6 | Efemp2 | Fas | Gpx3 | Tfrc | Lama5 | |
| Cpa6 | Col8a1 | Cstb | Eng | Fbln2 | Grem2 | Thbs1 | Lamb2 | |
| Cpe | Comp | Ctsb | F3 | Fgf10 | Grn | Timp1 | Lingo1 | |
| Cpm | Cpa4 | Ctsd | F5 | Fgf2 | Gsn | Timp3 | Lipg | |
| Cpn1 | Cpa6 | Ctsf | Fbln2 | Fjx1 | Hist1h2bc | Tinagl1 | Lmcd1 | |
| Cpq | Cpe | Ctsh | Fgf2 | Flrt2 | Hist1h2bg | Tnc | Ltbp2 | |
| Cpxm1 | Cpm | Ctsk | Fjx1 | Fn1 | Htra1 | Tnfrsf11b | Man2b1 | |
| Cpxm2 | Cpn1 | Ctsl | Flrt2 | Fstl3 | Igfbp3 | Tnfsf15 | Man2b2 | |
| Creg1 | Cpq | Ctss | Fmod | Fuca2 | Igfbp4 | Vcan | Manba | |
| Crlf1 | Cpxm1 | Ctsz | Fn1 | Gas6 | Igfbp7 | Wisp1 | Masp1 | |
| Crtap | Creg1 | Cxcl14 | Fst13 | Gba | Inhba | Wisp2 | Mgp | |
| Csf1 | Crlf1 | Cxcl16 | Fuca2 | Gdf15 | Itm2b | Apobr | Nbl1 | |
| Csn3 | Crtap | Dag1 | Gas6 | Glb1 | Klk8 | Clca1 | Nog | |
| Cst3 | Csf1 | Dpysl3 | Gba | Gldn | Lamb1 | Coch | Npc2 | |
| Cst6 | Cst3 | Ecm1 | Gdf15 | Gpc4 | Lgals3bp | Dpep1 | Nppa | |
| Cstb | Cst6 | Edn1 | Gla | Gpx3 | Loxl2 | Hspa4 | Nppb | |
| Ctgf | Cstb | Efemp1 | Glb1 | Grem2 | Loxl3 | Snx18 | Nrn1 | |
| Ctsb | Ctsb | Efemp2 | Gldn | Grn | Ltbp2 | | Ogn | |
| Ctsd | Ctsd | Eng | Gpc4 | Gsn | Man2b1 | | Olfm1 | |
| Ctsf | Ctsf | Ereg | Gpx3 | Hist1h2bc | Man2b2 | | Pam | |
| Ctsh | Ctsh | F3 | Grem2 | Hist1h2be | Manba | | Pamr1 | |
| Ctsk | Ctsk | F5 | Grn | Hist1h2bg | Mcpt8 | | Pappa | |
| Ctsl | Ctsl | Fabp3 | Gsn | Htra1 | Mfap5 | | Pcsk5 | |
| Ctss | Ctss | Fas | Hbegf | Icam1 | Mfge8 | | Pcsk6 | |
| Ctsw | Ctsz | Fbln2 | Hist1h2bc | Igf1 | Mmp19 | | Plau | |
| Ctsz | Cxcl14 | Fgf2 | Hist1h2bg | Igfbp3 | Mmp2 | | Pm20d1 | |
| Cxcl14 | Cxcl15 | Fjx1 | Hspg2 | Igfbp4 | Nbl1 | | Ppt1 | |
| Cxcl16 | Cxcl16 | Fn1 | Htra1 | Igfbp7 | Ngf | | Prelp | |
| Cyr61 | Cyp4a12b | Fst13 | Igfbp2 | Il18 | Nid1 | | Prg4 | |
| Dag1 | Dag1 | Fuca2 | Igfbp3 | Il6 | Npc2 | | Prss23 | |
| Dbi | Dcn | Gas6 | Igfbp4 | Inhba | Nppb | | Psap | |
| Dpys13 | Dkk2 | Gba | Igfbp6 | Itgbl1 | Olfm1 | | Reln | |
| Ecm1 | Dpysl3 | Gdf15 | Igfbp7 | Itm2b | Pappa | | Rnase4 | |
| Edn1 | Ecm1 | Ghr | Il34 | Klk8 | Pcsk5 | | S100a13 | |
| Edn2 | Edn1 | Gla | Inhba | Lamb1 | Pdgfa | | S100a16 | |
| Efemp1 | Efemp1 | Glb1 | Itm2b | Lamp2 | Pdgfd | | Scube1 | |
| Efemp2 | Efemp2 | Gldn | Klk8 | Lgals3bp | Pgf | | Scube3 | |
| Eng | Eng | Gpc4 | Lama2 | Lingo1 | Pla1a | | Sema3c | |
| Ereg | Epdr1 | Grem2 | Lamb1 | Loxl2 | Pla2g15 | | Sema3d | |
| F3 | Ereg | Grn | Lgals3bp | Loxl3 | Plau | | Serpinb8 | |
| F5 | F3 | Gsn | Lipg | Lrrn1 | Pon3 | | Serpinb9b | |
| Fabp3 | F5 | Gusb | Lox | Ltbp2 | Prss23 | | Serpine2 | |
| Fas | F8 | Hbegf | Loxl2 | Man2b1 | Psap | | Slmap | |
| Fbln2 | Fabp3 | Hist1h2bc | Loxl3 | Man2b2 | Qsox1 | | Slurp1 | |
| Fgf2 | Fam19a3 | Hist1h2be | Ltbp2 | Manba | Rnase4 | | Smpd1 | |
| Fjx1 | Fam20b | Htra1 | Man2b1 | Mcam | S100a13 | | Smpdl3a | |
| Fmod | Fas | Hyal1 | Man2b2 | Mcpt8 | S100a16 | | Sod3 | |
| Fn1 | Fbln2 | Icam1 | Manba | Mfap5 | Selp | | Sorl1 | |
| Fstl3 | Fgf1 | Igf2r | Masp1 | Mfge8 | Serpinb2 | | St14 | |
| Fuca2 | Fgf10 | Igfbp3 | Mcpt8 | Mmp19 | Serpinb6a | | Sulf2 | |
| Gas6 | Fgf2 | Igfbp4 | Metrnl | Mmp2 | Serpinb6b | | Tcn2 | |
| Gba | Fgf9 | Igfbp7 | Mfap5 | Mmp9 | Serpinb9b | | Tgfb2 | |
| Gdf15 | Fjx1 | Il17d | Mfge8 | Nbl1 | Serpine1 | | Tgfb3 | |
| Gdf6 | Flrt2 | Il34 | Mmp13 | Ngf | Serping1 | | Thbd | |
| Ghr | Fn1 | Il7 | Mmp14 | Nid1 | Smpdl3a | | Thbs1 | |
| Gla | Fstl3 | Inhba | Mmp19 | Npc2 | Sparc | | Timp2 | |
| Glb1 | Fuca2 | Itgam | Mmp2 | Nppb | Srpx2 | | Timp3 | |
| Glbl1 | Gas6 | Itm2b | Nbl1 | Olfm1 | Tcn2 | | Tinagl1 | |
| Gldn | Gba | Jam3 | Nell2 | Pappa | Tfpi | | Tnfsf15 | |
| Gpc4 | Gdf15 | Klk8 | Ngf | Pcsk5 | Tgfb2 | | Vnn1 | |
| Grem2 | Ggh | Lamb1 | Nid1 | Pdgfa | Tgfb3 | | Wnt11 | |
| Grn | Ghr | Lamc2 | Nid2 | Pdgfd | Thbd | | Abca1 | |
| Gsn | Gla | Lamp2 | Npc2 | Pgf | Thbs1 | | Ahnak | |
| Gusb | Glb1 | Lgals3bp | Npnt | Pla1a | Timp1 | | Aqp1 | |
| Hbegf | Gldn | Lipg | Nppb | Pla2g15 | Timp2 | | Ccs | |
| Hist1h2bc | Gpc4 | Lox | Nucb1 | Pla2g7 | Timp3 | | Cd81 | |
| Hist1h2be | Gpx3 | Loxl2 | Ogn | Plau | Tinagl1 | | Gpc2 | |
| Hspg2 | Grem1 | Loxl3 | Olfm1 | Pon3 | Tnc | | Gpc6 | |
| Htra1 | Grem2 | Ltbp2 | Pappa | Prss23 | Tnfsf15 | | Igfbp5 | |
| Hyal1 | Grn | Lyz1 | Pcolce | Psap | Trf | | Itm2c | |
| Icam1 | Gsr | Lyz2 | Pcsk5 | Ptprz1 | Vegfa | | Pkd1 | |
| Igf2 | Gusb | Man2b1 | Pcsk6 | Qsox1 | Wisp1 | | Prrg2 | |
| Igf2r | Hbegf | Man2b2 | Pdgfa | Ramp1 | Wnt5a | | Rab5b | |
| Igfbp2 | Hexb | Manba | Pdgfd | Rnase4 | Wnt9a | | Serpinb10 | |
| Igfbp3 | Hilpda | Masp1 | Pgf | S100a13 | Cd81 | | Snx18 | |
| Igfbp4 | Hist1h2bc | Mcam | Pla1a | S100a16 | Clca1 | | | |
| Igfbp6 | Hist1h2be | Mcpt8 | Pla2g15 | Selp | Cubn | | | |
| Igfbp7 | Hist1h2bf | Mfap5 | Pla2g6 | Serpinb2 | Gpc6 | | | |
| Il17d | Hist1h2bg | Mfge8 | Plau | Serpinb6a | Itm2c | | | |
| Il34 | Hpgd | Mmp13 | Pon3 | Serpinb6b | Pcyox1 | | | |
| Il7 | Hpx | Mmp19 | Ppbp | Serpinb9 | Rab11a | | | |
| Inhba | Htra1 | Mmp2 | Prss23 | Serpinb9b | Rab5b | | | |
| Irak4 | Hyal1 | Mmp9 | Psap | Serpine1 | Sema7a | | | |
| Itgam | Icam1 | Nbl1 | Qsox1 | Serpine2 | Snx18 | | | |
| Itm2b | Igf1 | Nell2 | Rnase4 | Serping1 | | | | |
| Jam3 | Igf2r | Ngf | S100a13 | Serpini1 | | | | |
| Klk8 | Igfbp3 | Nid1 | S100a16 | Slmap | | | | |
| Lama2 | Igfbp4 | Nog | Selp | Smpdl3a | | | | |
| Lama5 | Igfbp7 | Npc2 | Sema3a | Sparc | | | | |
| Lamb1 | Il17d | Nppa | Sema3e | Srpx2 | | | | |
| Lamc2 | Il1rap | Nppb | Serpinb2 | Tcn2 | | | | |
| Lamp2 | Il34 | Nrn1 | Serpinb6a | Tfpi | | | | |
| Lgals3bp | Il4ra | Nucb1 | Serpinb6b | Tg | | | | |
| Lipg | Il6 | Nucb2 | Serpinb9b | Tgfb2 | | | | |
| Lox | Il7 | Ogn | Serpine1 | Tgfb3 | | | | |
| Loxl2 | Inhba | Olfm1 | Serpinf1 | Thbd | | | | |
| Loxl3 | Itgam | Pappa | Serping1 | Thbs1 | | | | |
| Ltbp2 | Itgbl1 | Pcsk6 | Smpdl3a | Timp1 | | | | |
| Lyz1 | Itm2b | Pdgfa | Sod1 | Timp2 | | | | |
| Lyz2 | Jam3 | Pdgfb | Soga1 | Timp3 | | | | |
| Man2b1 | Kcnk3 | Pdgfd | Sparc | Tinagl1 | | | | |
| Man2b2 | Klk8 | Pdia3 | Srpx2 | Tmsb4x | | | | |
| Manba | Lamb1 | Pgf | Tcn2 | Tnc | | | | |
| Masp1 | Lamc2 | Pla1a | Tfpi | Tnfsf15 | | | | |
| Mcam | Lamp2 | Pla2g15 | Tgfb2 | Tnfsf18 | | | | |
| Mcpt8 | Lepr | Pla2g6 | Tgfb3 | Trf | | | | |
| Metrnl | Lgals3 | Plau | Thbd | Vegfa | | | | |
| Mfap4 | Lgals3bp | Pm20d1 | Thbs1 | Vnn1 | | | | |
| Mfap5 | Lingo1 | Pon3 | Timp1 | Wisp1 | | | | |
| Mfge8 | Lipg | Ppbp | Timp2 | Wnt5a | | | | |
| Mmp13 | Lman2 | Ppt1 | Timp3 | Wnt9a | | | | |
| Mmp14 | Lox | Prelp | Tinagl1 | Cd81 | | | | |
| Mmp19 | Lox2 | Prss23 | Tnc | Clca1 | | | | |
| Mmp2 | Lox3 | Psap | Tnfsf15 | Cubn | | | | |
| Mmp9 | Lrrn1 | Ptprg | Trf | Gpc6 | | | | |
| Msln | Lrrn2 | Qsox1 | Vegfa | Itm2c | | | | |
| Nbl1 | Ltbp2 | Ramp1 | Wisp1 | Naglu | | | | |
| Nell2 | Lyz1 | Rnase4 | Wnt2b | Pcyox1 | | | | |
| Ngf | Lyz2 | S100a13 | Wnt5a | Rab11a | | | | |
| Nid1 | Man2b1 | S100a16 | Wnt9a | Rab5b | | | | |
| Nid2 | Man2b2 | Selp | Abca1 | Sema7a | | | | |
| Nog | Manba | Sema3c | Cd81 | Snx18 | | | | |
| Npc2 | Masp1 | Sema3f | Clca1 | | | | | |
| Npnt | Mcam | Serpinb2 | Coch | | | | | |
| Nppa | Mcpt8 | Serpinb6a | Cubn | | | | | |
| Nppb | Mfap5 | Serpinb6b | Gpc6 | | | | | |
| Nrn1 | Mfge8 | Serpinb8 | Itm2c | | | | | |
| Nucb1 | Mmp12 | Serpinb9b | Loxl4 | | | | | |
| Nucb2 | Mmp13 | Serpine1 | Pcyox1 | | | | | |
| Ogn | Mmp19 | Serping1 | Rab11a | | | | | |
| Olfm1 | Mmp2 | Slmap | Rab5b | | | | | |
| Pappa | Mmp3 | Smpd1 | Sema7a | | | | | |
| Pcsk6 | Mmp9 | Smpdl3a | Snx18 | | | | | |
| Pdgfa | Nbl1 | Sparc | | | | | | |
| Pdgfb | Nell2 | Spink8 | | | | | | |
| Pdgfd | Ngf | Srpx2 | | | | | | |
| Pdia3 | Nid1 | St14 | | | | | | |
| Pebp1 | Nog | Tcn2 | | | | | | |
| Pgf | Npc2 | Tfpi | | | | | | |
| Pla1a | Nppa | Tg | | | | | | |
| Pla2g15 | Nppb | Tgfb2 | | | | | | |
| Pla2g6 | Nrn1 | Tgfb3 | | | | | | |
| Plau | Nucb1 | Thbd | | | | | | |
| Pm20d1 | Nucb2 | Thbs1 | | | | | | |
| Pon3 | Ogn | Timp1 | | | | | | |
| Ppbp | Olfm1 | Timp2 | | | | | | |
| Ppp1r1a | Pam | Timp3 | | | | | | |
| Ppt1 | Pappa | Tinagl1 | | | | | | |
| Prelp | Pcsk5 | Tmsb4x | | | | | | |
| Prss23 | Pcsk6 | Tnc | | | | | | |
| Psap | Pdgfa | Tnfsf15 | | | | | | |
| Ptprg | Pdgfb | Trf | | | | | | |
| Ptprz1 | Pdgfd | Vnn1 | | | | | | |
| Qsox1 | Pdia3 | Wnt11 | | | | | | |
| Ramp1 | Pf4 | Wnt4 | | | | | | |
| Rnase4 | Pgf | Wnt9a | | | | | | |
| S100a13 | Pla1a | Abca1 | | | | | | |
| S100a16 | Pla2g15 | Ccs | | | | | | |
| S100a7a | Pla2g6 | Cd81 | | | | | | |
| Selp | Pla2g7 | Clca1 | | | | | | |
| Sema3a | Plau | Col20a1 | | | | | | |
| Sema3b | Pm20d1 | Cubn | | | | | | |
| Sema3c | Pon3 | Gpc6 | | | | | | |
| Sema3e | Ppbp | Itm2c | | | | | | |
| Sema3f | Ppt1 | Loxl4 | | | | | | |
| Sema4f | Prelp | Naglu | | | | | | |
| Serpinb2 | Prg4 | Pcyox1 | | | | | | |
| Serpinb6a | Prss23 | Rab11a | | | | | | |
| Serpinb6b | Psap | Rab4a | | | | | | |
| Serpinb8 | Ptprg | Rab5b | | | | | | |
| Serpinb9b | Qsox1 | Sema7a | | | | | | |
| Serpine1 | Ramp1 | Snx18 | | | | | | |
| Serping1 | Rnase4 | Sri | | | | | | |
| Slit1 | S100a13 | | | | | | | |
| Slmap | S100a16 | | | | | | | |
| Smpd1 | S100b | | | | | | | |
| Smpdl3a | Scube1 | | | | | | | |
| Sod1 | Scube3 | | | | | | | |
| Sparc | Selp | | | | | | | |
| Spink8 | Sema3c | | | | | | | |
| Spon1 | Sema3f | | | | | | | |
| Spon2 | Serpinb2 | | | | | | | |
| Srpx2 | Serpinb6a | | | | | | | |
| Ssc5d | Serpinb6b | | | | | | | |
| St14 | Serpinb8 | | | | | | | |
| Tcn2 | Serpinb9b | | | | | | | |
| Tfpi | Serpine1 | | | | | | | |
| Tg | Serpine2 | | | | | | | |
| Tgfb2 | Serping1 | | | | | | | |
| Tgfb3 | Serpini1 | | | | | | | |
| Thbd | Slmap | | | | | | | |
| Thbs1 | Smpd1 | | | | | | | |
| Timp1 | Smpdl3a | | | | | | | |
| Timp2 | Snca | | | | | | | |
| Timp3 | Sod3 | | | | | | | |
| Tinagl1 | Sparc | | | | | | | |
| Tmsb4x | Spink10 | | | | | | | |
| Tnc | Spink8 | | | | | | | |
| Tnfrsf1a | Spp1 | | | | | | | |
| Tnfsf15 | Srpx2 | | | | | | | |
| Trf | St14 | | | | | | | |
| Vgf | Tcn2 | | | | | | | |
| Vnn1 | Tfpi | | | | | | | |
| Wnt11 | Tg | | | | | | | |
| Wnt2b | Tgfb2 | | | | | | | |
| Wnt4 | Tgfb3 | | | | | | | |
| Wnt9a | Thbd | | | | | | | |
| Abca1 | Thbs1 | | | | | | | |
| Apobr | Timp1 | | | | | | | |
| Ccs | Timp2 | | | | | | | |
| Cd81 | Timp3 | | | | | | | |
| Clca1 | Tinagl1 | | | | | | | |
| Coch | Tmsb4x | | | | | | | |
| Col20a1 | Tnc | | | | | | | |
| Cubn | Tnfsf15 | | | | | | | |
| Dpep1 | Tnfsf18 | | | | | | | |
| Gars | Trf | | | | | | | |
| Gpc6 | Tsku | | | | | | | |
| Ist1 | Vegfc | | | | | | | |
| Itm2c | Vnn1 | | | | | | | |
| Loxl4 | Vwc2 | | | | | | | |
| Naglu | Wfdc1 | | | | | | | |
| Pcyox1 | Wisp1 | | | | | | | |
| Pkd1 | Wnt11 | | | | | | | |
| Prrg2 | Wnt4 | | | | | | | |
| Rab11a | Wnt5a | | | | | | | |
| Rab4a | Wnt9a | | | | | | | |
| Rab5b | Abca1 | | | | | | | |
| Sema7a | Ccs | | | | | | | |
| Snx18 | Cd81 | | | | | | | |
| Sri | Clca1 | | | | | | | |
| | Cntfr | | | | | | | |
| | Col20a1 | | | | | | | |
| | Cubn | | | | | | | |
| | Galnt2 | | | | | | | |
| | Gpc6 | | | | | | | |
| | Itga4 | | | | | | | |
| | Itm2c | | | | | | | |
| | Loxl4 | | | | | | | |
| | Ly96 | | | | | | | |
| | Msr1 | | | | | | | |
| | Naglu | | | | | | | |
| | Pcyox1 | | | | | | | |
| | Rab11a | | | | | | | |
| | Rab4a | | | | | | | |
| | Rab5b | | | | | | | |
| | Rap2b | | | | | | | |
| | Sdcbp | | | | | | | |
| | Sema7a | | | | | | | |
| | Serpina3h | | | | | | | |
| | Snx18 | | | | | | | |
| | Srgn | | | | | | | |
| | Sri | | | | | | | |

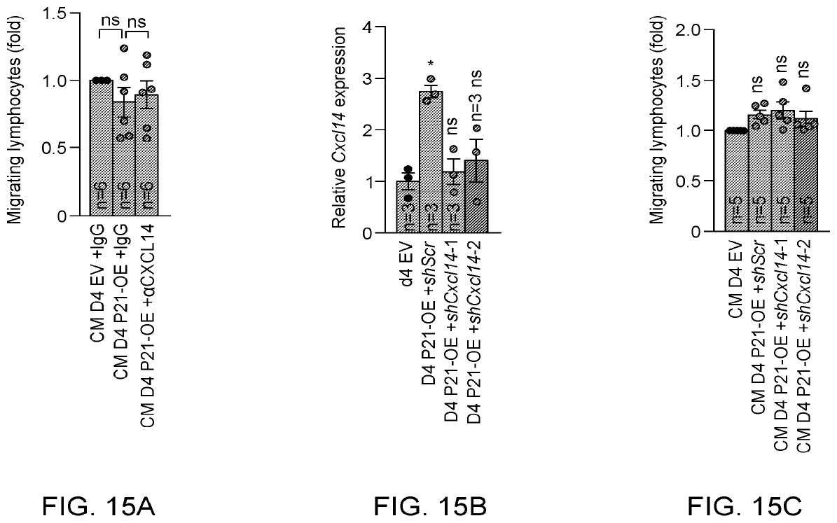

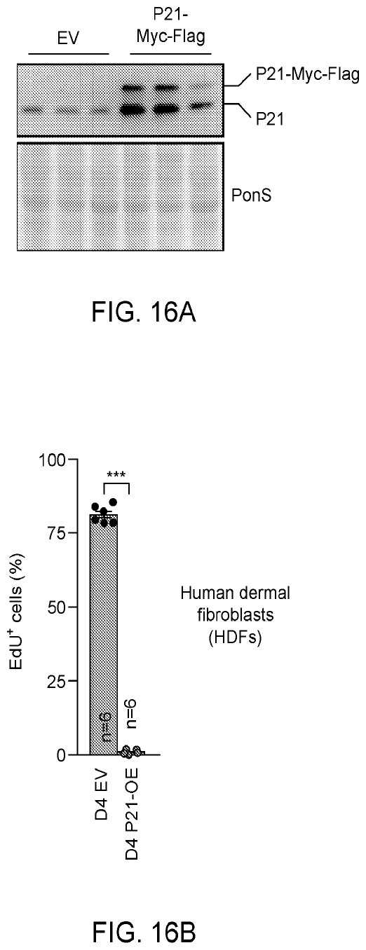

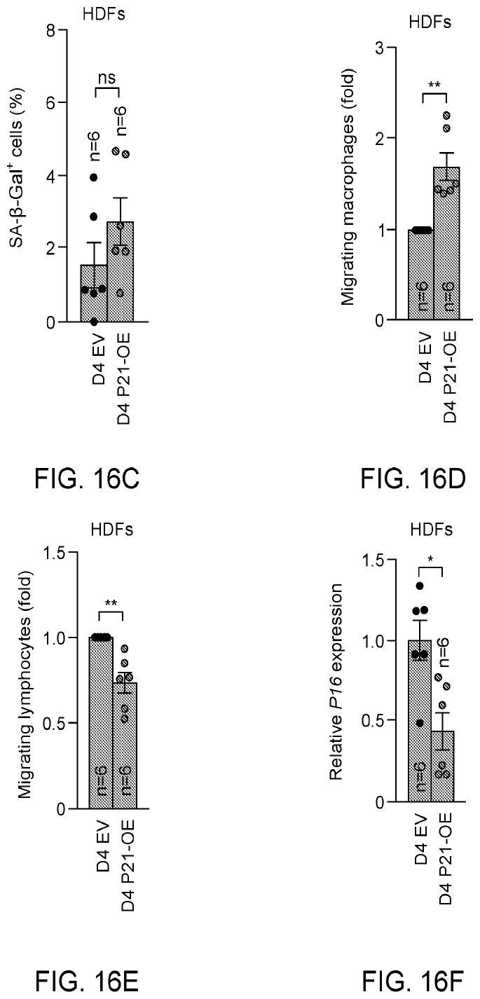

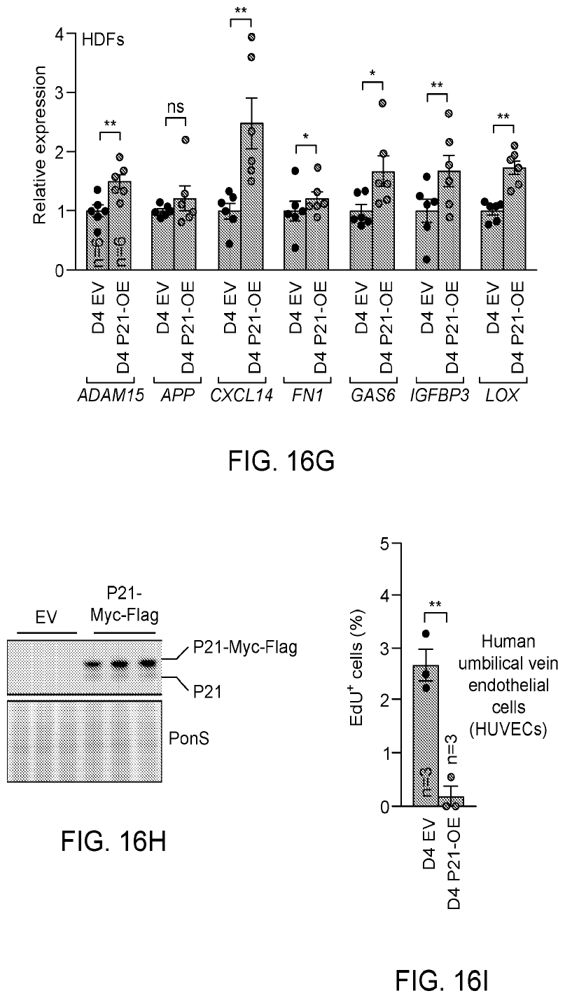

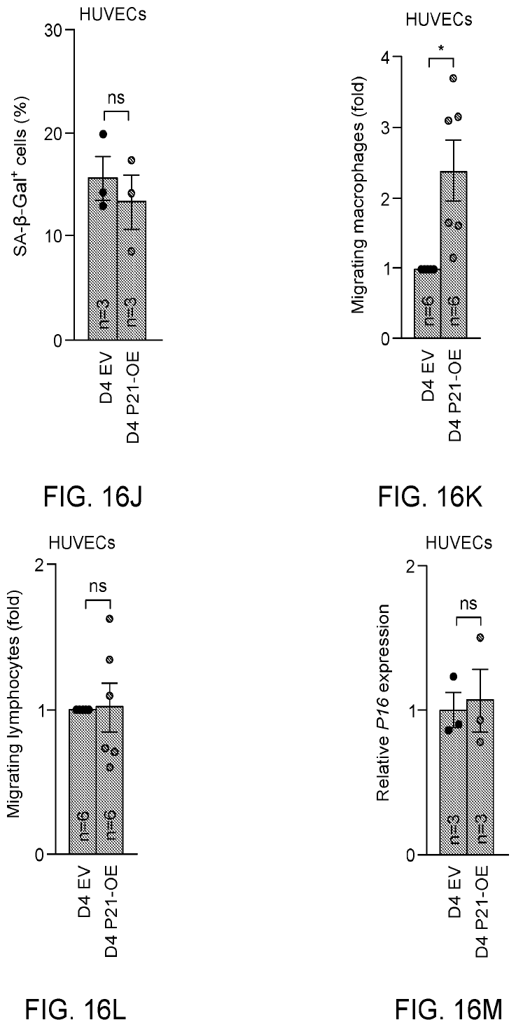

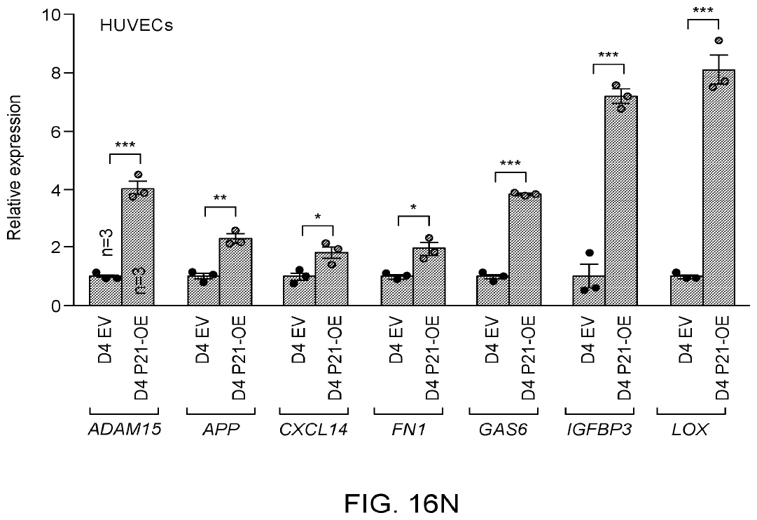

Functional annotation analysis on the PASP of D4 P21-OE MEFs suggested that it has similar biological properties as the PASP of D4 IR-MEFs (FIG. 14F). Indeed, CM from D4 P21-OE MEFs stimulated fibroblast migration in our scratch assay and macrophage migration in our transwell assay, and increased local macrophage numbers when intraperitoneally injected in mice (FIGS. 14G to 14N). MEF-derived PASPs consistently included CXCL14 (FIG. 1B and Table 3), a member of the CXC chemokine family that exerts chemo-attractive activity for monocytes, macrophages and dendritic cells. Addition of CXCL14-neutralizing antibodies to CM harvested from D4 P21-OE MEFs ablated stimulation of macrophage migration in our transwell assay, whereas control IgG did not (FIG. 2B and FIG. 15A). Moreover, CM from Cxcl14-depleted D4 P21-OE MEFs failed to evoke macrophage migration (FIG. 2C and FIGS. 15B and 15C), further indicating that CXCL14 is the key macrophage attractant of the PASP. Complementary experiments in human dermal fibroblasts (HDFs) and human umbilical vein endothelial cells (HUVECs) suggested that the PASP is a common feature of P21 induction and CXCL14 a signature PASP component (FIG. 16).

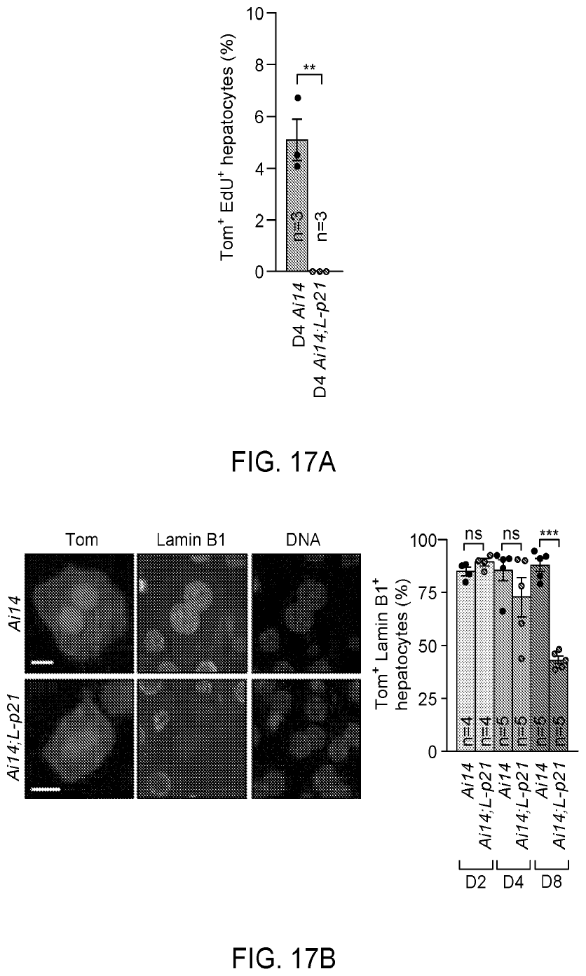

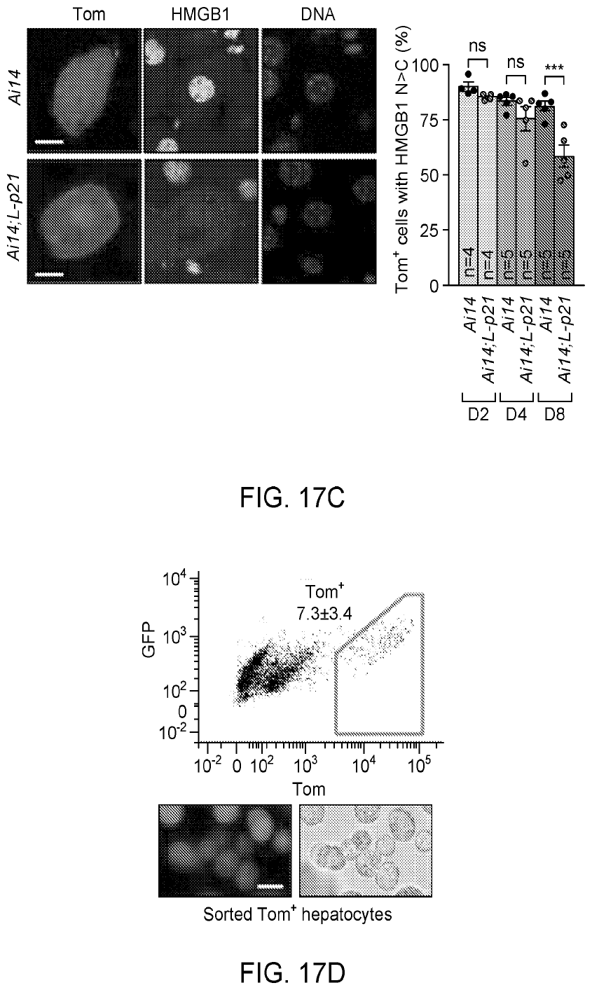

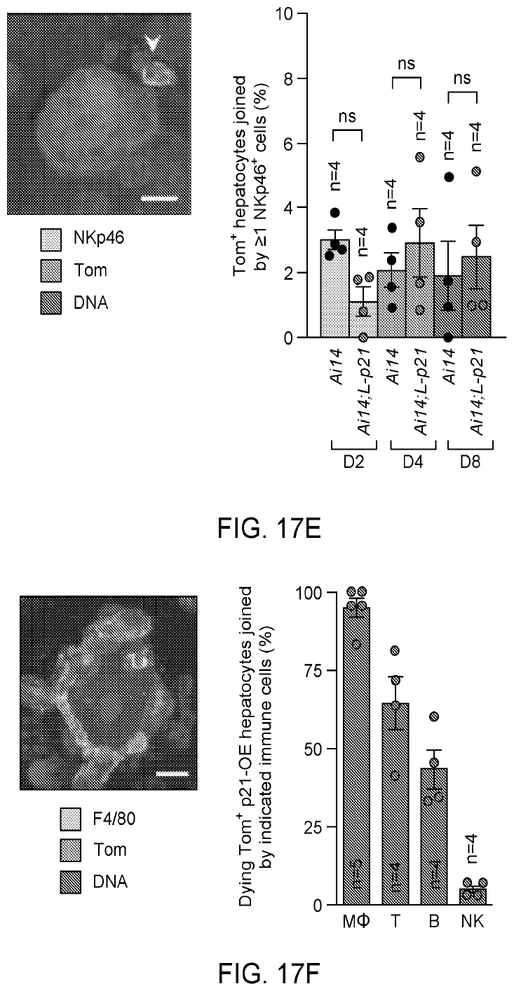

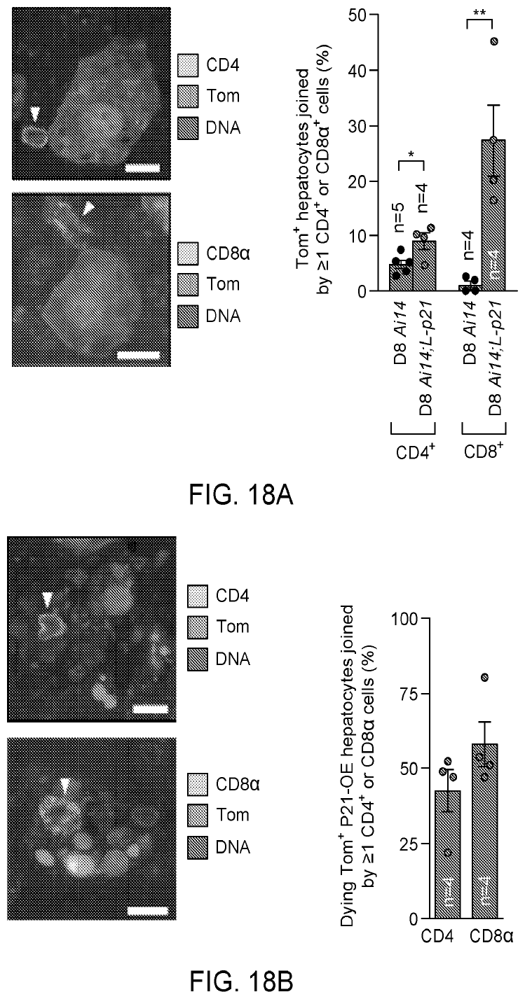

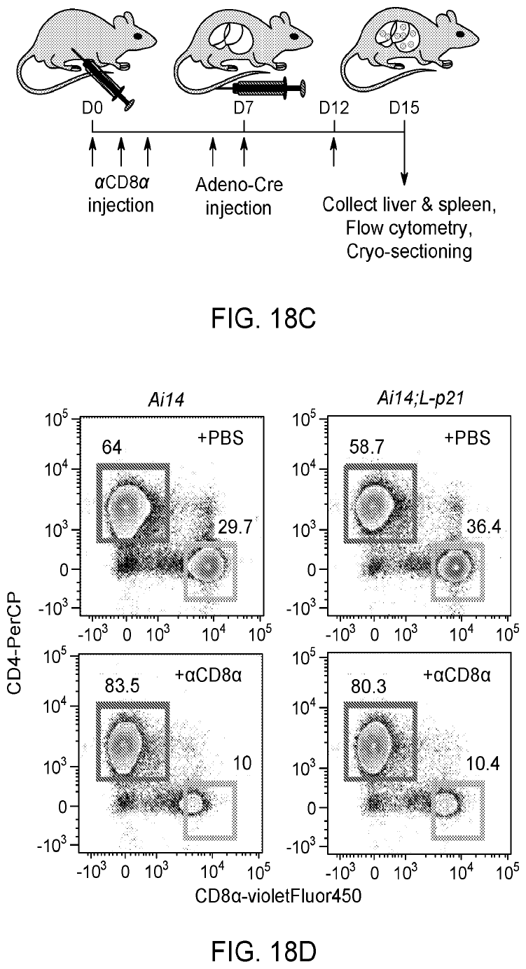

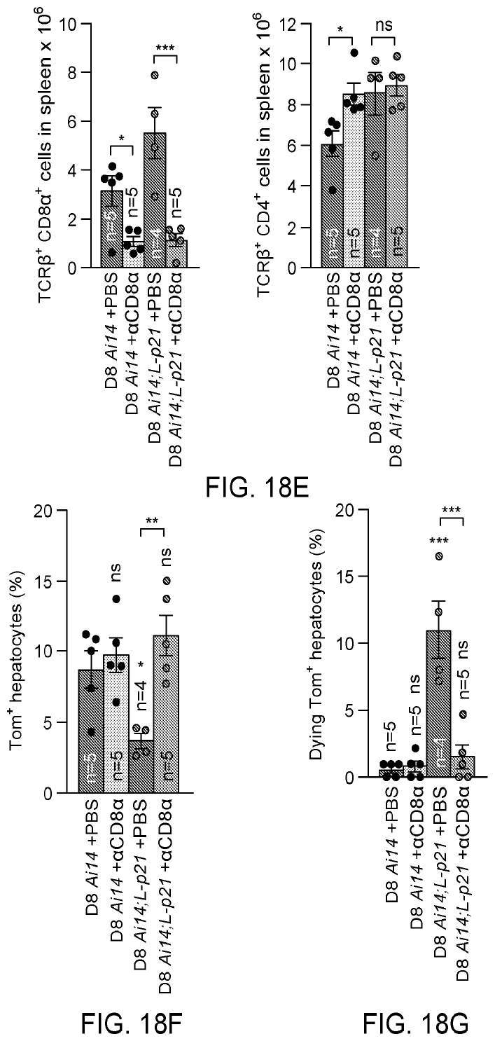

To study the PASP phenomenon at the organismal level, a transgenic mouse strain that allows for Cre-inducible overexpression of C-terminally Myc-Flag-tagged P21 through excision of a loxP-flanked transcriptional stop cassette was engineered (FIG. 2D). A Cre-inducible tdTomato (Tom) reporter transgene (Ai14) was crossed into this L-p2/strain to visualize and harvest P21-OE cells (FIG. 2D), and P21-OE was induced in ˜10% of hepatocytes by tail vein injection of adeno-Cre virus. P21-OE hepatocytes were growth-arrested at D4 post-injection and exhibited signs of senescence by D8 post-injection, as evidenced by loss of LaminB1 and nuclear extrusion of HMGB1 (FIGS. 17A to 17C). RNA extracted from FACS-sorted D4 Tom⁺ hepatocytes with or without P21-OE and used for RT-qPCR analysis of PASP factor gene transcripts indicated that P21-OE induces a PASP in vivo (FIG. 2E and FIG. 17D). Cxcl14 was among the upregulated PASP factors, prompting us to test whether P21-OE hepatocytes attract macrophages. Indeed, nearly 40% of P21-OE Tom⁺ hepatocytes were surrounded by three or more macrophages as early as D2 post-adeno-Cre injection versus ˜10% of Tom⁺ hepatocytes without P21-OE (FIG. 2F). Macrophage recruitment to P21-OE hepatocytes was CXCL14-dependent as assessed by injection of anti-CXCL14 antibodies (FIG. 2G). Lymphocytes were also recruited but later, with B and T cells surrounding P21-OE hepatocytes at D4 and D8, respectively (FIGS. 2H and 2I). P21-OE did not prompt recruitment of NK cells (FIG. 17E). The number of P21-OE hepatocytes sharply declined by D8, which coincided with a marked increase in dying P21-OE hepatocytes the presence of M1-differentiated macrophages in addition to the presence of both CD4⁺ and CD8⁺ T lymphocytes (FIGS. 2J to 2M, FIG. 17F, and FIGS. 18A and 18B). Administration of CD8α-neutralizing antibodies fully prevented the observed decline in D8 P21-OE hepatocytes (FIGS. 18C to 18G), indicating that their elimination is mediated by cytotoxic T cells.

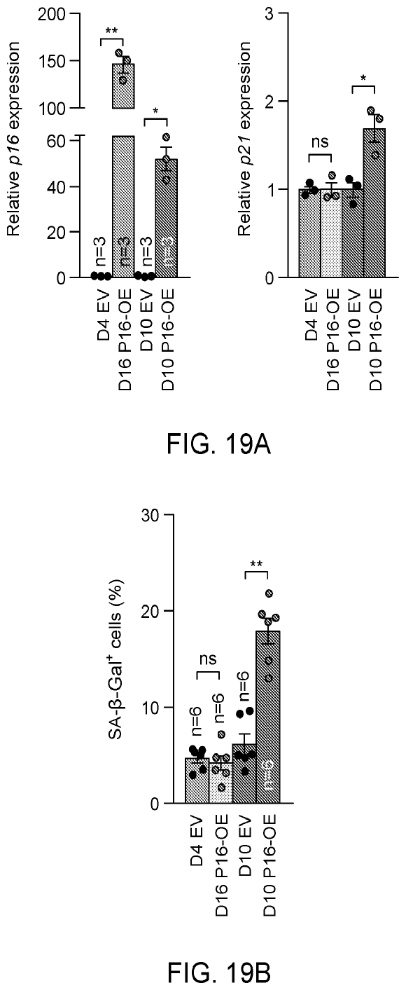

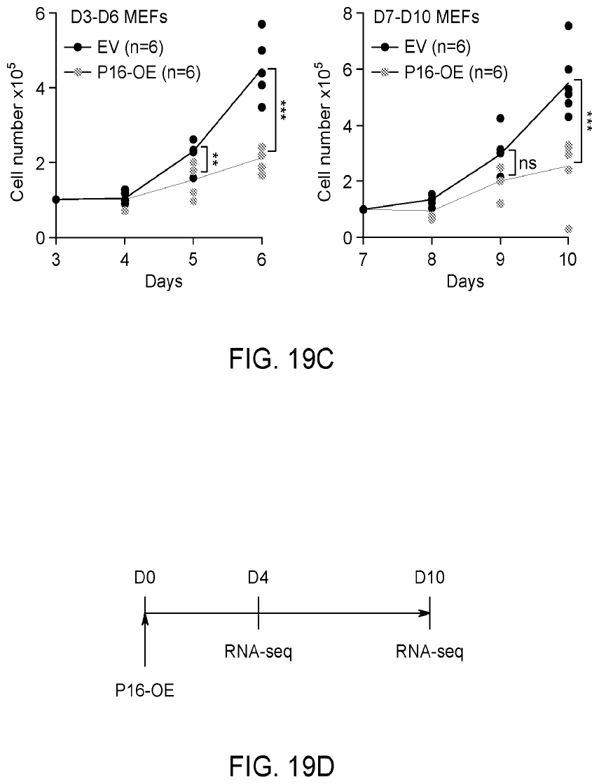

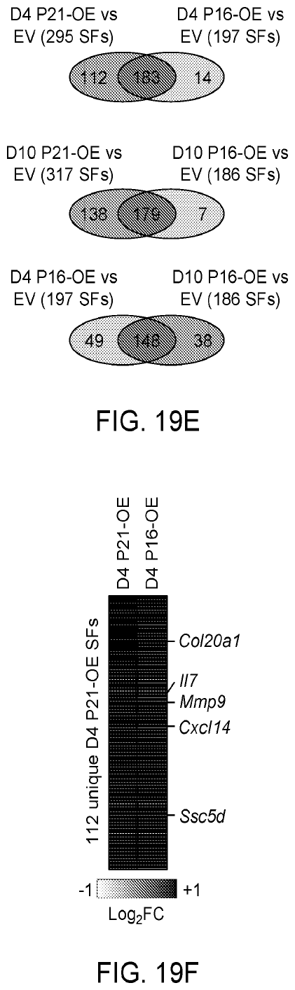

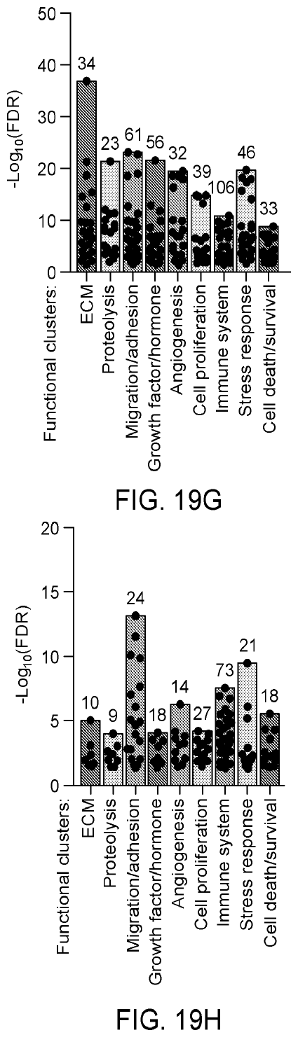

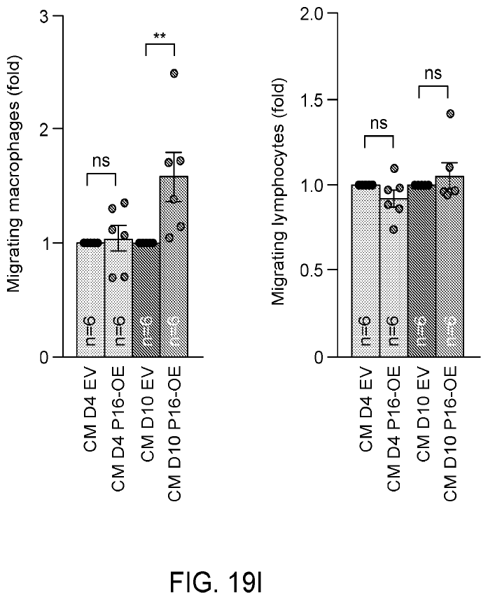

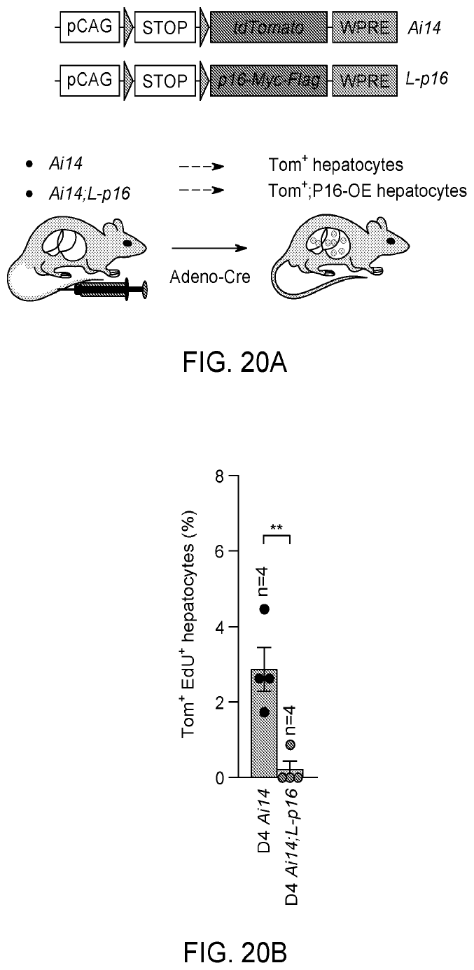

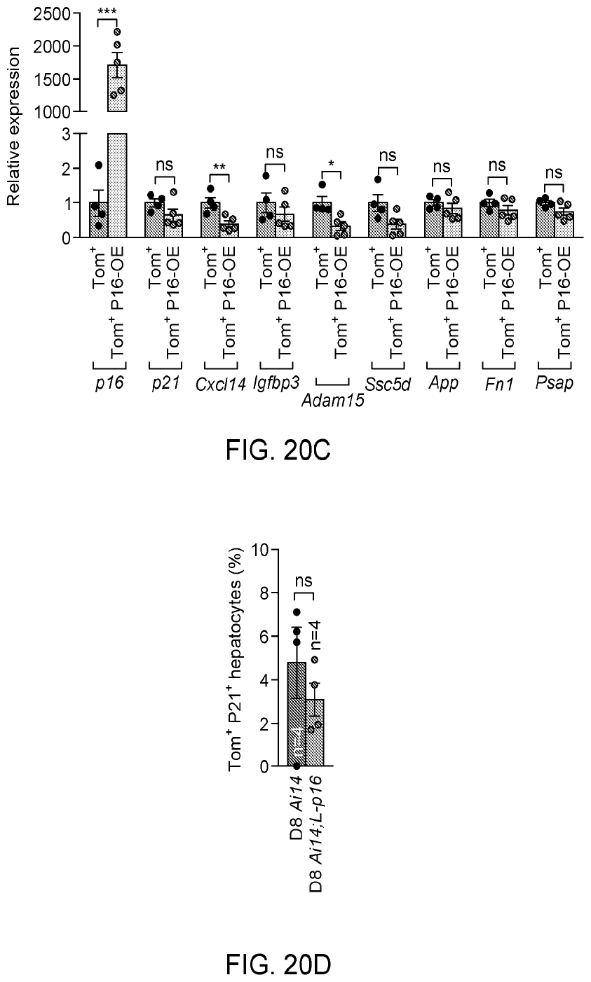

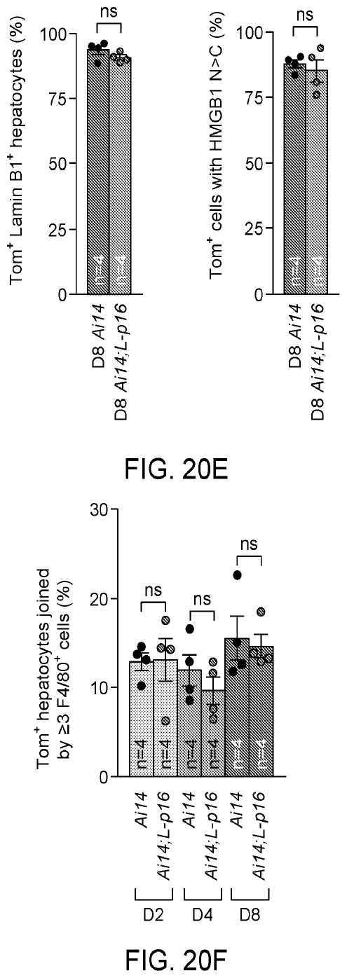

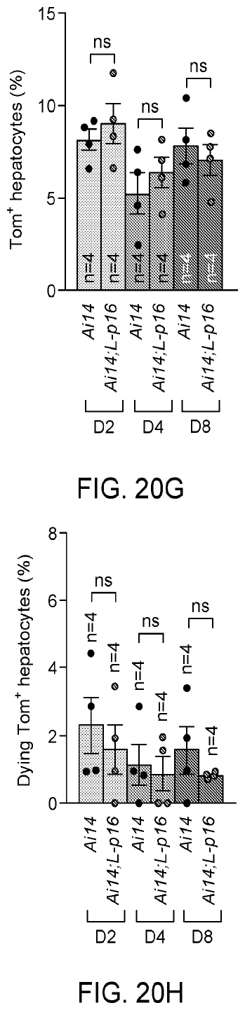

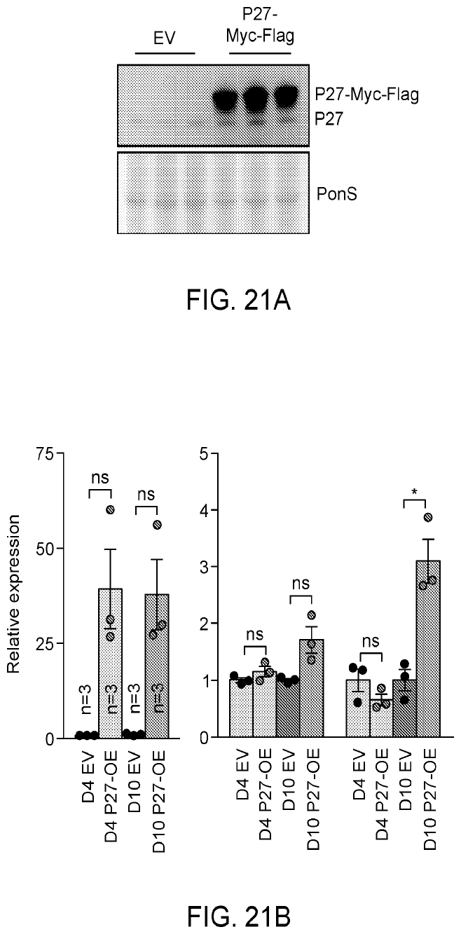

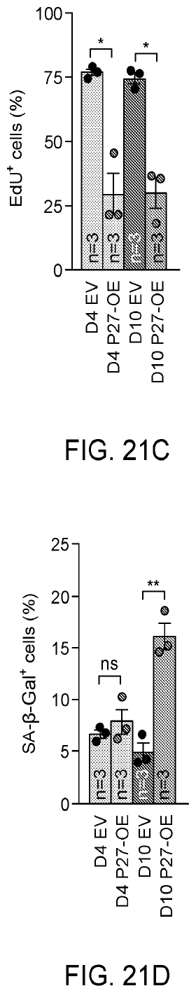

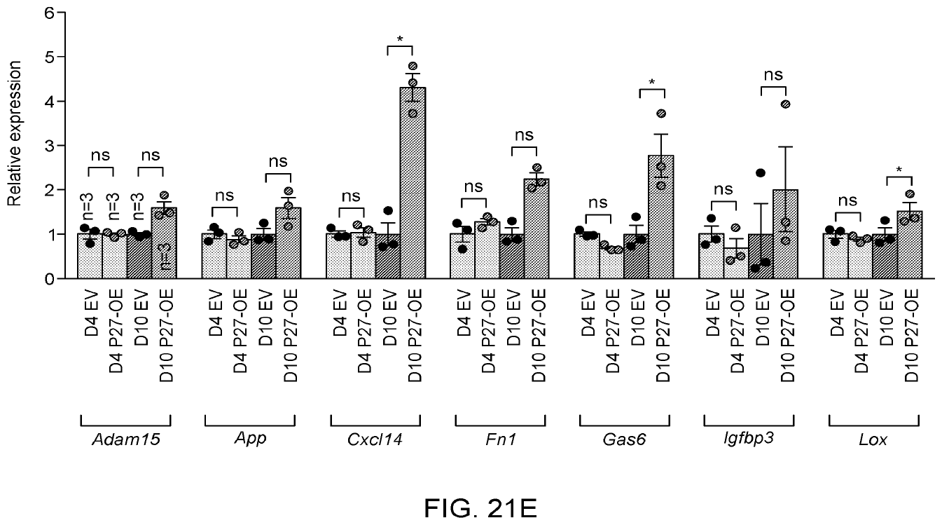

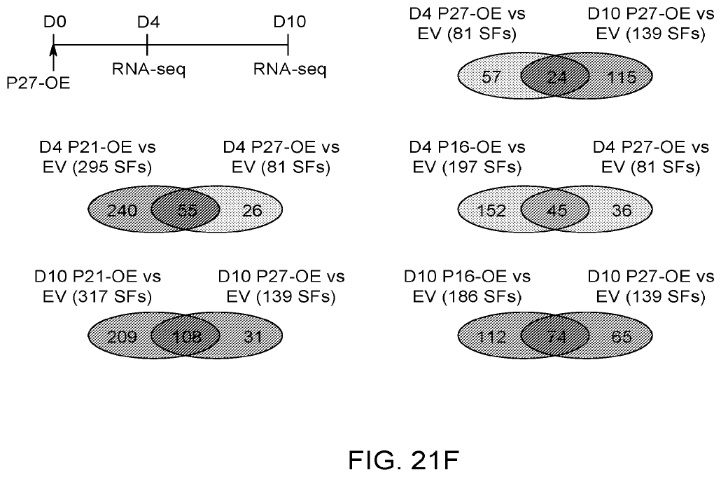

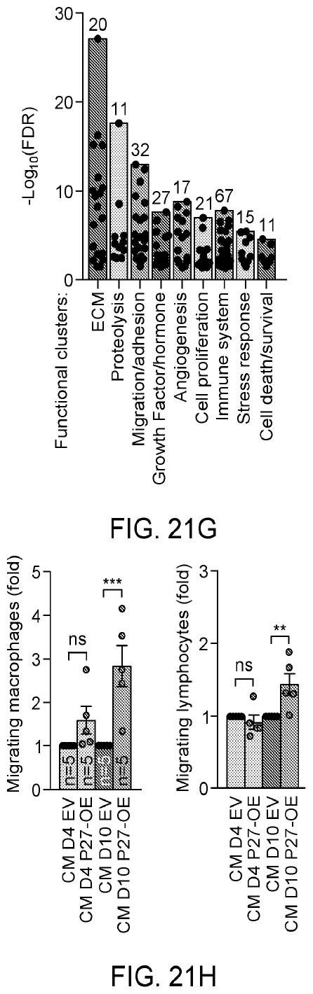

A comparative analysis for overexpression of p16, a more selective CDK inhibitor that unlike P21 only targets G1-CDK activity, was performed. D4 P16-OE MEFs were characterized by growth inhibition, normal P21 levels, and a secretome of 197 factors, 183 of which overlap with the PASP of D4 P21-OE MEFs (FIGS. 19A to 19F and Table 3). Pathway enrichment analyses on the P16-associated secretory phenotype suggested a high degree of similarity in biological properties with the PASP, although the immune system seemed to be impacted to a lesser extent (FIGS. 19G and 19H). CM of D4 P16-OE MEFs failed to promote migration of macrophages in our transwell assay, which correlated with a lack of Cxcl14 induction (FIG. 19I). Likewise, using the same transgenic approach as used for P21-OE in mice, P16-OE in hepatocytes was found to trigger cell-cycle arrest but not immunosurveillance, which coincided with a lack of P21 and PASP factor induction, including Cxcl14 (FIG. 20). Corresponding analyses of MEFs overexpressing P27, a CDK inhibitor that enables cell-cycle withdrawal during terminal differentiation, revealed that coordinated induction of growth arrest and immunosurveillance is a unique feature of P21 (FIG. 21 and Table 3).

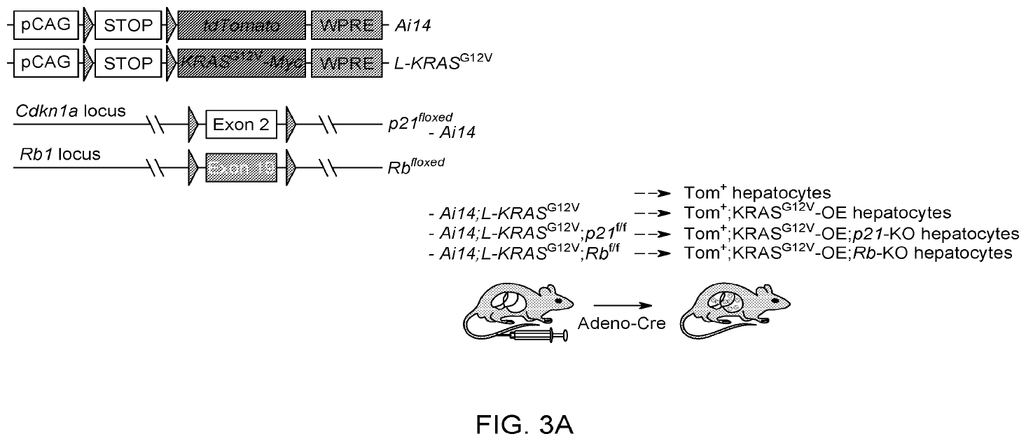

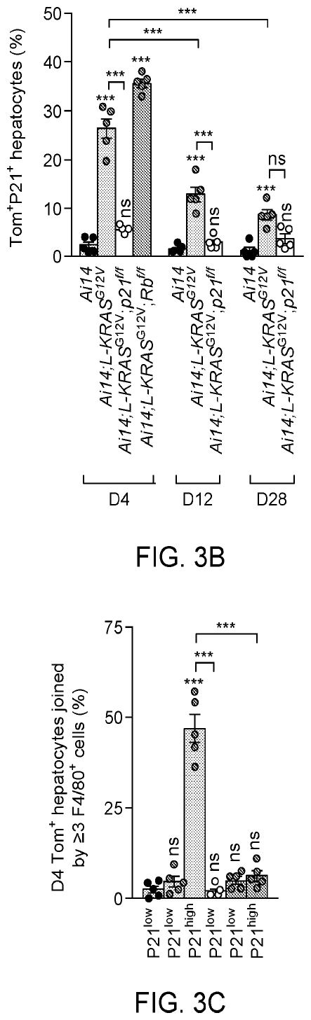

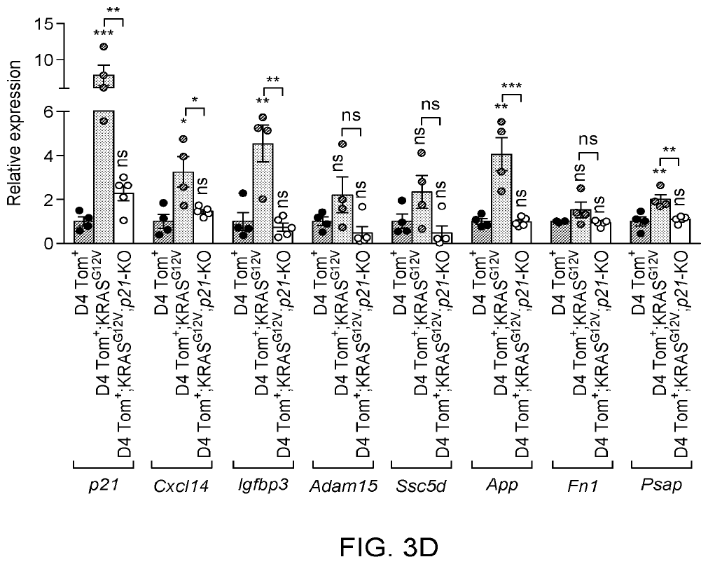

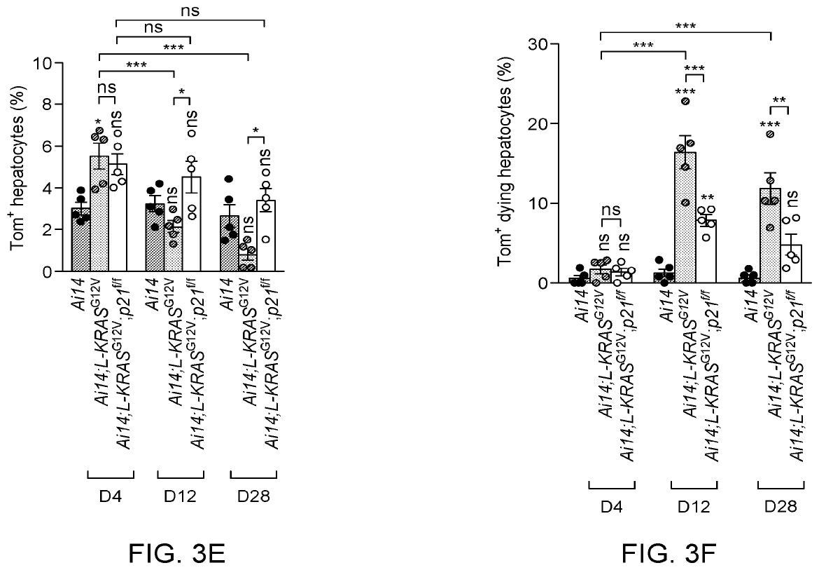

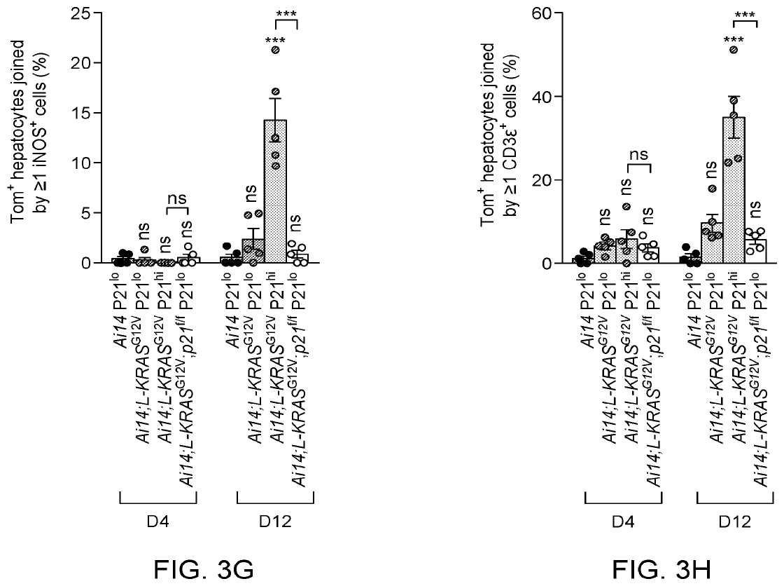

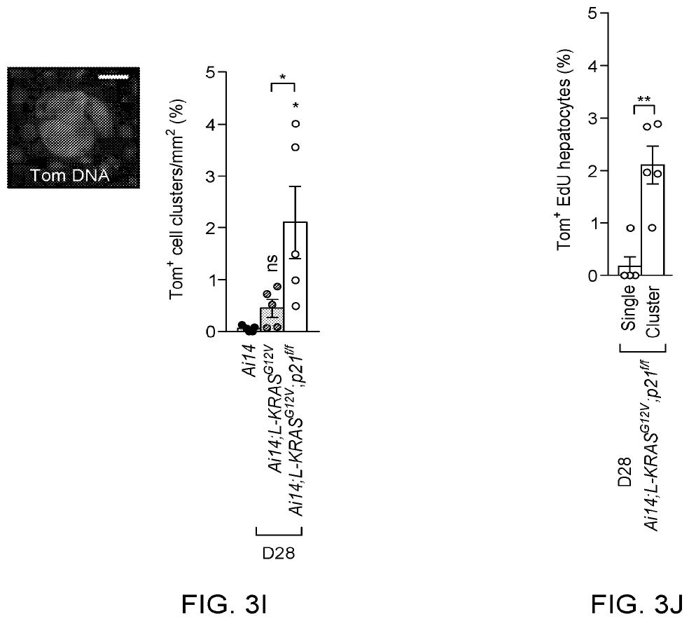

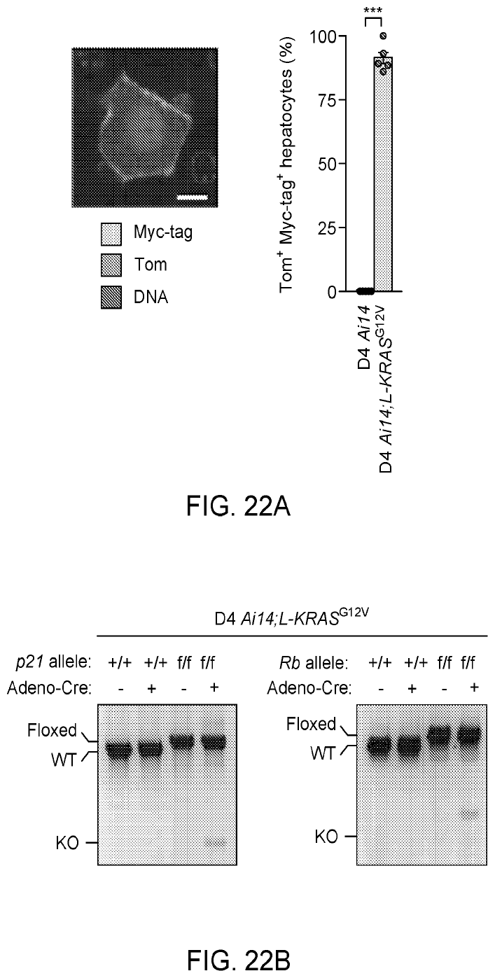

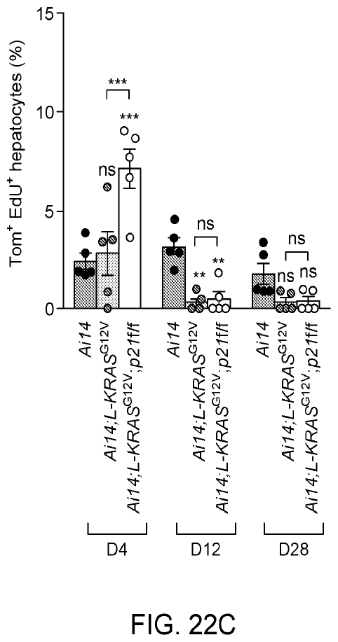

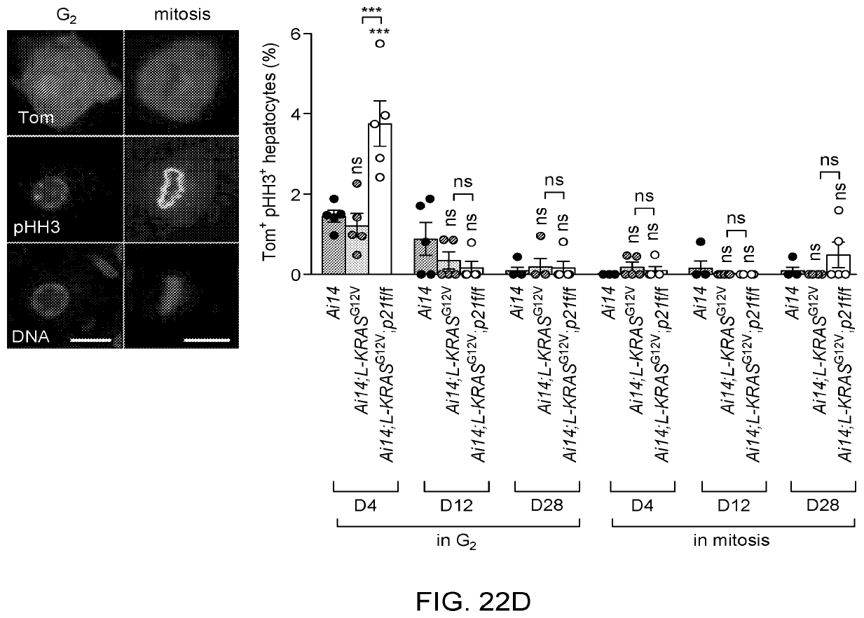

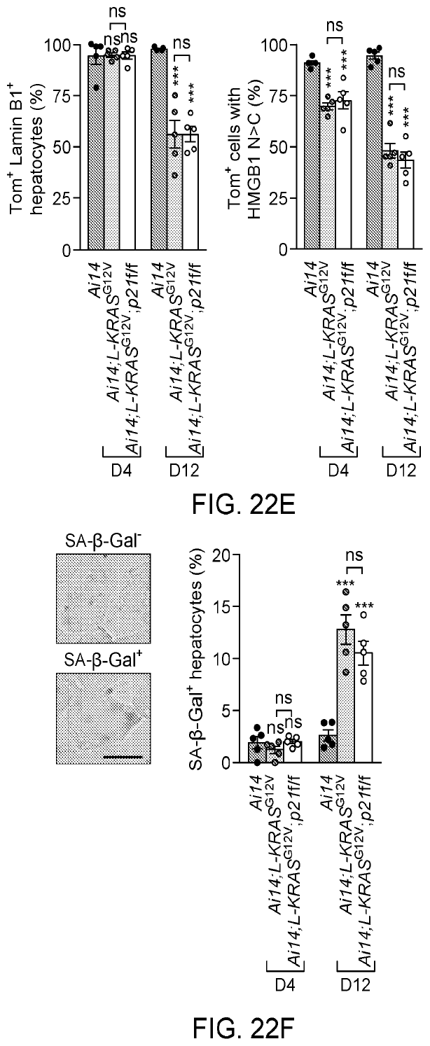

The physiological relevance of P21-dependent immunosurveillance in a cancer-related context was tested. To this end, the transgenic approach was adapted for co-induction of Tom and P21 in hepatocytes by replacing p2/with KRAS^{G12V} (FIG. 3A), an oncoprotein that can induce P21 via mitogenic stress. About 25% of D4 Tom⁺ KRAS^{G12V} hepatocytes had elevated P21 levels (FIG. 3B and FIG. 22A). These hepatocytes attracted macrophages, whereas those that failed to induce P21 did not (FIG. 3C). Use of a newly generated p21 conditional knock-out strain conclusively demonstrated that D4 Tom⁺ KRAS^{G12V} hepatocytes recruit macrophages in a P21-dependent manner (FIGS. 3A to 3C and FIG. 22B). Furthermore, D4 Tom⁺ KRAS^{G12V} hepatocytes in which Rb was conditionally knocked out retained P21 induction but nevertheless failed to attract macrophages, validating cell culture experiments indicating that P21 places cells under immunosurveillance in an RB-dependent fashion (FIGS. 3A to 3C and FIG. 22B).

D4 Tom⁺ KRAS^{G12V} hepatocytes had a PASP which they lost with conditional inactivation of p21 (FIG. 3D). The PASP included Cxcl14, explaining why D4 Tom⁺ KRAS^{G12V} hepatocytes attract macrophages and their counterparts lacking P21 do not. Tom⁺ hepatocytes numbers remained largely unchanged at D4, D12 and D28 post-induction when KRAS^{G12V} was absent (FIG. 3E), but progressively declined due to cell death when KRAS^{G12V} was co-expressed (FIGS. 3E and 3F). However, no such decline occurred when P21 was inactivated upon KRAS^{G12V} induction. This was not due to compensatory cell proliferation because P21 inactivation had no impact on the mitotic index of Tom⁺ KRAS^{G12V} hepatocytes (FIGS. 22C and 22D). Consistent with P21-dependent cell elimination, Tom⁺ KRAS^{G12V} hepatocytes with high P21 levels gradually decreased from D4 to D28 (FIG. 3B). D12 Tom⁺ KRAS^{G12V} hepatocytes were surrounded by M1 macrophages and T lymphocytes, whereas their D4 counterparts were not, indicating that cell elimination was executed by immune cells (FIGS. 3G and 3H).

Regardless of whether P21 was intact or inactivated, Tom⁺ KRAS^{G12V} hepatocytes hardly proliferated and showed signs of cellular senescence from D12 on (FIGS. 22C to 22F). However, small clusters of Tom⁺ KRAS^{G12V} hepatocytes were observed in D28 livers with much higher frequency when P21 was inactivated (FIG. 3I). Hepatocytes within these clusters were cycling at a markedly higher rate than corresponding hepatocytes located in isolation (FIG. 3J). Collectively, these findings indicate that P21-dependent immunoclearance of cells that experience oncogenic stress constitutes an important first line of defense against neoplastic growth.

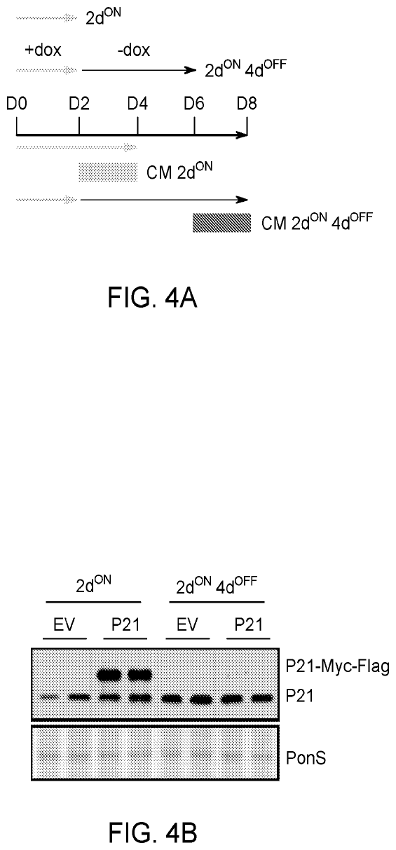

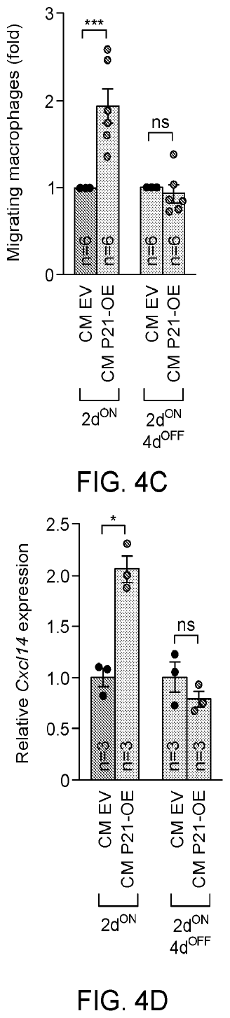

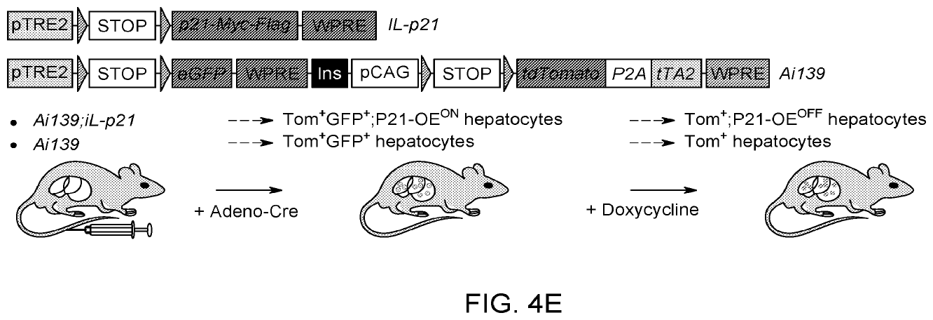

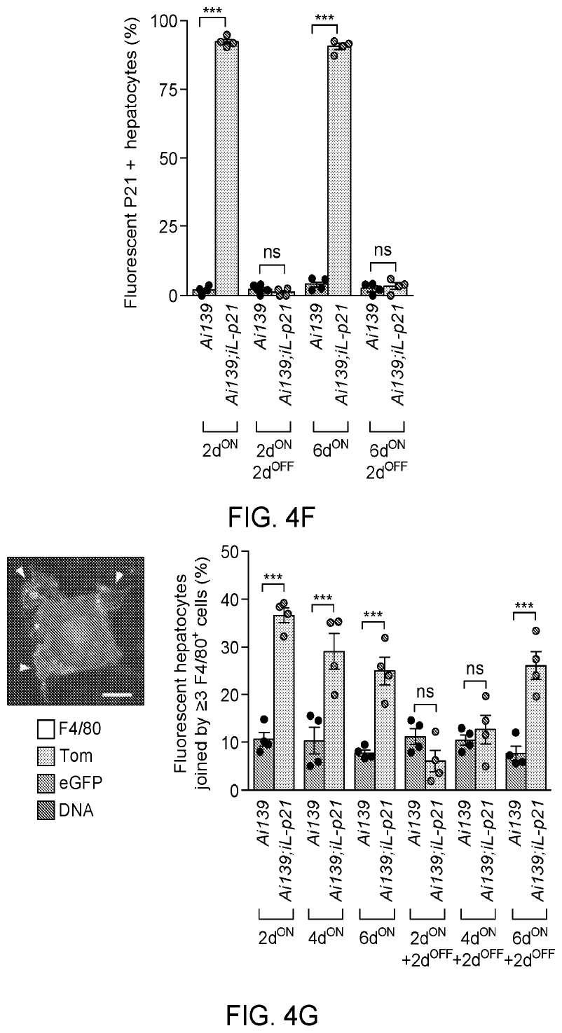

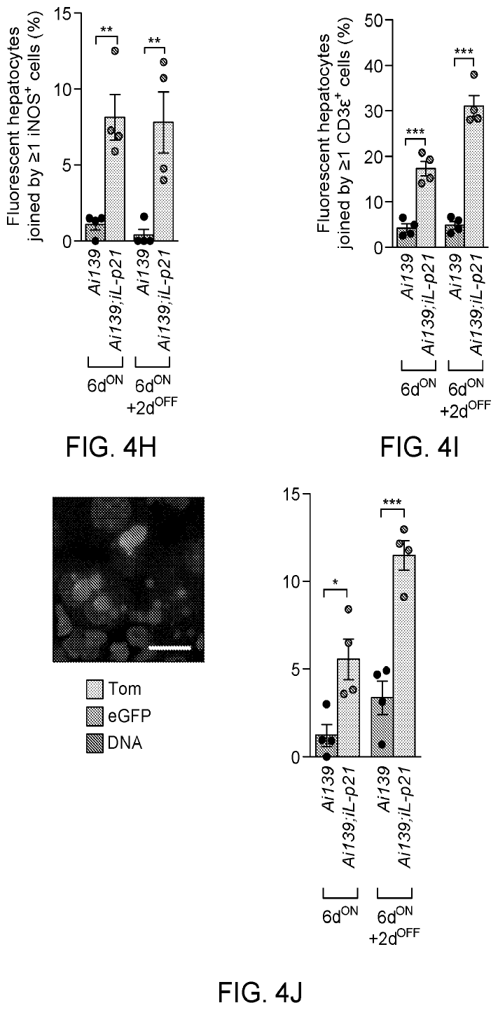

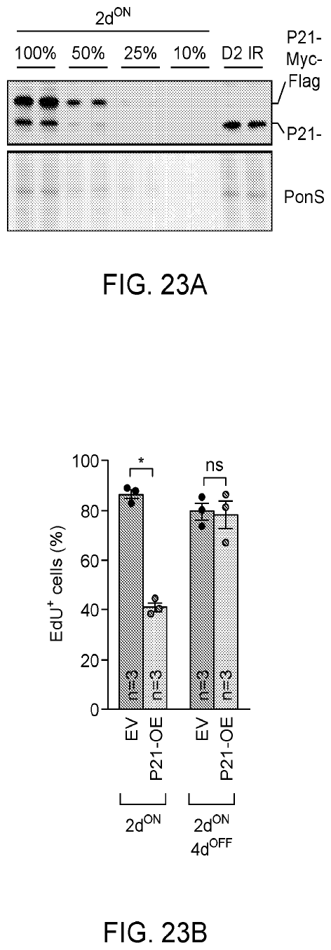

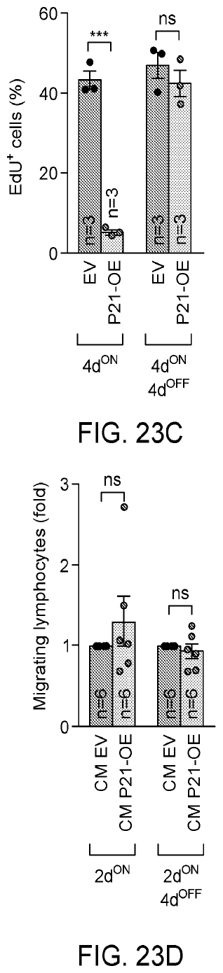

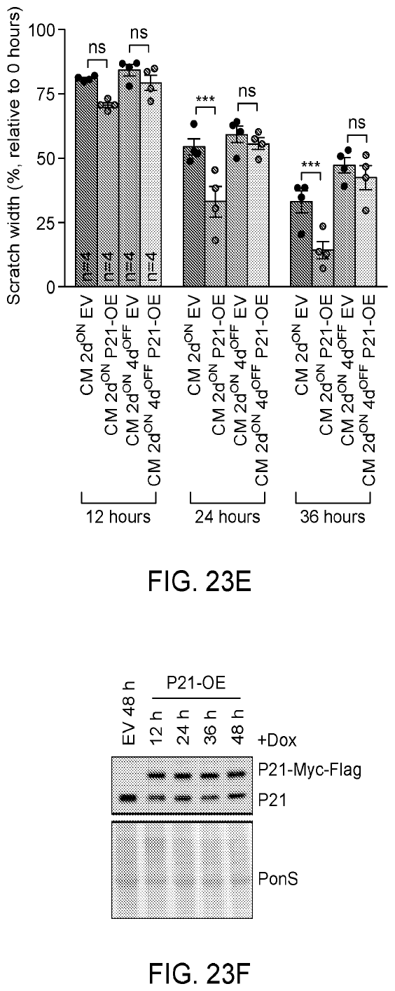

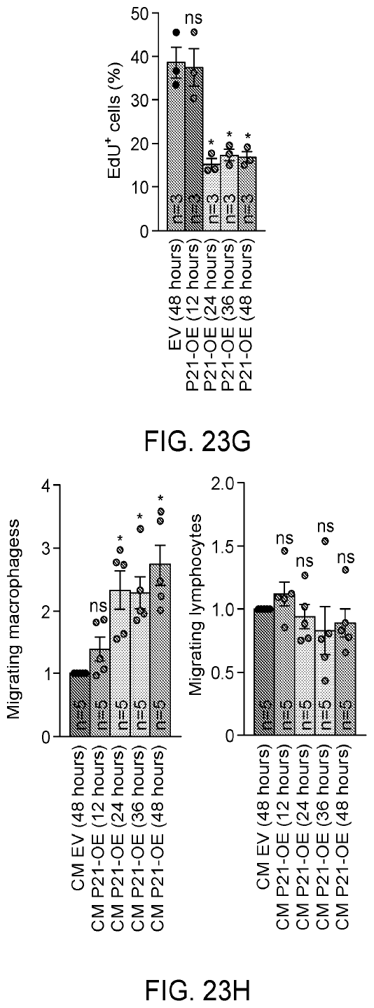

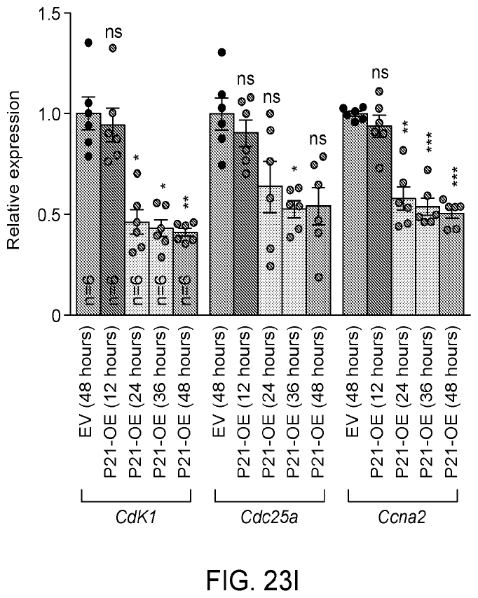

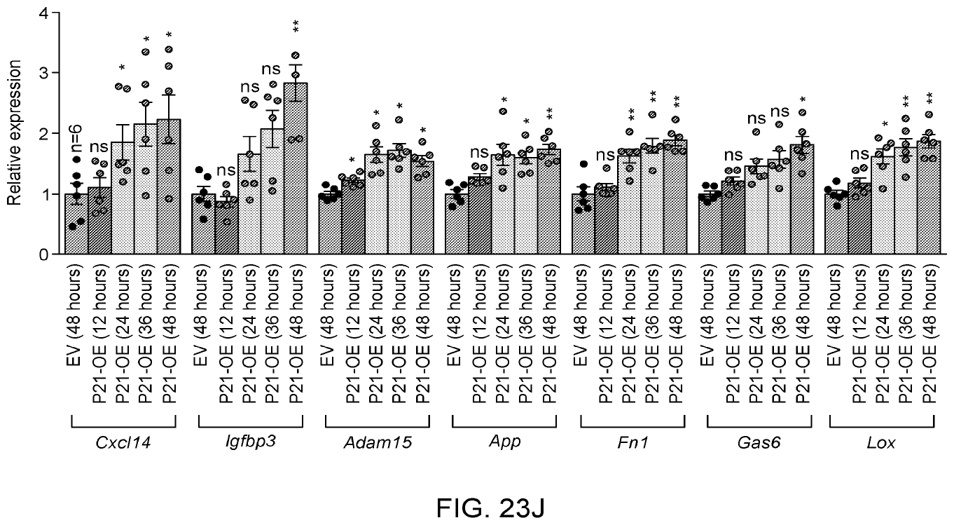

Stress-inducing oncogenic point mutations are irreparable, but many cellular stresses are transient or repairable. To determine whether stressed cells that recuperate and normalize P21 cease to produce a PASP and are released from immunosurveillance, MEFs containing a lentiviral construct that allows for doxycycline (dox)-inducible expression of p21-Myc-Flag were produced. These MEFs stopped proliferating within 2 days after dox administration, but were fully capable of resuming the cell cycle after dox withdrawal (FIGS. 4A and 4B and FIGS. 23A to 23C). CM harvested from D2 P21-OE MEFs stimulated fibroblasts migration in the scratch assay and macrophage migration in the transwell assay with peritoneal immune cells (FIG. 4C and FIGS. 23D and 23E). In contrast, however, CM prepared from MEFs that had been on dox for 2 days followed by 4 days off dox had no impact on cell migratory properties in these same assays. Cxcl14 expression followed the promigratory properties of P21-OE CM (FIG. 4D). Detailed analyses of P21-OE MEFs and CM thereof at 12 hour intervals after dox administration revealed that suppression of E2F target genes, proliferative arrest, induction of PASP genes and chemoattraction of macrophages are all occur within 24 hours after induction of P21 (FIGS. 23G to 23J), indicating that halting cell cycle progression and PASP-mediated immune surveillance occur simultaneously and rapidly.

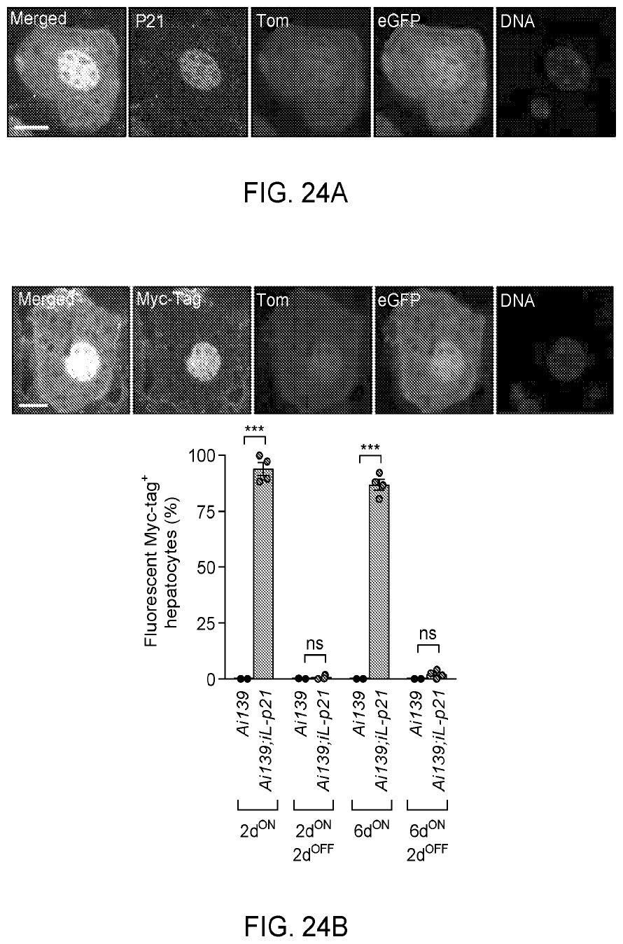

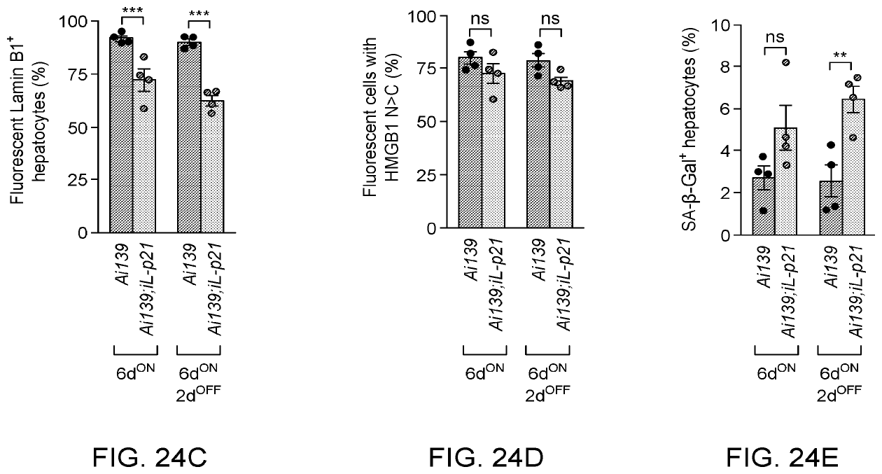

To determine the time damaged cells have to recuperate and avert elimination by immune cells under physiological conditions and to define the underlying timer mechanism, we created transgenic mice in which p21-Myc-Flag can be co-activated with GFP and Tom in hepatocytes with adeno-Cre injection and p21-Myc-Flag and GFP repressed by dox administration (FIG. 4E). Macrophages surrounding P21-OE hepatocytes at D2 and D4 withdrew within two days after suppressing transgenic P21 (FIGS. 4F and 4G and FIGS. 24A and 24B). Macrophages surrounding P21-OE hepatocytes at D6 did not disengage upon dox administration despite complete silencing of P21 and lack of endogenous P21 induction. Other distinctions of P21-OE hepatocytes at D6 were that adjoining macrophages had undergone M1 activation and that lymphocytes had been recruited, which, like the macrophages, did not disengage after normalization of P21 levels and were primed for target cell elimination (FIGS. 4H to 4J). D6 P21-OE hepatocytes were not yet senescent although some entry into the senescent state occurred during the 2-day off period (FIGS. 24C to 24E). Thus, P21-induction in stressed cells sets a timeframe for repair or adaptation that is defined by the time it takes for the immune system to transition from a cell-surveillance to a cell-clearance mode.

Together these studies revealed that P21 can respond to cellular stressors through a non-cell-autonomous mechanism by placing cells under immunosurveillance, and that P21 can do so concomitantly with halting cell cycle progression (FIG. 25). In probing the mechanism, it was discovered that the pool of hypophosphorylated RB that is created in response to P21 induction binds to chromatin to not only establish a cell cycle arrest but also to activate select SMAD and STAT TFs to create a bioactive secretome with diverse biological functions, including immunosurveillance. It was also found that CXCL14 within this secretome can attract macrophages to cells with elevated P21.

### Materials and Methods

### Mouse Strains

L-KRAS^{G12V} mice were generated from KH2 ES cells using a modified pS31 vector. Briefly, the tetracycline-inducible promoter and the SV40 polyA signal in the pBS31 were replaced by a CAG promoter-FRT-loxP-flanked STOP cassette (LoxP7-STOP-LoxP, L) and WPRE-bGH-polyA (WPRE-pA) from Ai9 (Addgene, #22799), respectively. The FRT site after the CAG promoter was deleted using site-directed mutagenesis and a multiple cloning site (MCS) was added between L and WPRE-pA. The Myc-tagged human KRAS^{G12V} was amplified from pBABE-KRAS^{G12V}-puro (Addgene, #9052) and inserted to the MCS. The resultant pBS31-CAG-L-KRAS^{G12V}-WPRE-pA plasmid was electroporated into KH2 ES cells and selected clones with Cre-inducible KRAS^{G12V} expression were used to generate L-KRAS^{G12V} mice according to standard procedures. The same strategy was used to generate L-p21 mice or L-p16 mice using Myc-Flag-tagged cDNAs for mouse Cdkn1a (encoding P21) or mouse Cdkn2a (encoding P16) obtained from Origene (#MR227529 or #MR227284, respectively). Obtained founder mice were backcrossed to C57BL6 at least twice before use for experimentation. To generate iL-p21 transgenic mice, the following targeting construct was cloned: pTRE2-LoxP-STOP-LoxP (LSL)-p21-Myc-Flag-WPRE-pA using the pTRE2 promoter and LSL from the Ai139 transgene (Addgene, #114426) and p21-Myc-Flag from the L-p21 transgene (Origene, #MR227529) as described above. Homology arms spanning 968 bp at the 5′ end and 937 bp at the 3′ end flanked by sgRNA target sites were used to target the construct into the Col1a1 locus of C57BL/6NHsd (Envigo) zygotes using CRISPR-Cas9-mediated gene editing with Cas9 mRNA (Trilink Biotechnologies, #L-7606) and Col1a1-specific sgRNA 5′-GAGGTTCATGAGCCCTCAAA-3′ (SEQ ID NO:13). Obtained founder mice were backcrossed to C57BL6 once before use for experimentation. To generate p21floxed mice, a targeting vector containing Cdkn1a exon 2 flanked by LoxP sites and homology arms spanning 861 bp at the 5′ end and 819 bp at the 3′ end flanked by sgRNA target sites (5′ sgRNA 5′-TCTTGGTGATTAACTCCATC-3′ (SEQ ID NO:14) and 3′ sgRNA 5′-CCATAGGCGTGGGACCTCGT-3′ (SEQ ID NO:15)) was cloned. The resultant targeting vector was used to target the construct into the Cdkn1a locus of C57BL/6NHsd (Envigo) zygotes using CRISPR-Cas9-mediated gene editing with Cas9 mRNA (Trilink Biotechnologies, #L-7606). Obtained founder mice were backcrossed to C57BL6 at least once before use for experimentation. Rb^{floxed} mice (#026563), Ai14 transgenic animals (#007914) and Ai139 transgenic mice (#030219) were purchased from The Jackson Laboratory. The following cohorts were generated for experimentation in this study: Ai14/+ and Ai14/+ L-KRAS^{G12V}/+ (FIG. S2), Ai14/+ and Ai14/+ L-p21/+ (FIG. 2, FIG. 17, and FIG. 18), Ai14/+ and Ai14/+ L-p16/+ (FIG. 20), Ai14/+ and Ai14/+ L-KRAS^{G12V}/+ and Ai14/+ L-^{KRASG12V}/+ p21^{floxed/floxed} and Ai14/+ L-KRAS^{G12V}/+ Rb^{floxed/floxed} (FIG. 3 and FIG. 22), Ai139/+ and Ai139/+ iL-p21. (FIG. 4 and FIG. 24). Mice were aged until 4 to 6 months of age before use for experimentation unless otherwise noted.

### Cell Culture

Mouse embryonic fibroblasts (MEFs) were generated as described previously with each line being derived from a separate C57BL/6 E13.5 embryo containing INK-ATTAC. MEFs were cultured in DMEM (Gibco, #11960) supplemented with 10% heat-inactivated fetal bovine serum, L-glutamine, non-essential amino-acids, sodium pyruvate, gentamicin and β-mercaptoethanol. These lines were expanded at 3% oxygen and used for experiments between passage (P)3 and P6. IMR-90 cells were purchased from ATCC (#CCL-186) at P10 and cultured in the same medium as used for MEFs. IMR-90 cells were used for experimentation between P14 and P18. HDFs were generated from human foreskin of young, healthy donors (2 days to 13 years of age). Each line was derived from a separate donor. HDFs were cultured in the same medium as used for MEFs and used for experimentation between P5 and P8. HUVECs were purchased from ATCC (#PCS-100-013) and were cultured in vascular cell basal medium (ATCC, #PCS-100-030) supplemented with endothelial growth factors (Endothelial Cell Growth Kit-VEGF, ATCC, #PCS-100-041). HUVECs were used for experimentation at P3 to P5.

### Generation of Senescent and Non-Senescent MEFs

For H3K27Ac-ChIP-seq experiments, two or three independent MEF lines were generated and induced to senesce via irradiation (IR), serial passaging (REP) or KRAS^{G12V}-overexpression (OI). For identification of IR-induced senescence-associated super enhancers the following three MEF cultures were established from each independent MEF line: proliferating P3 MEFs (to derive C1 MEFs); P6 MEFs exposed to 10 Gy γ-radiation (¹³⁷Caesium source) and cultured for two days (to derive C2 MEFs); and P6 MEFs exposed to 10 Gy γ-radiation and cultured for 10 days (to derive IR-senescent MEFs). For identification of REP-induced senescence-associated super enhancers, two MEF cultures were prepared from each independent MEF line: proliferating P3 MEFs (to derive C1 MEFs); and P10 MEFs cultured at 20% oxygen between P4 and P10 (to derive REP-senescent MEFs). To identify senescence-associated super enhancers in OI-induced senescent MEFs, cells were infected with a KRAS^{G12V}-containing lentivirus (prepared using the pLenti-PGK-ER-KRAS^{G12V} from Addgene #35635), selected with 250 μg/mL hygromycin B (EMD Millipore, #400052) and then harvested (to derive C1 MEFs) or treated with 200 nM 4-hydroxytamoxifen (4′-OHT, 1:50,000 from stock in ethanol, Sigma H7904) to induce KRAS^{G12V} for 2 days (to derive C2 MEFs) or 10 days (to derive OI-induced senescent MEFs). IR-, REP- and OI-induced senescent MEFs were enriched by sterile FACS using a BD FACSAria 4-laser digital flow cytometer with FACSDiva v8.0.1 software with 488 nm laser. Sorted cells were pelleted, resuspended in fresh culture medium, counted and used for ChIP-seq and RNA extraction. Small amounts of the sorted cells were reseeded to assess the proportion of cell that was SNCs. Samples with ˜70% or more SNCs were used for H3K27ac-ChIP-seq experiments. C1 and C2 MEFs cultures were also subjected to FACS but here fractions devoid of SNCs were collected. For all other experiments involving REP-induced SNCs, SNCs were prepared as described above. FACS-enriched SNCs were cultured for at least 24 hours before further use. OI-induced senescent MEFs were also prepared as described above, but instead of the lentiviral KRAS^{G12V} expression system MEFs derived from L-KRAS^{G12V} mice were used. These MEFs were infected with pTSIN-Cre-PGK-puro2 lentivirus to induce KRAS^{G12V} expression. These MEFs were then cultured for days and subject to FACS enrichment of SNCs (the first two days in medium containing 2 μg/mL puromycin).

### Generation of IR-Senescent and Control IMR-90 Cells

H3K27ac-ChIP-seq experiments and matched RNA-sequencing experiments were conducted in triplicate using three technical replicates. IMR-90 cells were expanded at 3% oxygen and used for experiments at P18. For identification of IR-induced senescence-associated super enhancers the following three cultures from each of the replicates were established: proliferating P18 IMR-90 cells (to derive control 1 (C1) cells); P18 IMR-90 cells exposed to 10 Gy γ-radiation (¹³⁷Caesium source) and cultured for 2 days (to derive control 2 (C2) cells); and P18 IMR-90 cells exposed to 10 Gy γ-radiation and cultured for 10 days (to derive IR-senescent IMR-90 cells). Cells were trypsinized and reseeded to assess the proportion of cells that were senescent. Samples with >80% IR-SNCs were used for H3K27ac ChIP-seq experiments.

### ChIP-Seq Analyses and SE Identification in Cultured Cells

FACS-enriched MEF or IMR-90 suspensions were pelleted, resuspended in medium, and counted. 2-10×10⁵ cells were fixed with 1% paraformaldehyde (PFA) for 10 minutes and then subjected to ChIP-seq as using a rabbit anti-H3K27ac antibody (Abcam, ab4729, Lot GR150367). Chromatin immunoprecipitation-sequencing (ChIP-seq) libraries were prepared from 1-5 ng precipitated chromatin or input DNA using the Ovation ultralow DR Multiplex kit (NuGEN) or the ThruPLEX DNA-seq Kit V2 (Rubicon Genomics). ChIP enrichment was validated in library DNAs by performing quantitative PCR in the indicated genomic loci using following primers: mouse mPabpc1-TSS (F): 5′-ATCCCACAGCTTGTGGCGGG-3′ (SEQ ID NO:16); (R): 5′-TCTCGCCATCGGTCGCTCTC-3′ (SEQ ID NO:17); mIntergenic (F): 5′-CCT-GCTGCCTTGTCTCTCTC-3′ (SEQ ID NO:154); (R): 5′-ATGGCCTAGGGATTCCAGCA-3′ (SEQ ID NO: 155). The ChIP-seq libraries were sequenced to 51 bp from both ends on an Illumina HiSeq 2000 or HiSeq 4000 instrument.

Fastq files of pair-end reads were mapped with Bowtie 1.1.2 using parameters-k 1-m 1-e 70-151 (mm10 for mouse, hg19 for human). MACS 1.4.2 was used to identify peaks for each sample against the background using a p-value cutoff of 10-5. All other parameters were left at default. To identify super enhancers (SEs), neighboring peaks were first stitched together to create a single region capturing these signals as a whole. Peaks occurring within 12.5 kb from each other were combined into stitched enhancers while excluding regions that were within ±2,000 bps from any transcription start site (TSS). These stitched enhancers were then ranked by background-subtracted ChIP-seq occupancy ascendingly, and the occupancy was plotted in the unit of reads per million per base pair. From the plot, the point where occupancy started increasing faster was identified by first scaling the x- and y-axes into [0, 1] and then finding the point where a line with a slope of 1 was tangential to the curve. Occupancy increased slowly below but rapidly above this point. The stitched enhancers above this point were defined as SEs. All the above procedures were performed using ROSE. In order to determine differential binding for SE between treatment and control samples, SE regions from all samples were first merged into a set of merged regions covering all SE regions in all samples. Tag counts at each merged region were then extracted and differential analysis on the tag counts were performed using R package DESeq2 1.10.1 using the same settings as described below (see RNA-sequencing). Senescence-associated super enhancers were defined as SEs with lfcMLE (unshrunk log 2 fold change produced by DESeq2) in tag counts ≥0.3 for both senescent vs. proliferating (C1) and senescent vs. induced, non-senescent (C2). SEs were assigned to genes within ±50 kb of the SE by calculating the distance between either end of each SE and TSS of each gene. Only SEs±50 kb from at least one TSS were considered in downstream analyses. For downstream validation, only senescence-associated super enhancer-controlled genes that were differentially expressed with false discovery rate (FDR)<0.05 in at least two of three senescence mechanisms were considered. BigWig files of H3K27Ac occupancy profiles were generated using deepTools 3.1.0 by first normalizing each ChIP-seq sample and its matching input to cpm (counts per million mapped reads) and then subtracting the input signal from each ChIP sample. H3K27ac occupancy plots were generated via Integrative Genomics Viewer (IGV). To identify RB peaks at promoters of secreted factors, published RB ChIP-seq data from OI-senescent, quiescent and non-senescent IMR-90 cells were analyzed (GSE19899). Peaks were annotated to genes within 50 kb from either end of any peak. The peak sequences of SASP genes associated to any RB peak with 2.5 kb padding from each end were used as input to MEME-ChIP to detect enriched motifs using the HOCOMOCO database. FIMO was used to locate occurrences of motifs in each input sequence.

### ChIP on Senescent Liver Cells

FACS-enriched Tom⁺ cell suspensions from Ai14;L-KRAS^{G12V} or Ai14 control livers (see below) were pelleted, resuspended in medium, and counted. 1-4×10⁵ cells were fixed with 1% PFA for 10 minutes and then subjected to H3K27ac-ChIP using a rabbit anti-H3K27ac antibody (Abcam, ab4729, Lot GR150367) or rabbit, IgG (Millipore, #12-370) according to the manufacturers protocol (Active Motif, #53084). Precipitated chromatin or input DNA was subjected to quantitative PCR in the indicated genomic regions in the senescence-associated super enhancer of the Cdkn1a locus using primers indicated in Table

TABLE 4
Listing of utilized primers and shRNA target sequences.

| | | | SEQ | | SEQ |
|---|---|---|---|---|---|
| | | | ID | | ID |
| RT-qPCR primers | | Primer forward (5′-3′) | NO: | Primer reverse (5′-3′) | NO: |
| mus | Tbp | GGCCTCTCAGAAGCATCACTA | 18 | GCCAAGCCCTGAGCATAA | 19 |
| musculus | p16 (Cdkn2a) | CCCAACGCCCCGAACT | 20 | GCAGAAGAGCTGCTACGTGAA | 21 |
| | p19 (Cdkn2a) | GCCGCACCGGAATCCT | 22 | TTGAGCAGAAGAGCTGCTACGT | 23 |
| | p21 (Cdknla) | GTCCAATCCTGGTGATGTCC | 24 | GTTTTCGGCCCTGAGATGT | 25 |
| | p27 (Cdkn1b) | AGTGTCCAGGGATGAGGAAG | 26 | GGGGAACCGTCTGAAACATT | 27 |
| | Rb | GAACAGATTTGTCCTTCCCG | 28 | CCATGATTCGATGCTCACAT | 29 |
| | Adam15 | ATGGCACCCGAATGGTCAG | 30 | CTCCAGTGTATAGCCTCTCTCTG | 31 |
| | App | AGCTTGGCACTGCTCCTG | 32 | GTTTACCACAGAACATGGCG | 33 |
| | Ccl2 | ATTGGGATCATCTTGCTGGT | 34 | CCTGGTGTTCACAGTTGCC | 35 |
| | Ccl7 | CCTGGGAAGCTGTTATCTTCAAG | 36 | CCTCCTCGACCCACTTCTGA | 37 |
| | Cxcl1 | GTGCCATCAGAGCAGTCTGT | 38 | ACCCAAACCGAAGTCATAGC | 39 |
| | Cxcl14 | GAAGATGGTTATCGTCACCACC | 40 | CGTTCCAGGCATTGTACCACT | 41 |
| | Cxcl15 | CCATGGGTGAAGGCTACTGT | 42 | AGAGGCTTTTCATGCTCAACA | 43 |
| | Fn1 | GGGAGAAGTTTGTGCATGGT | 44 | CTGGGGGTCTCCGTGATAAT | 45 |
| | Gas6 | GACCCCGAGACGGAGTATTTC | 46 | TGCACTGGTCAGGCAAGTTC | 47 |
| | Igfbp2 | GGGTGCCAAACACCTCAG | 48 | AGGTTGTACCGGCCATGC | 49 |
| | Igfbp3 | TAAGAAGAAGCAGTGCCGCC | 50 | TTTCCCCTTGGTGTCGTAGC | 51 |
| | Lox | ACTTCCAGTACGGTCTCCCG | 52 | GCAGCGCATCTCAGGTTGT | 53 |
| | Mmp2 | TGCAGGAGACAAGTTCTGGA | 54 | GACGGCATCCAGGTTATCAG | 55 |
| | Mmp19 | CCTGGTCCCATGCCAAACC | 56 | CCCTTGAAAGCATAAGTCTTCCC | 57 |
| | Tnf | CAGCCTCTTCTCATTCCTGC | 58 | AGGGTCTGGGCCATAGAACT | 59 |
| | Wnt5a | ATGCAGTACATTGGAGAAGGTG | 60 | CGTCTCTCGGCTGCCTATTT | 61 |
| | Pdgfa | GTGCGACCTCCAACCTGA | 62 | GGCTCATCTCACCTCACATCT | 63 |
| | Psap | CTCTTCGCCAGCCTTCTG | 64 | TTGCTCCAGACCATCTGCT | 65 |
| | Ssc5d | GGCTGGAAGGCCCATATCTG | 66 | CAGCAGGGACATTGAATCTTCT | 67 |
| | Il1a | TCAACCAAACTATATATCAGGATGTGG | 68 | CGAGTAGGCATACATGTCAAATTTTAC | 69 |
| | Il6 | GCTACCAAACTGGATATAATCAGGA | 70 | CCAGGTAGCTATGGTACTCCAGAA | 71 |
| | Ccl3 | CTCCCAGCCAGGTGTCATTTT | 72 | CTTGGACCCAGGTCTCTTTGG | 73 |
| | Ccl20 | ACTGTTGCCTCTCGTACATACA | 74 | GAGGAGGTTCACAGCCCTTTT | 75 |
| | Cxcl2 | CATCCAGAGCTTGAGTGTGACG | 76 | GGCTTCAGGGTCAAGGCAAACT | 77 |
| | Cxcl3 | TGAGACCATCCAGAGCTTGACG | 78 | CCTTGGGGGTTGAGGCAAACTT | 79 |
| | Cxcl5 | TGCCCTACGGTGGAAGTCATA | 80 | TGCATTCCGCTTAGCTTTCTTT | 81 |
| | Cxcr4 | GACTGGCATAGTCGGCAATGGA | 82 | CAAAGAGGAGGTCAGCCACTGA | 83 |
| | Icam1 | AAACCAGACCCTGGAACTGCAC | 84 | GCCTGGCATTTCAGAGTCTGCT | 85 |
| | Rela | GAGTCTCCATGCAGCTACGG | 86 | CGCTTCTCTTCAATCCGGT | 87 |
| | Cdk1 | TCCGTCGTAACCTGTTGAGT | 88 | TGGCCAGTGACTCTGTGTCT | 89 |
| | Cdc25a | GGCTGTTTGACTCCCCTTC | 90 | GGGCACACTCTTCCTCCTCT | 91 |
| | Ccna2 | GGCCAGCTGAGCTTAAAGAA | 92 | GTGGTGATTCAAAACTGCCA | 93 |
| homo | TBP | GCCAGCTTCGGAGAGTTCTGGGATT | 94 | CGGGCACGAAGTGCAATGGTCTTTA | 95 |
| sapiens | P16 (CDKN2A) | GTGAGAGTGGCGGGGTC | 96 | CCCAACGCACCGAATAGTTA | 97 |
| | P21 (CDKNIA) | GCCATTAGCGCATCACAGT | 98 | ACCGAGGCACTCAGAGGAG | 99 |
| | RB | TTGGATCACAGCGATACAAACTT | 100 | AGCGCACGCCAATAAAGACAT | 101 |
| | ADAM15 | GCTCCCAAATATAGGTGGCACT | 102 | CCAACTTGATCCTCAGGGGC | 103 |
| | APP | AGGGACCAAAACCTGCATTGA | 104 | ACTCACCAACTAAGCAGCGG | 105 |
| | CXCL14 | AAGCCAAAGTACCCGCACTG | 106 | GACCTCGGTACCTGGACACG | 107 |
| | FN1 | AGGAGAATGGACCTGCAAGC | 108 | GAAGTGCAAGTGATGCGTCC | 109 |
| | GAS6 | CTCGTGCAGCCTATAAACCCT | 110 | TCCTCGTGTTCACTTTCACCG | 111 |
| | IGFBP3 | GTGGATCCCTCAACCAAGAA | 112 | TAGGTTCCCAGAGTGCCCTA | 113 |
| | LOX | CGGCGGAGGAAAACTGTCT | 114 | TCGGCTGGGTAAGAAATCTGA | 115 |
| ChIP-qPCR primers | | Primer forward (5′-3′) | | Primer reverse (5′-3′) | |
| mus | Cdkn1a region 1 | TGAGGAGGAGCATGAATGGAG | 116 | TTCTGCTGGCAAAGTGGGAC | 117 |
| musculus | Cdkn1a region 2 | GTGCAATGGTGTGCCTGACTA | 118 | AAGTCTGGGACTACTCAGTCTTTC | 119 |
| | Cdkn1a region 3 | GCTCTGGGAAGCCAGAAGTT | 120 | GACCTCCTGTGCCTTTACCC | 121 |
| | Cdkn1a region 4 | TTTTGACATCCTGTGCTGGC | 122 | CCAGTCCCTGCATCCAAGTC | 123 |
| | Cdkn1a region 5 | GGTGATCTCAGATAGCTCAGGC | 124 | AATCACGGTACTTGGGAGGC | 125 |
| | Cdkn1a region 6 | TGCTTAGCTGAGATGGTGGTCT | 126 | CAGTCTTGTTACACGATCCAGCC | 127 |
| | Cdkn1a region 7 | \|ACTGCTATGTCTGTCAGGAACA | 128 | CCAAGATCCAGACAGTCCACTAAA | 129 |
| shRNA target sequences | | target sequence (shRNA-1) | | target sequence (shRNA-2) | |
| mus | p21 (Cdkn1a) | GACCAGCCTGACAGATTTCTA | 130 | CTATCACTCCAAGCGCAGATT | 131 |
| musculus | Rb | CGCTATGAAGAAGTTTATCTT | 132 | CCGTGGATTCTGAACGTACTT | 133 |
| | Stat1 | CCGAAGAACTTCACTCTCTTA | 134 | ACGCCTTTGGGAAGTATTATT | 135 |
| | Stat6 | CCACAGTCCATCCACTCATTT | 136 | CGTCTCAACTGTTCCTTGGTT | 137 |
| | Smad2 | CCCATCAAAGACTCGCTGTAA | 138 | CGGTTAGATGAGCTTGAGAAA | 139 |
| | Smad3 | CTGTCCAATGTCAACCGGAAT | 140 | ACGTGAACACCAAGTGCATTA | 141 |
| | Cxcl14 | GCTGGAAATGAAGCCAAAGTA | 142 | CTGCGAGGAGAAGATGGTTAT | 143 |
| | Rela | AGAAGACATTGAGGTGTATTT | 144 | GGAGTACCCTGAAGCTATAAC | 145 |
| homo | P21 (CDKN1A) | TCACTGTCTTGTACCCTTGT | 146 | GAGGTTCCTAAGAGTGCTGG | 147 |
| sapiens | RB | CAGAGATCGTGTATTGAGATT | 148 | AAGAACGATTATCCATTCAAA | 149 |

Bolded sequence indicates shRNA used for mechanistic experiments in which one hairpin was used.

### RNA Isolation and RT-qPCR

MEFs or IMR-90 cells, or flow-sorted liver cells were lysed in RLT buffer supplemented with β-mercaptoethanol according to the RNA extraction protocol. RNA extraction (Qiagen, RNeasy Mini kit, #74104, or RNeasy Micro kit, #74004), cDNA synthesis (Invitrogen, SuperScript III First-Strand Synthesis, #18080051), and real-time quantitative PCR (RT-qPCR) analysis (Applied Biosystem, SYBR Green Real-Time PCR Master Mix, #4309155) were performed according to manufacturer's instructions. The on-column DNase digestion step was avoided during the RNA extraction procedure unless RNA was used for RNA-sequencing purposes. Primers were optimized via cDNA dilution series. Tbp (TBP in human) was used as a reference gene for RT-qPCR in mouse and human samples. Primer sequences are listed in Table 4.

### RNA-Sequencing

Equal amounts of high-quality RNA (100-200 ng) were subjected to library preparation using the TruSeq RNA Library Prep Kit v2 (Illumina, #RS-122-2001) according to the manufacturer's instructions. Libraries were sequenced following Illumina's standard protocol using the Illumina cBot and HiSeq 3000/4000 PE Cluster Kit. Flow cells were sequenced as 100×2 paired end reads on an Illumina HiSeq 4000 using HiSeq 3000/4000 sequencing kit and HCS 3.3.20 collection software. Base-calling was performed using Illumina's RTA 2.5.2 software.

Fastq files of pair-end RNA-seq reads were aligned with Tophat 2.0.14 to the reference genome (mm10 for mouse, hg19 for human) using Bowtie2 2.2.6 with default parameters. Gene level counts were obtained using FeatureCounts 1.4.6 from the SubRead package with gene models from corresponding UCSC annotation packages. Differential expression analysis was performed using R package DESeq2 1.10.1 after removing genes with average raw counts less than 10. During the DESeq2 analysis thresholding on Cook's distance for outliers and independent filtering were turned off so that all genes passed to DESeq2 were assigned p-values for significance of differential expression. Genes with FDR <0.05 were considered significantly differentially expressed. Hierarchical clustering of samples was performed using DESeq2-normalized counts with 1-Pearson correlation as distance and average linkage using R function hclust. Gene Set Enrichment Analysis (GSEA) was performed as previously described against mouse genesets from Enrichment map using gene lists ranked by lfcMLE, which was the unshrunk log₂ fold change produced by DESeq2, in descending order. Functional annotation analyses were performed via String database v11 focusing on GO BP annotations, KEGG pathways and Reactome pathways with FDR <0.05. Overrepresentation analysis for transcription regulatory targets of individual TFs was performed using the Fisher's exact test method for selected gene lists against the mouse gene sets from ENCODE and MSigDB collections. Mouse TF targets were mapped to human orthologs using MGI's Vertebrate Homology database and used for overrepresentation analyses in human datasets. Putative SASP factor genes were extracted from Gene Ontology Consortium (Mus musculus MGI and Homo sapiens GO Annotations EBI) and QuickGO database for the annotation GO: 0005615 “Extracellular Space”. Gene lists from both reference databases were merged resulting in the identification of 1845 or 3513 factors for mouse or human, respectively. Heatmaps were generated with Morpheus, Broad Institute (software.broadinstitute.org/morpheus). For gene expression heatmaps based on RNA-seq data, lfcMLE values and −log₁₀ of FDR values were used.

Adeno-Virus Injection into Mice and Isolation of Liver Cells

To generate in vivo OI-senescent liver cells, 4-month-old Ai14;L-KRAS^{G12V} or Ai14 control mice we used and adeno-Cre-EGFP virus (University of Iowa, Vector Labs, #VVC-U of Iowa-1174) at 10⁹ pfu/100 μl in 0.9% NaCl was injected into the tail vein. Eight days post-injection, livers were harvested and the peri-venous half of the left lateral lobe was fixed with 4% PFA in PBS for 2 hours and soaked in 30% sucrose overnight. These livers were embedded in OCT (1 Sakura, #4583) and used for cryosectioning and confocal imaging. To assess proliferation rates in these mice, 50 mg/kg EdU (5-ethynyl-2′-deoxyuridine, Carbosynth, #NE08701) was IP injected on day 6 and day 7 post adeno-Cre injection for a total of 48 hours before euthanasia of mice. EdU staining was performed on cyrosections with the same kit and protocol used in vitro (see below). To isolate Tom⁺ liver cells, livers of Ai14;L-KRAS^{G12V} or Ai14 control mice 8 days post-injection were perfused with collagenase. Because the parenchymal fraction of Ai14;L-KRAS^{G12V} was not viable, the non-parenchymal fraction was subjected to FACS as described above with appropriate lasers and filters. For in vivo P21-OE and P16-OE studies, Ai14;L-p21 or Ai14;L-p16 or Ai14 control mice were injected with adeno-Cre-EGFP virus (University of Iowa, Vector Labs) at 10⁸ pfu/100 μL 0.9% NaCl into the tail vein. Two, 4 or 8 days post-injection, livers were harvested and fixed as described above. To assess proliferation rates in these mice, 50 mg/kg EdU was injected intra-peritoneally on day 2 and day 3 post-injection for a total of 48 hours before euthanasia of mice. For in vivo KRAS^{G12V}-OE studies, Ai14;L-KRAS^{G12V}, Ai14;L-KRAS^{G12V} p21^{floxed/floxed} Ai14;L-KRAS^{G12V} Rbfloxed floxed or Ai14 control mice were injected with adeno-Cre-EGFP virus (University of Iowa, Vector Labs) at 0.25×10⁸ pfu/100 μL 0.9% NaCl into the tail vein. Four, 12 or 28 days post-injection, livers were harvested and fixed as described above. To assess proliferation rates in these mice, 50 mg/kg EdU was injected intra-peritoneally on 2 days and 1 day for a total of 48 hours before euthanasia of mice. To isolate Tom⁺ hepatocytes for expression analyses, livers were perfused with collagenase and the parenchymal fraction was subjected to FACS as described above. For in vivo inducible P21-OE studies, Ai139;iL-p21 or Ai139 control mice were injected with adeno-Cre-EGFP virus (University of Iowa, Vector Labs) at 10⁸ pfu/100 μL 0.9% NaCl into the tail vein. At indicated timepoints (“ON”), livers were harvested and fixed as described above. To suppress P21-OE (“OFF”), mice were treated with Doxycycline (dox, Letco, #690902) at 100 mg/kg in water via gavage every 24 hours (for a total of 48 hours) until euthanasia and liver collection.

### DNA Isolation and PCR for Recombined Conditional Alleles

Livers of A14i;L-KRAS^{G12V}, A14i;L-KRAS^{G12V}; p2^{floxed/floxed} or A14i; L-KRAS^{G12V}; Rbfloxed floxed mice that received 0.25×10⁸ pfu adeno-Cre virus (containing ˜5% Tom⁺ hepatocytes) or did not receive virus were flash frozen and stored at −80° C. These livers were homogenized via mortar and pestle and DNA was isolated through phenol-chloroform extraction. PCR analysis of Cdkn1a (P21) exon 2 was done using the following primers: (F) 5′-GTATCCCAAAGTCCAGGGCACT-3′ (SEQ ID NO:150) and (R) 5′-TGCCAAGGGGAAGGACATCATT-3′ (SEQ ID NO:151) generating 1446 bp, 1549 bp and 609 bp products for the wild type, unrecombined-floxed and recombined-floxed alleles, respectively. PCR analysis of Rb exon 19 was done using the following primers Rb18 (F) 5′-GGCGTGTGCATCAATG-3′ (SEQ ID NO:152) and Rb212 (R) 5′-GAAAGGAAAGTCAGGGACATTGGG-3′ (SEQ ID NO:153) generating 698 bp, 746 bp and 260 bp products for the wild type, unrecombined-floxed and recombined-floxed alleles, respectively.

### Neutralizing Antibody Experiments in Mice

To deplete CD8⁺ T cells, Ai14;L-p21 and Ai14 mice were IP injected with 500 μg rat anti-CD8a antibody (clone 53-6.7, BioXcell, #BE0004-1) in 200 μL PBS or 200 μL PBS (as control) each day for 3 consecutive days and again on D6. On the day 7, 10⁸ pfu adeno-Cre virus in 100 μL 0.9% NaCl was injected intravenously as described above. On D12 mice were IP injected once more with anti-CD8a antibody or PBS, mice were euthanized and livers and spleens were collected at D15 (corresponding to D8 post-adeno-Cre injection). Spleens were processed freshly to isolate cells for flow cytometry. Spleens were crushed between 2 frosted slides, the cell suspension was filtered through a 70 μm filter and spun at 1,500 rpm for 5 minutes. Red blood cells were removed via ACK lysis for 8 minutes on ice. Tubes were filled with PBS, spun again, resuspended and total cell numbers were counted. For flow cytometry assessments, 100,000 cells were used for antibody staining using the following antibodies: hamster anti-TCRb-FITC (Tonbo Biosciences, #35-5961, 1:500), rat anti-CD4-PerCP (BioLegend, #100538, 1:500) and rat anti-CD8α-violetFluor450 (clone 2.43, Tonbo biosciences, #75-1886, 1:500) and viability dye Ghost Dye Red 780 (Tonbo biosciences, #13-0865, 1:1,000). Total CD4⁺ or CD8α⁺ T cells were calculated using flow cytometry quantifications and the previously noted total cell numbers per spleens.

To neutralize CXCL14, Ai14;L-p21 and Ai14 mice were IP injected with the following antibodies in 200 μL PBS: 500 μg rat anti-CXCL14 antibody (R&D Systems, #MAb730), 500 μg mouse anti-CXCL14 antibody (R&D Systems, #MAb866), 500 μg mouse IgG2a isotype control (BioXcell, #BE0085 as control for MAb730) or 500 μg rat IgG2b isotype control (BioXcell, #BE0090 as control for MAb866). The next day, antibody injection was repeated and mice were also injected with 10⁸ pfu adeno-Cre virus in 100 μL 0.9% NaCl intravenously as described above. The following day, antibody injection was repeated once more. Mice were euthanized and livers were collected the next day (D3, corresponds to D2 post-adeno-Cre injection).

### Cryosectioning and Immunofluorescence on Liver Tissue

OCT-embedded livers were sectioned using a Cryostat (CM 1900, Leica) to generate 20 μm-tick frozen sections. Sections were washed with PBS and permeabilized with 0.5% Triton-X-100 for 20 minutes. Sections were blocked with 5% BSA/PBS for 1 hour and subsequently incubated overnight with primary antibodies rabbit anti-F4/80 (Cell Signaling, #70076; 1:250), rat anti-B220/CD45R-FITC (BD BioSciences; #553088; 1:50), rat anti-NKp46/CD335-FITC (Biolegend, #580756; 1:50), rabbit anti-CD38 (Cell Signaling, #99940; 1:50), rabbit anti-CD4-biotin (BioLegend, #100508, 1:50; in combination with Streptavidin-FITC, BioLegend, #405201, 1:100), rabbit anti-CD8a (Cell Signaling, #98941, 1:20), rabbit anti-iNOS (Abcam, ab15323, 1:100), rabbit anti-Lamin B1 (Abcam, ab16048, 1:500) or rabbit anti-HMGB1 (Abcam, ab18256, 1:1 500), rabbit anti-P21 (Abcam, ab188224, 1:100 or 1:250), rabbit anti-Myc-tag (Cell Signaling, #2272, 1:100), mouse anti-Myc-tag (Cell Signaling, #2276, 1:100; in combination with goat anti-mouse IgG2a AlexaFluor647 secondary antibody, Invitrogen, #A21241, 1:100), rabbit anti-phospho-Histone H3 (Ser10) (pHH3, Millipore, #06-570, 1:250) or rat anti-F4/80-AlexaFluor488 (Bio-Rad, #MCA497A488T, 1:100; used for co-immunofluorescence in combination with rabbit anti-P21 staining) diluted in 5% BSA/PBS and secondary antibodies goat anti-rabbit AlexaFluor488 (Invitrogen, #A11034; 1:250) or goat anti-rabbit-AlexaFluor647 (Invitrogen, #A21244; 1:100) for 3 hours. Incubation with secondary antibodies was avoided if the primary antibody was conjugated to FITC or AlexaFluor-fluorophores. Washings between incubations were performed in PBS (three washings of 5 minutes each). Cells were counterstained with Hoechst. A laser-scanning microscope (LSM 880; Zeiss) with an inverted microscope (Axiovert 100 M; Zeiss) was used to capture z-stack images with 2 μm step size (F4/80, iNOS, NKp46, CD38, CD4, CD8a and B220 stainings). The percentage of Lamin B1⁺ nuclei was determined as the percentage of Tom⁺ hepatocytes with Lamin B1-staining versus Tom⁺ hepatocytes without Lamin B1 staining. At least 50 hepatocytes or 2 sections were counted. For HMGB1 staining, the localization of nuclear versus cytoplasmic staining was examined per Tom⁺ hepatocyte and percentage of Tom⁺ hepatocytes with nuclear HMGB1 (N>C) was determined compared to Tom⁺ hepatocytes with loss of nuclear HMGB1 and gain of cytoplasmic staining (N<C). At least 50 hepatocytes or 2 sections were counted. To determine the proportion of P21-induced hepatocytes, the percentage of Tom⁺ hepatocytes with nuclear P21-staining versus Tom⁺ hepatocytes without nuclear P21 were quantified. At least 100 hepatocytes or 2 sections were counted. Similar analyses were done to quantify Myc-tag-induced hepatocytes of Ai14;L-p21 mice. To determine the proportion of Myc-tag-induced Ai14;L-KRAS^{G12V} hepatocytes, the percentage of Tom⁺ hepatocytes with Myc-tag-staining at the plasma membrane versus Tom⁺ hepatocytes without Myc-tag staining were quantified. To count the number of macrophages/Kupffer cells, B cells, T cells or NK cells associated per Tom⁺ hepatocyte, the number of F4/80⁺ cells, B220⁺, CD3ε⁺ or NKp46⁺ cells, respectively, immediately adjacent to Tom⁺ hepatocytes was counted. At least 100 hepatocytes or 2 sections were counted. Similar quantifications were done for the M1 macrophage marker iNOS and T cell subset markers CD4 and CD8α. To assess the proportion of Tom⁺ hepatocytes actively progressing through the cell cycle, Tom⁺ hepatocytes with nuclear pHH3 staining versus Tom⁺ hepatocytes without pHH3 signal were quantified. Cells with pHH3 staining were sub-divided into Tom⁺ pHH3⁺ before nuclear envelop breakdown as determined via Hoechst signal (considered G2 cells) and after nuclear envelop breakdown (considered mitotic cells). To determine the percentage of Tom⁺ hepatocytes, at least 400 hepatocytes were scored and the percentage of Tom⁺ versus Tom⁺ hepatocytes (as determined by nuclear and cellular shape) were determined. To assess the number of dying hepatocytes, at least 100 Tom⁺ hepatocytes were examined for cellular health and cells with overtly fragmented cytoplasm were considered as dying. Tom⁺ hepatocyte clusters were defined as 3 or more Tom⁺ hepatocytes being immediately adjacent, while Tom⁺ single hepatocytes were assessed when having no other Tom⁺ hepatocyte immediately adjacent. To quantify Tom⁺ hepatocyte clusters, large tile images were captured, assessed for the number of Tom⁺ hepatocyte clusters and normalized to the area of the tile image. Three sections were analyzed and averaged. For all quantifications involving Ai139;iL-p21 or Ai139 mice, similar staining regiments and quantifications were performed, but with the following modifications. In samples without doxycycline (“ON”) Tom⁺ eGFP⁺ hepatocytes were selected for quantification, whereas in the presence of doxycycline (“OFF”) Tom⁺ hepatocytes were selected. At least 50 Tom⁺ hepatocytes were examined.

### Immunostaining and Confocal Microscopy (Cells)

For P21 or 53BP1 immunostainings, flow-sorted MEFs were seeded on 10-well chambered slides (HTC supercured, Thermo Fisher Scientific, #30966S Black) at 2,000 cells/well. The next day, cells were fixed in PBS/4% PFA for 15 minutes, permeabilized in PBS/0.2% Triton X-100 for 15 minutes and blocked in PBS/5% BSA for 1 hour. Primary antibodies mouse anti-P21 (Santa Cruz, sc-53870; 1:200) or rabbit anti-53BP1 (Novus Biological, #NB100-305; 1:200) were diluted in PBS/5% BSA and subsequently incubated with primary antibodies overnight and secondary antibodies (goat anti-rabbit AlexaFluor488, Invitrogen, #A11034; 1:250) for 3 hours. Washings between incubations were performed in PBS (three washings of 5 minutes each). Cells were counterstained with Hoechst and the percentage of P21⁺ nuclei was determined. For 53BP1 staining, the number of clearly visible 53BP1 foci per cell was counted and percentage of 53BP1⁺ cells with more than 1 focus was determined. At least 100 cells or 50 cells per sample were counted for P21- or 53BP1-staining, respectively. A laser-scanning microscope (LSM 880, Zeiss) with an inverted microscope (Axiovert 100 M, Zeiss) was used to capture images.

### Plasmid Constructs

ShRNA oligo sequences were obtained from the RNAi Consortium (TRC, Broad Institute) and cloned into pLKO.1 vector (Addgene, #10878). Per gene, 4-5 shRNAs were tested for their knockdown potential and the two most efficient shRNAs were used in experiments. The non-targeting TRC2 shRNA (referred to as scrambled shRNA. shScr, Sigma-Aldrich, #SCH202) was used as a negative control. For shRNA sequences see Table 4. The Myc-Flag-tagged cDNA for mouse Cdkn1a was obtained from Origene (#MR227529) and subcloned into the lentiviral pTSIN-PGK-puro2 backbone or dox-inducible pTRIPZ-PKG-puro backbone (modified from GE Dharmacon). Similarly, the Myc-Flag-tagged cDNAs for mouse Cdkn2a (P16, Origene, #MR227284) and mouse Cdkn1b (P27, Origene, #MR201957) were also subcloned into the lentiviral pTSIN-PGK-puro2 backbone.

### Lentivirus Production and Cell Transduction

Lentiviral particles were produced in HEK-293T cells using Lipofectamine 2000 (Invitrogen, #11668) and appropriate helper plasmids: pLP1, pLP2, VSV-G (pLKO.1 vectors and pLenti vectors), VSV-G and pHR′-CMV8.9 (for pTSIN vectors) or Trans-lentiviral packaging mix (GE Dharmacon, #TLP4606) (for pTRIPZ vectors). After 48 hours, virus supernatant was harvested by filtration of HEK-293T supernatant through a 0.45 μm syringe filter. Virus was frozen at −80° C. in small aliquots and freshly thawed for each infection cycle.

### SA-β-Gal Staining

MEFs and IMR-90 cells were seeded on 10-well chambered slides (HTC supercured, Thermo Fisher Scientific, #30966S Black) at 2,000 cells/well. Flow-sorted cells were fixed the next day and stained. To assess senescence induction kinetics after irradiation or gene overexpression or gene knockdown, cells were irradiated with 10 Gy or infected twice with appropriate virus supernatants. At indicated times, cells were fixed and stained for SA-β-Gal activity according to manufacturer's protocol (Cell Signaling, #9860S). MEFs were stained for 24 hours, whereas human cells were stained for 12 hours. To quantify SA-β-Gal⁺ MEFs, cells were counterstained with Hoechst and the percentage of SA-β-Gal⁺ cells was determined. At least 100 cells per sample were counted. To determine the proportion of SA-β-Gal⁺ hepatocytes in adeno-Cre induced livers, 8 μm thick cryosections were cut and stained. Briefly, sections were fixed for 10 minutes according to manufacturer's protocol (Cell Signaling, #9860S) and staining was performed for 14 hours. Sections were counterstained with Hoechst. At least 200 hepatocytes (as determined by cell and nuclear shape) were examined for SA-β-Gal⁺ staining.

### Growth Curves

Growth curves were generated using senescent MEFs as well as their respective proliferating controls (P5 non-irradiated for IR, P3 for REP, pLenti-PGK-ER-KRAS^{G12V}-infected, ethanol-treated cells for OI). At DO, flow-sorted cells were plated in a 12-well plate at a density of 25,000 cells/well in duplicates. At D4, sub-confluent cultures were trypsinized, counted, and re-seeded at 25,000 cells/well. Counting was repeated at D7. Duplicate measures were averaged and cumulative cell number was calculated according to the following formula Tx=Tx-1*Nx/NO, where T is the cumulative cell number, x the passage number, Nx the counted cell number at passage x, and NO the initially seeded cell number. For growth curves of P21-OE or P16-OE MEFs, P3 cells were infected with pTSIN empty, pTSIN-p21-Myc-Flag or pTSIN-p16-Myc-Flag on two consecutive days. The next day (D3) cells were trypsinized, counted, and re-seeded at 100,000 cells/6-well in three separate wells per condition. Cells were counted every 24 hours until day 6. In parallel, cells were selected with puromycin, re-seeded at D7 and counting was continued.

### EdU Incorporation Assay

Sorted senescent and non-SNCs were seeded on 10-well chambered slides at 2,000 cells/well. The next day medium was replaced with medium containing 1 μM EdU (5-ethynyl-2′-deoxyuridine, 1:10,000 dilution, stock in DMSO) and cells were allowed to incorporate EdU for 48 hours. Cells were then fixed and subjected to EdU staining according to the manufacturer's instructions (Thermo Scientific, Click-iT Plus EdU Alexa Fluor 555 Imaging Kit, C10637). To assess DNA reduplication after knockdown of senescence-associated super enhancer-controlled genes, senescent MEFs were seeded on 10-well chambered slides at 2,000 cells/well and infected with shRNA-containing virus on the two following consecutive days. Forty-eight hours after the first infection, medium was replaced with medium containing 1 μM EdU for 48 hours. Four days after the first infection, cells were fixed and subjected to EdU staining. To assess proliferation of irradiated, non-senescent, P3 MEFs were seeded at 2,000 cells/well. The next day, cells were irradiated with 10 Gy. Two days post-IR, EdU was added for 24 hours, or cells were infected with shRNA-virus on two consecutive days. On day 4 post-IR, EdU was added for 24 hours. To assess proliferation of P21-OE human cells or P27-OE MEFs, cycling cells were infected with appropriate virus supernatants for 2 consecutive days as described above, selected for the next 48 hours with 2 μg/mL puromycin. At D4, cells were re-seeded at 2,000 cells/well and EdU was allowed to be incorporated for 24 hours. For inducible P21-overexpression, stably virus-infected cells were re-seeded at 2,000 cells/well and 4 μg/mL dox was added the next day. At indicated days, EdU was added for 24 hours, except for short P21-OE induction experiments represented in FIG. 23G where EdU was allowed to be incorporated for 12 hours. To quantify the EdU⁺ cell fraction, cells were counterstained with Hoechst and percentage of EdU⁺ cells was determined. At least 100 cells were counted.

### Western Blot Analysis and Co-Immunoprecipitation

Co-immunoprecipitations and western blot analysis were performed. Subcellular fractionation for co-immunoprecipitations on chromatin fractions was performed using the Subcellular Protein Fractionation Kit (Thermo Scientific, #78840) according to the manufacturer's instructions. Primary antibodies used were as follows: mouse anti-P21 (Santa Cruz, sc-53870; 1:8,000 used for both mouse and human samples), rabbit anti-Myc-tag (Cell Signaling, #2272; 1:1,000); rabbit anti-RB (Abcam, ab181616; 1:2,000), rabbit anti-STAT1 (Abcam, ab92506; 1:1,000), rabbit anti-STAT6 (Cell Signaling, #5397; 1:1,000), rabbit anti-SMAD2 (Cell Signaling, #5339, 1:1,000), rabbit anti-SMAD3 (Cell Signaling, #9513; 1:1,000), mouse anti-P27 (BD Biosciences, #610242, 1:1,000). All antibodies were detected with secondary HRP-conjugated goat anti-mouse or anti-rabbit antibodies (Jackson Immunoresearch; 1:10,000). PonS staining (0.2% w/v in 5% glacial acetic acid, Sigma-Aldrich, #P3504) served as a loading control. Western blot data are representative of at least two independent experiments.

### Conditioned Medium

To generate CM from IR-induced cells, MEFs were seeded in T75 flasks at low density. The next day, cells were exposed to 10 Gy IR. Two days post-IR, cells were infected with shRNA virus as described above. At day 4 post-IR, these cells as well as cycling control cells of similar density were washed twice and 5 mL of culture medium as added. After 48 hours of conditioning, CM was harvested, filtered through a 0.2 μm syringe filter, and stored in small aliquots at −80° C. To generate CM from IR-SNCs, cells 10 days after IR were used and treated the same way. To produce CM from gene overexpressing MEFs, cells were seeded in T75 flasks, infected with appropriate virus supernatants on the next two consecutive days. Cells were selected with puromycin until day 4 or day 10 post-infection. Again, cells were washed twice before adding of 5 mL culture medium. CM was harvested as described above. For inducible pTRIPZ-p21-Flag-Myc overexpression, 4 μg/mL dox was added to cells for 48 hours, then cells were washed and were subjected to conditioning in the presence of dox, or cells were washed twice immediately and regular culture medium was added. These cells were washed twice a day to remove any residual dox and conditioning of medium was started 4 days after removal of dox. For short-term P21-OE overexpression experiments shown in FIG. 23, medium was allowed to be conditioned for 12 hours. For CM from shCxcl14 knockdown cells, cycling cells were first infected with P21-OE virus for two days, followed by infection with shCxcl14 virus for the next two consecutive days after which, on day 4, conditioning was started.

### Scratch Assays

Cycling P3 MEFs were seeded in 24-well plates and grown to confluence for ˜3 days. Medium was removed and CM was added. Immediately afterwards, using a P20 pipette tip a linear vertical scratch was made from the top well center to the bottom well center. Cells were promptly imaged to document the initial scratch width (0 hours). Cells were grown in regular 3% O₂ incubators until 2 hours, 12 hours, 24 hours, and 48 hours post-scratch when cells were imaged again. To count cells emigrating from the cell dense area into the scratch space, three to six 10× fields were quantified and invading cell number was normalized to scratch length which these cells occupied. The average scratch width was measured from two 4× fields and at least 10 horizontal measurements (spaced 200 μm apart) from scratch edge to scratch edge.

### Isolation and Characterization of Peritoneal Immune Cells

Two- to four-month old wildtype mice were used to collect the peritoneal lavage using 10 mL ice cold PBS applied via a 20G needle. The lavage was centrifuged at 500 g for 10 minutes at 4° C. Cells were counted and subjected to transwell migration assays or used for flow cytometry. Peritoneal immune cells from wildtype control mice or wildtype mice injected with CM were resuspended in 300 μL DMEM. One-hundred μL cell suspension was used for antibody staining using CD11B-eFluor450 (eBioscience, #48-0112; 1:100), B220/CD45R-FITC (BD BioSciences; #553088; 1:100) and TCRb-APC (BD BioSciences; #553174; 1:100) antibodies. Cells were stained 20 minutes on ice in the dark, after which 200 μL DMEM was added and cells were analyzed via a FACSCanto X (BD BioSciences). Cell counts within 60 seconds was noted and referred to the cell numbers of non-injected control mice.

### Transwell Migration Assay

To perform transwell migration assays using peritoneal immune cells, 500 μL CM was added to a 24-well plate. A transwell inset (3 μm pore size, Costar, #3415 or #3472) was loaded with ˜200,000 peritoneal immune cells in 100 μL medium (matching the medium used for CM production). Cells were allowed to migrate for 12 hours. Then, the transwell was carefully removed and the medium containing suspension cells was collected. Attached cells on the well bottom were washed twice with PBS, trypsinized and scraped. Suspension cells and attached cells were spun at 500 g for 10 minutes, resuspended and counted. Cell counts were normalized to cell numbers of control condition (CM cycling cells or CM EV) for each mouse separately. For CXCL14 neutralization experiments, CM from EV- or P21-OE cells was added to a 24-well plate together with 20 μg/mL goat, anti-CXCL14 (R&D Systems, #AF866) or 20 μg/mL goat, anti-IgG (R&D Systems, #AB-108-C) (57). Transwell migration assays were performed as described above.

### Injection of CM in Wildtype Mice

To determine the immune cell-eliciting potential of CM, CM was generated as described above except that culture medium with 0.5% FBS was used. One mL of CM was aspirated with a 25G needle and 3 ml syringe. The needle was switched to 27G and CM was slowly injected into the peritoneum of 8-10-week-old wildtype mice. Four days post-injection, the peritoneal lavage was harvested and subjected to antibody staining and flow cytometry as described above.

### Statistical Analysis

Prism software (GraphPad Software) was used for statistical analyses. Unless otherwise stated, student's two-tailed paired t-tests (in MEFs and HDFs) or student's two-tailed unpaired 1-tests (in IMR-90 cells and HUVECs) were used for pairwise significance involving two groups. For all experiments involving three or more groups, one-way analysis of variance (ANOVA) with Sidak's correction or two-way ANOVA with Sidak's or Bonferroni correction for multiple comparisons were performed. In these comparisons, the following denotes significance in all figures: *P<0.05, **P<0.01 and ***P<0.001.

### Data Availability

ChIP-seq and RNA-seq data sets have been deposited in the Gene Expression Omnibus: the following secure token has been created to allow review of record GSE117278 while it remains in private status: sbwvqaqinlyjtsz.

### Example 2: Treating Breast Cancer

A biological sample (e.g., tumor biopsy) is obtained from a human suspected of having a breast cancer. The obtained sample is examined for the presence of a reduced level of CXCL14 polypeptide expression. In some cases, an IHC assay is performed to detect the presence of a reduced level of CXCL14 polypeptide expression. In some cases, a MS assay is performed to detect the presence of a reduced level of CXCL14 polypeptide expression. If a reduced level of CXCL14 polypeptide expression is detected in the sample, as compared to a control level, then the human is administered a conjugate described herein (e.g., a conjugate containing a CXCL14 polypeptide and a targeting moiety such as an antibody that binds to MUC-1⁺ breast cancer cells). The administered conjugate can induce surveillance against MUC-1⁺ breast cancer cells and reduce the number of MUC-1⁺ breast cancer cells within the human.

### Example 3: Treating Colon Cancer

A biological sample (e.g., tumor biopsy) is obtained from a human suspected of having a colon cancer. The obtained sample is examined for the presence of a reduced level of CXCL14 polypeptide expression. In some cases, an IHC assay is performed to detect the presence of a reduced level of CXCL14 polypeptide expression. In some cases, a MS assay is performed to detect the presence of a reduced level of CXCL14 polypeptide expression. If a reduced level of CXCL14 polypeptide expression is detected in the sample, as compared to a control level, then the human is administered a conjugate described herein (e.g., a conjugate containing a CXCL14 polypeptide and a targeting moiety such as an antibody that binds to MUC-1⁺ colon cancer cells). The administered conjugate can induce surveillance against MUC-1⁺ colon cancer cells and reduce the number of MUC-1⁺ colon cancer cells within the human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for inducing immune surveillance against a cancer cell within a mammal having cancer, wherein said method comprises administering to said mammal a composition comprising a chemokine (C-X-C motif) ligand 14 (CXCL14) polypeptide and a targeting moiety, wherein said targeting moiety targets said composition to said cancer cell.

2. A method for inducing immune surveillance against a cancer cell within a mammal having cancer, wherein said method comprises administering to said mammal a composition comprising a targeting moiety and nucleic acid encoding a CXCL14 polypeptide, wherein said targeting moiety targets said composition to said cancer cell, and wherein said cancer cell expresses said CXCL14 polypeptide, thereby inducing immune surveillance against said cancer cell.

3. A method for inducing immune surveillance against a cancer cell within a mammal having cancer, wherein said method comprises administering to said mammal a composition comprising:
(a) nucleic acid encoding a fusion polypeptide comprising a deactivated Cas (dCas) polypeptide and a transcriptional activator polypeptide;
(b) nucleic acid encoding a helper activator polypeptide;
(c) nucleic acid encoding a nucleic acid molecule comprising (i) a nucleic acid sequence that is complementary to a target sequence that encodes at least a portion of a CXCL14 polypeptide, and (ii) a nucleic acid sequence that can bind said helper activator polypeptide; and
(d) a targeting moiety,
wherein said targeting moiety targets said composition to said cancer cell, and wherein said cancer cell increases expression of an endogenous CXCL14 polypeptide.

4. A method for treating cancer in a mammal, wherein said method comprises administering to said mammal a composition comprising a CXCL14 polypeptide and a targeting moiety, wherein said targeting moiety targets said composition to a cancer cell within said mammal.

5. A method for treating cancer in a mammal, wherein said method comprises administering to said mammal a composition comprising a targeting moiety and nucleic acid encoding a CXCL14 polypeptide, wherein said targeting moiety targets said composition to a cancer cell within said mammal, and wherein said cancer cell expresses said CXCL14 polypeptide.

6. A method for treating cancer in a mammal, wherein said method comprises administering to said mammal a composition comprising:
(a) nucleic acid encoding a fusion polypeptide comprising a dCas polypeptide and a transcriptional activator polypeptide;
(b) nucleic acid encoding a helper activator polypeptide;
(c) nucleic acid encoding a nucleic acid molecule comprising (i) a nucleic acid sequence that is complementary to a target sequence within a Cxcl14 gene, and (ii) a nucleic acid sequence that can bind said helper activator polypeptide; and
(d) a targeting moiety,
wherein said targeting moiety targets said composition to a cancer cell within said mammal, and wherein said cancer cell increases expression of an endogenous CXCL14 polypeptide.

7. A method for inducing immune surveillance against a cancer cell within a mammal having cancer, wherein said method comprises administering to said mammal a composition comprising a p21 polypeptide and a targeting moiety, wherein said targeting moiety targets said composition to said cancer cell.

8. A method for inducing immune surveillance against a cancer cell within a mammal having cancer, wherein said method comprises administering to said mammal a composition comprising a targeting moiety and nucleic acid encoding a p21 polypeptide, wherein said targeting moiety targets said composition to said cancer cell.

9. A method for inducing immune surveillance against a cancer cell within a mammal having cancer, wherein said method comprises administering to said mammal a composition comprising a targeting moiety and an inhibitor of phosphorylation of a RB polypeptide, wherein said targeting moiety targets said composition to said cancer cell.

10. The method of claim 9, wherein said inhibitor of phosphorylation of a RB polypeptide is an inhibitor of a CDK2 polypeptide.

11. The method of claim 10, wherein said inhibitor of said CDK2 polypeptide is selected from the group consisting of dinaciclib, GW8510, and seliciclib.

12. A method for inducing immune surveillance against a cancer cell within a mammal having cancer, wherein said method comprises administering to said mammal a composition comprising a hypophosphorylated RB polypeptide and a targeting moiety, wherein said targeting moiety targets said composition to said cancer cell.

13. A method for treating cancer in a mammal, wherein said method comprises administering to said mammal a composition comprising a p21 polypeptide and a targeting moiety, wherein said targeting moiety targets said composition to a cancer cell within said mammal.

14. A method for treating cancer in a mammal, wherein said method comprises administering to said mammal a composition comprising a targeting moiety and nucleic acid encoding a p21 polypeptide, wherein said targeting moiety targets said composition to a cancer cell within said mammal, and wherein said cancer cell expresses said p21 polypeptide.

15. A method for treating cancer in a mammal, wherein said method comprises administering to said mammal a composition comprising a targeting moiety and an inhibitor of phosphorylation of a RB polypeptide, wherein said targeting moiety targets said composition to a cancer cell within said mammal.

16. The method of claim 15, wherein said inhibitor of phosphorylation of a RB polypeptide is an inhibitor of a CDK2 polypeptide.

17. The method of claim 16, wherein said inhibitor of said CDK2 polypeptide is selected from the group consisting of dinaciclib, GW8510, and seliciclib.

18. A method for treating cancer in a mammal, wherein said method comprises administering to said mammal a composition comprising a targeting moiety and a hypophosphorylated RB polypeptide, wherein said targeting moiety targets said composition to a cancer cell within said mammal.

19. A method for inducing immune surveillance against a cancer cell within a mammal having cancer, wherein said method comprises administering to said mammal a composition comprising a CXCL14 polypeptide, an IL-34 polypeptide, and a targeting moiety, wherein said targeting moiety targets said composition to said cancer cell.

20. A method for treating cancer in a mammal, wherein said method comprises administering to said mammal a composition comprising a CXCL14 polypeptide, an IL-34 polypeptide, and a targeting moiety, wherein said targeting moiety targets said composition to a cancer cell within said mammal.

21. The method of claim 1, wherein said mammal is a human.

22. The method of claim 1, wherein said cancer is selected from the group consisting of liver cancer, colorectal cancer, breast cancer, head and neck cancer, and cervical cancer.

23. The method of claim 1, wherein said targeting moiety comprises an antibody or a single-chain variable fragment (scFv).

24. The method of claim 1, wherein said cancer cell comprises a mutant p53 gene.

25. The method of claim 1, wherein said method comprises identifying said mammal as having cancer cells comprising a mutant p53 gene.

26. The method of claim 1, wherein said cancer cell comprises a decreased level of expression of a PASP polypeptide.

27. The method of claim 26, wherein said PASP polypeptide is selected from the group consisting of a CXCL14 polypeptide, an IL-34 polypeptide, an IL-7 polypeptide, and a CCL17 polypeptide.

28. The method of claim 1, wherein said method comprises identifying said mammal as having cancer cells comprising a decreased level of expression of a PASP polypeptide.

29. The method of claim 1, wherein said method comprises identifying said mammal as having cancer cells comprising a decreased level of a CXCL14 polypeptide.

30. The method of claim 1, wherein said composition is in the form of a viral vector, a conjugate, a liposome, a polymeric micelle, a microsphere, or a nanoparticle.

31. The method of claim 1, wherein the components of said composition are covalently attached.

32. The method of claim 1, wherein the components of said composition are non-covalently attached.
